(19) United States
(12) Patent Application Publication
(10) Pub. No.: US 2025/0197507 A1
(43) Pub. Date: Jun. 19, 2025

(54) CD161 BINDING PROTEINS AND RELATED METHODS

(71) Applicant: Immunitas Therapeutics, Inc., Waltham, MA (US)

(72) Inventors: Frano-Joseph IRVINE, Medford, MA (US); Alison TISDALE, Belmont, MA (US)

(21) Appl. No.: 18/980,324

(22) Filed: Dec. 13, 2024

Related U.S. Application Data
(60) Provisional application No. 63/610,044, filed on Dec. 14, 2023.

(51) Int. Cl. C07K 16/28; A61K 39/00; A61P 35/00; G01N 33/574; G01N 33/68

(52) U.S. Cl. CPC C07K 16/2851; A61P 35/00; G01N 33/57492; G01N 33/6872; A61K 2039/505; C07K 2317/24; G01N 2333/70596

(57) ABSTRACT

Provided herein are CD161 binding proteins (e.g., antibodies) and compositions (e.g., pharmaceutical compositions) comprising the same; as well as methods of making the CD161 binding proteins (e.g., antibodies) and compositions. Further provided herein are kits comprising the CD161 binding proteins (e.g., antibodies) described herein. The CD161 binding proteins provided herein are useful in e.g., in therapeutic, diagnostic, screening methods.

### RELATED APPLICATIONS

This application claims priority to U.S. Ser. No. 63/610,044, filed Dec. 14, 2023, the entire contents of which is incorporated herein by reference.

### SEQUENCE LISTING

The instant application contains a Sequence Listing which has been submitted electronically in XML format and is hereby incorporated by reference in its entirety. Said XML copy, created on Dec. 11, 2024, is named 63340_26US01_SL.xml and is 172,032 bytes in size.

### 1. FIELD

This disclosure relates to CD161 binding proteins and nucleic acid molecules encoding the same. This disclosure is further related to methods of manufacturing and utilizing the same, including e.g., methods of patient selection, methods of treatment of a disease (e.g., cancer).

### 2. BACKGROUND

CD161 is a C-type lectin-like type-II transmembrane protein. CD161 is encoded by the killer cell lectin like receptor B1 (KLRB1) gene located within the natural killer (NK) cell gene complex on chromosome 12. CD161 is expressed by e.g., NK cells and subsets of both CD4+ and CD8+ T cells. CD161 binds CLEC2D, also a C-type lectin transmembrane protein. CLEC2D is expressed, e.g., on the surface of both malignant cells and immune cells including germinal center B cells, activated T cells, and tumor associated macrophages. CLEC2D/CD161 interactions play a role in regulating immune responses in various contexts, including, e.g., infectious diseases, autoimmunity, inflammatory conditions, and cancer.

### 3. SUMMARY

Provided herein are, inter alia, CD161 binding proteins and nucleic acid molecules encoding the same; fusions and conjugates comprising the CD161 binding proteins; methods of manufacturing; pharmaceutical compositions; and methods of use including e.g., methods of treating diseases (e.g., cancer), methods of inhibiting binding of CD161 to CLEC2D, and diagnostics.

Accordingly, in one aspect provided herein are CD161 binding proteins (e.g., antibodies) comprising a binding domain that specifically binds CD161 (e.g., hCD161), wherein the binding domain comprises: (a) a VH region comprising (i) a CDR-H1 comprising the amino acid sequence set forth in SEQ ID NO: 169, or the amino acid sequence set forth in SEQ ID NO: 169 comprising 1, 2, or 3 amino acid variations (e.g., substitutions, deletions, additions); (ii) a CDR-H2 comprising the amino acid sequence set forth in SEQ ID NO: 170, or the amino acid sequence set forth in SEQ ID NO: 170 comprising 1, 2, or 3 amino acid variations (e.g., substitutions, deletions, additions); and (iii) a CDR-H3 comprising the amino acid sequence set forth in SEQ ID NO: 171, or the amino acid sequence set forth in SEQ ID NO: 171 comprising 1, 2, or 3 amino acid variations (e.g., substitutions, deletions, additions); and (b) a VL region comprising (i) a CDR-L1 comprising the amino acid sequence set forth in SEQ ID NO: 172, or the amino acid sequence set forth in SEQ ID NO: 172 comprising 1, 2, or 3 amino acid variations (e.g., substitutions, deletions, additions); (ii) a CDR-L2 comprising the amino acid sequence set forth in SEQ ID NO: 173, or the amino acid sequence set forth in SEQ ID NO: 173 comprising 1, 2, or 3 amino acid variations (e.g., substitutions, deletions, additions); and (iii) a CDR-L3 comprising the amino acid sequence set forth in SEQ ID NO: 174, or the amino acid sequence set forth in SEQ ID NO: 174 comprising 1, 2, or 3 amino acid variations (e.g., substitutions, deletions, additions).

In some embodiments, the binding domain comprises: (a) a VH region comprising (i) a CDR-H1 comprising the amino acid sequence set forth in SEQ ID NO: 169; (ii) a CDR-H2 comprising the amino acid sequence set forth in SEQ ID NO: 170; and (iii) a CDR-H3 comprising the amino acid sequence set forth in SEQ ID NO: 171; and (b) a VL region comprising (i) a CDR-L1 comprising the amino acid sequence set forth in SEQ ID NO: 172; (ii) a CDR-L2 comprising the amino acid sequence set forth in SEQ ID NO: 173; and (iii) a CDR-L3 comprising the amino acid sequence set forth in SEQ ID NO: 174.

In one aspect provided herein are CD161 binding proteins (e.g., antibodies) comprising a binding domain that specifically binds CD161 (e.g., hCD161), wherein the binding domain comprises: (a) a VH region comprising (i) a CDR-H1 comprising the amino acid sequence of a CDR-H1 set forth in Table 2, or the amino acid sequence of a CDR-H1 set forth in Table 2 comprising 1, 2, or 3 amino acid variations (e.g., substitutions, deletions, additions); (ii) a CDR-H2 comprising the amino acid sequence of a CDR-H2 set forth in Table 2, or the amino acid sequence of a CDR-H2 set forth in Table 2 comprising 1, 2, or 3 amino acid variations (e.g., substitutions, deletions, additions); and (iii) a CDR-H3 comprising the amino acid sequence of a CDR-H3 set forth in Table 2, or the amino acid sequence of a CDR-H3 set forth in Table 2 comprising 1, 2, or 3 amino acid variations (e.g., substitutions, deletions, additions); and (b) a VL region comprising (i) a CDR-L1 comprising the amino acid sequence of a CDR-L1 set forth in Table 2, or the amino acid sequence of a CDR-L1 set forth in Table 2 comprising 1, 2, or 3 amino acid variations (e.g., substitutions, deletions, additions); (ii) a CDR-L2 comprising the amino acid sequence of a CDR-L2 set forth in Table 2, or the amino acid sequence of a CDR-L2 set forth in Table 2 comprising 1, 2, or 3 amino acid variations (e.g., substitutions, deletions, additions); and (iii) a CDR-L3 comprising the amino acid sequence of a CDR-L3 set forth in Table 2, or the amino acid sequence of a CDR-L3 set forth in Table 2 comprising 1, 2, or 3 amino acid variations (e.g., substitutions, deletions, additions).

In some embodiments, the binding domain comprises: (a) a VH region comprising (i) a CDR-H1 comprising the amino acid sequence of a CDR-H1 set forth in Table 2; (ii) a CDR-H2 comprising the amino acid sequence of a CDR-H2 set forth in Table 2; and (iii) a CDR-H3 comprising the amino acid sequence of a CDR-H3 set forth in Table 2; and (b) a VL region comprising (i) a CDR-L1 comprising the amino acid sequence of a CDR-L1 set forth in Table 2; (ii) a CDR-L2 comprising the amino acid sequence of a CDR-L2 set forth in Table 2; and (iii) a CDR-L3 comprising the amino acid sequence of a CDR-L3 set forth in Table 2.

In one aspect provided herein are CD161 binding proteins (e.g., antibodies) comprising a binding domain that specifically binds CD161 (e.g., hCD161), wherein the binding domain comprises: (a) a VH region comprising (i) a CDR-H1 comprising the amino acid sequence of CDR-H1 of CD161 Binding Protein A set forth in Table 2, or the amino acid sequence of a CDR-H1 of CD161 Binding Protein A set forth in Table 2 comprising 1, 2, or 3 amino acid variations (e.g., substitutions, deletions, additions); (ii) a CDR-H2 comprising the amino acid sequence of a CDR-H2 of CD161 Binding Protein A set forth in Table 2, or the amino acid sequence of a CDR-H2 of CD161 Binding Protein A set forth in Table 2 comprising 1, 2, or 3 amino acid variations (e.g., substitutions, deletions, additions); and (iii) a CDR-H3 comprising the amino acid sequence of a CDR-H3 of CD161 Binding Protein A set forth in Table 2, or the amino acid sequence of a CDR-H3 of CD161 Binding Protein A set forth in Table 2 comprising 1, 2, or 3 amino acid variations (e.g., substitutions, deletions, additions); and (b) a VL region comprising (i) a CDR-L1 comprising the amino acid sequence of a CDR-L1 of CD161 Binding Protein A set forth in Table 2, or the amino acid sequence of a CDR-L1 of CD161 Binding Protein A set forth in Table 2 comprising 1, 2, or 3 amino acid variations (e.g., substitutions, deletions, additions); (ii) a CDR-L2 comprising the amino acid sequence of a CDR-L2 of CD161 Binding Protein A set forth in Table 2, or the amino acid sequence of a CDR-L2 of CD161 Binding Protein A set forth in Table 2 comprising 1, 2, or 3 amino acid variations (e.g., substitutions, deletions, additions); and (iii) a CDR-L3 comprising the amino acid sequence of a CDR-L3 of CD161 Binding Protein A set forth in Table 2, or the amino acid sequence of a CDR-L3 of CD161 Binding Protein A set forth in Table 2 comprising 1, 2, or 3 amino acid variations (e.g., substitutions, deletions, additions).

In some embodiments, the binding domain comprises: (a) a VH region comprising (i) a CDR-H1 comprising the amino acid sequence of CDR-H1 of CD161 Binding Protein A set forth in Table 2; (ii) a CDR-H2 comprising the amino acid sequence of a CDR-H2 of CD161 Binding Protein A set forth in Table 2; and (iii) a CDR-H3 comprising the amino acid sequence of a CDR-H3 of CD161 Binding Protein A set forth in Table 2; and (b) a VL region comprising (i) a CDR-L1 comprising the amino acid sequence of a CDR-L1 of CD161 Binding Protein A set forth in Table 2; (ii) a CDR-L2 comprising the amino acid sequence of a CDR-L2 of CD161 Binding Protein A set forth in Table 2; and (iii) a CDR-L3 comprising the amino acid sequence of a CDR-L3 of CD161 Binding Protein A set forth in Table 2.

In one aspect provided herein are CD161 binding proteins (e.g., antibodies) comprising a binding domain that specifically binds CD161 (e.g., hCD161), wherein the binding domain comprises: (a) a VH region comprising a CDR-H1, CDR-H2, and CDR-H3 of SEQ ID NO: 175; and (b) a VL region comprising a CDR-L1, CDR-L2, and CDR-L3 of SEQ ID NO: 176.

In one aspect provided herein are CD161 binding proteins (e.g., antibodies) comprising a binding domain that specifically binds CD161 (e.g., hCD161), wherein the binding domain comprises: (a) a VH region comprising a CDR-H1, CDR-H2, and CDR-H3 of a VH region set forth in Table 2; and (b) a VL region comprising a CDR-L1, CDR-L2, and CDR-L3 of a VL region set forth in Table 2.

In one aspect provided herein are CD161 binding proteins (e.g., antibodies) comprising a binding domain that specifically binds CD161 (e.g., hCD161), wherein the binding domain comprises: (a) a VH region comprising a CDR-H1, CDR-H2, and CDR-H3 of the VH region of CD161 Binding Protein A set forth in Table 2; and (b) a VL region comprising a CDR-L1, CDR-L2, and CDR-L3 of the VL region of CD161 Binding Protein A set forth in Table 2.

In some embodiments, (a) the amino acid sequence of the VH region is at least 85%, 86%, 87%, 88%, 89%, 90%, 91%, 92%, 93%, 94%, 95%, 96%, 97%, 98%, 99%, or 100% identical to the amino acid sequence of a VH region set forth in Table 2; and (b) the amino acid sequence of the VL region is at least 85%, 86%, 87%, 88%, 89%, 90%, 91%, 92%, 93%, 94%, 95%, 96%, 97%, 98%, 99%, or 100% identical to the amino acid sequence of a VL region set forth in Table 2.

In some embodiments, (a) the amino acid sequence of the VH region is at least 85%, 86%, 87%, 88%, 89%, 90%, 91%, 92%, 93%, 94%, 95%, 96%, 97%, 98%, 99%, or 100% identical to the amino acid sequence of the VH region of CD161 Binding Protein A set forth in Table 2; and (b) the amino acid sequence of the VL region is at least 85%, 86%, 87%, 88%, 89%, 90%, 91%, 92%, 93%, 94%, 95%, 96%, 97%, 98%, 99%, or 100% identical to the amino acid sequence of the VL region of CD161 Binding Protein A set forth in Table 2.

In some embodiments, (a) the amino acid sequence of the VH region is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 175; and (b) the amino acid sequence of the VL region is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 176.

In one aspect provided herein are CD161 binding proteins (e.g., antibodies) comprising a binding domain that specifically binds CD161 (e.g., hCD161), wherein the binding domain comprises: (a) a VH region comprising an amino acid sequence is at least 85%, 86%, 87%, 88%, 89%, 90%, 91%, 92%, 93%, 94%, 95%, 96%, 97%, 98%, 99%, or 100% identical to the amino acid sequence of a VH region set forth in Table 2; and (b) a VL region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a VL region set forth in Table 2.

In one aspect provided herein are CD161 binding proteins (e.g., antibodies) comprising a binding domain that specifically binds CD161 (e.g., hCD161), wherein the binding domain comprises: (a) a VH region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of the VH region of CD161 Binding Protein A set forth in Table 2; and (b) a VL region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of the VL region of CD161 Binding Protein A set forth in Table 2.

In one aspect provided herein are CD161 binding proteins (e.g., antibodies) comprising a binding domain that specifically binds CD161 (e.g., hCD161), wherein the binding domain comprises: (a) a VH region comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 175; and (b) a VL region comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 176.

In some embodiments, the CD161 binding protein comprises (a) a heavy chain (HC); and (b) a light chain (LC).

In some embodiments, (a) the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 2; and (b) the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 2.

In some embodiments, (a) the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the HC of CD161 Binding Protein A set forth in Table 2; and (b) the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the LC of CD161 Binding Protein A set forth in Table 2.

In some embodiments, (a) the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 177; and (b) the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 178.

In one aspect provided herein are CD161 binding proteins (e.g., antibodies) comprising a binding domain that specifically binds CD161 (e.g., hCD161), wherein the binding domain comprises: (a) a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 2; and (b) a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 2.

In one aspect provided herein are CD161 binding proteins (e.g., antibodies) comprising a binding domain that specifically binds CD161 (e.g., hCD161), wherein the binding domain comprises: (a) a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of the HC of CD161 Binding Protein A set forth in Table 2; and (b) a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of the LC of CD161 Binding Protein A set forth in Table 2.

In one aspect provided herein are CD161 binding proteins (e.g., antibodies) comprising a binding domain that specifically binds CD161 (e.g., hCD161), wherein the binding domain comprises: (a) a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 177; and (b) a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 178.

For the sake of clarity, it is to be understood that the following embodiments are describing embodiments for any of the foregoing aspects as if each was individually recited below each of the foregoing aspects in this section.

In some embodiments, the CD161 binding protein (or the binding domain) comprises an antibody (e.g., an IgG (e.g., IgG₁, IgG₂ (e.g., IgG₂ₐ or IgG_{2b}), IgG₃, IgG₄), IgE, IgM, IgD, or IgA (e.g., IgA₁ or IgA₂) antibody). In some embodiments, the CD161 binding protein (or the binding domain) comprises one or more of a monoclonal antibody, monospecific antibody, multispecific antibody, human antibody, humanized antibody, chimeric antibody, and/or murine antibody, or a functional fragment or functional variant of any of the foregoing. In some embodiments, the CD161 binding protein (or the binding domain) is a humanized version of an antibody set forth in table 2. In some embodiments, the CD161 binding protein (or the binding domain) comprises one or more of a full-length antibody, scFv, Fab, F(ab′)₂, Fab′, Fv, single domain antibody (e.g., a VHH), scFv-Fc, Fab-Fc, and/or single domain antibody-Fc (e.g., VHH-Fc). In some embodiments, the CD161 binding protein comprises an Ig Fe (e.g., an Ig Fe, a mIg Fc) region. In some embodiments, the Ig Fe (e.g., an Ig Fe, a mIg Fc) region comprises at least a portion of a hinge region, a CH2 region, and a CH3 region. In some embodiments, the Ig Fe (e.g., an Ig Fe, a mIg Fc) region comprises a hinge region, a CH2 region, and a CH3 region. In some embodiments, the Ig Fe (e.g., an Ig Fc) region is an IgG (e.g., IgG1, IgG₂, IgG₃, IgG₄) or a mIgG (e.g., IgG1, IgG_{2b}, IgG₂ₐ or IgG₂c, or IgG₃). In some embodiments, the Ig Fe (e.g., an Ig Fe, a mIg Fc) region is part of a full-length antibody.

In some embodiments, the CD161 binding protein further comprises a heterologous moiety (e.g., a heterologous protein). In some embodiments, the CD161 binding protein further comprises 2, 3, 4, or 5 or more heterologous moieties. In some embodiments, the heterologous moiety is attached to the N-terminus, C-terminus, and/or internally between the N- and C-terminus of the CD161 binding protein. In some embodiments, the heterologous moiety (e.g., heterologous polypeptide) is directly attached to the CD161 binding protein. In some embodiments, the heterologous moiety (e.g., heterologous polypeptide) is indirectly attached to the CD161 binding protein. In some embodiments, the heterologous moiety (e.g., heterologous polypeptide) is indirectly attached to the CD161 binding protein via a linker. In some embodiments, the heterologous moiety is a peptide, polypeptide, protein, nucleic acid molecule (e.g., DNA, RNA), carbohydrate, lipid, polymer, or small molecule. In some embodiments, the heterologous moiety is a detectable tag (e.g., a fluorescent protein).

In some embodiments, the CD161 binding protein is isolated. In some embodiments, the CD161 binding protein is recombinant.

In one aspect, provided herein are conjugates comprising a CD161 binding protein described herein and a heterologous moiety.

In some embodiments, the conjugate further comprises 2, 3, 4, or 5 or more heterologous moieties. In some embodiments, the heterologous moiety is attached to the N-terminus, C-terminus, and/or internally between the N- and C-terminus of the CD161 binding protein. In some embodiments, the heterologous moiety (e.g., heterologous polypeptide) is directly attached to the CD161 binding protein. In some embodiments, the heterologous moiety (e.g., heterologous polypeptide) is indirectly attached to the CD161 binding protein. In some embodiments, the heterologous moiety (e.g., heterologous polypeptide) is indirectly attached to the CD161 binding protein via a linker. In some embodiments, the heterologous moiety is a peptide, polypeptide, carbohydrate, lipid, polymer, or small molecule. In some embodiments, the heterologous moiety is a detectable tag (e.g., a fluorescent protein).

In one aspect, provided herein are fusion proteins comprising a CD161 binding protein described herein and a heterologous protein. In some embodiments, the heterologous protein comprises a cytokine (or functional fragment or functional variant thereof), a chemokine (or a functional fragment or functional variant thereof), or an antibody (or a functional fragment or functional variant thereof). In some embodiments, the heterologous protein is fused to the N-terminus, C-terminus, and/or internally between the N- and C-terminus of the CD161 binding protein. In some embodiments, the heterologous protein is fused directly to the CD161 binding protein. In some embodiments, the heterologous protein is fused indirectly to the CD161 binding protein. In some embodiments, the heterologous protein is fused indirectly to the CD161 binding protein via a peptide linker. In some embodiments, the heterologous protein is a detectable tag (e.g., a fluorescent protein).

In one aspect, provided herein are nucleic acid molecules encoding s CD161 binding protein described herein or a fusion protein described herein. In some embodiments, the nucleic acid molecule is a DNA or RNA (e.g., mRNA) molecule. In some embodiments, the nucleic acid molecule is codon optimized. In some embodiments, the nucleic acid molecule further comprises one or more transcription or translation regulatory elements (e.g., promoter, enhancer (e.g., cell or tissue specific transcription regulatory elements).

In one aspect, provided herein are vectors comprising a nucleic acid molecule described herein. In some embodiments, the vector is a viral vector or a non-viral vector (e.g., plasmid, minicircle). In some embodiments, the vector is a viral vector (e.g., an adeno associated viral (AAV) vector, a lentiviral vector, an adenoviral vector).

In one aspect, provided herein are carriers comprising a CD161 binding protein described herein, a fusion protein described herein, a conjugate described herein, a nucleic acid molecule described herein, a vector described herein, a cell described herein, or a pharmaceutical composition described herein. In some embodiments, the carrier is a nanoparticle, polymer, virus (e.g., a recombinant virus), virus like particle, virosome, fusosome, vesicle, or lipid-based carrier (e.g., a lipid nanoparticle (LNP), liposome, lipoplex, nanoliposome, an exosome, or a micelle).

In one aspect, provided herein are cells comprising a CD161 binding protein described herein, a fusion protein described herein, a conjugate described herein, a nucleic acid molecule described herein, a vector described herein, a pharmaceutical composition described herein, or a carrier described herein.

In one aspect, provided herein are pharmaceutical compositions comprising a CD161 binding protein described herein, a fusion protein described herein, a conjugate described herein, a nucleic acid molecule described herein, a vector described herein, a cell described herein, or a carrier described herein, and a pharmaceutically acceptable excipient.

In one aspect, provided herein are kits comprising a CD161 binding protein described herein, a fusion protein described herein, a conjugate described herein, a nucleic acid molecule described herein, a vector described herein, a cell described herein, a pharmaceutical composition described herein, or a carrier described herein; and optionally instructions for using any one or more of the foregoing.

In one aspect, provided herein are kits comprising a CD161 binding protein described herein, a fusion protein described herein, a conjugate described herein, a nucleic acid molecule described herein, a vector described herein, a cell described herein, a pharmaceutical composition described herein, or a carrier described herein; and a labeled secondary antibody that specifically binds a CD161 binding protein described herein, a fusion protein described herein, or a conjugate described herein.

In one aspect, provided herein are methods of manufacturing a CD161 binding protein described herein, a fusion protein described herein, or a conjugate described herein, the method comprising: introducing into a cell a nucleic acid molecule described herein, a vector described herein, or a carrier described herein; culturing the cell under conditions that allow for expression of the CD161 binding protein, the conjugate, or the fusion protein; and optionally recovering the expressed the CD161 binding protein, the conjugate, or the fusion protein from the culture; and optionally purifying the expressed the CD161 binding protein, the conjugate, or the fusion protein from the culture.

In one aspect, provided herein are methods of delivering a CD161 binding protein, fusion protein, a conjugate, nucleic acid molecule, vector, carrier, composition, pharmaceutical composition, or system to a cell, the method comprising, delivering to the cell a CD161 binding protein described herein, a fusion protein described herein, a conjugate described herein, a nucleic acid molecule described herein, a vector described herein, a pharmaceutical composition described herein, or a carrier described herein, to thereby deliver the CD161 binding protein, the fusion protein, the conjugate protein, the nucleic acid molecule, the vector, the carrier, or the pharmaceutical composition to the cell.

In one aspect, provided herein are methods of delivering a CD161 binding protein, fusion protein, a conjugate, nucleic acid molecule, vector, carrier, cell, composition, pharmaceutical composition, or system to a subject, the method comprising, administering to the subject a CD161 binding protein described herein, a fusion protein described herein, a conjugate described herein, a nucleic acid molecule described herein, a vector described herein, a cell described herein, a pharmaceutical composition described herein, or a carrier described herein, to thereby deliver the CD161 binding protein, the fusion protein, the conjugate protein, the nucleic acid molecule, the vector, the carrier, the cell, or the pharmaceutical composition to the subject.

In one aspect, provided herein are methods of delivering a CD161 binding protein, fusion protein, a conjugate, nucleic acid molecule, vector, carrier, composition, pharmaceutical composition, or system to a cell in a subject, the method comprising, administering to the subject a CD161 binding protein described herein, a fusion protein described herein, a conjugate described herein, a nucleic acid molecule described herein, a vector described herein, a pharmaceutical composition described herein, or a carrier described herein, to thereby deliver the CD161 binding protein, the fusion protein, the conjugate protein, the nucleic acid molecule, the vector, the carrier, or the pharmaceutical composition to the cell in the subject.

In one aspect, provided herein are methods of inhibiting binding of CLEC2D to CD161 in a subject, the method comprising administering to the subject a CD161 binding protein described herein, a fusion protein described herein, a conjugate described herein, a nucleic acid molecule described herein, a vector described herein, a pharmaceutical composition described herein, or a carrier described herein, to thereby inhibit binding of CLEC2D to CD161.

In one aspect, provided herein are methods of treating, ameliorating, or preventing a disease in a subject in need thereof, the method comprising administering to the subject a CD161 binding protein described herein, a fusion protein described herein, a conjugate described herein, a nucleic acid molecule described herein, a vector described herein, a pharmaceutical composition described herein, or a carrier described herein, to thereby treat, ameliorate, or prevent the CD161 associated disease in the subject.

In one aspect, provided herein are methods of treating, ameliorating, or preventing cancer in a subject in need thereof, the method comprising administering to the subject a CD161 binding protein described herein, a fusion protein described herein, a conjugate described herein, a nucleic acid molecule described herein, a vector described herein, a pharmaceutical composition described herein, or a carrier described herein, to thereby treat, ameliorate, or prevent the cancer in the subject.

In one aspect, provided herein are methods of treating cancer in a subject in need thereof, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein, a fusion protein described herein, or a conjugate described herein; and (b) administering an agent (e.g., anti-CD161 antibody (e.g., an anti-CD161 antibody described herein)) that inhibits the interaction of CD161 to CLEC2D to the subject if expression of CD161 is detected.

In one aspect, provided herein are methods of treating cancer in a subject in need thereof, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein, a fusion protein described herein, or a conjugate described herein; and (b)(i) administering an agent (e.g., anti-CD161 antibody (e.g., an anti-CD161 antibody described herein)) that inhibits the interaction of CD161 to CLEC2D to the subject if expression of CD161 is detected; or (b)(ii) withholding administration of an agent (e.g., anti-CD161 antibody) that inhibits the interaction of CD161 to CLEC2D to the subject if expression of CD161 is not detected and optionally administering a different therapeutic agent to treat the cancer that is not an agent that that inhibits the interaction of CD161 to CLEC2D.

In one aspect, provided herein are methods of treating cancer in a subject in need thereof, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein, a fusion protein described herein, or a conjugate described herein; and (b) administering an agent (e.g., anti-CD161 antibody (e.g., an anti-CD161 antibody described herein)) that inhibits the interaction of CD161 to CLEC2D to the subject based at least in part on the detection of CD161 expression in the sample from the subject.

In one aspect, provided herein are methods of identifying a subject having cancer for treatment with an agent (e.g., anti-CD161 antibody (e.g., an anti-CD161 antibody described herein)) that inhibits the interaction of CD161 to CLEC2D, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein, a fusion protein described herein, or a conjugate described herein; (b) identifying the subject as a subject for treatment with an agent (e.g., anti-CD161 antibody (e.g., an anti-CD161 antibody described herein)) that inhibits the interaction of CD161 to CLEC2D if expression of CD161 is detected; and (c) optionally administering an agent (e.g., anti-CD161 antibody (e.g., an anti-CD161 antibody described herein)) that inhibits the interaction of CD161 to CLEC2D to the subject if the subject is identified as a subject for treatment with an agent (e.g., anti-CD161 antibody) that inhibits the interaction of CD161 to CLEC2D.

In one aspect, provided herein are methods of identifying a subject having cancer who is likely to respond to a treatment with an agent (e.g., anti-CD161 antibody (e.g., an anti-CD161 antibody described herein)) that inhibits the interaction of CD161 to CLEC2D, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein, a fusion protein described herein, or a conjugate described herein; (b) identifying the subject as a subject likely to respond to treatment with an agent (e.g., anti-CD161 antibody (e.g., an anti-CD161 antibody described herein)) that inhibits the interaction of CD161 to CLEC2D if expression of CD161 is detected; and (c) optionally administering an agent (e.g., anti-CD161 antibody (e.g., an anti-CD161 antibody described herein)) that inhibits the interaction of CD161 to CLEC2D to the subject if the subject is identified as a subject for treatment with an agent (e.g., anti-CD161 antibody) that inhibits the interaction of CD161 to CLEC2D.

In one aspect, provided herein are methods of selecting a therapy for a subject cancer, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein, a fusion protein described herein, or a conjugate described herein; (b) selecting a therapy comprising an agent (e.g., anti-CD161 antibody (e.g., an anti-CD161 antibody described herein)) that inhibits the interaction of CD161 to CLEC2D if expression of CD161 is detected; and (c) optionally administering the therapy comprising an agent (e.g., anti-CD161 antibody) that inhibits the interaction of CD161 to CLEC2D to the subject if the therapy is selected.

In one aspect, provided herein are methods of characterizing a population of (e.g., immune cells (e.g., T cells, NK cells (e.g., tumor infiltrating T cells, tumor infiltrating NK cells))) in a sample (e.g., a tissue sample, e.g., a tumor tissue sample) from a subject having cancer, the method comprising (a) isolating (and optionally purifying) a population of cells (or having a population of cells isolated and optionally purified)) (e.g., immune cells (e.g., T cells, NK cells (e.g., tumor infiltrating T cells, tumor infiltrating NK cells))) from a sample (e.g., a tissue sample, e.g., a tumor tissue sample) from the subject; (b) analyzing the population of cells (e.g., immune cells (e.g., T cells, NK cells (e.g., tumor infiltrating T cells, tumor infiltrating NK cells))); and (c) determining the expression of CD161, wherein the determining comprises the use of a CD161 binding protein described herein, a fusion protein described herein, or a conjugate described herein.

In one aspect, provided herein are methods of characterizing a population of (e.g., immune cells (e.g., T cells, NK cells (e.g., tumor infiltrating T cells, tumor infiltrating NK cells))) in a sample (e.g., a tissue sample, e.g., a tumor tissue sample) from a subject having cancer, the method comprising (a) isolating (and optionally purifying) a population of cells (or having a population of cells isolated and optionally purified)) (e.g., immune cells (e.g., T cells, NK cells (e.g., tumor infiltrating T cells, tumor infiltrating NK cells))) from a sample (e.g., a tissue sample, e.g., a tumor tissue sample) from the subject; (b) detecting the expression of CD161, wherein the detecting comprises the use of a CD161 binding protein described herein, a fusion protein described herein, or a conjugate described herein; and (c) characterizing the cancer as CD161 associated based on detection of CD161 expression or CD161 non-associated based on no detection of CD161 expression.

In one aspect, provided herein are in vitro methods for detecting a population of CD161-expressing cells (e.g., a population of cells (e.g., immune cells (e.g., T cells, NK cells (e.g., tumor infiltrating T cells, tumor infiltrating NK cells))) expressing CD161 on the cell surface) in a subject, the method comprising (a) contacting a sample (e.g., a tissue sample, e.g., a tumor tissue sample) comprising a population of cells (e.g., immune cells (e.g., T cells, NK cells (e.g., tumor infiltrating T cells, tumor infiltrating NK cells))) from the subject with a CD161 binding protein described herein, a fusion protein described herein, or a conjugate described herein; and (b) detecting the binding of the CD161 binding protein, the conjugate, or the fusion protein to a population of cells in the sample; wherein the binding of the CD161 binding protein, the conjugate, or the fusion protein to a population of cells within the sample indicates the presence of a population of CD161-expressing cells (e.g., a population of cells expressing CD161 on the cell surface).

For the sake of clarity, it is to be understood that the following embodiments are describing embodiments for any of the foregoing method aspects as if each was individually recited below each of the foregoing aspects in this section.

In some embodiments, the presence of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody (e.g., an anti-CD161 antibody described herein)) that inhibits the interaction of CD161 to CLEC2D (e.g., an anti-CD161 antibody). In some embodiments, the presence of a percentage of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody) that inhibits the interaction of CD161 to CLEC2D (e.g., anti-CD161 antibody (e.g., an anti-CD161 antibody described herein)).

In some embodiments, the method comprises withholding administration of an agent (e.g., anti-CD161 antibody) that inhibits the interaction of CD161 to CLEC2D (e.g., anti-CD161 antibody (e.g., an anti-CD161 antibody described herein)) to the subject if the presence of CD161 expressing cells in the population of immune cells (e.g., T cells, NK cells (e.g., tumor infiltrating T cells, NK cells)) in the sample is not detected. In some embodiments, the method comprises administering an different therapeutic agent for treatment of the cancer that is not an agent (e.g., anti-CD161 antibody (e.g., an anti-CD161 antibody described herein)) that inhibits the interaction of CD161 to CLEC2D (e.g., anti-CD161 antibody (e.g., an anti-CD161 antibody described herein)) to the subject if the presence of CD161 expressing cells in the population of cells (e.g., immune cells (e.g., T cells, NK cells (e.g., tumor infiltrating T cells, tumor infiltrating NK cells))) in the sample is not detected. In some embodiments, the different therapeutic agent is a standard of care agent for the cancer. In some embodiments, the subject is undergoing or has undergone treatment with a different therapeutic agent for the cancer that is not an agent (e.g., anti-CD161 antibody) that inhibits the interaction of CD161 to CLEC2D (e.g., an anti-CD161 antibody). In some embodiments, the treatment with the different therapeutic agent is discontinued if the presence of CD161 expressing cells in the population of cells (e.g., immune cells (e.g., T cells, NK cells (e.g., tumor infiltrating T cells, tumor infiltrating NK cells))) in the sample is detected and/or the agent (e.g., anti-CD161 antibody) that inhibits the interaction of CD161 to CLEC2D (e.g., an anti-CD161 antibody).

In some embodiments, the method comprises obtaining (or having obtained) the sample from the subject.

In some embodiments, the sample comprises a cell, a population of cells, and/or a tissue. In some embodiments, the sample comprises a tumor biopsy. In some embodiments, the sample is a tissue sample (e.g., tumor biopsy) that comprises tumor cells and immune cells (e.g., T cells, NK cells (e.g., tumor infiltrating T cells, NK cells)). In some embodiments, the sample is a formalin-fixed paraffin-embedded (FFPE) tissue sample.

In some embodiments, the method comprises processing (or having processed) the sample. In some embodiments, processing comprises any one or more of: fixing the sample (e.g., formalin fixation); and/or embedding the sample (e.g., paraffin embedding).

In some embodiments, the cancer is a solid tumor or a hematological malignancy.

In some embodiments, the cancer is a solid tumor. In some embodiments, the solid tumor is a carcinoma. In some embodiments, the solid tumor is a head and neck cancer, lung cancer, breast cancer, small bowel cancer, esophageal cancer, or colorectal cancer. In some embodiments, the solid tumor is non-small cell lung cancer (NSCLC) (e.g., NSCLC-squamous cell carcinoma (SCC); NSCLC-adenocarcinoma (ADC)); head & neck squamous cell carcinoma (HNSCC) (e.g., HPV negative); triple negative breast cancer (TNBC); hormone receptor positive breast carcinoma; or cutaneous squamous cell carcinoma (CSCC).

In some embodiments, the cancer is a hematological malignancy. In some embodiments, the hematological malignancy is a lymphoma. In some embodiments, the lymphoma is follicular lymphoma, diffuse large B cell lymphoma, Hodgkin's lymphoma, Burkitt Lymphoma, or T-cell Lymphoma. In some embodiments, the lymphoma is relapsed DLBCL that had been previously treated with an R-CHOP (rituximab, cyclophosphamide, doxorubicin, vincristine, and prednisolone) regimen.

In some embodiments, the agent (e.g., anti-CD161 antibody) that inhibits the interaction of CD161 to CLEC2D (e.g., an anti-CD161 antibody) is an antibody or a functional fragment or variant thereof described herein. In some embodiments, the agent (e.g., anti-CD161 antibody) that inhibits the interaction of CD161 to CLEC2D (e.g., an anti-CD161 antibody) is an antibody or a functional fragment or variant thereof set forth in Table 3.

In some embodiments, the agent (e.g., anti-CD161 antibody) that inhibits the interaction of CD161 to CLEC2D (e.g., an anti-CD161 antibody) is an antibody that comprises a VH that comprises: a CDR-H1, a CDR-H2, and a CDR-H3; and a VL that comprises: a CDR-L1, a CDR-L2, and a CDR-L3. In some embodiments, the amino acid sequence of CDR-H1 comprises or consists of the amino acid sequence set forth in SEQ ID NO: 47, or the amino acid sequence set forth in SEQ ID NO: 47 comprising 1, 2, or 3 amino acid variations (e.g., substitution, deletion, addition, etc.); the amino acid sequence of CDR-H2 comprises or consists of the amino acid sequence set forth in SEQ ID NO: 48, or the amino acid sequence set forth in SEQ ID NO: 48 comprising 1, 2, or 3 amino acid variations (e.g., substitution, deletion, addition, etc.); the amino acid sequence of CDR-H3 comprises or consists of the amino acid sequence set forth in SEQ ID NO: 42, or the amino acid sequence set forth in SEQ ID NO: 42 comprising 1, 2, or 3 amino acid variations (e.g., substitution, deletion, addition, etc.); the amino acid sequence of CDR-L1 comprises or consists of the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises or consists of the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises or consists of the amino acid sequence set forth in SEQ ID NO: 49, or the amino acid sequence set forth in SEQ ID NO: 49 comprising 1, 2, or 3 amino acid variations (e.g., substitution, deletion, addition, etc.).

In some embodiments, the amino acid sequence of CDR-H1 comprises or consists of the amino acid sequence set forth in SEQ ID NO: 47, or the amino acid sequence set forth in SEQ ID NO: 47 comprising no more than 1, 2, or 3 amino acid variations (e.g., substitution, deletion, addition, etc.); the amino acid sequence of CDR-H2 comprises or consists of the amino acid sequence set forth in SEQ ID NO: 48, or the amino acid sequence set forth in SEQ ID NO: 48 comprising no more than 1, 2, or 3 amino acid variations (e.g., substitution, deletion, addition, etc.); the amino acid sequence of CDR-H3 comprises or consists of the amino acid sequence set forth in SEQ ID NO: 42, or the amino acid sequence set forth in SEQ ID NO: 42 comprising no more than 1, 2, or 3 amino acid variations (e.g., substitution, deletion, addition, etc.); the amino acid sequence of CDR-L1 comprises or consists of the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises or consists of the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises or consists of the amino acid sequence set forth in SEQ ID NO: 49, or the amino acid sequence set forth in SEQ ID NO: 49 comprising 1, 2, or 3 no more than amino acid variations (e.g., substitution, deletion, addition, etc.).

In some embodiments, the amino acid sequence of CDR-H1 comprises the amino acid sequence set forth in SEQ ID NO: 47; the amino acid sequence of CDR-H2 comprises the amino acid sequence set forth in SEQ ID NO: 48; the amino acid sequence of CDR-H3 comprises the amino acid sequence set forth in SEQ ID NO: 42; the amino acid sequence of CDR-L1 comprises the amino acid sequence set forth in SEQ ID NO: 12; the amino acid sequence of CDR-L2 comprises the amino acid sequence set forth in SEQ ID NO: 7; and the amino acid sequence of CDR-L3 comprises the amino acid sequence set forth in SEQ ID NO: 49.

In some embodiments, the amino acid sequence of CDR-H1 consists of the amino acid sequence set forth in SEQ ID NO: 47; the amino acid sequence of CDR-H2 consists of the amino acid sequence set forth in SEQ ID NO: 48; the amino acid sequence of CDR-H3 consists of the amino acid sequence set forth in SEQ ID NO: 42; the amino acid sequence of CDR-L1 consists of the amino acid sequence set forth in SEQ ID NO: 12; the amino acid sequence of CDR-L2 consists of the amino acid sequence set forth in SEQ ID NO: 7; and the amino acid sequence of CDR-L3 consists of the amino acid sequence set forth in SEQ ID NO: 49.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 50.

In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 51. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 50; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 51.

### 4. BRIEF DESCRIPTION OF THE DRAWINGS

FIG. 1 is an image of a western blot showing the detection of human CD161 (hCD161) utilizing the indicated hCD161 binding protein (hCD161 binding protein Ab-1 (SEQ ID NOS: 177-178) (see Table 2 herein). The arrow indicates the expected hCD161 band at about 40KD.

FIG. 2A is a representative immunohistochemistry (IHC) image of normal human tonsil tissue stained with an anti-hCLEC2D antibody. The image shows staining in the germinal center within the secondary lymphoid follicle. FIG. 2B is a representative IHC image of normal human tonsil tissue stained with hCD161 binding protein Ab-1 (SEQ ID NOS: 177-178) (see Table 2 herein). The image shows staining of T cells and NK cells in the T cell zone around the lymphoid follicle in the tonsil tissue.

FIG. 3 is a graph showing the percent of hCD161 expressing cells in total DAPI+ cells (which includes the malignant cell population) for follicular lymphoma, DLBCL, and Hodgkin's lymphoma (as determined by IHC utilizing hCD161 binding protein Ab-1 (SEQ ID NOS: 177-178) (see Table 2 herein)).

FIG. 4 is a graph showing the percent of CD161 expressing cells in total DAPI+ cells in pre- and post- R-CHOP treatment relapsed DLBCL (as determined by IHC hCD161 binding protein Ab-1 (SEQ ID NOS: 177-178) (see Table 2 herein)).

FIG. 5A is an IHC image (40×) showing the CD161 staining (using Ab-1 described herein) of a human colorectal cancer sample pre-treatment with anti-CD161 antibody Ab-_described herein. Post-treatment the subject was deemed to be a responder to the anti-CD161 antibody Ab-10 (see Table 3 herein) therapy. The IHC image shows 50% tumor infiltrating lymphocytes (TILs) expressing CD161. The sample was determined to have a CD161 score of 2. FIG. 5B is an IHC image (40×) showing the CD161 staining (using Ab-1 described herein) of a human colorectal cancer sample pre-treatment with anti-CD161 antibody Ab-10 described herein. Post-treatment the subject was deemed to be a non-responder to the anti-CD161 antibody Ab-10 (see Table 3 herein) therapy. The IHC image shows 10% tumor infiltrating lymphocytes (TILs) expressing CD161. The sample was determined to have a CD161 score of 1.

FIG. 6 is a series of bar graphs that shows the number of CD161 expressing immune cells (CD4+ T cells—left; CD8+ T cells—middle; and NK cells—left) in human colorectal cancer samples (as determined by IHC utilizing Ab-1 described herein) in a subject confirmed to be a responder to treatment with anti-CD161 antibody Ab-10 (see Table 3 herein) (PR 1) and a non-responder (Non-R 1).

FIG. 7 is a bar graph showing the average CD161+ immune cell abundance score (range 0-2) across the indicated solid or hematological cancer.

FIG. 8 is a series of exemplary IHC images showing CD161 expression by immune cells (detected using Ab-1 disclosed herein) in human colorectal (CRC) and human esophageal cancer samples.

FIG. 9 is a table showing the breakdown of CD161 immune cell staining abundance score (range 0-2) at different reactivity thresholds in evaluable samples by tumor type.

### 5. DETAILED DESCRIPTION

The inventors have, inter alia, generated novel CD161 binding proteins that, inter alia, can be utilized in screening methods for patient selection (e.g., for treatment with an agent that specifically binds CD161 (e.g., an anti-CD161 antibody) (that, e.g., inhibits the interaction between CD161 and CLEC2D)). Accordingly, the novel CD161 binding proteins disclosed herein are good candidates for use in diagnostics, for the treatment of diseases (e.g., cancer), etc. As such, the current disclosure provides, inter alia, novel CD161 binding proteins for use in e.g., patient stratification, patient selection (e.g., for treatment with an agent that specifically binds CD161 (e.g., an anti-CD161 antibody) (that, e.g., inhibits the interaction between CD161 and CLEC2D)).

### TABLE OF CONTENTS

5.1 Definitions
5.2 CD161 Binding Proteins
5.3 Exemplary Properties of CD161 Binding Proteins and Fusions and Conjugates Thereof
5.3.1 Affinity of CD161 Binding Proteins
5.3.2 Inhibition of CD161 Binding to CLEC2D
5.4 Conjugates and Fusion Proteins
5.4.1 Conjugates
5.4.2 Fusion Proteins
5.4.3 Linkers
5.4.3.1 Non-peptide Linkers
5.4.3.2 Peptide Linkers
5.4.4 Orientation
5.5 Multimeric CD161 Binding Proteins
5.6 Ig Constant Regions
5.7 Ig Effector Function
5.7.1 Reduced Ig Effector Function
5.7.2 Enhanced Ig Effector Function
5.8 Signal Peptides
5.9 Nucleic Acid Molecules, Vectors, Cells, & Carriers
5.10 Methods of Making Proteins
5.11 Pharmaceutical Compositions
5.12 Methods of Use
5.12.1 Samples
5.12.2 Cancers
5.12.3 Agents that Inhibits the Interaction of CD161 to CLEC2D
5.12.4 CD161+ Immune Cell Abundance Score
5.12.5 Methods of Delivery
5.12.6 Methods of Inhibiting Binding of CD161 to CLEC2D
5.12.7 Methods of Treating a Disease
5.12.8 Methods of Treating Cancer
5.12.9 Methods of Identifying a Subject for Treatment
5.12.10 Methods of Identifying a Subject as a Responder
5.12.11 Methods of Selecting a Therapy
5.12.12 Methods of Stratifying Patient Populations
5.12.13 Methods of Characterizing a Population of Cells in a Sample
5.13 Assays
5.14 Kits

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this disclosure, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided in the disclosure, unless otherwise stated, the meaning of “about” should be assumed to be within an acceptable error range for that particular value or composition.

Where proteins are described herein, it is understood that nucleic acid molecules (e.g., RNA (e.g., mRNA) or DNA nucleic acid molecules) encoding the protein are also provided herein.

Where nucleic acid molecules (e.g., RNA (e.g., mRNA) or DNA nucleic acid molecules) encoding are described herein, it is understood that vectors comprising the nucleic acid molecules are also provided herein.

Where proteins, nucleic acid molecules, vectors, carriers, etc. are described herein, it is understood that isolated forms of the proteins, nucleic acid molecules, vectors, carriers, etc. are also provided herein.

Where proteins, nucleic acid molecules, etc. are described herein, it is understood that recombinant forms of the proteins, nucleic acid molecules, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

Where proteins are described herein, it is understood that polypeptides comprising the same amino acid sequence either linear or folded into their three-dimensional structure (i.e., tertiary or quaternary structure) are also provided herein.

As used herein, the term “administering” refers to the physical introduction of an agent (e.g., an agent described herein), e.g., a therapeutic agent (or a precursor of an agent (e.g., a precursor of a therapeutic agent) that is metabolized or altered within the body of the subject to produce the agent (e.g., therapeutic agent) in vivo) to a subject, using any of the various methods and delivery systems known to those skilled in the art. Administering can also be performed, for example, once, a plurality of times, and/or over one or more extended periods. Administration includes both self-administration by the subject and administration to the subject by another.

The terms “agent” and “moiety” are used interchangeably herein and are used generically to describe any macro or micro molecule (and any combination thereof). Exemplary agents include, but are not limited proteins, peptides, nucleic acid molecules (e.g., DNA, RNA), small molecules, carbohydrates, lipids, synthetic polymers (e.g., polymers of PEG), conjugates (e.g., described herein), and any combination of the foregoing. Agents may contain more than one individual agent (wherein the individual agents are the same or different).

As used herein, the term “affinity” refers to the strength of the binding of one protein (e.g., an Antibody) to another protein (e.g., an Antigen). The affinity of a protein is measured by the dissociation constant Kd, defined as [Antibody]×[Antigen]/[Antibody-Antigen]where [Antibody-Antigen] is the molar concentration of the Antibody-Antigen complex, [Antibody] is the molar concentration of the unbound Antibody and [Ligand] is the molar concentration of the unbound Antigen. The affinity constant Ka is defined by 1/Kd. Standard methods of measuring affinity are known to the person of ordinary skill in the art. Exemplary methods of measuring affinity include, surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes).

As used herein, the term “antibody” or “antibodies” is used in the broadest sense and encompasses various immunoglobulin (Ig) (e.g., human Ig (Ig), murine Ig (mIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, a variable domain of a new antigen receptor beta-lactamase (VNAR fragments), affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂—Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In certain embodiments, antibodies described herein are mIgG antibodies, or a class (e.g., mIgG1 or mIgG2a) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methods known in the art, e.g., recombinant production in host cells, see, e.g., § 5.10; or synthetic production. The term “anti-X antibody” as used herein refers to an antibody that specifically binds X, wherein X is a protein (e.g., an anti-CD161 antibody refers to an antibody that specifically binds CD161).

As used herein, the term “antibody dependent cell mediated cytotoxicity” or “ADCC” refers to an immune mechanism leading to the lysis of antibody (or an Fc region containing protein) (e.g., an Ig Fc containing fusion protein described herein)-coated target cells by immune effector cells (e.g., NK cells). As used herein, the term “reduced ADCC” and the like refers to either a reduction in the number of target cells that are lysed in a given time, at a given concentration of antibody (or an Ig Fc region containing protein) (e.g., an Fc region containing fusion protein described herein) in the medium surrounding the target cells, by the mechanism of ADCC defined above, and/or an increase in the concentration of antibody (or an Fc region containing protein) (e.g., an Fc containing fusion protein described herein) in the medium surrounding the target cells, required to achieve the lysis of a given number of target cells in a given time, by the mechanism of ADCC defined above. The reduction in ADCC is relative to the ADCC mediated by the same antibody (or an Fc region containing protein) (e.g., an Fc containing fusion protein described herein) produced by the same type of host cells, using the same standard production, purification, formulation and storage methods (which are known to those skilled in the art), but that has not been engineered (e.g., does not comprise one or more amino acid variation, e.g., amino acid substitution, that mediates a decrease in ADCC). For example the reduction in ADCC mediated by an antibody (or an Fc region containing protein) (e.g., an Fc containing fusion protein described herein) comprising in its Fc region an amino acid substitution that reduces ADCC, is relative to the ADCC mediated by the same antibody (or an Fc region containing protein) (e.g., an Fc containing fusion protein described herein) without said amino acid substitution in the Fc region.

The terms “cancer” and “tumor” are used interchangeably herein and refer to a broad group of various diseases characterized by the uncontrolled growth of abnormal cells in the body. Unregulated cell division and growth results in the formation of malignant tumors that can invade neighboring tissues and may also metastasize to distant parts of the body through, e.g., the lymphatic system or bloodstream.

As used herein, the term “CD161” refers to the type II transmembrane C-type lectin-like receptor expressed, e.g., by natural killer cells. CD161 is also commonly referred to in the art as Killer cell lectin-like receptor subfamily B member 1 (KLRB1). The amino acid sequence of a reference human CD161 (hCD161) protein is set forth in SEQ ID NO: 1 (UniProt Ref.: Q12918-1).

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), the entire contents of each of which is incorporated herein by reference for all purposes. Unless otherwise specified, the term “CDR” is a CDR as defined by Kabat et al., J. Biol. Chem. 252, 6609-6616 (1977) and Kabat et al., Sequences of protein of immunological interest. (1991). A person of ordinary skill in the art would be able to determine the CDRs as defined by another scheme, e.g., Chothia, IMGT, using ordinary methods known in the art. The three sequential CDRs of the VH region are typically termed “CDR-H1”, “CDR-H2”, and “CDR-H3” herein. The three sequential CDRs of the VL region are typically termed “CDR-L1”, “CDR-L2”, and “CDR-L3” herein. As is common in the art.

The terms “CH1” and “CH1 region” are used interchangeably herein and refer to the first constant region of an immunoglobulin heavy chain. The amino acid sequence of an exemplary reference IgG1 CH1 region is set forth in SEQ ID NO: 119; and the amino acid sequence of an exemplary reference IgG4 CH1 region is set forth in SEQ ID NO: 132.

The terms “CH2” and “CH2 region” are used interchangeably herein and refer to the second constant region of an immunoglobulin heavy chain. The amino acid sequence of an exemplary reference IgG1 CH2 region is set forth in SEQ ID NO: 121; and the amino acid sequence of an exemplary reference IgG4 CH2 region is set forth in SEQ ID NO: 134.

The terms “CH3” and “CH3 region” are used interchangeably herein and refer to the third constant region of an immunoglobulin heavy chain. The amino acid sequence of an exemplary reference IgG1 CH3 region is set forth in SEQ ID NO: 122; and the amino acid sequence of an exemplary reference IgG4 CH3 region is set forth in SEQ ID NO: 135.

The terms “constant region” and “constant domain” are used interchangeably herein and refer to a carboxyl terminal portion of a light and/or heavy chain of a full-length antibody which is not directly involved in binding of an antibody to antigen, but which can exhibit various effector functions, such as interaction with an Ig Fc receptor (e.g., Fc gamma receptor). The constant region of an Ig molecule generally has a more conserved amino acid sequence relative to an Ig variable domain.

As used herein, the term “CLEC2D” or “C-type lectin domain family 2 member D” refers to the C type lectin receptor that, inter alia, binds histones released upon necrotic cell death. The amino acid sequence of a reference huma CLEC2D (hCLEC2D) isoform is set forth in SEQ ID NO: 2 (UniProt Ref.: Q9UHP7-1). Multiple isoforms of hCLEC2D are known produced by alternative splicing.

As used herein, the term “conjugation” refers to chemical conjugation of a protein with a moiety (e.g., small molecule, polypeptide, nucleic acid molecule, carbohydrate, lipid, synthetic polymer (e.g., polymers of polyethylene glycol (PEG)), etc.). The moiety can be directly connected to the protein or indirectly connected through a linker, e.g., as described herein. Chemical conjugation methods are well known in the art, as are commercially available conjugation reagents and kits, with detailed instructions for their use readily available from the commercial suppliers.

As used herein, the term “derived from,” with reference to a nucleic acid molecule refers to a nucleic acid molecule that has at least 70% sequence identity to a reference nucleic acid molecule (e.g., a naturally occurring nucleic acid molecule) or a fragment thereof. The term “derived from,” with reference to a protein refers to a protein that comprises an amino acid sequence that has at least 70% sequence identity to the amino acid sequence of a reference protein (e.g., a naturally occurring protein). The term “derived from” as used herein does not denote any specific process or method for obtaining the nucleic acid molecule, polypeptide, or protein. For example, the nucleic acid molecule, polypeptide, or protein can be recombinantly produced or chemically synthesized.

As used herein, the term “diagnosing” or “diagnosis” refers to a determination of the presence, absence, severity, or course of treatment of a disease (e.g., cancer, pro-inflammatory disease, autoimmune disease). The term “diagnosing” encompasses an initial determination as well as subsequent determinations (e.g., monitoring) after the initial determination.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “effector function” when used in reference to an Ig Fc region or a protein comprising an Ig Fc region (e.g., a full-length antibody) refers to those biological activities attributable to the Ig Fc region of a typical full-length antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and C1q binding.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, the entire contents of each of which is incorporated herein by reference for all purposes.

As used herein, the term “Fab” refers to an antigen binding domain that comprises a Fab heavy chain that comprises from N- to C-terminus a VH region and a CH1 region; and a light chain comprising from N- to C-terminus a VL region and a CL region; and wherein the Fab heavy chain and the light chain associate to form an antigen binding domain.

The term “Fab-Fc” as used herein refers to an antibody that comprises a Fab operably linked to an Fc region.

As used herein, the term “Fe region” refers to the C-terminal region of a Ig (e.g., a human Ig) heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region (e.g., comprises one or more amino acid variation), see, e.g., § 5.7. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated herein by reference for all purposes).

As used herein, the terms “first” and “second” with respect to Fc regions etc., are used for convenience of distinguishing when there is more than one of each type of moiety. Use of these terms is not intended to confer a specific order or orientation in the protein unless explicitly so stated. For example, an antibody described herein (e.g., in the case of a full-length antibody) may contain two Fc regions that associate e.g., via one or more covalent (e.g., disulfide) bond.

As used herein, the term “framework region” or “FR region” refers to the amino acid residues that are part of the variable region of an antibody, but are not part of the CDRs (e.g., using the Kabat definition of CDRs).

As used herein, the term “full-length antibody” refers to an antibody having a structure substantially similar to a native antibody structure (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the two heavy chains comprise a substantially identical amino acid sequence; and the two light chains comprise a substantially identical amino acid sequence. In some embodiments, the two heavy chains comprise a substantially identical amino acid sequence except for one or more amino acid variations that promote heterodimerization of the correct heavy chains (e.g., as described herein); and the two light chains comprise a substantially identical amino acid sequence. Antibody chains may be substantially identical but not entirely identical if they differ due to post-translational variations, such as C-terminal cleavage of lysine residues, alternative glycosylation patterns, etc.

The term “functional variant” as used herein in reference to a protein refers to a protein that comprises at least one but no more than 20%, not more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively reduced or eliminated. In some embodiments, the reference protein is a wild type protein.

The term “functional fragment” as used herein in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein.

As used herein, the term “fuse” and grammatical equivalents thereof refer to the operable connection of at least a first polypeptide to a second polypeptide, wherein the first and second polypeptides are not naturally found operably connected together. For example, the first and second polypeptides are derived from different proteins. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the first and second polypeptides are not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “half-life extension moiety” refers to a moiety (e.g., small molecule, polypeptide, nucleic acid molecule, carbohydrate, lipid, synthetic polymer (e.g., polymers of PEG), etc.) that when conjugated or otherwise operably connected (e.g., fused) to a protein (the subject protein), increases the half-life of the subject protein in vivo when administered to a subject (e.g., a human subject). The pharmacokinetic properties of the protein can be evaluated utilizing in vivo models known in the art.

As used herein, the term “half-life extension polypeptide” or “half-life extension protein” refers to a protein that when operably connected to another protein (the subject protein), increases the half-life of the subject protein in vivo when administered to a subject (e.g., a human subject). The pharmacokinetic properties of the protein can be evaluated utilizing in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, nucleic acid molecule, carbohydrate, lipid, synthetic polymer (e.g., polymers of PEG), etc.) that is not joined to the polypeptide in nature.

As used herein, the term “heavy chain” refers to the portion of an immunoglobulin (e.g., a human Ig) that typically comprises from N- to C-terminus a heavy chain variable region (VH), a CH1 region, a hinge region, a CH2 region, and a CH3 region. The constant regions of the heavy chain (i.e., the CH1 region, the hinge region, the CH2 region, and the CH3 region) can be any distinct isotype, for example, human alpha (a), delta (6), epsilon (F), gamma (γ), and mu (p), based on the amino acid sequence of the constant domain, which give rise to the IgA, IgD, IgE, IgG, and IgM classes of human antibodies, respectively, including subclasses of IgG, e.g., IgG₁, IgG₂, IgG₃, and IgG₄. As used herein, the term “heavy chain” when used in reference to a human antibody can refer to any distinct type, e.g., alpha (a), delta (6), epsilon (F), gamma (γ), and mu (p), based on the amino acid sequence of the constant domain, which give rise to human IgA, IgD, IgE, IgG, and IgM classes of antibodies, respectively, including subclasses of human IgG, e.g., IgG₁, IgG₂, IgG₃, and IgG₄.

The terms “hinge” or “hinge region” are used interchangeably herein and refer to the hinge region of an immunoglobulin heavy chain. The amino acid sequence of an exemplary reference IgG₁ hinge region is set forth in SEQ ID NO: 120; and the amino acid sequence of an exemplary reference IgG₄ hinge region is set forth in SEQ ID NO: 133.

As used herein, the term “isolated” with reference to an agent (e.g., a protein, nucleic acid molecule, etc.) refers to an agent (e.g., a protein, nucleic acid molecule, etc.) that is substantially free of other cellular components with which it is associated in the natural state.

The terms “nucleic acid molecule,” “polynucleotide,” and “oligonucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleoside linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “obtaining a sample” refers to the acquisition of a sample. The term includes the direct acquisition from a subject and the indirect acquisition through one or more third parties wherein one of the third parties directly acquired the sample from the subject.

As used herein, the term “operably connected” refers to the linkage of two agents in a functional relationship. For example, a polypeptide is operably connected to another polypeptide when they are linked (either directly or indirectly via a peptide linker) in frame such that both polypeptides are functional. Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” also refers for example to the conjugation of a first agent (e.g., an antibody) to a second agent wherein the first and second agent are both capable of mediating their function.

The determination of “percent identity” between two sequences (e.g., protein (amino acid sequences) or oligonucleotide (nucleic acid sequences)) can be accomplished using a mathematical algorithm. Determinations of identity (as described herein) are independent of nucleotide chemical variations (e.g., as described herein). For example, (mC) is identical to (C) for the purposes of determining identity. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, the term “plurality” means 2 or more (e.g., 3 or more, 4 or more, 5 or more, 6 or more, 7 or more, 9 or more, or 10 or more).

As used herein, the terms “protein”, “polypeptide”, and “peptide” refer to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where polypeptides (e.g., in a linear (i.e., primary) structure are contemplated herein, it should be understood that proteins folded into their three-dimensional structure (i.e., tertiary or quaternary structure) are also provided herein and vice versa. Proteins include e.g., naturally occurring proteins, variant (e.g., functional variants) of naturally occurring proteins, fragments (e.g., functional fragments) of naturally occurring proteins, and synthetic proteins (i.e., not naturally occurring proteins).

A “prophylactic” treatment is a treatment administered to a subject who does not exhibit signs of a disease or exhibits only early signs for the purpose of decreasing the risk of developing pathology.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleoside linkages, such as a phosphoroamidate linkage or a phosphorothioate linkage, instead of the phosphodiester found between the nucleotides of an unmodified nucleic acid molecule.

As used herein, the term “sample” encompass a variety of biological specimens obtained from a subject. Exemplary sample types include, e.g., blood and other liquid samples of biological origin (including, but not limited to, whole-blood, peripheral blood mononuclear cells (PBMCs), serum, plasma, urine, saliva, amniotic fluid, stool, synovial fluid, etc.), nasopharyngeal swabs, biopsies, solid tissue samples such as biopsies (or cells derived therefrom and the progeny thereof), tissue cultures (or cells derived therefrom and the progeny thereof), and cell cultures (or cells derived therefrom and the progeny thereof). The term also includes samples that have been manipulated in any way after their procurement from a subject, such as by centrifugation, filtration, washing, precipitation, dialysis, chromatography, lysis, treatment with reagents, enriched for certain cell populations, refrigeration, freezing, staining, etc.

The term “scFv” or “single chain variable fragment” refers to an antibody that comprises a VH region operably connected via a peptide linker to a VL region, wherein the VH and VL regions associate to specifically bind an antigen (e.g., form an antigen binding domain). In some embodiments, the scFv comprises from N- to C-terminus an VH region, a peptide linker, and an VL region. In some embodiments, the scFv comprises from N- to C-terminus an VL region, a peptide linker, and an VH region.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide.

As used herein, the term “specifically binds” and the like refers to preferential interaction, i.e., significantly higher binding affinity, between a first agent (e.g., protein (e.g., an antibody)) and a second agent (e.g., protein (e.g., an antigen)) relative to other agents (e.g., other amino acid sequences). For example, in some embodiments, specifically binds and the like refers to preferential interaction, i.e., significantly higher binding affinity, between a first protein (e.g., an antibody) and a second protein (e.g., an antigen) relative to other amino acid sequences. Herein, when a first protein is said to “specifically bind” to a second protein, it is understood that the first protein specifically binds to an epitope of the second protein. The term “epitope” refers to the portion of the second protein that the first protein specifically recognizes. The term specifically binds includes molecules that are cross reactive with the same epitope of a different species. For example, an antibody that specifically binds human CD161 may be cross reactive with CD161 of another species (e.g., cynomolgus, murine, etc.), and still be considered herein to specifically bind huma CD161. A protein can specifically bind more than one different protein. Specific binding can be measured, e.g., through measuring binding affinity (e.g., using standard methods known in the art and described herein (e.g., surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes).

As used herein, the term “stratification” and the like refers to the classifying of subjects (e.g., human subjects) into groups based on the one or more specified criteria. For example, criteria can include, e.g., a determination from a method or assay described herein (e.g., a determination of the expression of CD161 by cells in a sample (e.g., a tumor biopsy)).

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutic agent” refers to an agent (e.g., an antibody described herein) capable of achieving a desired therapeutic result in a subject or ex vivo (e.g., capable of treating a disease as defined herein) when administered at a therapeutically effective amount.

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of e.g., a conjugate described herein (or a carrier, pharmaceutical composition, etc. comprising the same).

As used herein, the term “variation” or “variant” or use the like in reference to a nucleotide or nucleic acid sequence refers to a nucleic acid molecule that comprises at least one substitution, addition, deletion, or inversion of one or more nucleotide compared to a reference nucleic acid molecule. Likewise, as used herein, the term “variation” or “variant” or use the like with reference to a peptide or protein refers to a peptide or protein that comprises at least one substitution, addition, deletion, or inversion of an amino acid residue compared to a reference peptide or protein.

As used herein, the term “variable region” refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL region” are used interchangeably to refer to an immunoglobulin light chain variable region. A VL region can be incorporated into an antibody, e.g., a scFv, a Fab, a full-length antibody. For example, a scFv comprises a VL region operably connected via a peptide linker to a VH region.

The terms “VH” and “VH region” are used interchangeably to refer to an immunoglobulin heavy chain variable region. A VH region can be incorporated into an antibody, e.g., a scFv, a Fab, a full-length antibody. For example, a scFv comprises a VH region operably connected via a peptide linker to a VL region.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The term “(VHH)₂” as used herein refers to an antibody that comprises a first and a second VHH operably connected (e.g., via a peptide linker). The first and the second VHH can specifically bind the same or different antigens. In some embodiments, the first and second VHH are operably connected by a peptide linker.

The term “VHH-Fc” as used herein refers to an antibody that comprises a VHH operably linked (e.g., via a peptide linker) to an Fc domain or a subunit of an Fc domain. In some embodiments, a VHH is operably connected to only a first Fc domain of a first and a second Fc domain pair. In some embodiments, a first VHH is operably connected to a first Fc domain and a second VHH is operably connected to a second Fc domain of a first Fc and a second Fc pair.

The term “(VHH)₂—Fc” as used herein refers to (VHH)₂ operably linked (e.g., via a peptide linker) to an Fc domain or a subunit of an Fc domain. In some embodiments, a (VHH)₂ is operably connected to only a first Fc domain of a first and a second Fc domain pair. In some embodiments, a first (VHH)₂ is operably connected to a first Fc domain and a second (VHH)₂ is operably connected to a second Fc domain of a first Fc and a second Fc pair.

### 5.2 CD161 Binding Proteins

As described above, provided herein are, inter alia, proteins (e.g., antibodies (and functional fragments and/or functional variants thereof)) that specifically bind CD161 (e.g., human CD161 (hCD161)).

CD161 is a C-type lectin-like receptor, which is expressed e.g., on NK cells, CD4+, CD8+ T cells, and macrophages. CD161 binds, e.g., CLEC2D). CLEC2D) is expressed, e.g., on the surface of both malignant cells and immune cells including germinal center B cells, activated T cells and tumor associated macrophages. The amino acid sequence of a reference hCD161 protein is set forth in SEQ ID NO: 1. Multiple isoforms of human CLEC2D) (hCLEC2D) are known to be produced by alternative splicing, with isoformn 1 being the only isoformn predominantly expressed at the cell surface. The amino acid sequence of a reference hCLEC2D isoformn 1 protein is set forth in SEQ ID NO: 2. See Table 1, herein.

TABLE 1
The Amino Acid Sequence of a Reference hCD161 and hCLEC2D Protein.

| | | SEQ |
|---|---|---|
| Description | Amino Acid Sequence | ID NO |
| hCD161 | MERLWGLFQRAQQLSPRSSQTVYQRVEGPRKGHLEEEEEDGEEGAETLA | 1 |
| UniProt | HFCPMELRGPEPLGSRPRQPNLIPWAAAGRRAAPYLVLTALLIFTGAFL | |
| Accession | LGYVAFRGSCQACGDSVLVVSEDVNYEPDLDFHQGRLYWSDLQAMFLQF | |
| Q12918-1 | LGEGRLEDTIRQTSLRERVAGSAGMAALTQDIRAALSRQKLDHVWIDTH | |
| | YVGLQFPDPAHPNTLHWVDEAGKVGEQLPLEDPDVYCPYSAIGNVTGEL | |
| | VYAHYGRPEDLQDLRARGVDPVGRLLLVRVGVISFAQKVTNAQDEGAQG | |
| | VLIYPEPADESQDPPKPSLSSQQAVYGHVHLGTGDPYTPGFPSFNQTQF | |
| | PPVASSGLPSIPAQPISADIASRLLRKLKGPVAPQEWQGSLLGSPYHLG | |
| | PGPRLRLVVNNHRTSTPINNIFGCIEGRSEPDHYVVIGAQRDAWGPGAA | |
| | KSAVGTAILLELVRTFSSMVSNGFRPRRSLLFISWDGGDEGSVGSTEWL | |
| | EGYLSVLHLKAVVYVSLDNAVLGDDKFHAKTSPLLTSLIESVLKQVDSP | |
| | NHSGQTLYEQVVFTNPSWDAEVIRPLPMDSSAYSFTAFVGVPAVEFSEM | |
| | EDDQAYPFLHTKEDTYENLHKVLQGRLPAVAQAVAQLAGQLLIRLSHDR | |
| | LLPLDFGRYGDVVLRIGNLNEFSGDLKARGLTLQWVYSARGDYIRAAEK | |
| | LRQEIYSSEERDERLTRMYNVRIMRVEFYFLSQYVSPADSPFRHIEMGR | |
| | GDHTLGALLDHLRLLRSNSSGTPGATSSTGFQESRFRRQLALLTWTLQG | |
| | AANALSGDVWNIDNNE | |
| hCLEC2D | MMDQARSAFSNLEGGEPLSYTRESLARQVDGDNSHVEMKLAVDEEENAD | 2 |
| UniProt | NNTKANVTKPKRCSGSICYGTIAVIVFFLIGFMIGYLGYCKGVEPKTEC | |
| Accession | ERLAGTESPVREEPGEDEPAARRLYWDDLKRKLSEKLDSTDFTGTIKLL | |
| Q9UHP7-1 | NENSYVPREAGSQKDENLALYVENQFREFKLSKVWRDQHFVKIQVKDSA | |
| | QNSVIIVDKNGRLVYLVENPGGYVAYSKAATVTGKLVHANFGTKKDFED | |
| | LYTPVNGSIVIVRAGKITFAEKVANAESLNAIGVLIYMDQTKEPIVNAE | |
| | LSFFGHAHLGTGDPYTPGFPSENHTQFPPSRSSGLPNIPVQTISRAAAE | |
| | KLFGNMEGDCPSDWKTDSTCRMVTSESKNVKLTVSNVLKEIKILNIFGV | |
| | IKGFVEPDHYVVVGAQRDAWGPGAAKSGVGTALLLKLAQMESDMVLKDG | |
| | FQPSRSIIFASWSAGDEGSVGATEWLEGYLSSLHLKAFTYINLDKAVLG | |
| | TSNFKVSASPLLYTLIEKTMQNVKHPVTGQFLYQDSNWASKVEKLTLDN | |
| | AAFPFLAYSGIPAVSFCFCEDTDYPYLGTTMDTYKELIERIPELNKVAR | |
| | AAAEVAGQFVIKLTHDVELNLDYERYNSQLLSFVRDLNQYRADIKEMGL | |
| | SLQWLYSARGDFFRATSRLTTDEGNAEKTDRFVMKKLNDRVMRVEYHEL | |
| | SPYVSPKESPFRHVFWGSGSHTLPALLENLKLRKQNNGAFNETLERNQL | |
| | ALATWTIQGAANALSGDVWDIDNEF | |

In some embodiments, the CD161 binding protein comprises an antibody. In some embodiments, the CD161 binding protein comprises a full-length antibody, Fab, Fab′, F(ab′)2, Fab-Fe, scFv, scFv-Fc, (scFv)₂-Fc, Fv, a single domain antibody (sdAb) (e.g., a VHH), a sdAb-Fc (e.g., a VHH-Fc), (sdAb)₂ (e.g., a (VHH)₂, or a (sdAb)₂-Fc (e.g., (VHH)₂—Fc). In some embodiments, the CD161 binding protein comprises a full-length antibody, Fab, Fab′, F(ab′)2, Fab-Fc, scFv, scFv-Fc, (scFv)₂-Fc, sdAb-Fc (e.g., a VHH-Fc), or (sdAb)₂-Fc (e.g., (VHH)₂—Fc). In some embodiments, the CD161 binding protein comprises a full-length antibody. In some embodiments, the CD161 binding protein comprises a Fab. In some embodiments, the antibody comprises a F(ab′)2. In some embodiments, the CD161 binding protein comprises a Fab-Fc. In some embodiments, the antibody comprises a scFv-Fc. In some embodiments, the CD161 binding protein comprises a (scFv)₂-Fc. In some embodiments, the CD161 binding protein comprises a sdAb-Fc (e.g., a VHH-Fc). In some embodiments, the CD161 binding protein comprises a (sdAb)₂-Fc (e.g., (VHH)₂—Fc).

In some embodiments, the CD161 binding protein consists of an antibody. In some embodiments, the CD161 binding protein consists of a full-length antibody, Fab, Fab′, F(ab′)2, Fab-Fc, scFv, scFv-Fc, (scFv)₂-Fc, Fv, a single domain antibody (sdAb) (e.g., a VHH), a sdAb-Fc (e.g., a VHH-Fc), (sdAb)₂ (e.g., a (VHH)₂, or a (sdAb)₂-Fc (e.g., (VHH)₂—Fc). In some embodiments, the CD161 binding protein consists of a full-length antibody, Fab, Fab′, F(ab′)2, Fab-Fc, scFv, scFv-Fc, (scFv)₂-Fc, sdAb-Fc (e.g., a VHH-Fc), or (sdAb)₂-Fc (e.g., (VHH)₂—Fc). In some embodiments, the CD161 binding protein consists of a full-length antibody. In some embodiments, the CD161 binding protein consists of a Fab. In some embodiments, the antibody consists of a F(ab′)2. In some embodiments, the CD161 binding protein consists of a Fab-Fc. In some embodiments, the antibody consists of a scFv-Fc. In some embodiments, the CD161 binding protein consists of a (scFv)₂-Fc. In some embodiments, the CD161 binding protein consists of a sdAb-Fc (e.g., a VHH-Fc). In some embodiments, the CD161 binding protein consists of a (sdAb)₂-Fc (e.g., (VHH)₂—Fc).

In some embodiments, the CD161 binding protein is an antibody. In some embodiments, the CD161 binding protein is a full-length antibody, Fab, Fab′, F(ab′)2, Fab-Fc, scFv, scFv-Fc, (scFv)₂-Fc, Fv, a single domain antibody (sdAb) (e.g., a VHH), a sdAb-Fc (e.g., a VHH-Fc), (sdAb)₂ (e.g., a (VHH)₂, or a (sdAb)₂-Fc (e.g., (VHH)₂—Fc). In some embodiments, the CD161 binding protein is a full-length antibody, Fab, Fab′, F(ab′)2, Fab-Fc, scFv, scFv-Fc, (scFv)₂-Fe, sdAb-Fc (e.g., a VHH-Fc), or (sdAb)₂-Fc (e.g., (VHH)₂—Fc). In some embodiments, the CD161 binding protein is a full-length antibody. In some embodiments, the CD161 binding protein is a Fab. In some embodiments, the antibody is a F(ab′)2. In some embodiments, the CD161 binding protein is a Fab-Fc. In some embodiments, the antibody is a scFv-Fc. In some embodiments, the CD161 binding protein is a (scFv)₂-Fc. In some embodiments, the CD161 binding protein is a sdAb-Fc (e.g., a VHH-Fc). In some embodiments, the CD161 binding protein is a (sdAb)₂-Fc (e.g., (VHH)₂—Fc).

In some embodiments, the CD161 binding protein comprises a full-length antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the amino acid sequence of the first heavy chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the first light chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the VH region of the first heavy chain is 100% identical to the amino acid sequence of the VH region of the second heavy chain; and the amino acid sequence of the first heavy chain outside of the VH region is at least 85%, 86%, 87%, 88%, 89%, 90%, 91%, 92%, 93%, 94%, 95%, 96%, 97%, 98%, 99%, or 100% identical to the amino acid sequence of the second heavy chain outside of the VH region of the second heavy chain. In some embodiments, the amino acid sequence of the VL region of the first light chain is 100% identical to the amino acid sequence of the VL region of the second light chain; and the amino acid sequence of the first light chain outside of the VL region is at least 85%, 86%, 87%, 88%, 89%, 90%, 91%, 92%, 93%, 94%, 95%, 96%, 97%, 98%, 99%, or 100% identical to the amino acid sequence of the second light chain outside of the VL region of the second light chain.

In some embodiments, the antibody is an IgG1, IgG2, IgG3, or IgG4 antibody. In some embodiments, the antibody is an IgG1 or IgG4 antibody. In some embodiments, the antibody is an IgG1 antibody. In some embodiments, the antibody is an IgG4 antibody. In some embodiments, the antibody is a IgG1, IgG2, IgG3, or IgG4 antibody. In some embodiments, the antibody is a IgG1 or IgG4 antibody. In some embodiments, the antibody is a IgG1 antibody. In some embodiments, the antibody is a IgG4 antibody. In some embodiments, the antibody is human antibody. In some embodiments, the antibody is a non-human mammalian antibody. In some embodiments, the antibody is a murine antibody. In some embodiments, the antibody is a humanized antibody. In some embodiments, the antibody is a chimeric antibody.

In some embodiments, the CD161 binding protein (e.g., antibody) is monospecific. In some embodiments, the CD161 binding protein (e.g., antibody) is multispecific (e.g., bispecific, trispecific). In some embodiments, the CD161 binding protein (e.g., antibody) is multispecific comprising at least one moiety that specifically binds another antigen (i.e., not CD161). In some embodiments, the CD161 binding protein (e.g., antibody) is bispecific comprising at least one moiety that specifically binds another antigen (i.e., not CD161).

In some embodiments, the CD161 binding proteins (e.g., described herein) (or conjugates or fusions comprising the same) (e.g., antibodies) are multimeric (e.g., dimeric, trimeric, tetrameric, etc.) comprising at least two, three, or four polypeptides.

In some embodiments, the CD161 binding protein comprises at least two, three, or four polypeptides. In some embodiments, the CD161 binding protein is dimeric (i.e., comprises two polypeptides). In some embodiments, the CD161 binding protein is trimeric (i.e., comprises three polypeptides). In some embodiments, the CD161 binding protein is tetrameric (i.e., comprises four polypeptides). In some embodiments, two of the polypeptides associate via covalent or non-covalent interactions. In some embodiments, two of the polypeptides associate via at least one covalent interaction. In some embodiments, two of the polypeptides associate via one or more disulfide bond. In some embodiments, two of the polypeptides associate via 1, 2, 3, 4, or more disulfide bonds.

The amino acid sequence of hCD161 binding proteins (e.g., anti-hCD161 antibodies) of the present disclosure is provided in Table 2. The CDRs of the antibodies in Table 2, are denoted according to Kabat. A person of ordinary skill in the art would be able to determine the CDRs as defined by another scheme, e.g., Chothia, IMGT, using ordinary methods known in the art.

TABLE 2
The Amino Acid Sequence of hCD161 Binding Proteins.

| | | SEQ | |
|---|---|---|---|
| Description | | ID NO | Amino Acid Sequence |
| Ab-1 | CDR-H1 | 169 | SYALS |
| | CDR-H2 | 170 | YISSGGDYIYYADTVKG |
| | CDR-H3 | 171 | DEYYAVDY |
| | CDR-L1 | 172 | KSSQSLLYSSNQKNYLA |
| | CDR-L2 | 173 | WTSTRES |
| | CDR-L3 | 174 | QQYYSYPIT |
| | VH | 175 | DVKLVESGEGLVKPGGSLKLSCAASGFTFGSYALSWVRQTPEKRLEWVAY |
| | | | ISSGGDYIYYADTVKGRFTISRDNARNTLYLQMSSLKSEDTAMYYCTRDE |
| | | | YYAVDYWGQGTSVTVSS |
| | VL | 176 | DIVMSQSPSSLAVSVGEKITMSCKSSQSLLYSSNQKNYLAWYQQKPGQSP |
| | | | KLLIYWTSTRESGVPDRFTGSGSGTDFTLTISSVKAEDLAVYYCQQYYSY |
| | | | PITFGSGTKLEIK |
| | HC | 177 | DVKLVESGEGLVKPGGSLKLSCAASGFTFGSYALSWVRQTPEKRLEWVAY |
| | | | ISSGGDYIYYADTVKGRFTISRDNARNTLYLQMSSLKSEDTAMYYCTRDE |
| | | | YYAVDYWGQGTSVTVSSAKTTPPSVYPLAPGSAAQTNSMVTLGCLVKGYF |
| | | | PEPVTVTWNSGSLSSGVHTFPAVLQSDLYTLSSSVTVPSSPRPSETVTCN |
| | | | VAHPASSTKVDKKIVPRDCGCKPCICTVPEVSSVFIFPPKPKDVLTITLT |
| | | | PKVTCVVVDISKDDPEVQFSWFVDDVEVHTAQTQPREEQFNSTERSVSEL |
| | | | PIMHQDWLNGKEFKCRVNSAAFPAPIEKTISKTKGRPKAPQVYTIPPPKE |
| | | | QMAKDKVSLTCMITDFFPEDITVEWQWNGQPAENYKNTQPIMNINGSYFV |
| | | | YSKLNVQKSNWEAGNTFTCSVLHEGLHNHHTEKSLSHSPGK |
| | LC | 178 | DIVMSQSPSSLAVSVGEKITMSCKSSQSLLYSSNQKNYLAWYQQKPGQSP |
| | | | KLLIYWTSTRESGVPDRFTGSGSGTDFTLTISSVKAEDLAVYYCQQYYSY |
| | | | PITFGSGTKLEIKRADAAPTVSIFPPSSEQLTSGGASVVCFLNNFYPKDI |
| | | | NVKWKIDGSERQNGVLNSWTDQDSKDSTYSMSSTLTLTKDEYERHNSYTC |
| | | | EATHKTSTSPIVKSENRNEC |

In some embodiments, the CD 161 binding protein comprises a CD 161 binding protein provided in Table 2. In some embodiments, the CD161 binding protein comprises CD161 binding protein Ab-1 (see Table 2).

In some embodiments, the CD161 binding protein (e.g., anti-CD161 antibody) comprises a VH that comprises: a CDR-H1, a CDR-H-2, and a CDR-H3. In some embodiments, the CD161 binding protein comprises a VL that comprises: CDR-L1, CDR-L2, and CDR-L3. In some embodiments, the CD161 binding protein comprises a VH that comprises: a CDR-H1, a CDR-H-2, and a CDR-H3; and a VL that comprises: CDR-L1, CDR-L2, and CDR-L3.

In some embodiments, the amino acid sequence of CDR-H1 comprises the amino acid sequence of CDR-H1 of Ab-1, or the amino acid sequence of the CDR-H1 of Ab-1 comprising 1, 2, or 3 amino acid variations (e.g., substitution, deletion, addition, etc.); the amino acid sequence of CDR-H2 comprises the amino acid sequence of CDR-H2 of Ab-1, or the amino acid sequence of CDR-H2 of Ab-1 comprising 1, 2, or 3 amino acid variations (e.g., substitution, deletion, addition, etc.); the amino acid sequence of CDR-H3 comprises the amino acid sequence of CDR-H3 of Ab-1, or the amino acid sequence of CDR-H3 of Ab-1 comprising 1, 2, or 3 amino acid variations (e.g., substitution, deletion, addition, etc.); the amino acid sequence of CDR-L1 comprises the amino acid sequence of CDR-L1 of Ab-1, or the amino acid sequence of CDR-L1 of Ab-1 comprising 1, 2, or 3 amino acid variations (e.g., substitution, deletion, addition, etc.); the amino acid sequence of CDR-L2 comprises the amino acid sequence of a CDR-L2 of Ab-1, or the amino acid sequence of a CDR-L2 of Ab-1 comprising 1, 2, or 3 amino acid variations (e.g., substitution, deletion, addition, etc.); and the amino acid sequence of CDR-L3 comprises the amino acid sequence of CDR-L3 of Ab-1, or the amino acid sequence of CDR-L3 of Ab-1 comprising 1, 2, or 3 amino acid variations (e.g., substitution, deletion, addition, etc.).

In some embodiments, the amino acid sequence of CDR-H1 comprises the amino acid sequence of CDR-H1 of Ab-1, or the amino acid sequence of CDR-H1 of Ab-1 comprising no more than 1, 2, or 3 amino acid variations (e.g., substitution, deletion, addition, etc.); the amino acid sequence of CDR-H2 comprises the amino acid sequence of CDR-H2 of Ab-1, or the amino acid sequence of CDR-H2 of Ab-1 comprising no more than 1, 2, or 3 amino acid variations (e.g., substitution, deletion, addition, etc.); the amino acid sequence of CDR-H3 comprises of CDR-H3 of Ab-1, or the amino acid sequence of CDR-H3 of Ab-1 comprising no more than 1, 2, or 3 amino acid variations (e.g., substitution, deletion, addition, etc.); the amino acid sequence of CDR-L1 comprises the amino acid sequence of CDR-L1 of Ab-1, or the amino acid sequence of CDR-L1 of Ab-1 comprising no more than 1, 2, or 3 amino acid variations (e.g., substitution, deletion, addition, etc.); the amino acid sequence of CDR-L2 comprises the amino acid sequence of CDR-L2 of Ab-1, or the amino acid sequence of CDR-L2 of Ab-1 comprising no more than 1, 2, or 3 amino acid variations (e.g., substitution, deletion, addition, etc.); and the amino acid sequence of CDR-L3 comprises the amino acid sequence of CDR-L3 of Ab-1, or the amino acid sequence of CDR-L3 of Ab-1 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-1; the amino acid sequence of CDR-H2 comprises the amino acid sequence of CDR-H2 of Ab-1; the amino acid sequence of CDR-H3 comprises CDR-H3 of Ab-1; the amino acid sequence of CDR-L1 comprises the amino acid sequence of CDR-L1 of Ab-1; the amino acid sequence of CDR-L2 comprises the amino acid sequence of CDR-L2 of Ab-1; and the amino acid sequence of CDR-L3 comprises the amino acid sequence of CDR-L3 of Ab-1.

In some embodiments, the amino acid sequence of CDR-H1 comprises the amino acid sequence of CDR-H1 of Ab-1 comprising no more than 1, 2, or 3 amino acid variations (e.g., substitution, deletion, addition, etc.); the amino acid sequence of CDR-H2 comprises the amino acid sequence CDR-H2 of Ab-1 comprising no more than 1, 2, or 3 amino acid variations (e.g., substitution, deletion, addition, etc.); the amino acid sequence of CDR-H3 comprises the amino acid sequence CDR-H3 of Ab-1 comprising no more than 1, 2, or 3 amino acid variations (e.g., substitution, deletion, addition, etc.); the amino acid sequence of CDR-L1 comprises the amino acid sequence CDR-L1 of Ab-1 comprising no more than 1, 2, or 3 amino acid variations (e.g., substitution, deletion, addition, etc.); the amino acid sequence of a CDR-L2 comprises the amino acid sequence CDR-L2 of Ab-1 comprising no more than 1, 2, or 3 amino acid variations (e.g., substitution, deletion, addition, etc.); and the amino acid sequence of a CDR-L3 comprises the amino acid sequence CDR-L3 of Ab-1 comprising no more than 1, 2, or 3 amino acid variations (e.g., substitution, deletion, addition, etc.).

In some embodiments, the amino acid sequence of CDR-H1 consists of the amino acid sequence of CDR-H1 of Ab-1, or the amino acid sequence of CDR-H1 of Ab-1 comprising 1, 2, or 3 amino acid variations (e.g., substitution, deletion, addition, etc.); the amino acid sequence of CDR-H2 consists of the amino acid sequence of CDR-H2 of Ab-1, or the amino acid sequence of CDR-H2 of Ab-1 comprising 1, 2, or 3 amino acid variations (e.g., substitution, deletion, addition, etc.); the amino acid sequence of CDR-H3 consists of the amino acid sequence of CDR-H3 of Ab-1, or the amino acid sequence of CDR-H3 of Ab-1 comprising 1, 2, or 3 amino acid variations (e.g., substitution, deletion, addition, etc.); the amino acid sequence of CDR-L1 consists of the amino acid sequence of CDR-L1 of Ab-1, or the amino acid sequence of CDR-L1 of a Ab-1 comprising 1, 2, or 3 amino acid variations (e.g., substitution, deletion, addition, etc.); the amino acid sequence of CDR-L2 consists of the amino acid sequence of CDR-L2 of Ab-1, or the amino acid sequence of CDR-L2 of Ab-1 comprising 1, 2, or 3 amino acid variations (e.g., substitution, deletion, addition, etc.); and the amino acid sequence of CDR-L3 consists of the amino acid sequence of CDR-L3 of Ab-1, or the amino acid sequence of CDR-L3 of Ab-1 comprising 1, 2, or 3 amino acid variations (e.g., substitution, deletion, addition, etc.).

In some embodiments, the amino acid sequence of CDR-H1 consists of the amino acid sequence of CDR-H1 of Ab-1, or the amino acid sequence of CDR-H1 of Ab-1 comprising no more than 1, 2, or 3 amino acid variations (e.g., substitution, deletion, addition, etc.); the amino acid sequence of CDR-H2 consists of the amino acid sequence of CDR-H2 of Ab-1, or the amino acid sequence of CDR-H2 of Ab-1 comprising no more than 1, 2, or 3 amino acid variations (e.g., substitution, deletion, addition, etc.); the amino acid sequence of CDR-H3 consists of the amino acid sequence of CDR-H3 of Ab-1, or the amino acid sequence of CDR-H3 of Ab-1 comprising no more than 1, 2, or 3 amino acid variations (e.g., substitution, deletion, addition, etc.); the amino acid sequence of CDR-L1 consists of the amino acid sequence of CDR-L1 of Ab-1, or the amino acid sequence of CDR-L1 of Ab-1 comprising no more than 1, 2, or 3 amino acid variations (e.g., substitution, deletion, addition, etc.); the amino acid sequence of CDR-L2 consists of the amino acid sequence of CDR-L2 of Ab-1, or the amino acid sequence of CDR-L2 of Ab-1 comprising no more than 1, 2, or 3 amino acid variations (e.g., substitution, deletion, addition, etc.); and the amino acid sequence of CDR-L3 consists of the amino acid sequence of CDR-L3 of Ab-1, or the amino acid sequence of CDR-L3 of Ab-1 comprising no more than 1, 2, or 3 amino acid variations (e.g., substitution, deletion, addition, etc.).

In some embodiments, the amino acid sequence of CDR-H1 consists of the amino acid sequence of CDR-H1 of Ab-1; the amino acid sequence of CDR-H2 consists of the amino acid sequence of CDR-H2 of Ab-1; the amino acid sequence of CDR-H3 consists of CDR-H3 of Ab-1; the amino acid sequence of CDR-L1 consists of the amino acid sequence of CDR-L1 of Ab-1; the amino acid sequence of CDR-L2 consists of the amino acid sequence of CDR-L2 of Ab-1; and the amino acid sequence of CDR-L3 consists of the amino acid sequence of CDR-L3 of Ab-1.

In some embodiments, the amino acid sequence of CDR-H1 consists of the amino acid sequence of CDR-H1 of Ab-1 comprising no more than 1, 2, or 3 amino acid variations (e.g., substitution, deletion, addition, etc.); the amino acid sequence of CDR-H2 consists of the amino acid sequence of CDR-H2 of Ab-1 comprising no more than 1, 2, or 3 amino acid variations (e.g., substitution, deletion, addition, etc.); the amino acid sequence of CDR-H3 consists of the amino acid sequence of CDR-H3 of Ab-1 comprising no more than 1, 2, or 3 amino acid variations (e.g., substitution, deletion, addition, etc.); the amino acid sequence of CDR-L1 consists of the amino acid sequence of CDR-L1 of Ab-1 comprising no more than 1, 2, or 3 amino acid variations (e.g., substitution, deletion, addition, etc.); the amino acid sequence of CDR-L2 consists of the amino acid sequence of CDR-L2 of Ab-1 comprising no more than 1, 2, or 3 amino acid variations (e.g., substitution, deletion, addition, etc.); and the amino acid sequence of CDR-L3 consists of the amino acid sequence of CDR-L3 of Ab-1 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-1; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-1. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-1; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-1. In some embodiments, the amino acid sequence of the VH comprises an amino acid sequence at least 95%, 96%, 97%, 98%, 99%, or 100% identical to the amino acid sequence of the VH of Ab-1; and the amino acid sequence of the VL comprises an amino acid sequence at least 95%, 96%, 97%, 98%, 99%, or 100% identical to the amino acid sequence of the VL of Ab-1. In some embodiments, the amino acid sequence of the VH comprises the amino acid sequence of the VH of Ab-1; and the amino acid sequence of the VL comprises the amino acid sequence of the VL of Ab-1.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-1; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-1. In some embodiments, the amino acid sequence of the VH consists of an amino acid sequence at least 90%, 91%, 92%, 93%, 94%, 95%, 96%, 97%, 98%, 99%, or 100% identical to the amino acid sequence of the VH of Ab-1; and the amino acid sequence of the VL consists of an amino acid sequence at least 90%, 91%, 92%, 93%, 94%, 95%, 96%, 97%, 98%, 99%, or 100% identical to the amino acid sequence of the VL of Ab-1. In some embodiments, the amino acid sequence of the VH consists of an amino acid sequence at least 95%, 96%, 97%, 98%, 99%, or 100% identical to the amino acid sequence of the VH of Ab-1; and the amino acid sequence of the VL consists of an amino acid sequence at least 95%, 96%, 97%, 98%, 99%, or 100% identical to the amino acid sequence of the VL of Ab-1. In some embodiments, the amino acid sequence of the VH consists of the amino acid sequence of the VH of Ab-1; and the amino acid sequence of the VL consists of the amino acid sequence of the VL of Ab-1.

In some embodiments, the amino acid sequence of CDR-H1 comprises the amino acid sequence set forth in SEQ ID NO: 169, or the amino acid sequence set forth in SEQ ID NO: 169 comprising 1, 2, or 3 amino acid variations (e.g., substitution, deletion, addition, etc.); the amino acid sequence of CDR-H2 comprises the amino acid sequence set forth in SEQ ID NO: 170, or the amino acid sequence set forth in SEQ ID NO: 170 comprising 1, 2, or 3 amino acid variations (e.g., substitution, deletion, addition, etc.); the amino acid sequence of CDR-H3 comprises the amino acid sequence set forth in SEQ ID NO: 171, or the amino acid sequence set forth in SEQ ID NO: 171 comprising 1, 2, or 3 amino acid variations (e.g., substitution, deletion, addition, etc.); the amino acid sequence of CDR-L1 comprises the amino acid sequence set forth in SEQ ID NO: 172, or the amino acid sequence set forth in SEQ ID NO: 172 comprising 1, 2, or 3 amino acid variations (e.g., substitution, deletion, addition, etc.); the amino acid sequence of CDR-L2 comprises the amino acid sequence set forth in SEQ ID NO: 173, or the amino acid sequence set forth in SEQ ID NO: 173 comprising 1, 2, or 3 amino acid variations (e.g., substitution, deletion, addition, etc.); and the amino acid sequence of CDR-L3 comprises the amino acid sequence set forth in SEQ ID NO: 174, or the amino acid sequence set forth in SEQ ID NO: 174 comprising 1, 2, or 3 amino acid variations (e.g., substitution, deletion, addition, etc.).

In some embodiments, the amino acid sequence of CDR-H1 comprises the amino acid sequence set forth in SEQ ID NO: 169, or the amino acid sequence set forth in SEQ ID NO: 169 comprising no more than 1, 2, or 3 amino acid variations (e.g., substitution, deletion, addition, etc.); the amino acid sequence of CDR-H2 comprises the amino acid sequence set forth in SEQ ID NO: 170, or the amino acid sequence set forth in SEQ ID NO: 170 comprising no more than 1, 2, or 3 amino acid variations (e.g., substitution, deletion, addition, etc.); the amino acid sequence of CDR-H3 comprises the amino acid sequence set forth in SEQ ID NO: 171, or the amino acid sequence set forth in SEQ ID NO: 171 comprising no more than 1, 2, or 3 amino acid variations (e.g., substitution, deletion, addition, etc.); the amino acid sequence of CDR-L1 comprises the amino acid sequence set forth in SEQ ID NO: 172, or the amino acid sequence set forth in SEQ ID NO: 172 comprising no more than 1, 2, or 3 amino acid variations (e.g., substitution, deletion, addition, etc.); the amino acid sequence of CDR-L2 comprises the amino acid sequence set forth in SEQ ID NO: 173, or the amino acid sequence set forth in SEQ ID NO: 173 comprising no more than 1, 2, or 3 amino acid variations (e.g., substitution, deletion, addition, etc.); and the amino acid sequence of CDR-L3 comprises the amino acid sequence set forth in SEQ ID NO: 174, or the amino acid sequence set forth in SEQ ID NO: 174 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 169, or the amino acid sequence set forth in SEQ ID NO: 169 comprising no more than 1, 2, or 3 amino acid substitutions; the amino acid sequence of CDR-H2 comprises the amino acid sequence set forth in SEQ ID NO: 170, or the amino acid sequence set forth in SEQ ID NO: 170 comprising no more than 1, 2, or 3 amino acid substitutions; the amino acid sequence of CDR-H3 comprises the amino acid sequence set forth in SEQ ID NO: 171, or the amino acid sequence set forth in SEQ ID NO: 171 comprising no more than 1, 2, or 3 amino acid substitutions; the amino acid sequence of CDR-L1 comprises the amino acid sequence set forth in SEQ ID NO: 172, or the amino acid sequence set forth in SEQ ID NO: 172 comprising no more than 1, 2, or 3 amino acid substitutions; the amino acid sequence of CDR-L2 comprises the amino acid sequence set forth in SEQ ID NO: 173, or the amino acid sequence set forth in SEQ ID NO: 173 comprising no more than 1, 2, or 3 amino acid substitutions; and the amino acid sequence of CDR-L3 comprises the amino acid sequence set forth in SEQ ID NO: 174, or the amino acid sequence set forth in SEQ ID NO: 174 comprising no more than 1, 2, or 3 amino acid substitutions.

In some embodiments, the amino acid sequence of CDR-H1 comprises the amino acid sequence set forth in SEQ ID NO: 169; the amino acid sequence of CDR-H2 comprises the amino acid sequence set forth in SEQ ID NO: 170; the amino acid sequence of CDR-H3 comprises the amino acid sequence set forth in SEQ ID NO: 171; the amino acid sequence of CDR-L1 comprises the amino acid sequence set forth in SEQ ID NO: 172; the amino acid sequence of CDR-L2 comprises the amino acid sequence set forth in SEQ ID NO: 173; and the amino acid sequence of CDR-L3 comprises the amino acid sequence set forth in SEQ ID NO: 174.

In some embodiments, the amino acid sequence of CDR-H1 comprises the amino acid sequence set forth in SEQ ID NO: 169 comprising no more than 1, 2, or 3 amino acid variations (e.g., substitution, deletion, addition, etc.); the amino acid sequence of CDR-H2 comprises the amino acid sequence set forth in SEQ ID NO: 170 comprising no more than 1, 2, or 3 amino acid variations (e.g., substitution, deletion, addition, etc.); the amino acid sequence of CDR-H3 comprises the amino acid sequence set forth in SEQ ID NO: 171 comprising no more than 1, 2, or 3 amino acid variations (e.g., substitution, deletion, addition, etc.); the amino acid sequence of CDR-L1 comprises the amino acid sequence set forth in SEQ ID NO: 172 comprising no more than 1, 2, or 3 amino acid variations (e.g., substitution, deletion, addition, etc.); the amino acid sequence of CDR-L2 comprises the amino acid sequence set forth in SEQ ID NO: 173 comprising no more than 1, 2, or 3 amino acid variations (e.g., substitution, deletion, addition, etc.); and the amino acid sequence of CDR-L3 comprises the amino acid sequence set forth in SEQ ID NO: 174 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 169 comprising no more than 1, 2, or 3 amino acid substitutions; the amino acid sequence of CDR-H2 comprises the amino acid sequence set forth in SEQ ID NO: 170 comprising no more than 1, 2, or 3 amino acid substitutions; the amino acid sequence of CDR-H3 comprises the amino acid sequence set forth in SEQ ID NO: 171 comprising no more than 1, 2, or 3 amino acid substitutions; the amino acid sequence of CDR-L1 comprises the amino acid sequence set forth in SEQ ID NO: 172 comprising no more than 1, 2, or 3 amino acid substitutions; the amino acid sequence of CDR-L2 comprises the amino acid sequence set forth in SEQ ID NO: 173 comprising no more than 1, 2, or 3 amino acid substitutions; and the amino acid sequence of CDR-L3 comprises the amino acid sequence set forth in SEQ ID NO: 174 comprising no more than 1, 2, or 3 amino acid substitutions.

In some embodiments, the amino acid sequence of CDR-H1 consists of the amino acid sequence set forth in SEQ ID NO: 169, or the amino acid sequence set forth in SEQ ID NO: 169 comprising 1, 2, or 3 amino acid variations (e.g., substitution, deletion, addition, etc.); the amino acid sequence of CDR-H2 consists of the amino acid sequence set forth in SEQ ID NO: 170, or the amino acid sequence set forth in SEQ ID NO: 170 comprising 1, 2, or 3 amino acid variations (e.g., substitution, deletion, addition, etc.); the amino acid sequence of CDR-H3 consists of the amino acid sequence set forth in SEQ ID NO: 171, or the amino acid sequence set forth in SEQ ID NO: 171 comprising 1, 2, or 3 amino acid variations (e.g., substitution, deletion, addition, etc.); the amino acid sequence of CDR-L1 consists of the amino acid sequence set forth in SEQ ID NO: 172, or the amino acid sequence set forth in SEQ ID NO: 172 comprising 1, 2, or 3 amino acid variations (e.g., substitution, deletion, addition, etc.); the amino acid sequence of CDR-L2 consists of the amino acid sequence set forth in SEQ ID NO: 173, or the amino acid sequence set forth in SEQ ID NO: 173 comprising 1, 2, or 3 amino acid variations (e.g., substitution, deletion, addition, etc.); and the amino acid sequence of CDR-L3 consists of the amino acid sequence set forth in SEQ ID NO: 174, or the amino acid sequence set forth in SEQ ID NO: 174 comprising 1, 2, or 3 amino acid variations (e.g., substitution, deletion, addition, etc.).

In some embodiments, the amino acid sequence of CDR-H1 consists of the amino acid sequence set forth in SEQ ID NO: 169, or the amino acid sequence set forth in SEQ ID NO: 169 comprising no more than 1, 2, or 3 amino acid variations (e.g., substitution, deletion, addition, etc.); the amino acid sequence of CDR-H2 consists of the amino acid sequence set forth in SEQ ID NO: 170, or the amino acid sequence set forth in SEQ ID NO: 170 comprising no more than 1, 2, or 3 amino acid variations (e.g., substitution, deletion, addition, etc.); the amino acid sequence of CDR-H3 consists of the amino acid sequence set forth in SEQ ID NO: 171, or the amino acid sequence set forth in SEQ ID NO: 171 comprising no more than 1, 2, or 3 amino acid variations (e.g., substitution, deletion, addition, etc.); the amino acid sequence of CDR-L1 consists of the amino acid sequence set forth in SEQ ID NO: 172, or the amino acid sequence set forth in SEQ ID NO: 172 comprising no more than 1, 2, or 3 amino acid variations (e.g., substitution, deletion, addition, etc.); the amino acid sequence of CDR-L2 consists of the amino acid sequence set forth in SEQ ID NO: 173, or the amino acid sequence set forth in SEQ ID NO: 173 comprising no more than 1, 2, or 3 amino acid variations (e.g., substitution, deletion, addition, etc.); and the amino acid sequence of CDR-L3 consists of the amino acid sequence set forth in SEQ ID NO: 174, or the amino acid sequence set forth in SEQ ID NO: 174 comprising no more than 1, 2, or 3 amino acid variations (e.g., substitution, deletion, addition, etc.).

In some embodiments, the amino acid sequence of CDR-H1 consists of the amino acid sequence set forth in SEQ ID NO: 169, or the amino acid sequence set forth in SEQ ID NO: 169 comprising no more than 1, 2, or 3 amino acid substitutions; the amino acid sequence of CDR-H2 consists of the amino acid sequence set forth in SEQ ID NO: 170, or the amino acid sequence set forth in SEQ ID NO: 170 comprising no more than 1, 2, or 3 amino acid substitutions; the amino acid sequence of CDR-H3 consists of the amino acid sequence set forth in SEQ ID NO: 171, or the amino acid sequence set forth in SEQ ID NO: 171 comprising no more than 1, 2, or 3 amino acid substitutions; the amino acid sequence of CDR-L1 consists of the amino acid sequence set forth in SEQ ID NO: 172, or the amino acid sequence set forth in SEQ ID NO: 172 comprising no more than 1, 2, or 3 amino acid substitutions; the amino acid sequence of CDR-L2 consists of the amino acid sequence set forth in SEQ ID NO: 173, or the amino acid sequence set forth in SEQ ID NO: 173 comprising no more than 1, 2, or 3 amino acid substitutions; and the amino acid sequence of CDR-L3 consists of the amino acid sequence set forth in SEQ ID NO: 174, or the amino acid sequence set forth in SEQ ID NO: 174 comprising no more than 1, 2, or 3 amino acid substitutions.

In some embodiments, the amino acid sequence of CDR-H1 consists of the amino acid sequence set forth in SEQ ID NO: 169; the amino acid sequence of CDR-H2 consists of the amino acid sequence set forth in SEQ ID NO: 170; the amino acid sequence of CDR-H3 consists of the amino acid sequence set forth in SEQ ID NO: 171; the amino acid sequence of CDR-L1 consists of the amino acid sequence set forth in SEQ ID NO: 172; the amino acid sequence of CDR-L2 consists of the amino acid sequence set forth in SEQ ID NO: 173; and the amino acid sequence of CDR-L3 consists of the amino acid sequence set forth in SEQ ID NO: 174.

In some embodiments, the amino acid sequence of CDR-H1 consists of the amino acid sequence set forth in SEQ ID NO: 169 comprising no more than 1, 2, or 3 amino acid variations (e.g., substitution, deletion, addition, etc.); the amino acid sequence of CDR-H2 consists of the amino acid sequence set forth in SEQ ID NO: 170 comprising no more than 1, 2, or 3 amino acid variations (e.g., substitution, deletion, addition, etc.); the amino acid sequence of CDR-H3 consists of the amino acid sequence set forth in SEQ ID NO: 171 comprising no more than 1, 2, or 3 amino acid variations (e.g., substitution, deletion, addition, etc.); the amino acid sequence of CDR-L1 consists of the amino acid sequence set forth in SEQ ID NO: 172 comprising no more than 1, 2, or 3 amino acid variations (e.g., substitution, deletion, addition, etc.); the amino acid sequence of CDR-L2 consists of the amino acid sequence set forth in SEQ ID NO: 173 comprising no more than 1, 2, or 3 amino acid variations (e.g., substitution, deletion, addition, etc.); and the amino acid sequence of CDR-L3 consists of the amino acid sequence set forth in SEQ ID NO: 174 comprising no more than 1, 2, or 3 amino acid variations (e.g., substitution, deletion, addition, etc.).

In some embodiments, the amino acid sequence of CDR-H1 consists of the amino acid sequence set forth in SEQ ID NO: 169 comprising no more than 1, 2, or 3 amino acid substitutions; the amino acid sequence of CDR-H2 consists of the amino acid sequence set forth in SEQ ID NO: 170 comprising no more than 1, 2, or 3 amino acid substitutions; the amino acid sequence of CDR-H3 consists of the amino acid sequence set forth in SEQ ID NO: 171 comprising no more than 1, 2, or 3 amino acid substitutions; the amino acid sequence of CDR-L1 consists of the amino acid sequence set forth in SEQ ID NO: 172 comprising no more than 1, 2, or 3 amino acid substitutions; the amino acid sequence of CDR-L2 consists of the amino acid sequence set forth in SEQ ID NO: 173 comprising no more than 1, 2, or 3 amino acid substitutions; and the amino acid sequence of CDR-L3 consists of the amino acid sequence set forth in SEQ ID NO: 174 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 175; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 176. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 175; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 176. In some embodiments, the amino acid sequence of the VH comprises an amino acid sequence at least 95%, 96%, 97%, 98%, 99%, or 100% identical to the amino acid sequence set forth in SEQ ID NO: 175; and the amino acid sequence of the VL comprises an amino acid sequence at least 95%, 96%, 97%, 98%, 99%, or 100% identical to the amino acid sequence set forth in SEQ ID NO: 176. In some embodiments, the amino acid sequence of the VH comprises the amino acid sequence set forth in SEQ ID NO: 175; and the amino acid sequence of the VL comprises the amino acid sequence set forth in SEQ ID NO: 176.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 175; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 176. In some embodiments, the amino acid sequence of the VH consists of an amino acid sequence at least 90%, 91%, 92%, 93%, 94%, 95%, 96%, 97%, 98%, 99%, or 100% identical to the amino acid sequence set forth in SEQ ID NO: 175; and the amino acid sequence of the VL consists of an amino acid sequence at least 90%, 91%, 92%, 93%, 94%, 95%, 96%, 97%, 98%, 99%, or 100% identical to the amino acid sequence set forth in SEQ ID NO: 176. In some embodiments, the amino acid sequence of the VH consists of an amino acid sequence at least 95%, 96%, 97%, 98%, 99%, or 100% identical to the amino acid sequence set forth in SEQ ID NO: 175; and the amino acid sequence of the VL consists of an amino acid sequence at least 95%, 96%, 97%, 98%, 99%, or 100% identical to the amino acid sequence set forth in SEQ ID NO: 176. In some embodiments, the amino acid sequence of the VH consists of the amino acid sequence set forth in SEQ ID NO: 175; and the amino acid sequence of the VL consists of the amino acid sequence set forth in SEQ ID NO: 176.

In some embodiments, the amino acid sequence of the heavy chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 177; and the amino acid sequence of the light chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 178. In some embodiments, the amino acid sequence of the heavy chain comprises an amino acid sequence at least 90%, 91%, 92%, 93%, 94%, 95%, 96%, 97%, 98%, 99%, or 100% identical to the amino acid sequence set forth in SEQ ID NO: 177; and the amino acid sequence of the light chain comprises an amino acid sequence at least 90%, 91%, 92%, 93%, 94%, 95%, 96%, 97%, 98%, 99%, or 100% identical to the amino acid sequence set forth in SEQ ID NO: 178. In some embodiments, the amino acid sequence of the heavy chain comprises an amino acid sequence at least 95%, 96%, 97%, 98%, 99%, or 100% identical to the amino acid sequence set forth in SEQ ID NO: 177; and the amino acid sequence of the light chain comprises an amino acid sequence at least 95%, 96%, 97%, 98%, 99%, or 100% identical to the amino acid sequence set forth in SEQ ID NO: 178. In some embodiments, the amino acid sequence of the heavy chain comprises the amino acid sequence set forth in SEQ ID NO: 177; and the amino acid sequence of the light chain comprises the amino acid sequence set forth in SEQ ID NO: 178.

In some embodiments, the amino acid sequence of the heavy chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 177; and the amino acid sequence of the light chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 178. In some embodiments, the amino acid sequence of the heavy chain consists of an amino acid sequence at least 90%, 91%, 92%, 93%, 94%, 95%, 96%, 97%, 98%, 99%, or 100% identical to the amino acid sequence set forth in SEQ ID NO: 177; and the amino acid sequence of the light chain consists of an amino acid sequence at least 90%, 91%, 92%, 93%, 94%, 95%, 96%, 97%, 98%, 99%, or 100% identical to the amino acid sequence set forth in SEQ ID NO: 178. In some embodiments, the amino acid sequence of the heavy chain consists of an amino acid sequence at least 95%, 96%, 97%, 98%, 99%, or 100% identical to the amino acid sequence set forth in SEQ ID NO: 177; and the amino acid sequence of the light chain consists of an amino acid sequence at least 95%, 96%, 97%, 98%, 99%, or 100% identical to the amino acid sequence set forth in SEQ ID NO: 178. In some embodiments, the amino acid sequence of the heavy chain consists of the amino acid sequence set forth in SEQ ID NO: 177; and the amino acid sequence of the light chain consists of the amino acid sequence set forth in SEQ ID NO: 178.

Additional exemplary CD161 binding proteins that (e.g., inhibit the interaction of CD161 and CLEC2D) are known in the art, see, e.g., WO2023028501, the entire contents of which are incorporated herein by reference for all purposes. For example, exemplary CD161 binding proteins that (e.g., inhibit the interaction of CD161 and CLEC2D) are set forth in Table 4 of WO2023028501, Table 4 of WO2023028501 is incorporated herein by reference for all purposes.

The amino acid sequence of additional exemplary hCD161 binding proteins (e.g., anti-hCD161 antibodies (e.g., therapeutic agents, agents that inhibit the interaction between CD161 and CLEC2D)) of the present disclosure is provided in Table 3. The CDRs of the antibodies in Table 3, are denoted according to Kabat. A person of ordinary skill in the art would be able to determine the CDRs as defined by another scheme, e.g., Chothia, IMGT, using ordinary methods known in the art.

TABLE 3
The Amino Acid Sequence of Exemplary hCD161 Binding Proteins.

| | | SEQ | Amino Acid Sequence |
|---|---|---|---|
| Description | | ID NO | |
| Ab-2 | CDR-H1 | 3 | TYAMS |
| | CDR-H2 | 4 | AISAAGGTTYYADSVKG |
| | CDR-H3 | 5 | PLDSSLWADEDL |
| | CDR-L1 | 6 | RASQGIDSWLA |
| | CDR-L2 | 7 | AASSLQS |
| | CDR-L3 | 8 | QQASVLPIT |
| | VH | 9 | EVQLLESGGGLVQPGGSLRLSCAASGFAFSTYAMSWVRQAPGKGLE |
| | | | WVSAISAAGGTTYYADSVKGRFTISRDNSKNTLYLQMNSLRAEDTA |
| | | | VYYCAKPLDSSLWADFDLWGRGTLVTVSS |
| | VL | 10 | DIQMTQSPSSVSASVGDRVTITCRASQGIDSWLAWYQQKPGKAPKL |
| | | | LIYAASSLQSGVPSRESGSGSGTDFTLTISSLQPEDFATYYCQQAS |
| | | | VLPITFGGGTKVEIK |
| Ab-3 | CDR-H1 | 3 | TYAMS |
| | CDR-H2 | 11 | AISGVGGTTYYADSVKG |
| | CDR-H3 | 5 | PLDSSLWADEDL |
| | CDR-L1 | 12 | RASQGISSWLA |
| | CDR-L2 | 13 | YASSLQD |
| | CDR-L3 | 8 | QQASVLPIT |
| | VH | 14 | EVQLLESGGGLVQPGGSLRLSCAASGFAFSTYAMSWVRQAPGKGLE |
| | | | WVSAISGVGGTTYYADSVKGRFTISRDNSKNTLYLQMNSLRAEDTA |
| | | | VYYCAKPLDSSLWADFDLWGRGTLVTVSS |
| | VL | 15 | DIQLTQSPSSVSASVGDRVTITCRASQGISSWLAWYQQKPGKAPKL |
| | | | LIYYASSLQDGVPSRFSGSGSGTDFTLTISSLQPEDFATYYCQQAS |
| | | | VLPITFGGGTKVEIK |
| Ab-4 | CDR-H1 | 16 | RYAMS |
| | CDR-H2 | 4 | AISAAGGTTYYADSVKG |
| | CDR-H3 | 17 | PLDSSQWADFDL |
| | CDR-L1 | 18 | RASQDISSWLA |
| | CDR-L2 | 19 | AASALQS |
| | CDR-L3 | 20 | QQALVLPIT |
| | VH | 21 | EVQLLESGGGLVQPGGSLRLSCAASGFTFERYAMSWVRQAPGKGLE |
| | | | WVSAISAAGGTTYYADSVKGRFTISRDNSKNTLYLQMNSLRAEDTA |
| | | | VYYCAKPLDSSQWADFDLWGRGTLVTVSS |
| | VL | 22 | DIQLTQSPSSVSASVGDRVTITCRASQDISSWLAWYQQKPGKAPKE |
| | | | LIYAASALQSGVPSRFSGSGSGTDFTLTISSLQPEDFATYYCQQAL |
| | | | VLPITFGGGTKVEIK |
| Ab-5 | CDR-H1 | 16 | RYAMS |
| | CDR-H2 | 23 | AISAVGGTTKYADSVKG |
| | CDR-H3 | 24 | PLDSSLWADEDA |
| | CDR-L1 | 12 | RASQGISSWLA |
| | CDR-L2 | 25 | AASGLQS |
| | CDR-L3 | 26 | QQASYLPIT |
| | VH | 27 | EVQLLESGGGLVQPGGSLRLSCAASGETFERYAMSWVRQAPGKGLE |
| | | | WVSAISAVGGTTKYADSVKGRFTISRDNSKNTLYLQMNSLRAEDTA |
| | | | VYYCAKPLDSSLWADEDAWGRGTLVTVSS |
| | VL | 28 | DIQLTQSPSSVSASVGDRVTITCRASQGISSWLAWYQQKPGKAPKL |
| | | | LIYAASGLQSGVPSRFSGSGSGTDFTLTISSLQPEDFATYYCQQAS |
| | | | YLPITFGGGTKVEIK |
| Ab-6 | CDR-H1 | 29 | QYAMS |
| | CDR-H2 | 30 | AISAVGGTTAYADSVKG |
| | CDR-H3 | 31 | PLDSSLWADFQL |
| | CDR-L1 | 18 | RASQDISSWLA |
| | CDR-L2 | 32 | FASSLQS |
| | CDR-L3 | 33 | QQASKLPIT |
| | VH | 34 | EVQLLESGGGLVQPGGSLRLSCAASGFTFGQYAMSWVRQAPGKGLE |
| | | | WVSAISAVGGTTAYADSVKGRFTISRDNSKNTLYLQMNSLRAEDTA |
| | | | VYYCAKPLDSSLWADFQLWGRGTLVTVSS |
| | VL | 35 | DIQLTQSPSSVSASVGDRVTITCRASQDISSWLAWYQQKPGKAPKL |
| | | | LIYFASSLQSGVPSRESGSGSGTDFTLTISSLQPEDFATYYCQQAS |
| | | | KLPITFGGGTKVEIK |
| Ab-7 | CDR-H1 | 29 | QYAMS |
| | CDR-H2 | 4 | AISAAGGTTYYADSVKG |
| | CDR-H3 | 17 | PLDSSQWADFDL |
| | CDR-L1 | 12 | RASQGISSWLA |
| | CDR-L2 | 7 | AASSLQS |
| | CDR-L3 | 36 | QQAWVLPIT |
| | VH | 37 | EVQLLESGGGLVQPGGSLRLSCAASGFTFGQYAMSWVRQAPGKGLE |
| | | | WVSAISAAGGTTYYADSVKGRFTISRDNSKNTLYLQMNSLRAEDTA |
| | | | VYYCAKPLDSSQWADFDLWGRGTLVTVSS |
| | VL | 38 | DIQLTQSPSSVSASVGDRVTITCRASQGISSWLAWYQQKPGKAPKL |
| | | | LIYAASSLQSGVPSRESGSGSGTDFTLTINSLQPEDFATYYCQQAW |
| | | | VLPITFGGGTKVEIK |
| Ab-8 | CDR-H1 | 29 | QYAMS |
| | CDR-H2 | 4 | AISAAGGTTYYADSVKG |
| | CDR-H3 | 17 | PLDSSQWADEDL |
| | CDR-L1 | 12 | RASQGISSWLA |
| | CDR-L2 | 39 | AASFLQS |
| | CDR-L3 | 8 | QQASVLPIT |
| | VH | 37 | EVQLLESGGGLVQPGGSLRLSCAASGFTFGQYAMSWVRQAPGKGLE |
| | | | WVSAISAAGGTTYYADSVKGRFTISRDNSKNTLYLQMNSLRAEDTA |
| | | | VYYCAKPLDSSQWADFDLWGRGTLVTVSS |
| | VL | 40 | DIQLTQSPSSVSASVGDRVTITCRASQGISSWLAWYQQKPGKAPKL |
| | | | LIYAASFLQSGVPSRESGSGSGTDFTLTISSLQPEDFATYYCQQAS |
| | | | VLPITFGGGTKVEIK |
| Ab-9 | CDR-H1 | 41 | TEAMS |
| | CDR-H2 | 11 | AISGVGGTTYYADSVKG |
| | CDR-H3 | 42 | PLDSSFWADEDL |
| | CDR-L1 | 43 | RASQTISSWLA |
| | CDR-L2 | 7 | AASSLQS |
| | CDR-L3 | 44 | QQQSVLPIT |
| | VH | 45 | EVQLLESGGGLVQPGGSLRLSCAASGFTFGTFAMSWVRQAPGKGLE |
| | | | WVSAISGVGGTTYYADSVKGRFTISRDNSKNTLYLQMNSLRAEDTA |
| | | | VYYCAKPLDSSFWADFDLWGRGTLVTVSS |
| | VL | 46 | DIQLTQSPSSVSASVGDRVTITCRASQTISSWLAWYQQKPGKAPKL |
| | | | LIYAASSLQSGVPSRESGSGSGTDFTLTISSLQPEDFATYYCQQQS |
| | | | VLPITFGGGTKVEIK |
| Ab-10 | CDR-H1 | 47 | PYAMS |
| | CDR-H2 | 48 | AISASGGTTYYADSVKG |
| | CDR-H3 | 42 | PLDSSFWADEDL |
| | CDR-L1 | 12 | RASQGISSWLA |
| | CDR-L2 | 7 | AASSLQS |
| | CDR-L3 | 49 | QQHSVLPIT |
| | VH | 50 | EVQLLESGGGLVQPGGSLRLSCAASGFTFSPYAMSWVRQAPGKGLE |
| | | | WVSAISASGGTTYYADSVKGRFTISRDNSKNTLYLQMNSLRAEDTA |
| | | | VYYCAKPLDSSFWADFDLWGRGTLVTVSS |
| | VL | 51 | DIQLTQSPSSVSASVGDRVTITCRASQGISSWLAWYQQKPGKAPKL |
| | | | LIYAASSLQSGVPSRESGSGSGTDETLTISSLQPEDFATYYCQQHS |
| | | | VLPITFGGGTKVEIK |
| Ab-11 | CDR-H1 | 29 | QYAMS |
| | CDR-H2 | 52 | AISAVGGSTYYADSVKG |
| | CDR-H3 | 17 | PLDSSQWADEDL |
| | CDR-L1 | 18 | RASQDISSWLA |
| | CDR-L2 | 19 | AASALQS |
| | CDR-L3 | 53 | QQADVLPIT |
| | VH | 54 | EVQLLESGGGLVQPGGSLRLSCAASGFTFSQYAMSWVRQAPGKGLE |
| | | | WVSAISAVGGSTYYADSVKGRFTISRDNSKNTLYLQMNSLRAEDTA |
| | | | VYYCAKPLDSSQWADFDLWGRGTLVTVSS |
| | VL | 55 | DIQLTQSPSSVSASVGDRVTITCRASQDISSWLAWYQQKPGKAPKL |
| | | | LIYAASALQSGVPSRESGSGSGTDFTLTISSLQPEDFATYYCQQAD |
| | | | VLPITFGGGTKVEIK |
| Ab-12 | CDR-H1 | 29 | QYAMS |
| | CDR-H2 | 4 | AISAAGGTTYYADSVKG |
| | CDR-H3 | 17 | PLDSSQWADEDL |
| | CDR-L1 | 56 | RASQGIYSWLA |
| | CDR-L2 | 7 | AASSLQS |
| | CDR-L3 | 57 | QQASDLPIT |
| | VH | 58 | EVQLLESGGGLVQPGGSLRLSCAASGFTFSQYAMSWVRQAPGKGLE |
| | | | WVSAISAAGGTTYYADSVKGRFTISRDNSKNTLYLQMNSLRAEDTA |
| | | | VYYCAKPLDSSQWADFDLWGRGTLVTVSS |
| | VL | 59 | DIQLTQSPSSVSASVGDRVTITCRASQGIYSWLAWYQQKPGKAPKL |
| | | | LIYAASSLQSGVPSRESGSGSGTDFTLTISSLQPEDFATYYCQQAS |
| | | | DLPITFGGGTKVEIK |
| Ab-13 | CDR-H1 | 60 | QYYMS |
| | CDR-H2 | 61 | YISPSGSTIAYADSVKG |
| | CDR-H3 | 62 | SLMATGTHLYFDL |
| | CDR-L1 | 18 | RASQDISSWLA |
| | CDR-L2 | 7 | AASSLQS |
| | CDR-L3 | 63 | QQVTSFPPYT |
| | VH | 64 | QVQLVESGGGLVKPGGSLRLSCAASGFTFAQYYMSWIRQAPGKGLE |
| | | | WVSYISPSGSTIAYADSVKGRFTISRDNAKNSLYLQMNSLRAEDTA |
| | | | VYYCARSLMATGTHLYFDLWGRGTLVTVSS |
| | VL | 65 | DIQLTQSPSSVSASVGDRVTITCRASQDISSWLAWYQQKPGKAPKL |
| | | | LIYAASSLQSGVPSRFSGSGSGTDFTLTISSLQPEDFATYYCQQVT |
| | | | SFPPYTFGGGTKVEIK |
| Ab-14 | CDR-H1 | 66 | NYYMS |
| | CDR-H2 | 67 | YISPSGATIAYADSVKG |
| | CDR-H3 | 62 | SLMATGTHLYFDL |
| | CDR-L1 | 68 | RASSGISSWLA |
| | CDR-L2 | 69 | AASELQS |
| | CDR-L3 | 70 | QQATSFPPYT |
| | VH | 71 | QVQLVESGGGLVKPGGSLRLSCAASGFTFANYYMSWIRQAPGKGLE |
| | | | WVSYISPSGATIAYADSVKGRFTISRDNAKNSLYLQMNSLRAEDTA |
| | | | VYYCARSLMATGTHLYFDLWGRGTLVTVSS |
| | VL | 72 | DIQLTQSPSSVSASVGDRVTITCRASSGISSWLAWYQQKPGKAPKL |
| | | | LIYAASELQSGVPSRESGSGSGTDFTLTISSLQPEDFATYYCQQAT |
| | | | SFPPYTFGGGTKVEIK |
| Ab-15 | CDR-H1 | 60 | QYYMS |
| | CDR-H2 | 67 | YISPSGATIAYADSVKG |
| | CDR-H3 | 73 | SLMSTGTHLYFDL |
| | CDR-L1 | 74 | RASQGISDWLA |
| | CDR-L2 | 7 | AASSLQS |
| | CDR-L3 | 63 | QQVTSFPPYT |
| | VH | 75 | QVQLVESGGGLVKPGGSLRLSCAASGFTFGQYYMSWIRQAPGKGLE |
| | | | WVSYISPSGATIAYADSVKGRETISRDNAKNSLYLQMNSLRAEDTA |
| | | | VYYCARSLMSTGTHLYFDLWGRGTLVTVSS |
| | VL | 76 | DIQLTQSPSSVSASVGDRVTITCRASQGISDWLAWYQQKPGKAPKL |
| | | | LIYAASSLQSGVPSRFSGSGSGTDFTLTISSLQPEDFATYYCQQVT |
| | | | SFPPYTFGGGTKVEIK |
| Ab-16 | CDR-H1 | 60 | QYYMS |
| | CDR-H2 | 77 | YISPSGATIYYADSVKG |
| | CDR-H3 | 73 | SLMSTGTHLYFDL |
| | CDR-L1 | 12 | RASQGISSWLA |
| | CDR-L2 | 7 | AASSLQS |
| | CDR-L3 | 78 | QQVTSTPPYT |
| | VH | 79 | QVQLVESGGGLVKPGGSLRLSCAASGFTFPQYYMSWIRQAPGKGLE |
| | | | WVSYISPSGATIYYADSVKGRFTISRDNAKNSLYLQMNSLRAEDTA |
| | | | VYYCARSLMSTGTHLYFDLWGRGTLVTVSS |
| | VL | 80 | DIQLTQSPSSVSASVGDRVTITCRASQGISSWLAWYQQKPGKAPKL |
| | | | LIYAASSLQSGVPSRESGSGSGTDFTLTISSLQPEDFATYYCQQVT |
| | | | STPPYTFGGGTKVEIK |
| Ab-17 | CDR-H1 | 81 | DYYMS |
| | CDR-H2 | 67 | YISPSGATIAYADSVKG |
| | CDR-H3 | 62 | SLMATGTHLYFDL |
| | CDR-L1 | 12 | RASQGISSWLA |
| | CDR-L2 | 25 | AASGLQS |
| | CDR-L3 | 82 | QQSTSFPPYT |
| | VH | 83 | QVQLVESGGGLVKPGGSLRLSCAASGFTFSDYYMSWIRQAPGKGLE |
| | | | WVSYISPSGATIAYADSVKGRFTISRDNSKNTLYLQMNSLRAEDTA |
| | | | VYYCARSLMATGTHLYFDLWGRGTLVTVSS |
| | VL | 84 | DIQLTQSPSSVSASVGDRVTITCRASQGISSWLAWYQQKPGKAPKL |
| | | | LIYAASGLQSGVPSRESGSGSGTDFTLTISSLQPEDFATYYCQQST |
| | | | SFPPYTFGGGTKVEIK |
| Ab-18 | CDR-H1 | 81 | DYYMS |
| | CDR-H2 | 77 | YISPSGATIYYADSVKG |
| | CDR-H3 | 73 | SLMSTGTHLYFDL |
| | CDR-L1 | 12 | RASQGISSWLA |
| | CDR-L2 | 7 | AASSLQS |
| | CDR-L3 | 85 | QQVTSVPPYT |
| | VH | 86 | QVQLVESGGGLVKPGGSLRLSCAASGFTFSDYYMSWIRQAPGKGLE |
| | | | WVSYISPSGATIYYADSVKGRFTISRDNAKNSLYLQMNSLRAEDTA |
| | | | VYYCARSLMSTGTHLYFDLWGRGTLVTVSS |
| | VL | 87 | DIQLTQSPSSVSASVGDRVTITCRASQGISSWLAWYQQKPGKAPKL |
| | | | LIYAASSLQSGVPSRESGSGSGTDFTLTISSLQPEDFATYYCQQVT |
| | | | SVPPYTFGGGTKVEIK |
| Ab-19 | CDR-H1 | 60 | QYYMS |
| | CDR-H2 | 67 | YISPSGATIAYADSVKG |
| | CDR-H3 | 62 | SLMATGTHLYFDL |
| | CDR-L1 | 12 | RASQGISSWLA |
| | CDR-L2 | 88 | AAESLQS |
| | CDR-L3 | 89 | QQVTSQPPYT |
| | VH | 90 | QVQLVESGGGLVKPGGSLRLSCAASGFTFSQYYMSWIRQAPGKGLE |
| | | | WVSYISPSGATIAYADSVKGRFTISRDNAKNSLYLQMNSLRAEDTA |
| | | | VYYCARSLMATGTHLYFDLWGRGTLVTVSS |
| | VL | 91 | DIQLTQSPSSVSASVGDRVTITCRASQGISSWLAWYQQKPGKAPKL |
| | | | LIYAAESLQSGVPSRESGSGSGTDFTLTISSLQPEDFATYYCQQVT |
| | | | SQPPYTFGGGTKVEIK |
| Ab-20 | CDR-H1 | 60 | QYYMS |
| | CDR-H2 | 77 | YISPSGATIYYADSVKG |
| | CDR-H3 | 62 | SLMATGTHLYFDL |
| | CDR-L1 | 12 | RASQGISSWLA |
| | CDR-L2 | 19 | AASALQS |
| | CDR-L3 | 92 | QQVTSAPPYT |
| | VH | 93 | QVQLVESGGGLVKPGGSLRLSCAASGFTFSQYYMSWIRQAPGKGLE |
| | | | WVSYISPSGATIYYADSVKGRFTISRDNAKNSLYLQMNSLRAEDTA |
| | | | VYYCARSLMATGTHLYFDLWGRGTLVTVSS |
| | VL | 94 | DIQLTQSPSSVSASVGDRVTITCRASQGISSWLAWYQQKPGKAPKL |
| | | | LIYAASALQSGVPSRESGSGSGTDFTLTISSLQPEDFATYYCQQVT |
| | | | SAPPYTFGGGTKVEIK |
| Ab-21 | CDR-H1 | 60 | QYYMS |
| | CDR-H2 | 67 | YISPSGATIAYADSVKG |
| | CDR-H3 | 73 | SLMSTGTHLYFDL |
| | CDR-L1 | 12 | RASQGISSWLA |
| | CDR-L2 | 95 | AASVLQS |
| | CDR-L3 | 96 | QQVTSFLPYT |
| | VH | 97 | QVQLVESGGGLVQPGGSLRLSCAASGFTFSQYYMSWIRQAPGKGLE |
| | | | WVSYISPSGATIAYADSVKGRFTISRDNAKNSLYLQMNSLRAEDTA |
| | | | VYYCARSLMSTGTHLYFDLWGRGTLVTVSS |
| | VL | 98 | DIQLTQSPSSVSASVGDRVTITCRASQGISSWLAWYQQKPGKAPKL |
| | | | LIYAASVLQSGVPSRESGSGSGTDFTLTISSLQPEDFATYYCQQVT |
| | | | SFLPYTFGGGTKVEIK |

### 5.3 Exemplary Properties of CD161 Binding Proteins and Fusions and Conjugates Thereof

### 5.3.1 Affinity of CD161 Binding Proteins

In some embodiments, the CD161 binding protein (e.g., hCD161 binding protein) (e.g., described herein)) exhibits a dissociation constant (Kd) for CD161 (e.g., hCD161) binding of <10 μM, <100 μM, <10 μM, <1 μM, <100 nM, <10 nM, <1 nM, <0.1 nM, <0.01 nM, or <0.001 nM, and/or >0.01 μM, 0.1 μM, or 1 μM (e.g., 10-5 μM or less, 10-6 M or less, 10-8 μM or less, e.g., from 1 μM to 10 μM, e.g., from 0.1 μM to 10 μM, e.g., from 10-6 M to 10-9 μM, e.g., from 10-8 M to 10-13 μM, e.g., from 10-9 μM to 10-13 M) (e.g., as measured by SPR).

In some embodiments, the CD161 binding protein (e.g., hCD161 binding protein) (e.g., described herein)) exhibits a Kd for CD161 (e.g., hCD161) binding of <1 nM (e.g., as measured by SPR). In some embodiments, the CD161 binding protein (e.g., hCD161 binding protein) (e.g., described herein)) exhibits a Kd for CD161 (e.g., hCD161) binding of less than 1 nM (e.g., as measured by SPR). In some embodiments, the CD161 binding protein (e.g., hCD161 binding protein) (e.g., described herein)) exhibits a Kd for CD161 (e.g., hCD161) binding of from about 0.5 nM-1 nM (e.g., as measured by SPR).

In some embodiments, the CD161 binding protein ((e.g., hCD161 binding protein) (e.g., described herein)) exhibits a Kd for CD161 (e.g., hCD161) binding of <1 nM. In some embodiments, the CD161 binding protein (e.g., hCD161 binding protein) (e.g., described herein)) exhibits a Kd for CD161 (e.g., hCD161) binding of less than 1 nM. In some embodiments, the CD161 binding protein (e.g., hCD161 binding protein) (e.g., described herein)) exhibits a Kd for CD161 (e.g., hCD161) binding of from about 0.5 nM-1 nM.

Binding affinity can be measured by standard assays known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip. In some embodiments, the affinity of a CD161 binding protein for CD161 (e.g., KD) is measured by SPR.

Other suitable assays for measuring the binding affinity include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.3.2 Inhibition of CD161 Binding to CLEC2D

In some embodiments, the CD161 binding protein (e.g., hCD161 binding protein) (e.g., described herein)) inhibits (e.g., partially, substantially, or fully) binding of CD161 to CLEC2D. In some embodiments, the CD161 binding protein (e.g., hCD161 binding protein) (e.g., described herein)) partially inhibits binding of CD161 to CLEC2D. In some embodiments, the CD161 binding protein (e.g., hCD161 binding protein) (e.g., described herein)) substantially inhibits binding of CD161 to CLEC2D. In some embodiments, the CD161 binding protein (e.g., hCD161 binding protein) (e.g., described herein)) fully inhibits binding of CD161 to CLEC2D.

In some embodiments, the CD161 binding protein (e.g., hCD161 binding protein) (e.g., described herein)) inhibits (e.g., partially, substantially, or fully) binding of hCD161 to hCLEC2D. In some embodiments, the CD161 binding protein (e.g., hCD161 binding protein) (e.g., described herein)) partially inhibits binding of hCD161 to hCLEC2D. In some embodiments, the CD161 binding protein (e.g., hCD161 binding protein) (e.g., described herein)) substantially inhibits binding of hCD161 to hCLEC2D. In some embodiments, the CD161 binding protein (e.g., hCD161 binding protein) (e.g., described herein)) fully inhibits binding of hCD161 to hCLEC2D.

In some embodiments, the CD161 binding protein (e.g., hCD161 binding protein) (e.g., described herein)) inhibits (e.g., partially, substantially, or fully) binding of CD161 to CLEC2D and exhibits an IC50 of <10 μM, <100 μM, <10 μM, <1 μM, <100 nM, <10 nM, <1 nM, <0.1 nM, <0.01 nM, or <0.001 nM, and/or >0.01 μM, 0.1 μM, or 1 μM (e.g., 10-5 μM or less, 10-6 M or less, 10-8 μM or less, e.g., from 1 μM to 10 μM, e.g., from 0.1 μM to 10 μM, e.g., from 10-6 M to 10-9 μM, e.g., from 10-8 μM to 10-13 μM, e.g., from 10-9 μM to 10-13 M) (e.g., as measured by SPR).

In some embodiments, the (e.g., hCD161 binding protein) (e.g., described herein)) inhibits (e.g., partially, substantially, or fully) binding of CD161 to CLEC2D and exhibits an IC50 of <10 nM. In some embodiments, the CD161 binding protein (e.g., hCD161 binding protein) (e.g., described herein)) inhibits (e.g., partially, substantially, or fully) binding of CD161 to CLEC2D and exhibits an IC50 of less than 5 nM. In some embodiments, the CD161 binding protein (e.g., hCD161 binding protein) (e.g., described herein)) inhibits (e.g., partially, substantially, or fully) binding of CD161 to CLEC2D and exhibits an IC50 of less than 2 nM. In some embodiments, the CD161 binding protein (e.g., hCD161 binding protein) (e.g., described herein)) exhibits a Kd for CD161 (e.g., hCD161) binding of from about 0.5 nM-2 nM.

In some embodiments, the CD161 binding protein (e.g., hCD161 binding protein) (e.g., described herein)) inhibits (e.g., partially, substantially, or fully) binding of CD161 to CLEC2D and exhibits an IC50 of <10 nM. In some embodiments, the CD161 binding protein (e.g., hCD161 binding protein) (e.g., described herein)) inhibits (e.g., partially, substantially, or fully) binding of CD161 to CLEC2D and exhibits an IC50 of less than 5 nM (e.g., as measured by SPR). In some embodiments, the CD161 binding protein (e.g., hCD161 binding protein) (e.g., described herein)) inhibits (e.g., partially, substantially, or fully) binding of CD161 to CLEC2D and exhibits an IC50 of less than 2 nM. In some embodiments, the CD161 binding protein (e.g., hCD161 binding protein) (e.g., described herein)) exhibits a Kd for CD161 (e.g., hCD161) binding of from about 0.5 nM-2 nM.

Standard binding assays to measure binding of CD161 to CLEC2D are known in the art and described herein. See, e.g., Wade M, Méndez J, Coussens N P, et al. Inhibition of Protein-Protein Interactions: Cell-Based Assays. 2017 Nov. 20. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK464632/; Arkin M R, Glicksman M A, Fu H, et al. Inhibition of Protein-Protein Interactions: Non-Cellular Assay Formats. 2012 Mar. 18 [Updated 2012 Oct. 1]. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK92000/; the entire contents of each of which are incorporated herein by reference for all purposes. For example, non-cell-based assays include, but are not limited to, ELISA. For further example, cell-based assays include, but are not limited to, energy transfer (Förster resonance energy transfer and bioluminescence resonance energy transfer) and protein complementation (fluorescence or enzymatic, e.g., luciferase).

### 5.4 Conjugates and Fusion Proteins

In some embodiments, a CD161 binding protein described herein is operably connected to a heterologous moiety (e.g., a heterologous protein) forming a conjugate or a fusion protein.

In some embodiments, the CD161 binding protein comprises 1, 2, 3, 4, or 5 or more heterologous moieties. In some embodiments, the CD161 binding protein comprises at least 1, 2, 3, 4, or 5 or more heterologous moieties. In some embodiments, CD161 binding protein comprises no more than 1, 2, 3, 4, or 5 or more heterologous moieties.

In some embodiments, the heterologous moiety is a peptide, polypeptide, protein, carbohydrate, nucleic acid molecule (e.g., DNA, RNA), lipid, polymer, or small molecule. In some embodiments, the heterologous moiety is a therapeutic agent. In some embodiments, the heterologous moiety is a targeting agent. In some embodiments, the heterologous moiety is a cytotoxic agent.

In some embodiments, the heterologous moiety is a protein, peptide, polynucleotide (e.g., DNA, RNA, DNA/RNA hybrid), small molecule, carbohydrate, lipid, or synthetic polymer.

In some embodiments, the heterologous protein comprises an IgG (e.g., IgG1, IgG2 (e.g., IgG2a or IgG2b), IgG3, IgG4), IgE, IgM, IgD, or IgA (e.g., IgA1 or IgA2) antibody. In some embodiments, the heterologous polypeptide is an antibody (e.g., a full-length antibody, scFv, Fab, F(ab′)2, Fab′, Fv, single domain antibody (e.g., a VHH), scFv-Fc, Fab-Fc, and/or single domain antibody-Fc (e.g., VHH-Fc)). In some embodiments, the heterologous protein comprises one or more of a monoclonal antibody, monospecific antibody, multispecific antibody, human antibody, humanized antibody, chimeric antibody, and/or murine antibody, or a functional fragment or functional variant of any of the foregoing. In some embodiments, the antibody is a chimeric antigen receptor (e.g., described herein). In some embodiments, the antibody is a T-cell engager (e.g., described herein).

In some embodiments, the heterologous moiety is a therapeutic agent. In some embodiments, the heterologous moiety is a chemotherapeutic agent, a cytotoxic agent, an anti-cancer agent, or a radioactive isotope, or any combination thereof. In some embodiments, the heterologous moiety is a targeting moiety.

In some embodiments, the heterologous moiety is a detectable moiety. In some embodiments, the heterologous moiety is a diagnostic moiety (e.g., a fluorescent protein, tag, or reporter). In some embodiments, the heterologous moiety is a fluorescent moiety. Fluorescent or chemiluminescent labels include fluorophores such as rare earth chelates, fluorescein and its derivatives, rhodamine and its derivatives, isothiocyanate, phycoerythrin, phycocyanin, allophycocyanin, o-phthaladehyde, fluorescamine, 152Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

In some embodiments, the heterologous moiety is a half-life extension moiety. Exemplary half-life extension moieties include, but are not limited to, a human immunoglobulin (Ig), a fragment of an Ig, an Ig constant region (e.g., one or more Ig heavy chain constant region), a fragment of an Ig constant region (e.g., an Ig heavy chain constant region), an Ig Fc region, human transferrin, human serum albumin (HSA), an HSA binding protein or peptide, and polyethylene glycol (PEG) (and polymers thereof). In some embodiments, the heterologous polypeptide is a half-life extension polypeptide. Exemplary half-life extension polypeptides include, but are not limited to, an Ig, a fragment of an Ig (e.g., an Ig heavy chain constant region), one or more Ig heavy chain constant region (e.g., one or more Ig heavy chain constant region), a fragment of an Ig constant region (e.g., an Ig heavy chain constant region), an Ig Fc region, human transferrin, human serum albumin (HSA), and an HSA binding protein or peptide. The CD161 binding protein described herein fused or conjugated to a half-life extending moiety or a half-life extending moiety can be evaluated for their pharmacokinetic properties utilizing standard in vivo methods known in the art.

### 5.4.1 Conjugates

In one aspect, provided herein are conjugates comprising a CD161 binding protein described herein and a heterologous moiety. In some embodiments, the heterologous moiety is a peptide, polypeptide, protein, nucleic acid molecule (e.g., DNA, RNA), carbohydrate, lipid, polymer, or small molecule. In some embodiments, the heterologous moiety is a nucleic acid molecule (e.g., DNA, RNA), carbohydrate, lipid, polymer, or small molecule.

In some embodiments, the heterologous moiety is a therapeutic agent. In some embodiments, the heterologous moiety is a chemotherapeutic agent, a cytotoxic agent, an anti-cancer agent, or a radioactive isotope, or any combination thereof. In some embodiments, the heterologous moiety is a targeting moiety. In some embodiments, the heterologous moiety is a detectable moiety. In some embodiments, the heterologous moiety is a diagnostic moiety. In some embodiments, the heterologous moiety is a fluorescent moiety. Fluorescent or chemiluminescent labels include fluorophores such as rare earth chelates, fluorescein and its derivatives, rhodamine and its derivatives, isothiocyanate, phycoerythrin, phycocyanin, allophycocyanin, o-phthaladehyde, fluorescamine, 152Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

### 5.4.2 Fusion Proteins

In one aspect, provided herein are fusion proteins comprising a CD161 binding protein (e.g., described herein) and a heterologous protein. In some embodiments, the heterologous protein is a therapeutic agent. In some embodiments, the heterologous protein is a targeting agent. In some embodiments, the heterologous protein is a detectable protein. In some embodiments, the heterologous protein is a diagnostic protein. In some embodiments, the heterologous protein is a fluorescent protein.

In some embodiments, the heterologous protein comprises an IgG (e.g., IgG1, IgG2 (e.g., IgG2a or IgG2b), IgG3, IgG4), IgE, IgM, IgD, or IgA (e.g., IgA1 or IgA2) antibody. In some embodiments, the heterologous polypeptide is an antibody (e.g., a full-length antibody, scFv, Fab, F(ab′)2, Fab′, Fv, single domain antibody (e.g., a VHH), scFv-Fc, Fab-Fc, and/or single domain antibody-Fc (e.g., VHH-Fc)). In some embodiments, the heterologous protein comprises one or more of a monoclonal antibody, monospecific antibody, multispecific antibody, human antibody, humanized antibody, chimeric antibody, and/or murine antibody, or a functional fragment or functional variant of any of the foregoing.

### 5.4.3 Linkers

As described herein, the heterologous moiety of conjugates and fusion proteins and polypeptides described herein can be directly operably connected or indirectly operably connected to the CD161 binding protein (e.g., described herein).

In some embodiments, the heterologous moiety of conjugates and fusion proteins and polypeptides described herein is directly operably connected to the CD161 binding protein (e.g., described herein). In some embodiments, the heterologous moiety of conjugates and fusion proteins and polypeptides described herein is indirectly operably connected to the CD161 binding protein (e.g., described herein). In some embodiments, the heterologous moiety of conjugates and fusion proteins and polypeptides described herein is indirectly operably connected to the CD161 binding protein (e.g., described herein) through a linker.

Linkers can be peptide linkers or non-peptide linkers. In some embodiments, the linker is derived from a crosslinking reagent. In some embodiments, the linker is cleavable. In some embodiments, the linker is non-cleavable. In some embodiments, the linker is synthetic.

### 5.4.3.1 Non-Peptide Linkers

In some embodiments, the linker is a non-peptide linker. In some embodiments, the linker is a chemical linker. In some embodiments, the linker is synthetic. In some embodiments, the linker is derived from a crosslinking reagent. In some embodiments, the linker is cleavable. In some embodiments, the linker is non-cleavable.

A variety of non-peptide linkers are known in the art that can be utilized to conjugate a heterologous moiety to a CD161 binding protein described herein. Suitable linkers have two reactive termini, one for conjugation to a CD161 binding protein described herein and one for conjugation to the heterologous moiety.

The terminus of the linker that is conjugated to a protein CD161 binding protein described herein can be a site that is capable of conjugation to the protein through a cysteine thiol or lysine amine group on the protein, and as such comprises a thiol reactive group (e.g., as in maleimide, acrylamide, vinyl sulfone, bromide and tosylate linkers) or an amine-reactive group. The cysteine thiol or lysine amine group can be naturally occurring or engineered (e.g., proteins comprising engineered cysteine or lysine amino acid residues).

In some embodiments, the linker comprises one or more of polyethylene glycol (PEG), maleimide, acrylamide, vinyl sulfone, bromide or tosylate. In some embodiments, the linker is derived from a crosslinking reagent such as N-succinimidyl-3-(2-pyridyldithio)propionate (SPDP), N-succinimidyl 4-(2-pyridyldithio) pentanoate (SPP), N-succinimidyl 4-(2-pyridyldithio)butanoate (SPDB), N-succinimidyl-4-(2-pyridyldithio)-2-sulfo-butanoate (sulfo-SPDB), N-succinimidyl iodoacetate (SIA), N-succinimidyl (4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1 H-pyrrol-1-yl)-5,8,11,14-tetraoxo-4,7,10,13-tetraazaheptadecan-1-oate (CX1-1).

Exemplary linkers are for example described in e.g., Zheng Su et al., Antibody-drug conjugates: Recent advances in linker chemistry, Acta Pharmaceutica Sinica B, Volume 11, Issue 12, 3889-3907 (2021); and Sheyi, R.; de la Torre, B. G.; Albericio, F. Linkers: An Assurance for Controlled Delivery of Antibody-Drug Conjugate. Pharmaceutics 2022, 14, 396. https://doi.org/10.3390/pharmaceutics14020396, the entire contents of each of which is incorporated herein by reference for all purposes.

### 5.4.3.2 Peptide Linkers

In some embodiments, the heterologous moiety (e.g., heterologous protein) is directly operably connected to the CD161 binding protein (e.g., described herein) via a peptide bond. In some embodiment, the heterologous moiety is indirectly operably connected to the CD161 binding protein (e.g., described herein) via a peptide linker.

In some embodiments, the linker is a peptide linker. In some embodiments, the peptide linker is one or any combination of a cleavable linker, a non-cleavable linker, a flexible linker, a rigid linker, a helical linker, and/or a non-helical linker.

In some embodiments, the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises or consists of glycine, serine, or both glycine and serine amino acid residues. In some embodiments, the amino acid sequence of the peptide linker comprises or consists of glycine, serine, and proline amino acid residues.

The amino acid sequence of exemplary peptide linkers, which can be incorporated in one or more of the embodiments described herein (e.g., fusion proteins and polypeptides, conjugates), is set provided in Table 4.

TABLE 4
The Amino Acid Sequence of Exemplary
Peptide Linkers.

| | | SEQ |
|---|---|---|
| Descrip- | | ID |
| tion | Amino Acid Sequence | NO |
| L-1 | GGGS | 99 |
| L-2 | GGGSGGGS | 100 |
| L-3 | GGGSGGGSGGGS | 101 |
| L-4 | GGGSGGGSGGGSGGGS | 102 |
| L-5 | GGGGS | 103 |
| L-6 | GGGGSGGGGS | 104 |
| L-7 | GGGGSGGGGSGGGGS | 105 |
| L-8 | GGGGSGGGGSGGGGSGGGGS | 106 |
| L-9 | GGGGGGGS | 107 |
| L-10 | GGGGGGGSGGGGGGGS | 108 |
| L-11 | GGGGGGGSGGGGGGGSGGGGGGGS | 109 |
| L-12 | GGGGGGGSGGGGGGGGGGGGGGSG | 110 |
| | GGGGGGS | |
| L-13 | SGGGG | 111 |
| L-14 | SGGGGSGGGG | 112 |
| L-15 | SGGGGSGGGGSGGGG | 113 |
| L-16 | SGGGGSGGGGSGGGGSGGGG | 114 |
| L-17 | GGGGGGS | 115 |
| L-18 | GGGGGGSGGGGGGS | 116 |
| L-19 | GGGGGGSGGGGGGSGGGGGGS | 117 |
| L-20 | GGGGGGSGGGGGGSGGGGGGSGGGGGGS | 118 |

In some embodiments, the amino acid sequence of the peptide linker comprises or consists of the amino acid sequence of any one of the linkers set forth in Table 4. In some embodiments, the amino acid sequence of the peptide linker comprises or consists of the amino acid sequence of any one of the linkers set forth in Table 4, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of the linkers set forth in Table 4, comprising 1, 2, or 3 amino acid variations (e.g., substitutions, deletions, additions). In some embodiments, the amino acid sequence of the peptide linker comprises or consists of the amino acid sequence of any one of the linkers set forth in Table 4, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 4, comprising 1, 2, or 3 amino acid substitutions.

In some embodiments, the amino acid sequence of the peptide linker comprises or consists of the amino acid sequence of any one of SEQ ID NOS: 99-118. In some embodiments, the amino acid sequence of the peptide linker comprises or consists of the amino acid sequence of any one of SEQ ID NOS: 99-118, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of SEQ ID NOS: 99-118, comprising 1, 2, or 3 amino acid variations (e.g., substitutions, deletions, additions). In some embodiments, the amino acid sequence of the peptide linker comprises or consists of the amino acid sequence of any one of SEQ ID NOS: 99-118, and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 99-118, comprising 1, 2, or 3 amino acid substitutions.

### 5.4.4 Orientation

The heterologous moiety (e.g., heterologous polypeptide) and the CD161 binding protein (e.g., described herein) of a conjugate or fusion (e.g., described herein) can be arranged in any configuration or order as long as the CD161 binding protein (e.g., described herein) maintains the ability to mediate its function and the heterologous moiety (e.g., heterologous polypeptide) can mediate its function.

The heterologous moiety can be attached (e.g., conjugated or fused) to the N-terminus, C-terminus, or at an internal site (i.e., between the N- and C-terminus) of the CD161 binding protein (e.g., one or more polypeptide of a CD161 binding protein).

In some embodiments, the heterologous moiety is attached (e.g., conjugated or fused) to the N-terminus of the CD161 binding protein (e.g., one or more polypeptide of the CD161 binding protein) or polypeptide. In some embodiments, the heterologous moiety is attached (e.g., conjugated or fused) to the C-terminus of the CD161 binding protein (e.g., one or more polypeptide of the CD161 binding protein). In some embodiments, the heterologous moiety is attached (e.g., conjugated or fused) at an internal site (i.e., between the N- and C-terminus) of the CD161 binding protein (e.g., one or more polypeptide of the CD161 binding protein).

In some embodiments, the CD161 binding protein comprises an Fc region and the heterologous moiety is operably connected to the C-terminus of the Fc region. In some embodiments, the CD161 binding protein is a full-length antibody and the heterologous moiety is operably connected to the C-terminus of the heavy chain of the antibody.

### 5.5 Multimeric CD161 Binding Proteins

In some embodiments, the CD161 binding proteins (e.g., described herein) (or conjugates or fusions comprising the same) are multimeric (e.g., dimeric, trimeric, tetrameric, etc.) proteins comprising at least two, three, or four polypeptides.

In some embodiments, the CD161 binding protein comprises at least two, three, or four polypeptides. In some embodiments, the CD161 binding protein is dimeric (i.e., comprises two polypeptides). In some embodiments, the CD161 binding protein is trimeric (i.e., comprises three polypeptides). In some embodiments, the CD161 binding protein is tetrameric (i.e., comprises four polypeptides).

In some embodiments, two of the polypeptides associate via covalent or non-covalent interactions. In some embodiments, two of the polypeptides associate via at least one covalent interaction. In some embodiments, two of the polypeptides associate via one or more disulfide bond. In some embodiments, two of the polypeptides associate via 1, 2, 3, 4, or more disulfide bonds.

In some embodiments, the CD161 binding protein comprises a full-length antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the amino acid sequence of the first heavy chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the first light chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the VH region of the first heavy chain is 100% identical to the amino acid sequence of the VH region of the second heavy chain; and the amino acid sequence of the first heavy chain outside of the VH region is at least 85%, 86%, 87%, 88%, 89%, 90%, 91%, 92%, 93%, 94%, 95%, 96%, 97%, 98%, 99%, or 100% identical to the amino acid sequence of the second heavy chain outside of the VH region of the second heavy chain. In some embodiments, the amino acid sequence of the VL region of the first light chain is 100% identical to the amino acid sequence of the VL region of the second light chain; and the amino acid sequence of the first light chain outside of the VL region is at least 85%, 86%, 87%, 88%, 89%, 90%, 91%, 92%, 93%, 94%, 95%, 96%, 97%, 98%, 99%, or 100% identical to the amino acid sequence of the second light chain outside of the VL region of the second light chain.

### 5.6 Ig Constant Regions

In some embodiments, a CD161 binding protein described herein (or a conjugate or fusion protein comprising the same (e.g., described herein)) comprises one or more Ig (e.g., human Ig (Ig), murine Ig (mIg)) constant region (e.g., a CH1 region, a hinge region, a CH2 region, a CH3 region, an Fc region, λ, CL, κ CL).

In some embodiments, the CD161 binding protein (e.g., described herein) (or a conjugate or fusion protein comprising the same (e.g., described herein)) comprises one or more Ig (e.g., Ig, mIg) heavy chain constant region (e.g., a CH1 region, a hinge region, a CH2 region, a CH3 region, an Fc region). For example, a full-length antibody CD161 binding protein comprises a CH1 region, a hinge region, a CH2 region, and a CH3 region. Or for example, the CD161 binding protein may be fused to one or more Ig (e.g., Ig, mIg) heavy chain constant regions (e.g., a CH1 region, a hinge region, a CH2 region, a CH3 region, an Fc region).

In some embodiments, the Ig is a human IgG (IgG). In some embodiments, the IgG is IgG1, IgG2 (e.g., IgG2a or IgG2b), IgG3, or IgG4. In some embodiments, the IgG is IgG1 or IgG4. In some embodiments, the IgG is IgG1. In some embodiments, the IgG is IgG4.

In some embodiments, the CD161 binding protein (or one or more polypeptide thereof) (or a conjugate or fusion protein comprising the same (e.g., described herein)) comprises an Fc region. In some embodiments, the Fc region is part of a full-length antibody. In some embodiments, the Fc region comprises or consists of at least a portion of a hinge region, a CH2 region, and a CH3 region. In some embodiments, the Fc region comprises or consists of a hinge region, a CH2 region, and a CH3 region. In some embodiments, the Fc region comprises or consists of at least a portion of an IgG hinge region, an IgG CH2 region, and an IgG CH3 region. In some embodiments, the Fc region comprises or consists of an IgG hinge region, an IgG CH2 region, and an IgG CH3 region. In some embodiments, the Fc region comprises or consists of at least a portion of a IgG1 hinge region, a IgG1 CH2 region, and a IgG1 CH3 region. In some embodiments, the Fc region comprises or consists of a IgG1 hinge region, a IgG1 CH2 region, and a IgG1 CH3 region. In some embodiments, the Fc region comprises or consists of at least a portion of a IgG4 hinge region, a IgG4 CH2 region, and a IgG4 CH3 region. In some embodiments, the Fc region comprises or consists of a IgG4 hinge region, a IgG4 CH2 region, and a IgG4 CH3 region.

In some embodiments, the CD161 binding protein (or one or more polypeptide thereof) (e.g., described herein) (or a conjugate or fusion protein comprising the same (e.g., described herein)) comprises one or more Ig light chain constant regions (e.g., λCL, κCL). For example, a full-length antibody comprises two light chains each comprising a light chain constant region (e.g., λCL, κCL).

The amino acid sequence of exemplary reference human IgG1 and IgG4 heavy chain constant regions and Ig light chain constant regions, which can be incorporated in one or more of the embodiments described herein (e.g., CD161 binding proteins or polypeptides, conjugates, and fusion proteins and polypeptide), is provided in Table 5.

TABLE 5
The Amino Acid Sequence of Exemplary hIg Heavy and Light Chain Constant
Regions & Components.

| | | SEQ |
|---|---|---|
| Description | Amino Acid Sequence | ID NO |
| IgG1 CH1 Region | ASTKGPSVFPLAPSSKSTSGGTAALGCLVKDYFPEPVTVSWNSGA | 119 |
| | LTSGVHTFPAVLQSSGLYSLSSVVTVPSSSLGTQTYICNVNHKPS | |
| | NTKVDKKV | |
| IgG1 Hinge Region | EPKSCDKTHTCP | 120 |
| IgG1 CH2 Region | PCPAPELLGGPSVFLFPPKPKDTLMISRTPEVTCVVVDVSHEDPE | 121 |
| | VKFNWYVDGVEVHNAKTKPREEQYNSTYRVVSVLTVLHQDWLNGK | |
| | EYKCKVSNKALPAPIEKTISKAK | |
| IgG1 CH3 Region | GQPREPQVYTLPPSRDELTKNQVSLTCLVKGFYPSDIAVEWESNG | 122 |
| With C-terminal | QPENNYKTTPPVLDSDGSFFLYSKLTVDKSRWQQGNVESCSVMHE | |
| Lysine | ALHNHYTQKSLSLSPGK | |
| IgG1 CH3 Region | GQPREPQVYTLPPSRDELTKNQVSLTCLVKGFYPSDIAVEWESNG | 123 |
| Without C-terminal | QPENNYKTTPPVLDSDGSFFLYSKLTVDKSRWQQGNVESCSVMHE | |
| Lysine | ALHNHYTQKSLSLSPG | |
| IgG1 CH2 Region + | PCPAPELLGGPSVFLFPPKPKDTLMISRTPEVTCVVVDVSHEDPE | 124 |
| CH3 Region | VKFNWYVDGVEVHNAKTKPREEQYNSTYRVVSVLTVLHQDWLNGK | |
| With C-terminal | EYKCKVSNKALPAPIEKTISKAKGQPREPQVYTLPPSRDELTKNQ | |
| Lysine | VSLTCLVKGFYPSDIAVEWESNGQPENNYKTTPPVLDSDGSFFLY | |
| | SKLTVDKSRWQQGNVFSCSVMHEALHNHYTQKSLSLSPGK | |
| IgG1 CH2 Region + | PCPAPELLGGPSVFLFPPKPKDTLMISRTPEVTCVVVDVSHEDPE | 125 |
| CH3 Region | VKFNWYVDGVEVHNAKTKPREEQYNSTYRVVSVLTVLHQDWLNGK | |
| | EYKCKVSNKALPAPIEKTISKAKGQPREPQVYTLPPSRDELTKNQ | |
| Without C-terminal | VSLTCLVKGFYPSDIAVEWESNGQPENNYKTTPPVLDSDGSFFLY | |
| Lysine | SKLTVDKSRWQQGNVFSCSVMHEALHNHYTQKSLSLSPG | |
| IgG1 Partial Hinge | TCPPCPAPELLGGPSVELFPPKPKDTLMISRTPEVTCVVVDVSHE | 126 |
| Region + CH2 Region | DPEVKFNWYVDGVEVHNAKTKPREEQYNSTYRVVSVLTVLHQDWL | |
| + CH3 Region | NGKEYKCKVSNKALPAPIEKTISKAKGQPREPQVYTLPPSRDELT | |
| With C-terminal | KNQVSLTCLVKGFYPSDIAVEWESNGQPENNYKTTPPVLDSDGSF | |
| Lysine | FLYSKLTVDKSRWQQGNVFSCSVMHEALHNHYTQKSLSLSPGK | |
| IgG1 Partial Hinge | TCPPCPAPELLGGPSVFLFPPKPKDTLMISRTPEVTCVVVDVSHE | 127 |
| Region + CH2 Region | DPEVKENWYVDGVEVHNAKTKPREEQYNSTYRVVSVLTVLHQDWL | |
| + CH3 Region | NGKEYKCKVSNKALPAPIEKTISKAKGQPREPQVYTLPPSRDELT | |
| Without C-terminal | KNQVSLTCLVKGFYPSDIAVEWESNGQPENNYKTTPPVLDSDGSF | |
| Lysine | FLYSKLTVDKSRWQQGNVFSCSVMHEALHNHYTQKSLSLSPG | |
| IgG1 Hinge Region + | EPKSCDKTHTCPPCPAPELLGGPSVFLFPPKPKDTLMISRTPEVT | 128 |
| CH2 Region + CH3 | CVVVDVSHEDPEVKENWYVDGVEVHNAKTKPREEQYNSTYRVVSV | |
| Region | LTVLHQDWLNGKEYKCKVSNKALPAPIEKTISKAKGQPREPQVYT | |
| With C-terminal | LPPSRDELTKNQVSLTCLVKGFYPSDIAVEWESNGQPENNYKTTP | |
| Lysine | PVLDSDGSFFLYSKLTVDKSRWQQGNVFSCSVMHEALHNHYTQKS | |
| | LSLSPGK | |
| IgG1 Hinge Region + | EPKSCDKTHTCPPCPAPELLGGPSVELFPPKPKDTLMISRTPEVT | 129 |
| CH2 Region + CH3 | CVVVDVSHEDPEVKENWYVDGVEVHNAKTKPREEQYNSTYRVVSV | |
| Region | LTVLHQDWLNGKEYKCKVSNKALPAPIEKTISKAKGQPREPQVYT | |
| | LPPSRDELTKNQVSLTCLVKGFYPSDIAVEWESNGQPENNYKTTP | |
| Without C-terminal | PVLDSDGSFFLYSKLTVDKSRWQQGNVFSCSVMHEALHNHYTQKS | |
| Lysine | LSLSPG | |
| IgG1 CH1+ Hinge | ASTKGPSVFPLAPSSKSTSGGTAALGCLVKDYFPEPVTVSWNSGA | 130 |
| Region + CH2 Region | LTSGVHTFPAVLQSSGLYSLSSVVTVPSSSLGTQTYICNVNHKPS | |
| + CH3 Region | NTKVDKKVEPKSCDKTHTCPPCPAPELLGGPSVFLFPPKPKDTLM | |
| With C-terminal | ISRTPEVTCVVVDVSHEDPEVKENWYVDGVEVHNAKTKPREEQYN | |
| Lysine | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK | |
| IgG1 CH1 + Hinge | ASTKGPSVFPLAPSSKSTSGGTAALGCLVKDYFPEPVTVSWNSGA | 131 |
| Region + CH2 Region | LTSGVHTFPAVLQSSGLYSLSSVVTVPSSSLGTQTYICNVNHKPS | |
| + CH3 Region | NTKVDKKVEPKSCDKTHTCPPCPAPELLGGPSVFLFPPKPKDTLM | |
| Without C-terminal | ISRTPEVTCVVVDVSHEDPEVKENWYVDGVEVHNAKTKPREEQYN | |
| Lysine | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG | |
| IgG4 CH1 Region | ASTKGPSVFPLAPCSRSTSESTAALGCLVKDYFPEPVTVSWNSGA | 132 |
| | LTSGVHTFPAVLQSSGLYSLSSVVTVPSSSLGTKTYTCNVDHKPS | |
| | NTKVDKRV | |
| IgG4 Hinge Region | ESKYGPPCPSCP | 133 |
| IgG4 CH2 Region | APEFLGGPSVFLFPPKPKDTLMISRTPEVTCVVVDVSQEDPEVQF | 134 |
| | NWYVDGVEVHNAKTKPREEQFNSTYRVVSVLTVLHQDWLNGKEYK | |
| | CKVSNKGLPSSIEKTISKAK | |
| IgG4 CH3 Region | GQPREPQVYTLPPSQEEMTKNQVSLTCLVKGFYPSDIAVEWESNG | 135 |
| With C-terminal | QPENNYKTTPPVLDSDGSFFLYSRLTVDKSRWQEGNVESCSVMHE | |
| Lysine | ALHNHYTQKSLSLSLGK | |
| IgG4 CH3 Region | GQPREPQVYTLPPSQEEMTKNQVSLTCLVKGFYPSDIAVEWESNG | 136 |
| Without C-terminal | QPENNYKTTPPVLDSDGSFFLYSRLTVDKSRWQEGNVESCSVMHE | |
| Lysine | ALHNHYTQKSLSLSLG | |
| IgG4 CH2 Region + | APEFLGGPSVFLFPPKPKDTLMISRTPEVTCVVVDVSQEDPEVQF | 137 |
| CH3 Region | NWYVDGVEVHNAKTKPREEQFNSTYRVVSVLTVLHQDWLNGKEYK | |
| With C-terminal | CKVSNKGLPSSIEKTISKAKGQPREPQVYTLPPSQEEMTKNQVSL | |
| Lysine | TCLVKGFYPSDIAVEWESNGQPENNYKTTPPVLDSDGSFFLYSRL | |
| | TVDKSRWQEGNVFSCSVMHEALHNHYTQKSLSLSLGK | |
| IgG4 CH2 Region + | APEFLGGPSVELFPPKPKDTLMISRTPEVTCVVVDVSQEDPEVQF | 138 |
| CH3 Region | NWYVDGVEVHNAKTKPREEQFNSTYRVVSVLTVLHQDWLNGKEYK | |
| Without C-terminal | CKVSNKGLPSSIEKTISKAKGQPREPQVYTLPPSQEEMTKNQVSL | |
| Lysine | TCLVKGFYPSDIAVEWESNGQPENNYKTTPPVLDSDGSFFLYSRL | |
| | TVDKSRWQEGNVFSCSVMHEALHNHYTQKSLSLSLG | |
| IgG4 Partial Hinge | PCPSCPAPEFLGGPSVFLFPPKPKDTLMISRTPEVTCVVVDVSQE | 139 |
| Region + CH2 Region | DPEVQFNWYVDGVEVHNAKTKPREEQFNSTYRVVSVLTVLHQDWL | |
| + CH3 Region | NGKEYKCKVSNKGLPSSIEKTISKAKGQPREPQVYTLPPSQEEMT | |
| With C-terminal | KNQVSLTCLVKGFYPSDIAVEWESNGQPENNYKTTPPVLDSDGSF | |
| Lysine | FLYSRLTVDKSRWQEGNVFSCSVMHEALHNHYTQKSLSLSLGK | |
| IgG4 Partial Hinge | PCPSCPAPEFLGGPSVFLFPPKPKDTLMISRTPEVTCVVVDVSQE | 140 |
| Region + CH2 Region | DPEVQFNWYVDGVEVHNAKTKPREEQFNSTYRVVSVLTVLHQDWL | |
| + CH3 Region | NGKEYKCKVSNKGLPSSIEKTISKAKGQPREPQVYTLPPSQEEMT | |
| Without C-terminal | KNQVSLTCLVKGFYPSDIAVEWESNGQPENNYKTTPPVLDSDGSE | |
| Lysine | FLYSRLTVDKSRWQEGNVFSCSVMHEALHNHYTQKSLSLSLG | |
| IgG4 Hinge Region + | ESKYGPPCPSCPAPEFLGGPSVELFPPKPKDTLMISRTPEVTCVV | 141 |
| CH2 Region + CH3 | VDVSQEDPEVQFNWYVDGVEVHNAKTKPREEQFNSTYRVVSVLTV | |
| Region | LHQDWLNGKEYKCKVSNKGLPSSIEKTISKAKGQPREPQVYTLPP | |
| With C-terminal | SQEEMTKNQVSLTCLVKGFYPSDIAVEWESNGQPENNYKTTPPVL | |
| Lysine | DSDGSFFLYSRLTVDKSRWQEGNVESCSVMHEALHNHYTQKSLSL | |
| | SLGK | |
| IgG4 Hinge Region + | ESKYGPPCPSCPAPEFLGGPSVFLFPPKPKDTLMISRTPEVTCVV | 142 |
| CH2 Region + CH3 | VDVSQEDPEVQFNWYVDGVEVHNAKTKPREEQFNSTYRVVSVLTV | |
| Region | LHQDWLNGKEYKCKVSNKGLPSSIEKTISKAKGQPREPQVYTLPP | |
| Without C-terminal | SQEEMTKNQVSLTCLVKGFYPSDIAVEWESNGQPENNYKTTPPVL | |
| Lysine | DSDGSFFLYSRLTVDKSRWQEGNVESCSVMHEALHNHYTQKSLSL | |
| | SLG | |
| IgG4 CH1 + Hinge | ASTKGPSVFPLAPCSRSTSESTAALGCLVKDYFPEPVTVSWNSGA | 143 |
| Region + CH2 Region | LTSGVHTFPAVLQSSGLYSLSSVVTVPSSSLGTKTYTCNVDHKPS | |
| + CH3 Region | NTKVDKRVESKYGPPCPSCPAPEFLGGPSVELFPPKPKDTLMISR | |
| With C-terminal | TPEVTCVVVDVSQEDPEVQFNWYVDGVEVHNAKTKPREEQENSTY | |
| Lysine | RVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENN | |
| | YKTTPPVLDSDGSFFLYSRLTVDKSRWQEGNVFSCSVMHEALHNH | |
| | YTQKSLSLSLGK | |
| IgG4 CH1 + Hinge | ASTKGPSVFPLAPCSRSTSESTAALGCLVKDYFPEPVTVSWNSGA | 144 |
| Region + CH2 Region | LTSGVHTFPAVLQSSGLYSLSSVVTVPSSSLGTKTYTCNVDHKPS | |
| + CH3 Region | NTKVDKRVESKYGPPCPSCPAPEFLGGPSVFLFPPKPKDTLMISR | |
| Without C-terminal | TPEVTCVVVDVSQEDPEVQFNWYVDGVEVHNAKTKPREEQENSTY | |
| Lysine | RVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENN | |
| | YKTTPPVLDSDGSFFLYSRLTVDKSRWQEGNVFSCSVMHEALHNH | |
| | YTQKSLSLSLG | |
| Ig light chain kappa | RTVAAPSVFIFPPSDEQLKSGTASVVCLLNNFYPREAKVQWKVDN | 145 |
| constant region (KCL) | ALQSGNSQESVTEQDSKDSTYSLSSTLTLSKADYEKHKVYACEVT | |
| | HQGLSSPVTKSENRGEC | |
| Ig light chain kappa | GQPKANPTVTLFPPSSEELQANKATLVCLISDFYPGAVTVAWKAD | 146 |
| constant region (ACL) | GSPVKAGVETTKPSKQSNNKYAASSYLSLTPEQWKSHRSYSCQVT | |
| | HEGSTVEKTVAPTECS | |

In some embodiments, the CD161 binding protein (or a conjugate or fusion protein comprising the same) comprises an Ig constant region comprising an amino acid sequence set forth in Table 5. In some embodiments, the CD161 binding protein (or a conjugate or fusion protein comprising the same) comprises an Ig constant region comprising an amino acid sequence set forth in Table 5, and further comprises 1 or more but less than 15% (less than 12%, less than 10%, less than 8%), amino acid variations (e.g., amino acid substitutions, deletions, or additions). In some embodiments, the CD161 binding protein (or a conjugate or fusion protein comprising the same) comprises an Ig constant region comprising an amino acid sequence set forth in Table 5, comprising or consisting of at least about 1, 2, 3, 4, 5, 6, 7, 8, 9, 10, or more amino acid variations (e.g., amino acid substitutions, deletions, or additions). In some embodiments, the CD161 binding protein (or a conjugate or fusion protein comprising the same) comprises an Ig constant region comprising an amino acid sequence set forth in Table 5, comprising or consisting of about 1, 2, 3, 4, 5, 6, 7, 8, 9, 10, or more amino acid variations (e.g., amino acid substitutions, deletions, or additions). In some embodiments, the CD161 binding protein (or a conjugate or fusion protein comprising the same) comprises an Ig constant region comprising an amino acid sequence set forth in Table 5, comprising or consisting of about no more than 1, 2, 3, 4, 5, 6, 7, 8, 9, 10, or more amino acid variations (e.g., amino acid substitutions, deletions, or additions).

In some embodiments, the CD161 binding protein (or a conjugate or fusion protein comprising the same) comprises an Ig constant region comprising an amino acid sequence set forth in Table 5, and further comprises 1 or more but less than 15% (less than 12%, less than 10%, less than 8%), amino acid substitutions. In some embodiments, the CD161 binding protein (or a conjugate or fusion protein comprising the same) comprises an Ig constant region comprising an amino acid sequence set forth in Table 5, comprising or consisting of at least about 1, 2, 3, 4, 5, 6, 7, 8, 9, 10, or more amino acid substitutions. In some embodiments, the CD161 binding protein (or a conjugate or fusion protein comprising the same) comprises an Ig constant region comprising an amino acid sequence set forth in Table 5, comprising or consisting of about 1, 2, 3, 4, 5, 6, 7, 8, 9, 10, or more amino acid substitutions. In some embodiments, the CD161 binding protein (or a conjugate or fusion protein comprising the same) comprises an Ig constant region comprising an amino acid sequence set forth in Table 5, comprising or consisting of about no more than about 1, 2, 3, 4, 5, 6, 7, 8, 9, 10, or more amino acid substitutions.

In some embodiments, the CD161 binding protein (or a conjugate or fusion protein comprising the same) comprises an Ig constant region comprising the amino acid sequence of any one or more of SEQ ID NOS: 119-146. In some embodiments, the CD161 binding protein (or a conjugate or fusion protein comprising the same) comprises an Ig constant region comprising the amino acid sequence of any one or more of SEQ ID NOS: 119-146, and further comprises 1 or more but less than 15% (less than 12%, less than 10%, less than 8%), amino acid variations (e.g., amino acid substitutions, deletions, or additions). In some embodiments, the CD161 binding protein (or a conjugate or fusion protein comprising the same) comprises an Ig constant region comprising the amino acid sequence of any one or more of SEQ ID NOS: 119-146, comprising at least about 1, 2, 3, 4, 5, 6, 7, 8, 9, 10, or more amino acid variations (e.g., amino acid substitutions, deletions, or additions). In some embodiments, the CD161 binding protein (or a conjugate or fusion protein comprising the same) comprises an Ig constant region comprising the amino acid sequence of any one or more of SEQ ID NOS: 119-146, comprising or consisting about 1, 2, 3, 4, 5, 6, 7, 8, 9, or 10 amino acid variations (e.g., amino acid substitutions, deletions, or additions). In some embodiments, the CD161 binding protein (or a conjugate or fusion protein comprising the same) comprises an Ig constant region comprising the amino acid sequence of any one or more of SEQ ID NOS: 119-146, comprising or consisting of no more than about 1, 2, 3, 4, 5, 6, 7, 8, 9, or 10 amino acid variations (e.g., amino acid substitutions, deletions, or additions).

In some embodiments, the CD161 binding protein (or a conjugate or fusion protein comprising the same) comprises an Ig constant region comprising the amino acid sequence of any one or more of SEQ ID NOS: 119-146, and further comprises 1 or more but less than 15% (less than 12%, less than 10%, less than 8%), amino acid substitutions. In some embodiments, the CD161 binding protein (or a conjugate or fusion protein comprising the same) comprises an Ig constant region comprising the amino acid sequence of any one or more of SEQ ID NOS: 119-146. In some embodiments, the CD161 binding protein (or a conjugate or fusion protein comprising the same) comprises an Ig constant region comprising the amino acid sequence of any one or more of SEQ ID NOS: 119-146, comprising or consisting at least about 1, 2, 3, 4, 5, 6, 7, 8, 9, 10, or more amino acid substitutions. In some embodiments, the CD161 binding protein (or a conjugate or fusion protein comprising the same) comprises an Ig constant region comprising the amino acid sequence of any one or more of SEQ ID NOS: 119-146, comprising or consisting about 1, 2, 3, 4, 5, 6, 7, 8, 9, or 10 amino acid substitutions. In some embodiments, the CD161 binding protein (or a conjugate or fusion protein comprising the same) comprises an Ig constant region comprising the amino acid sequence of any one or more of SEQ ID NOS: 119-146, comprising or consisting of no more than about 1, 2, 3, 4, 5, 6, 7, 8, 9, or 10 amino acid substitutions.

In some embodiments, the CD161 binding protein (or one or more polypeptide thereof) (e.g., described herein) (or a conjugate or fusion protein comprising the same (e.g., described herein)) comprises one or more mIg heavy chain constant regions (e.g., a CH2 region, a CH3 region, a hinge region, an Fc region). In some embodiments, the mIg is mIgG (mIgG). In some embodiments, the mIgG is mIgG1, mIgG2a, mIgG2c, mIgG2b, or mIgG3. In some embodiments, the mIgG is mIgG1 or mIgG2a. In some embodiments, the mIgG is mIgG1. In some embodiments, the mIgG is mIgG2a.

In some embodiments, the CD161 binding protein (or one or more polypeptide thereof) (e.g., described herein) (or a conjugate or fusion protein comprising the same (e.g., described herein)) comprises of a mIgG CH2 region and a mIgG CH3 region. In some embodiments, the heterologous polypeptide comprises or consists of a partial mIgG hinge region, mIgG CH2 region, and mIgG CH3 region. In some embodiments, the CD161 binding protein (or one or more polypeptide thereof) (e.g., described herein) (or a conjugate or fusion protein comprising the same (e.g., described herein)) comprises a mIgG hinge region, mIgG CH2 region, and mIgG CH3 region. In some embodiments, the CD161 binding protein (or one or more polypeptide thereof) (e.g., described herein) (or a conjugate or fusion protein comprising the same (e.g., described herein)) comprises a mIgG1 CH2 region and a mIgG1 CH3 region. In some embodiments, the CD161 binding protein (or one or more polypeptide thereof) (e.g., described herein) (or a conjugate or fusion protein comprising the same (e.g., described herein)) comprises a partial mIgG1 hinge region, mIgG1 CH2 region, and mIgG1 CH3 region. In some embodiments, the CD161 binding protein (or one or more polypeptide thereof) (e.g., described herein) (or a conjugate or fusion protein comprising the same (e.g., described herein)) comprises a mIgG1 hinge region, mIgG1 CH2 region, and mIgG1 CH3 region. In some embodiments, the CD161 binding protein (or one or more polypeptide thereof) (e.g., described herein) (or a conjugate or fusion protein comprising the same (e.g., described herein)) comprises a mIgG2a CH2 region and a mIgG2a CH3 region. In some embodiments, the CD161 binding protein (or one or more polypeptide thereof) (e.g., described herein) (or a conjugate or fusion protein comprising the same (e.g., described herein)) comprises a partial mIgG2a hinge region, mIg2a CH2 region, and mIgG2a CH3 region. In some embodiments, the CD161 binding protein (or one or more polypeptide thereof) (e.g., described herein) (or a conjugate or fusion protein comprising the same (e.g., described herein)) comprises a mIgG2a hinge region, mIgG2a CH2 region, and mIgG2a CH3 region.

In some embodiments, the CD161 binding protein (or one or more polypeptide thereof) (e.g., described herein) (or a conjugate or fusion protein comprising the same (e.g., described herein)) comprises a mIg Fc region. In some embodiments, the mIg Fc region comprises or consists of at least a portion of a hinge region, a CH2 region, and a CH3 region. In some embodiments, the mIg Fe region comprises or consists of a hinge region, a CH2 region, and a CH3 region. In some embodiments, the mIg Fc region comprises or consists of at least a portion of a mIgG hinge region, a mIgG CH2 region, and a mIgG CH3 region. In some embodiments, the mIg Fc region comprises or consists of a mIgG hinge region, a mIgG CH2 region, and a mIgG CH3 region. In some embodiments, the mIg Fc region comprises or consists of at least a portion of a mIgG1 hinge region, a mIgG1 CH2 region, and a mIgG1 CH3 region. In some embodiments, the mg Fc region comprises or consists of a mIgG1 hinge region, a mIgG1 CH2 region, and a mIgG1 CH3 region. In some embodiments, the mIg Fc region comprises or consists of at least a portion of a mIgG2a hinge region, a mIgG2a CH2 region, and a mIgG2a CH3 region. In some embodiments, the mIg Fc region comprises or consists of a mIgG2a hinge region, a mIgG2a CH2 region, and a mIgG2a CH3 region.

The amino acid sequence of exemplary reference mIgG1 and mIgG2a heavy chain constant regions, which can be incorporated in one or more of the embodiments described herein (e.g., fusion proteins and polypeptide), is provided in Table 6.

TABLE 6
The Amino Acid Sequence of Exemplary mlg Heavy and Light Chain Constant
Region Components.

| | | SEQ |
|---|---|---|
| Description | Amino Acid Sequence | ID NO |
| mIgG1 Hinge Region | VPRDCGCKPCICT | 147 |
| mIgG1 CH2 Region | VPEVSSVFIFPPKPKDVLTITLTPKVTCVVVAISKDDPEVQFSWE | 148 |
| | VDDVEVHTAQTQPREEQFENSTERSVSELPIMHQDWLNGKEFKCRV | |
| | NSAAFPAPIEKTISKTK | |
| mIgG1 CH3 Region | GRPKAPQVYTIPPPKEQMAKDKVSLTCMITDFFPEDITVEWQWNG | 149 |
| With C-terminal | QPAENYKNTQPIMNINGSYFVYSKLNVQKSNWEAGNTFTCSVLHE | |
| Lysine | GLHNHHTEKSLSHSPGK | |
| mIgG1 CH3 Region | GRPKAPQVYTIPPPKEQMAKDKVSLTCMITDFFPEDITVEWQWNG | 150 |
| Without C-terminal | QPAENYKNTQPIMNTNGSYFVYSKLNVQKSNWEAGNTFTCSVLHE | |
| Lysine | GLHNHHTEKSLSHSPG | |
| mIgG1 CH2 Region + | VPEVSSVFIFPPKPKDVLTITLTPKVTCVVVAISKDDPEVQFSWE | 151 |
| CH3 Region | VDDVEVHTAQTQPREEQENSTERSVSELPIMHQDWLNGKEFKCRV | |
| With C-terminal | NSAAFPAPIEKTISKTKGRPKAPQVYTIPPPKEQMAKDKVSLTCM | |
| Lysine | ITDFFPEDITVEWQWNGQPAENYKNTQPIMNINGSYFVYSKLNVQ | |
| | KSNWEAGNTFTCSVLHEGLHNHHTEKSLSHSPGK | |
| mIgG1 CH2 Region + | VPEVSSVFIFPPKPKDVLTITLTPKVTCVVVAISKDDPEVQFSWF | 152 |
| CH3 Region | VDDVEVHTAQTQPREEQFNSTFRSVSELPIMHQDWLNGKEFKCRV | |
| Without C-terminal | NSAAFPAPIEKTISKTKGRPKAPQVYTIPPPKEQMAKDKVSLTCM | |
| Lysine | ITDFFPEDITVEWQWNGQPAENYKNTQPIMNINGSYFVYSKLNVQ | |
| | KSNWEAGNTFTCSVLHEGLHNHHTEKSLSHSPG | |
| mIgG1 Hinge Region | VPRDCGCKPCICTVPEVSSVFIFPPKPKDVLTITLTPKVTCVVVA | 153 |
| + CH2 Region + CH3 | ISKDDPEVQFSWFVDDVEVHTAQTQPREEQENSTERSVSELPIMH | |
| Region | QDWLNGKEFKCRVNSAAFPAPIEKTISKTKGRPKAPQVYTIPPPK | |
| With C-terminal | EQMAKDKVSLTCMITDFFPEDITVEWQWNGQPAENYKNTQPIMNT | |
| Lysine | NGSYFVYSKLNVQKSNWEAGNTFTCSVLHEGLHNHHTEKSLSHSP | |
| | GK | |
| mIgG1 Hinge Region | VPRDCGCKPCICTVPEVSSVFIFPPKPKDVLTITLTPKVTCVVVA | 154 |
| + CH2 Region + CH3 | ISKDDPEVQFSWFVDDVEVHTAQTQPREEQENSTERSVSELPIMH | |
| Region | QDWLNGKEFKCRVNSAAFPAPIEKTISKTKGRPKAPQVYTIPPPK | |
| Without C-terminal | EQMAKDKVSLTCMITDFFPEDITVEWQWNGQPAENYKNTQPIMNT | |
| Lysine | NGSYFVYSKLNVQKSNWEAGNTFTCSVLHEGLHNHHTEKSLSHSP | |
| | GK | |
| mIgG2a Hinge Region | EPRGPTIKPCPPCKCP | 155 |
| mIgG2a CH2 Region | APNAAGGPSVFIFLLKIKDVLMISLSPIVTCVVVDVSEDDPDVQI | 156 |
| | SWFVNNVEVHTAQTQTHREDYNSTLRVVSALPIQHQDWMSGKEFK | |
| | CKVNNKDLPAPIERTISKPKGSVRAPQVYVLPPPEEEMTKKQVT | |
| mIgG2a CH3 Region | LTCMVTDEMPEDIYVEWTNNGKTELNYKNTEPVLDSDGSYFMYSK | 157 |
| With C-terminal | LRVEKKNWVERNSYSCSVVHEGLHNHHTTKSESRTPGK | |
| Lysine | | |
| mIgG2a CH3 Region | LTCMVTDFMPEDIYVEWTNNGKTELNYKNTEPVLDSDGSYFMYSK | 158 |
| Without C-terminal | LRVEKKNWVERNSYSCSVVHEGLHNHHTTKSFSRTPG | |
| Lysine | | |
| mIgG2a CH2 Region | APNAAGGPSVFIFLLKIKDVLMISLSPIVTCVVVDVSEDDPDVQI | 159 |
| + CH3 Region | SWFVNNVEVHTAQTQTHREDYNSTLRVVSALPIQHQDWMSGKEFK | |
| With C-terminal | CKVNNKDLPAPIERTISKPKGSVRAPQVYVLPPPEEEMTKKQVTL | |
| Lysine | TCMVTDEMPEDIYVEWTNNGKTELNYKNTEPVLDSDGSYFMYSKL | |
| | RVEKKNWVERNSYSCSVVHEGLHNHHTTKSFSRTPGK | |
| mIgG2a CH2 Region | APNAAGGPSVFIFLLKIKDVLMISLSPIVTCVVVDVSEDDPDVQI | 160 |
| + CH3 Region | SWFVNNVEVHTAQTQTHREDYNSTLRVVSALPIQHQDWMSGKEFK | |
| Without C-terminal | CKVNNKDLPAPIERTISKPKGSVRAPQVYVLPPPEEEMTKKQVTL | |
| Lysine | TCMVTDEMPEDIYVEWTNNGKTELNYKNTEPVLDSDGSYFMYSKL | |
| | RVEKKNWVERNSYSCSVVHEGLHNHHTTKSFSRTPG | |
| mIgG2a Hinge Region | EPRGPTIKPCPPCKCPAPNAAGGPSVFIFLLKIKDVLMISLSPIV | 161 |
| + CH2 Region + CH3 | TCVVVDVSEDDPDVQISWFVNNVEVHTAQTQTHREDYNSTLRVVS | |
| Region | ALPIQHQDWMSGKEFKCKVNNKDLPAPIERTISKPKGSVRAPQVY | |
| With C-terminal | VLPPPEEEMTKKQVTLTCMVTDEMPEDIYVEWTNNGKTELNYKNT | |
| Lysine | EPVLDSDGSYFMYSKLRVEKKNWVERNSYSCSVVHEGLHNHHTTK | |
| | SFSRTPGK | |
| mIgG2a Hinge Region | EPRGPTIKPCPPCKCPAPNAAGGPSVFIFLLKIKDVLMISLSPIV | 162 |
| + CH2 Region + CH3 | TCVVVDVSEDDPDVQISWFVNNVEVHTAQTQTHREDYNSTLRVVS | |
| Region | ALPIQHQDWMSGKEFKCKVNNKDLPAPIERTISKPKGSVRAPQVY | |
| Without C-terminal | VLPPPEEEMTKKQVTLTCMVTDEMPEDIYVEWTNNGKTELNYKNT | |
| Lysine | EPVLDSDGSYFMYSKLRVEKKNWVERNSYSCSVVHEGLHNHHTTK | |
| | SFSRTPG | |
| Ig light chain kappa | RADAAPTVSIFPPSSEQLTSGGASVVCELNNFYPKDINVKWKIDG | 163 |
| constant region (KCL) | SERQNGVLNSWTDQDSKDSTYSMSSTLTLTKDEYERHNSYTCEAT | |
| | HKTSTSPIVKSENRNEC | |

In some embodiments, the CD161 binding protein (or a conjugate or fusion protein comprising the same) comprises an Ig constant region comprising an amino acid sequence set forth in Table 6. In some embodiments, the CD161 binding protein (or a conjugate or fusion protein comprising the same) comprises an Ig constant region comprising an amino acid sequence set forth in Table 6, and further comprises 1 or more but less than 15% (less than 12%, less than 10%, less than 8%), amino acid variations (e.g., amino acid substitutions, deletions, or additions). In some embodiments, the CD161 binding protein (or a conjugate or fusion protein comprising the same) comprises an Ig constant region comprising an amino acid sequence set forth in Table 6, comprising or consisting of at least about 1, 2, 3, 4, 5, 6, 7, 8, 9, 10, or more amino acid variations (e.g., amino acid substitutions, deletions, or additions). In some embodiments, the CD161 binding protein (or a conjugate or fusion protein comprising the same) comprises an Ig constant region comprising an amino acid sequence set forth in Table 6, comprising or consisting of about 1, 2, 3, 4, 5, 6, 7, 8, 9, 10, or more amino acid variations (e.g., amino acid substitutions, deletions, or additions). In some embodiments, the CD161 binding protein (or a conjugate or fusion protein comprising the same) comprises an Ig constant region comprising an amino acid sequence set forth in Table 6, comprising or consisting of about no more than 1, 2, 3, 4, 5, 6, 7, 8, 9, 10, or more amino acid variations (e.g., amino acid substitutions, deletions, or additions).

In some embodiments, the CD161 binding protein (or a conjugate or fusion protein comprising the same) comprises an Ig constant region comprising an amino acid sequence set forth in Table 6, and further comprises 1 or more but less than 15% (less than 12%, less than 10%, less than 8%), amino acid substitutions. In some embodiments, the CD161 binding protein (or a conjugate or fusion protein comprising the same) comprises an Ig constant region comprising an amino acid sequence set forth in Table 6, comprising or consisting of at least about 1, 2, 3, 4, 5, 6, 7, 8, 9, 10, or more amino acid substitutions. In some embodiments, the CD161 binding protein (or a conjugate or fusion protein comprising the same) comprises an Ig constant region comprising an amino acid sequence set forth in Table 6, comprising or consisting of about 1, 2, 3, 4, 5, 6, 7, 8, 9, 10, or more amino acid substitutions. In some embodiments, the CD161 binding protein (or a conjugate or fusion protein comprising the same) comprises an Ig constant region comprising an amino acid sequence set forth in Table 6, comprising or consisting of about no more than about 1, 2, 3, 4, 5, 6, 7, 8, 9, 10, or more amino acid substitutions.

In some embodiments, the CD161 binding protein (or a conjugate or fusion protein comprising the same) comprises an Ig constant region comprising the amino acid sequence of any one or more of SEQ ID NOS: 147-163. In some embodiments, the CD161 binding protein (or a conjugate or fusion protein comprising the same) comprises an Ig constant region comprising the amino acid sequence of any one or more of SEQ ID NOS: 147-163, and further comprises 1 or more but less than 15% (less than 12%, less than 10%, less than 8%), amino acid variations (e.g., amino acid substitutions, deletions, or additions). In some embodiments, the CD161 binding protein (or a conjugate or fusion protein comprising the same) comprises an Ig constant region comprising the amino acid sequence of any one or more of SEQ ID NOS: 147-163, comprising at least about 1, 2, 3, 4, 5, 6, 7, 8, 9, 10, or more amino acid variations (e.g., amino acid substitutions, deletions, or additions). In some embodiments, the CD161 binding protein (or a conjugate or fusion protein comprising the same) comprises an Ig constant region comprising the amino acid sequence of any one or more of SEQ ID NOS: 147-163, comprising or consisting about 1, 2, 3, 4, 5, 6, 7, 8, 9, or 10 amino acid variations (e.g., amino acid substitutions, deletions, or additions). In some embodiments, the CD161 binding protein (or a conjugate or fusion protein comprising the same) comprises an Ig constant region comprising the amino acid sequence of any one or more of SEQ ID NOS: 147-163, comprising or consisting of no more than about 1, 2, 3, 4, 5, 6, 7, 8, 9, or 10 amino acid variations (e.g., amino acid substitutions, deletions, or additions).

In some embodiments, the CD161 binding protein (or a conjugate or fusion protein comprising the same) comprises an Ig constant region comprising the amino acid sequence of any one or more of SEQ ID NOS: 147-163, and further comprises 1 or more but less than 15% (less than 12%, less than 10%, less than 8%), amino acid substitutions. In some embodiments, the CD161 binding protein (or a conjugate or fusion protein comprising the same) comprises an Ig constant region comprising the amino acid sequence of any one or more of SEQ ID NOS: 147-163. In some embodiments, the CD161 binding protein (or a conjugate or fusion protein comprising the same) comprises an Ig constant region comprising the amino acid sequence of any one or more of SEQ ID NOS: 147-163, comprising or consisting at least about 1, 2, 3, 4, 5, 6, 7, 8, 9, 10, or more amino acid substitutions. In some embodiments, the CD161 binding protein (or a conjugate or fusion protein comprising the same) comprises an Ig constant region comprising the amino acid sequence of any one or more of SEQ ID NOS: 147-163, comprising or consisting about 1, 2, 3, 4, 5, 6, 7, 8, 9, or 10 amino acid substitutions. In some embodiments, the CD161 binding protein (or a conjugate or fusion protein comprising the same) comprises an Ig constant region comprising the amino acid sequence of any one or more of SEQ ID NOS: 147-163, comprising or consisting of no more than about 1, 2, 3, 4, 5, 6, 7, 8, 9, or 10 amino acid substitutions.

### 5.7 Ig Effector Function

In some embodiments, the CD161 binding protein (or a conjugate or fusion protein comprising the same) comprises an Ig Fc region. In some embodiments, the Ig Fc region is part of a full-length antibody. In some embodiments, the Ig Fc region comprises or consists of at least a portion of a hinge region, a CH2 region, and a CH3 region. In some embodiments, the Ig Fc region comprises or consists of a hinge region, a CH2 region, and a CH3 region. In some embodiments, the Ig Fc region comprises or consists of at least a portion of an IgG hinge region, an IgG CH2 region, and an IgG CH3 region. In some embodiments, the Ig Fc region comprises or consists of an IgG hinge region, an IgG CH2 region, and an IgG CH3 region.

In some embodiments, the Ig Fc region comprises or consists of at least a portion of a hIgG1 hinge region, a hIgG1 CH2 region, and a hIgG1 CH3 region. In some embodiments, the Ig Fc region comprises or consists of a hIgG1 hinge region, a hIgG1 CH2 region, and a hIgG1 CH3 region. In some embodiments, the Ig Fc region comprises or consists of at least a portion of a hIgG4 hinge region, a hIgG4 CH2 region, and a hIgG4 CH3 region. In some embodiments, the Ig Fc region comprises or consists of a hIgG4 hinge region, a hIgG4 CH2 region, and a hIgG4 CH3 region.

In some embodiments, the Ig Fc region comprises or consists of at least a portion of a mIgG1 hinge region, a mIgG1 CH2 region, and a mIgG1 CH3 region. In some embodiments, the Ig Fc region comprises or consists of a mIgG1 hinge region, a mIgG1 CH2 region, and a mIgG1 CH3 region. In some embodiments, the Ig Fc region comprises or consists of at least a portion of a mIgG2a hinge region, a mIgG2a CH2 region, and a mIgG2a CH3 region. In some embodiments, the Ig Fc region comprises or consists of a mIgG2a hinge region, a mIgG2a CH2 region, and a mIgG2a CH3 region.

In some embodiments, the Ig Fc region exhibits an alteration (e.g., enhancement, reduction) in one or more Fc effector function relative to a reference (e.g., wild type) Ig Fc region. Exemplary Ig Fc effector functions include, but are not limited to, antibody dependent cellular cytotoxicity (ADCC), antibody dependent cellular phagocytosis (ADCP), complement dependent cytotoxicity (CDC), binding affinity to Clq, and binding affinity to one or more human Fc receptor (e.g., an Fc receptor (e.g., FcγRI, FcγRIIa, FcγRIIc, FcγRIIIa, and/or FcγRIIIb (e.g., FcγRI, FcγRIIa, and/or FcγRIIIa))).

Standard in vitro and/or in vivo assays known in the art can be conducted to evaluate Fc effector function, including, any one or more of ADCC, CDC, ADCP, Fc receptor (e.g., Fc receptor) binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (⁵¹Cr) assay. Briefly, ⁵¹Cr is pre-loaded into target cells expressing CD20, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide flurophore (Europium). see, e.g., Parekh, Bhavin S et al. “Development and validation of an antibody-dependent cell-mediated cytotoxicity-reporter gene assay.” mAbs vol. 4, 3 (2012): 310-8. Doi:10.4161/mabs.19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a fusion protein described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of a Ig fusion protein described herein to bind C1q (or bind with less affinity than a reference fusion protein) and hence lack (or have decreased) CDC activity. The binding of a Ig fusion protein described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127(6):681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells expressing CD20 and a fusion protein described herein. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi:10.3791/3588; the entire contents of each of which is incorporated by reference herein.

Binding of an Ig fusion protein described herein to an Ig Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4” Ed., Lippincott-Raven, Philadelphia (1999), the entire contents of which is incorporated by reference herein for all purposes.

### 5.7.1 Reduced Ig Effector Function

In some embodiments, the Ig Fc region of a CD161 binding protein (or a conjugate or fusion protein comprising the same) (e.g., described herein) exhibits a decrease in one or more Fc effector function relative to a reference (e.g., wild type) Ig Fc region. Exemplary Ig Fc effector functions include, but are not limited to, ADCC, ADCP, CDC, binding affinity to Clq, and binding affinity to one or more human Fc receptor (e.g., an Fc receptor (e.g., FcγRI, FcγRIIa, FcγRIIc, FcγRIIIa, and/or FcγRIIIb (e.g., FcγRI, FcγRIIa, and/or FcγRIIIa))).

In some embodiments, the Ig Fc region of a CD161 binding protein (or a conjugate or fusion protein comprising the same) (e.g., described herein) is modified (e.g., comprises one or more variation (e.g., one or more amino acid substitution, deletion, addition, etc.); altered glycosylation)) (referred to herein as a “modified Ig Fc”). In some embodiments, the one or more variation (e.g., the one or more variation (e.g., the one or more amino acid substitution, deletion, addition, etc.; the altered glycosylation)) decreases or abolishes one or more Fc effector function, relative to a reference Ig Fc that does not comprise the modification (e.g., the one or more variation (e.g., the one or more amino acid substitution, deletion, addition, etc.; the altered glycosylation)).

In some embodiments, the modified Ig Fc fusion protein exhibits no detectable or decreased ADCC compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits no detectable or decreased CDC compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits no detectable or decreased ADCP compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))) compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRI, FcγRIIa, and/or FcγRIIIa compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRI compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRIIa compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRIIIa compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to Clq compared to a reference fusion protein that does not comprise the Ig Fc modification (e.g., the one or more variation (e.g., the one or more amino acid substitution, deletion, addition, etc.; the altered glycosylation)).

Amino acid substitutions that decrease or abolish one or more Ig Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated by reference herein for all purposes, see more particularly for example, e.g., Table 3 of Saunders.

Table 7 below, provides exemplary amino acid substitutions (and combinations thereof) that can be utilized to reduce one or more Fc effector function. Amino acids in Table 7 are numbered according to the EU numbering scheme. The effects on effector function set forth in Table 7 are exemplary only and not intended to be limiting. The amino acid substitutions set forth in Table 7 (except where noted) are with reference to an IgG1 Fc region. However, a person of ordinary skill in the could identify the corresponding amino acid in a non-IgG1 Fc region, for example in an IgG2 or IgG4 Fc region, should the base amino acid be different between the IgG1 and non-IgG1 Fc region. The amino acid substitutions set forth in Table 7 (except where noted) are with reference to a human IgG1 Fc region. However, a person of ordinary skill in the could identify the corresponding amino acid in a non-human IgG1 Fc region, for example in a murine IgG1 or IgG2a Fc region, should the base amino acid be different between the human IgG1 and murine Fc region.

TABLE 7
Exemplary Ig Fc Variations and Glycoengineering to
Reduce Effector Function.

| | |
|---|---|
| | Exemplary Effects on |
| Change | Effector Function |

Amino Acid Substitutions

| | |
|---|---|
| L235E | Reduces ADCC |
| L234A/L235A | Reduces ADCC, ADCP, CDC |
| L234A/L235A/P329G | Reduces ADCC, ADCP |
| L234A/L235A/P329A | Reduces ADCC, ADCP |
| P331S/L234G/L235F | Reduces CDC |
| D265A | Reduces ADCC, ADCP |
| G237A | Reduces ADCP |
| E318A | Reduces ADCP |
| E233P | Reduces FcγRI, II and III |
| | binding |
| G236R/L328R | Reduces ADCC |
| D270A | Reduces CDC |
| K322A | Reduces CDC |
| P329A | Reduces CDC |
| P331A | Reduces CDC |
| V264A | Reduces CDC |
| F241A | Reduces CDC |
| N297A | Reduces CDC |
| N297G | Reduces CDC |
| N297Q | Reduces CDC |
| IgG4 - S228P/L235E | Reduces FcγRI binding |
| IgG4- S228P/F234A/L235A | Reduces ADCC, CDC |
| IgG4- S228P/F234A/L235G | Reduces ADCC, CDC |
| IgG4- S228P/F234A/L235E | Reduces ADCC, CDC |
| IgG2- H268Q/V309L/A330S/P331S | Reduces ADCC, ADCP, CDC |

Glycoengineering

| | |
|---|---|
| High Mannose | Reduces CDC |

In some embodiments, the Ig Fc (e.g., IgG1 Fc) region of a CD161 binding protein (or a conjugate or fusion protein comprising the same) (e.g., described herein) comprises any one or more of the amino acid substitutions set forth in Table 7 (set forth in any set). In some embodiments, the Ig Fc (e.g., IgG1 Fc) region of a CD161 binding protein (or a conjugate or fusion protein comprising the same) (e.g., described herein) comprises any one or more of the sets of amino acid substitutions set forth in Table 7. In some embodiments, the Ig Fc (e.g., IgG1 Fc) region of a CD161 binding protein (or a conjugate or fusion protein comprising the same) (e.g., described herein) comprises any one or more of the glycosylation changes set forth in Table 7.

In some embodiments, the Ig Fc (e.g., IgG1 Fc) region of a CD161 binding protein (or a conjugate or fusion protein comprising the same) (e.g., described herein) comprises an amino acid substitution at any one or more (e.g., 1, 2, 3, 4, 5, 6, 7, 8, 9, or 10, or more) of amino acid positions L234, L235, P329, P331, D265, G237, E318, E233, G236, L328, D270, K322, V264, F241, and/or N297. In some embodiments, the Ig Fc (e.g., IgG1 Fc) region of a CD161 binding protein (or a conjugate or fusion protein comprising the same) (e.g., described herein) comprises an amino acid substitution at from about 1-10 (e.g., 1-9, 1-8, 1-7, 1-6, 1-5, 1-4, 1-3, or 1-2) of the following amino acid positions L234, L235, P329, P331, D265, G237, E318, E233, G236, L328, D270, K322, V264, F241, and/or N297.

In some embodiments, the Ig Fe (e.g., IgG1 Fc) region of a CD161 binding protein (or a conjugate or fusion protein comprising the same) (e.g., described herein) comprises one or more (e.g., 1, 2, 3, 4, 5, 6, 7, 8, 9, or 10, or more) of the following amino acid substitutions L234A, L234G, L235F, L235E, L235A, P329G, P329A, P331S, D265A, G237A, E318A, E233P, G236R, L328R, D270A, K322A, V264A, F241A, N297A, N297G, and/or N297Q. In some embodiments, the Ig Fe (e.g., IgG1 Fc) region of a CD161 binding protein (or a conjugate or fusion protein comprising the same) (e.g., described herein) comprises from about 1-10 (e.g., 1-9, 1-8, 1-7, 1-6, 1-5, 1-4, 1-3, or 1-2) of the following amino acid substitutions L234A, L234G, L235F, L235E, L235A, P329G, P329A, P331S, D265A, G237A, E318A, E233P, G236R, L328R, D270A, K322A, V264A, F241A, N297A, N297G, or N297Q.

In some embodiments, the Ig Fc region of a CD161 binding protein (or a conjugate or fusion protein comprising the same) (e.g., described herein) comprises a IgG1 Fc region comprising one or more amino acid variation.

In some embodiments, the IgG1 Fc region comprises an amino acid substitution at amino acid position L234, EU numbering according to Kabat. In some embodiments, the IgG1 Fc region comprises and L234A or L234G amino acid substitution, EU numbering according to Kabat.

In some embodiments, the IgG1 Fc region comprises an amino acid substitution at amino acid position L235, EU numbering according to Kabat. In some embodiments, the IgG1 Fc region comprises and L235A, L235G, L235E, or L235F amino acid substitution, EU numbering according to Kabat.

In some embodiments, the IgG1 Fc region comprises an amino acid substitution at amino acid position P329, EU numbering according to Kabat. In some embodiments, the IgG1 Fc region comprises and P329A or P329G amino acid substitution, EU numbering according to Kabat.

In some embodiments, the IgG1 Fc region comprises an amino acid substitution at one or more (e.g., 1 or 2) of amino acid positions L234 and/or L235, EU numbering according to Kabat. In some embodiments, the IgG1 Fc region comprises one or more (e.g., 1, 2, or 3) of the following amino acid substitutions L234A and/or L235A, EU numbering according to Kabat.

In some embodiments, the IgG1 Fc region comprises an amino acid substitution at one or more (e.g., 1, 2, or 3) of amino acid positions L234, L235, and/or P329, EU numbering according to Kabat. In some embodiments, the IgG1 Fc region comprises one or more (e.g., 1, 2, or 3) of the following amino acid substitutions L234A, L235A, and/or P329G, EU numbering according to Kabat. In some embodiments, the IgG1 Fc region comprises one or more (e.g., 1, 2, or 3) of the following amino acid substitutions L234A, L235A, and/or P329A, EU numbering according to Kabat.

In some embodiments, the IgG1 Fc region comprises an amino acid substitution at one or more (e.g., 1, 2, or 3) of amino acid positions P331, L234, and/or L235, EU numbering according to Kabat. In some embodiments, the IgG1 Fc region comprises one or more (e.g., 1, 2, or 3) of the following amino acid substitutions P331S, L234G, and/or L235F, EU numbering according to Kabat.

In some embodiments, the IgG1 Fc region comprises an amino acid substitution at amino acid position D265, EU numbering according to Kabat. In some embodiments, the IgG1 Fc region comprises a D235A amino acid substitution, EU numbering according to Kabat.

In some embodiments, the IgG1 Fc region comprises an amino acid substitution at amino acid position G237, EU numbering according to Kabat. In some embodiments, the IgG1 Fc region comprises a G237A amino acid substitution, EU numbering according to Kabat.

In some embodiments, the IgG1 Fc region comprises an amino acid substitution at amino acid position E318, EU numbering according to Kabat. In some embodiments, the IgG1 Fc region comprises a E318A amino acid substitution, EU numbering according to Kabat.

In some embodiments, the IgG1 Fc region comprises an amino acid substitution at amino acid position E233, EU numbering according to Kabat. In some embodiments, the IgG1 Fc region comprises a E233P amino acid substitution, EU numbering according to Kabat.

In some embodiments, the IgG1 Fc region comprises an amino acid substitution at amino acid position D270, EU numbering according to Kabat. In some embodiments, the IgG1 Fc region comprises a D270A amino acid substitution, EU numbering according to Kabat.

In some embodiments, the IgG1 Fc region comprises an amino acid substitution at amino acid position K322, EU numbering according to Kabat. In some embodiments, the IgG1 Fc region comprises a K322A amino acid substitution, EU numbering according to Kabat.

In some embodiments, the IgG1 Fc region comprises an amino acid substitution at amino acid position P331, EU numbering according to Kabat. In some embodiments, the IgG1 Fc region comprises a P331A amino acid substitution, EU numbering according to Kabat.

In some embodiments, the IgG1 Fc region comprises an amino acid substitution at amino acid position F241, EU numbering according to Kabat. In some embodiments, the IgG1 Fc region comprises a F241A amino acid substitution, EU numbering according to Kabat.

In some embodiments, the IgG1 Fc region comprises an amino acid substitution at amino acid position N297, EU numbering according to Kabat. In some embodiments, the IgG1 Fc region comprises a N297A, N297G, or N297Q amino acid substitution, EU numbering according to Kabat.

In some embodiments, the IgG1 Fc region comprises an amino acid substitution at one or more (e.g., 1 or 2) of amino acid positions G236 and/or L328, EU numbering according to Kabat. In some embodiments, the IgG1 Fc region comprises one or more (e.g., 1, 2, or 3) of the following amino acid substitutions G236R and/or L328R, EU numbering according to Kabat.

In some embodiments, the IgG4 Fc region comprises an amino acid substitution at amino acid position S228, EU numbering according to Kabat. In some embodiments, the IgG1 Fc region comprises an S228P amino acid substitution, EU numbering according to Kabat.

In some embodiments, the IgG4 Fc region comprises an amino acid substitution at amino acid position F234, EU numbering according to Kabat. In some embodiments, the IgG1 Fc region comprises an F234A amino acid substitution, EU numbering according to Kabat.

In some embodiments, the IgG4 Fc region comprises an amino acid substitution at amino acid position L235A, EU numbering according to Kabat. In some embodiments, the IgG1 Fc region comprises an L235A, L235G, or L235E amino acid substitution, EU numbering according to Kabat.

In some embodiments, the IgG4 Fc region comprises an amino acid substitution at one or more (e.g., 1 or 2) of amino acid positions S228 and/or L235, EU numbering according to Kabat. In some embodiments, the IgG4 Fc region comprises one or more (e.g., 1, 2, or 3) of the following amino acid substitutions S228P and/or L235E, EU numbering according to Kabat.

In some embodiments, the IgG4 Fc region comprises an amino acid substitution at one or more (e.g., 1, 2, or 3) of amino acid positions S228, F234, and/or L235, EU numbering according to Kabat. In some embodiments, the IgG4 Fc region comprises one or more (e.g., 1, 2, or 3) of the following amino acid substitutions S228P, F234A, and/or L235A, EU numbering according to Kabat.

In some embodiments, the IgG4 Fc region comprises an amino acid substitution at one or more (e.g., 1, 2, or 3) of amino acid positions S228, F234, and/or L235, EU numbering according to Kabat. In some embodiments, the IgG4 Fc region comprises one or more (e.g., 1, 2, or 3) of the following amino acid substitutions S228P, F234A, and/or L235G, EU numbering according to Kabat.

In some embodiments, the IgG4 Fc region comprises an amino acid substitution at one or more (e.g., 1, 2, or 3) of amino acid positions S228, F234, and/or L235, EU numbering according to Kabat. In some embodiments, the IgG4 Fc region comprises one or more (e.g., 1, 2, or 3) of the following amino acid substitutions S228P, F234A, and/or L235E, EU numbering according to Kabat.

In some embodiments, the IgG2 Fc region comprises an amino acid substitution at one or more (e.g., 1, 2, 3, or 4) of amino acid positions H268, V309, A330, and/or P331, EU numbering according to Kabat. In some embodiments, the IgG2 Fc region comprises one or more (e.g., 1, 2, 3, or 4) of the following amino acid substitutions H268Q, V309L, A330S, and/or P331S, EU numbering according to Kabat.

In some embodiments, the Ig Fc region of a CD161 binding protein (or a conjugate or fusion protein comprising the same) (e.g., described herein) comprises one or more changes to the glycosylation. In some embodiments, the Ig Fc region of a CD161 binding protein (or a conjugate or fusion protein comprising the same) (e.g., described herein) has increased high mannose glycosylation.

### 5.7.2 Enhanced Ig Effector Function

In some embodiments, the Ig Fc region of a CD161 binding protein (or a conjugate or fusion protein comprising the same) described herein exhibits an enhancement (e.g., an increase) in one or more Fc effector function relative to a reference (e.g., wild type) Ig Fc region. Exemplary Ig Fc effector functions include, but are not limited to, ADCC, ADCP, CDC, binding affinity to Clq, and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))).

In some embodiments, the Ig Fc region of a CD161 binding protein (or a conjugate or fusion protein comprising the same) is modified (e.g., comprises one or more variation (e.g., one or more amino acid substitution, deletion, addition, etc.); altered glycosylation (e.g., afucosylation))) (referred to herein as a “modified Ig Fc”). In some embodiments, the modification (e.g., the variation (e.g., one or more amino acid substitution, deletion, addition, etc.); altered glycosylation (e.g., afucosylation))) enhances (e.g., increases) one or more Fc effector function, relative to a reference Ig Fc that does not comprise the modification (e.g., the one or more variation (e.g., the one or more amino acid substitution, deletion, addition, etc.; the altered glycosylation (e.g., afucosylation))).

In some embodiments, a CD161 binding protein (or a conjugate or fusion protein comprising the same) comprising a modified Ig Fc exhibits enhanced (e.g., increased) ADCC compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))). In some embodiments, a CD161 binding protein (or a conjugate or fusion protein comprising the same) comprising a modified Ig Fc exhibits enhanced (e.g., increased) CDC compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))). In some embodiments, a CD161 binding protein (or a conjugate or fusion protein comprising the same) comprising a modified Ig Fc exhibits enhanced (e.g., increased) ADCP compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))). In some embodiments, a CD161 binding protein (or a conjugate or fusion protein comprising the same) comprising a modified Ig Fc exhibits enhanced (e.g., increased) binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))) compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))). In some embodiments, a CD161 binding protein (or a conjugate or fusion protein comprising the same) comprising a modified Ig Fc exhibits enhanced (e.g., increased) binding affinity to FcγRI, FcγRIIa, and/or FcγRIIIa compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))). In some embodiments, a CD161 binding protein (or a conjugate or fusion protein comprising the same) comprising a modified Ig Fc exhibits enhanced (e.g., increased) binding affinity to FcγRI compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))). In some embodiments, the a CD161 binding protein (or a conjugate or fusion protein comprising the same) comprising a modified Ig Fc exhibits enhanced (e.g., increased) to FcγRIIa compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))). In some embodiments, a CD161 binding protein (or a conjugate or fusion protein comprising the same) comprising a modified Ig Fc exhibits enhanced (e.g., increased) binding affinity to FcγRIIIa compared to a reference CD161 binding protein (or a conjugate or fusion protein comprising the same) that does not comprise the Ig Fc modification (e.g., the one or more variation (e.g., the one or more variation (e.g., the one or more amino acid substitution, deletion, addition, etc.; the altered glycosylation (e.g., afucosylation))). In some embodiments, a CD161 binding protein (or a conjugate or fusion protein comprising the same) comprising a modified Ig Fc exhibits enhanced (e.g., increased) binding affinity to C1q compared to a reference CD161 binding protein (or a conjugate or fusion protein comprising the same) that does not comprise the Ig Fc modification (e.g., the one or more variation (e.g., the one or more amino acid substitution, deletion, addition, etc.; the altered glycosylation (e.g., afucosylation))).

Amino acid substitutions that enhance (e.g., increase) one or more Ig Fc effector function are known in the art. See for example, Liu R, Oldham R J, Teal E, Beers S A, Cragg M S. Fc-Engineering for Modulated Effector Functions-Improving Antibodies for Cancer Treatment. Antibodies (Basel). 2020; 9(4):64. Published 2020 Nov. 17. doi:10.3390/antib9040064; van der Horst H J, Nijhof I S, Mutis T, Chamuleau MED. Fc-Engineered Antibodies with Enhanced Fc-Effector Function for the Treatment of B-Cell Malignancies. Cancers (Basel). 2020; 12(10):3041. Published 2020 Oct. 19. Doi:10.3390/cancers12103041; and Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of each of which is incorporated by reference herein for all purposes.

Table 8 below, provides exemplary amino acid substitutions (and combinations thereof) that can be utilized to increase one or more Ig Fc effector function. Amino acids in Table 8 are numbered according to the EU numbering scheme. The effects on effector function set forth in Table 8 are exemplary only and not intended to be limiting. The amino acid substitutions set forth in Table 8 are with reference to an IgG1 Fc region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 8
Exemplary Ig Fc Variations and Glycoengineering to
Increase Effector Function.

| | |
|---|---|
| | Exemplary Effects on |
| Variation/Glycoengineering | Effector Function |

Amino Acid Substitutions

| S298A/E333A/K334A | | Increases ADCC |
|---|---|---|
| S239D/I332E | | Increases ADCC |
| P247I/A339Q | | Increases ADCC |
| S239D/A330L/I332E | | Increases ADCC |
| G236A/S239D/I332E | | Increases ADCC |
| F243L/R292P/Y300L/V305I/P396L | | Increases ADCC |
| L235V/F243L/R292P/Y300L/P396L | | Increases ADCC |
| One Heavy | Opposing Heavy | Increases ADCC |
| Chain: | Chain: | |
| L234Y/L235Q/G | D270E/K326D/A3 | |
| 236W/S239M/H | 30M/K334E | |
| 268D/D270E/ | | |
| S298A | | |

| | |
|---|---|
| F243L/R292P/Y300L/V305I/P396L | Increases ADCP |
| S239D/I332E/A330L | Increases ADCP |
| S239D/I332E/A330L/G236A | Increases ADCP |
| S239D/I332E/G326A | Increases ADCP |
| G236A | Increases ADCP |
| G236A/S239D/I332E | Increases ADCP |
| S239D/I332E | Increases ADCP |
| K326W/E333S | Increases C1q Binding and CDC |
| S267E/H268E/S324T | Increases C1q Binding and CDC |
| S298A/E333A/K334A | Enhances FcγRIIIa binding |
| S239D/I332E | Enhances FcγRIIIa binding |
| P247I/A339Q | Enhances FcγRIIIa binding |
| F243L/R292P/Y300L/V305I/P396L | Enhances FcγRIIa binding; |
| | decreases FcβRIIb binding |
| G236A | Enhances FcγRIIa binding |

Glycoengineering

| | |
|---|---|
| Afucosylation | Increases ADCC, Increases |
| | ADCP |
| Galactosylation | Increases CDC |

In some embodiments, the Ig Fe (e.g., IgG1 Fc) region of a CD161 binding protein (or a conjugate or fusion protein comprising the same) comprises any one or more of the amino acid substitutions set forth in Table 8 (i.e., any one or more amino acid substitution set forth in any set of amino acid substitutions set forth in Table 8). In some embodiments, the Ig Fe (e.g., IgG1 Fc) region of a CD161 binding protein (or a conjugate or fusion protein comprising the same) comprises any one or more of the sets of amino acid substitutions set forth in Table 8. In some embodiments, the Ig Fe (e.g., IgG1 Fc) region of a CD161 binding protein (or a conjugate or fusion protein comprising the same) comprises any one or more of the glycosylation changes set forth in Table 8.

In some embodiments, the Ig Fe (e.g., IgG1 Fc) region of a CD161 binding protein (or a conjugate or fusion protein comprising the same) comprises an amino acid substitution at any one or more (e.g., 1, 2, 3, 4, 5, 6, 7, 8, 9, or 10, or more) of amino acid positions S298, E333, K334, S239, 1332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334. In some embodiments, the Ig Fe (e.g., IgG1 Fc) region of a CD161 binding protein (or a conjugate or fusion protein comprising the same) comprises an amino acid substitution at from about 1-10 (e.g., 1-9, 1-8, 1-7, 1-6, 1-5, 1-4, 1-3, or 1-2) of the following amino acid positions S298, E333, K334, S239, 1332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334.

In some embodiments, the Ig Fe (e.g., IgG1 Fc) region of a CD161 binding protein (or a conjugate or fusion protein comprising the same) comprises one or more (e.g., 1, 2, 3, 4, 5, 6, 7, 8, 9, or 10, or more) of the following amino acid substitutions S298A, E333A, K334A, S239D, I332E, P247I, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the Ig Fe (e.g., IgG1 Fc) region of a CD161 binding protein (or a conjugate or fusion protein comprising the same) comprises from about 1-10 (e.g., 1-9, 1-8, 1-7, 1-6, 1-5, 1-4, 1-3, or 1-2) of the following amino acid substitutions S298A, E333A, K334A, S239D, I332E, P247I, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the Ig Fc region of a CD161 binding protein (or a conjugate or fusion protein comprising the same) comprises a IgG1 Fc region comprising one or more amino acid variation relative to a reference IgG1 Fc region.

In some embodiments, the IgG1 Fc region comprises an amino acid substitution at one or more (e.g., 1, 2, or 3) of amino acid positions S298, E333, K334, EU numbering according to Kabat. In some embodiments, the IgG1 Fc region comprises one or more (e.g., 1, 2, or 3) of the following amino acid substitutions S298A, E333A, and/or K334A, EU numbering according to Kabat.

In some embodiments, the IgG1 Fc region comprises an amino acid substitution at one or more (e.g., 1 or 2) of amino acid positions S239 and/or 1332, EU numbering according to Kabat. In some embodiments, the IgG1 Fc region comprises one or more (e.g., 1 or 2) of the following amino acid substitutions S239D and/or 1332E, EU numbering according to Kabat.

In some embodiments, the IgG1 Fc region comprises an amino acid substitution at one or more (e.g., 1 or 2) of amino acid positions P247 and/or A339, EU numbering according to Kabat. In some embodiments, the IgG1 Fc region comprises one or more (e.g., 1 or 2) of the following amino acid substitutions P247I and/or A339Q, EU numbering according to Kabat.

In some embodiments, the IgG1 Fc region comprises an amino acid substitution at one or more (e.g., 1, 2, or 3) of amino acid positions S239, A330, and/or 1332, EU numbering according to Kabat. In some embodiments, the IgG1 Fc region comprises one or more (e.g., 1, 2, or 3) of the following amino acid substitutions S239D, A330L, and/or 1332E, EU numbering according to Kabat.

In some embodiments, the IgG1 Fc region comprises an amino acid substitution at one or more (e.g., 1, 2, or 3) of amino acid positions G236, S239, and/or 1332, EU numbering according to Kabat. In some embodiments, the IgG1 Fc region comprises one or more (e.g., 1, 2, or 3) of the following amino acid substitutions G236A, S239D, and/or 1332E, EU numbering according to Kabat.

In some embodiments, the IgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the IgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the IgG1 Fc region comprises an amino acid substitution at one or more (e.g., 1, 2, 3, 4, or 5) of amino acid positions L235, F243, R292, Y300, and P396, EU numbering according to Kabat. In some embodiments, the IgG1 Fc region comprises one or more (e.g., 1, 2, 3, 4, or 5) of the following amino acid substitutions L235V, F243L, R292P, Y300L, and/or P396L, EU numbering according to Kabat.

In some embodiments, the IgG1 Fc region comprises an amino acid substitution at one or more (e.g., 1, 2, 3, 4, 5, 6, or 7) of amino acid positions L234, L235, G236, S239, H268, D270, and/or S298, EU numbering according to Kabat. In some embodiments, the IgG1 Fc region comprises one or more (e.g., 1, 2, 3, 4, 5, 6, or 7) of the following amino acid substitutions L234Y, L235Q, G236W, S239M, H268D, D270E, and/or S298A, EU numbering according to Kabat.

In some embodiments, the IgG1 Fc region comprises an amino acid substitution at one or more (e.g., 1, 2, 3, or 4) of amino acid positions D270, K326, A330, and/or K334, EU numbering according to Kabat. In some embodiments, the IgG1 Fc region comprises one or more (e.g., 1, 2, 3, or 4) of the following amino acid substitutions D270E, K326D, A330M, and/or K334E, EU numbering according to Kabat.

In some embodiments, the IgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the IgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the IgG1 Fc region comprises an amino acid substitution at one or more (e.g., 1, 2, or 3) of amino acid positions S239, 1332, and/or A330, EU numbering according to Kabat. In some embodiments, the IgG1 Fc region comprises one or more (e.g., 1, 2, or 3) of the following amino acid substitutions S239D, 1332E, and/or A330L, EU numbering according to Kabat.

In some embodiments, the IgG1 Fc region comprises an amino acid substitution at one or more (e.g., 1, 2, 3, or 4) of amino acid positions S239, 1332, A330, and/or G236, EU numbering according to Kabat. In some embodiments, the IgG1 Fc region comprises one or more (e.g., 1, 2, 3, or 4) of the following amino acid substitutions S239D, 1332E, A330L and/or G236A, EU numbering according to Kabat.

In some embodiments, the IgG1 Fc region comprises an amino acid substitution at one or more (e.g., 1, 2, or 3) of amino acid positions S239, 1332, and/or G326, EU numbering according to Kabat. In some embodiments, the IgG1 Fc region comprises one or more (e.g., 1, 2, or 3) of the following amino acid substitutions S239D, 1332E, and/or G326A, EU numbering according to Kabat.

In some embodiments, the IgG1 Fc region comprises an amino acid substitution at amino acid position G326, EU numbering according to Kabat. In some embodiments, the IgG1 Fc region comprises a G326A amino acid substitution, EU numbering according to Kabat.

In some embodiments, the IgG1 Fc region comprises an amino acid substitution at one or more (e.g., 1, 2, or 3) of amino acid positions G236, S239, and/or 1332, EU numbering according to Kabat. In some embodiments, the IgG1 Fc region comprises one or more (e.g., 1, 2, or 3) of the following amino acid substitutions G236A, S239D, and/or 1332E, EU numbering according to Kabat.

In some embodiments, the IgG1 Fc region comprises an amino acid substitution at one or more (e.g., 1 or 2) of amino acid positions S239 and/or 1332, EU numbering according to Kabat. In some embodiments, the IgG1 Fc region comprises one or more (e.g., 1 or 2) of the following amino acid substitutions S239D and/or 1332E, EU numbering according to Kabat.

In some embodiments, the IgG1 Fc region comprises an amino acid substitution at one or more (e.g., 1 or 2) of amino acid positions K326 and/or E333, EU numbering according to Kabat. In some embodiments, the IgG1 Fc region comprises one or more (e.g., 1 or 2) of the following amino acid substitutions K326W and/or E333S, EU numbering according to Kabat.

In some embodiments, the IgG1 Fc region comprises an amino acid substitution at one or more (e.g., 1, 2, or 3) of amino acid positions S267, H268, and/or S324, EU numbering according to Kabat. In some embodiments, the IgG1 Fc region comprises one or more (e.g., 1, 2, or 3) of the following amino acid substitutions S267E, H268E, and/or S324T, EU numbering according to Kabat.

In some embodiments, the IgG1 Fc region comprises an amino acid substitution at one or more (e.g., 1, 2, or 3) of amino acid positions S298, E333, and/or K334, EU numbering according to Kabat. In some embodiments, the IgG1 Fc region comprises one or more (e.g., 1, 2, or 3) of the following amino acid substitutions S298A, E333A, and/or K334A, EU numbering according to Kabat.

In some embodiments, the IgG1 Fc region comprises an amino acid substitution at one or more (e.g., 1 or 2) of amino acid positions S239 and/or 1332, EU numbering according to Kabat. In some embodiments, the IgG1 Fc region comprises one or more (e.g., 1 or 2) of the following amino acid substitutions S239D and/or 1332E, EU numbering according to Kabat.

In some embodiments, the IgG1 Fc region comprises an amino acid substitution at one or more (e.g., 1 or 2) of amino acid positions P247 and/or A339, EU numbering according to Kabat. In some embodiments, the IgG1 Fc region comprises one or more (e.g., 1 or 2) of the following amino acid substitutions P247I and/or A339Q, EU numbering according to Kabat.

In some embodiments, the Ig Fc region of a CD161 binding protein (or a conjugate or fusion protein comprising the same) comprises one or more changes to the glycosylation. In some embodiments, the Ig Fc region of a CD161 binding protein (or a conjugate or fusion protein comprising the same) is afucosylated. In some embodiments, the Ig Fc region of a CD161 binding protein (or a conjugate or fusion protein comprising the same) is afucosylated and a CD161 binding protein (or a conjugate or fusion protein comprising the same) comprising the afucosylated Ig Fc exhibits enhanced (e.g., increased) ADCC compared to a reference CD161 binding protein (or a conjugate or fusion protein comprising the same) that is not afucosylated.

### 5.8 Signal Peptides

In some embodiments, one or more polypeptide of a CD161 binding protein (or one or more polypeptide thereof) or polypeptide (or a conjugate or fusion protein comprising the same) (e.g., described herein) comprises a signal peptide operably connected to the N-terminus. Commonly utilized signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art. The amino acid sequence of exemplary signal peptides is provided in Table 9.

TABLE 9
The amino acid sequence of exemplary
signal peptides

| Description | Amino Acid Sequence | SEQ ID |
|---|---|---|
| | | NO |
| hIL-2 | MYRMQLLSCIALSLALVINS | 164 |
| hOSM | MGVLLTQRTLLSLVLALLFP | 165 |
| | SMASM | |
| hCTRB1 | MASLWLLSCESLVGAAFG | 166 |
| hTRY2 | MNLLLILTFVAAAVA | 167 |
| hINS | MALWMRLLPLLALLALWGPD | 168 |
| | PAAA | |

In some embodiments, the amino acid sequence of the signal peptide comprises or consists of the amino acid sequence of any one of the signal peptides set forth in Table 9. In some embodiments, the amino acid sequence of the signal peptide comprises or consists of the amino acid sequence of any one of the signal peptides set forth in Table 9, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence of any one of the signal peptides set forth in Table 9, comprising 1, 2, or 3 amino acid variations (e.g., substitutions, deletions, additions). In some embodiments, the amino acid sequence of the signal peptide comprises or consists of the amino acid sequence of any one of the signal peptides set forth in Table 9, and further comprises 1 or more but less than 15% (less than 12%, less than 10%, less than 8%), amino acid substitutions. In some embodiments, the amino acid sequence of the signal peptide comprises or consists of the amino acid sequence of any one of the signal peptides set forth in Table 9, comprising 1, 2, or 3 amino acid substitutions.

In some embodiments, the amino acid sequence of the signal peptide comprises or consists of the amino acid sequence of any one of SEQ ID NOS: 164-168. In some embodiments, the amino acid sequence of the signal peptide comprises or consists of the amino acid sequence of any one of SEQ ID NOS: 164-168, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence of any one of SEQ ID NOS: 164-168, comprising 1, 2, or 3 amino acid variations (e.g., substitutions, deletions, additions). In some embodiments, the amino acid sequence of the signal peptide comprises or consists of the amino acid sequence of any one of SEQ ID NOS: 164-168, and further comprises 1 or more but less than 15% (less than 12%, less than 10%, less than 8%), amino acid substitutions. In some embodiments, the amino acid sequence of the signal peptide comprises or consists of the amino acid sequence of any one of SEQ ID NOS: 164-168, comprising 1, 2, or 3 amino acid substitutions.

### 5.9 Nucleic Acid Molecules, Vectors, Cells, & Carriers

In one aspect, provided herein are nucleic acid molecules (e.g., DNA, RNA) encoding a CD161 binding protein (or a polypeptide thereof) described herein, a fusion protein described herein, or a conjugate described herein. In some embodiments, the nucleic acid molecule is a DNA molecule. In some embodiments, the nucleic acid molecule is an RNA nucleic acid molecule. In some embodiments, the nucleic acid molecule is an mRNA molecule.

In some embodiments, the nucleic acid molecule is a linear coding nucleic acid construct. In some embodiments, the nucleic acid molecule is contained within a vector non-viral vector (e.g., a plasmid), viral vector). In some embodiments, the nucleic acid molecule is contained within a non-viral vector (e.g., a plasmid). In some embodiments, the nucleic acid molecule is contained within a plasmid. In some embodiments, the nucleic acid molecule is contained within a viral vector.

In some embodiments, the nucleic acid molecule is modified (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc.

In some embodiments, the nucleic acid molecule is codon optimized. Codon optimization, may be used to match codon frequencies in target and host organisms to ensure proper folding; bias guanosine (G) and/or cytosine I content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

In one aspect, provided herein are vectors comprising a nucleic acid molecule (e.g., DNA, RNA (e.g., mRNA)) described herein. In some embodiments, the vector is a viral vector. In some embodiments, the vector is a non-viral vector (e.g., a plasmid, minicircle). In some embodiments, the vector is a plasmid. In some embodiments, the vector is a minicircle.

In one aspect, provided herein a cell (e.g., a host cell, a therapeutic cell) or a population of cells (e.g., a population of host cells, a population of therapeutic cells) comprising a CD161 binding protein (or a polypeptide thereof) described herein, a fusion protein described herein, a conjugate described herein, a nucleic acid molecule described herein, or a vector described herein (e.g., a vector comprising a nucleic acid molecule described herein). In some embodiments, the cell is a eukaryotic cell. In some embodiments, the cell is mammalian cell. In some embodiments, the cell is an animal cell. In some embodiments, the cell is a human cell. In some embodiments, the cell is in vitro. In some embodiments, the cell is in vivo. In some embodiments, the cell is ex vivo. Standard methods known in the art can be utilized to deliver any one of the foregoing (e.g., CD161 binding proteins, fusion protein, vector, nucleic acid molecule, carrier, etc.) into a cell (e.g., a host cell). Standard methods known in the art can be utilized to culture cells (e.g., host cells) in vitro or ex vivo.

In one aspect, provided herein are carriers comprising a CD161 binding protein (or a polypeptide thereof) or polypeptide described herein, a fusion protein described herein, or a conjugate described herein, a nucleic acid molecule described herein, or a vector described herein (e.g., a vector comprising a nucleic acid molecule described herein). Exemplary carriers include, but are not limited to, nanoparticles, polymers, viruses (e.g., a recombinant viruses), virus like particles, virosomes, fusosomes, vesicles, or lipid-based carriers (e.g., lipid nanoparticles (LNPs), liposomes, lipoplexes, nanoliposomes, exosomes, or micelles). In some embodiments, the carrier is a lipid-based carrier such as an LNP, liposome, lipoplex, or nanoliposome. In some embodiments, the carrier is an LNP.

### 5.10 Methods of Making Proteins

The CD161 binding proteins (and polypeptides thereof) and the fusion proteins described herein may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the CD161 binding protein (or polypeptide thereof) (or the fusion protein). Such general methods are common knowledge in the art. The expression vector typically contains an expression cassette that includes nucleic acid sequences capable of bringing about expression of the nucleic acid molecule encoding the protein of interest (e.g., encoding the CD161 binding protein (or polypeptide thereof) (or the fusion protein)), such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the immunogenic peptide or protein. Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. see, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes. The CD161 binding proteins (and polypeptides thereof) (and fusion proteins) described herein may be produced synthetically.

As such, in one aspect, described herein is a method of manufacturing a CD161 binding protein (or a polypeptide thereof) described herein (or a fusion protein described herein), the method comprising introducing into a cell a nucleic acid molecule described herein (e.g., a nucleic acid molecule encoding a CD161 binding protein described herein, a fusion protein described herein), a vector described herein (e.g., a vector comprising a nucleic acid molecule described herein), or a carrier described herein (e.g., a carrier comprising a nucleic acid molecule or a vector described herein); culturing the cell under conditions that allow for expression of the CD161 binding protein (or polypeptide thereof) (or the fusion protein); and optionally recovering the expressed the CD161 binding protein (or polypeptide thereof) (or the fusion protein) from the culture; and optionally purifying the expressed the CD161 binding protein (or polypeptide thereof) (or the fusion protein) from the culture.

In some embodiments, the disclosure features methods of making the CD161 binding proteins described herein (and fusion proteins described herein). The method includes (a) recombinantly expressing a CD161 binding protein (or a fusion protein comprising the same) described herein; (b) enriching, e.g., purifying, the CD161 binding protein (or the fusion protein comprising the same); (c) evaluating the CD161 binding protein (or the fusion protein comprising the same) described herein for the presence of a process impurity or contaminant, and (d) formulating the CD161 binding protein (or the fusion protein comprising the same) as a pharmaceutical composition if the CD161 binding protein (or the fusion protein comprising the same) meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

In some embodiments, the cell cannot mediate fucosylation of produced CD161 binding protein. Exemplary cells that cannot mediate fucosylation are known in the art. See, e.g., Pereira, Natasha A et al. “The “less-is-more” in therapeutic antibodies: Afucosylated anti-cancer antibodies with enhanced antibody-dependent cellular cytotoxicity.” mAbs vol. 10, 5 (2018): 693-711. doi:10.1080/19420862.2018.1466767, the entire contents of which is incorporated herein by reference for all purposes.

In some embodiments, the disclosure features methods of making the CD161 binding proteins described herein (and fusion proteins described herein). The method includes (a) recombinantly expressing a CD161 binding protein (or a fusion protein comprising the same) described herein; (b) enriching, e.g., purifying, the CD161 binding protein (or the fusion protein comprising the same); (c) evaluating the CD161 binding protein (or the fusion protein comprising the same) described herein for the presence of a process impurity or contaminant, and (d) formulating the CD161 binding protein (or the fusion protein comprising the same) as a pharmaceutical composition if the CD161 binding protein (or the fusion protein comprising the same) meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.11 Pharmaceutical Compositions

In one aspect, provided herein are pharmaceutical compositions comprising a CD161 binding protein described herein, a fusion protein described herein, a conjugate described herein, a nucleic acid molecule described herein, a vector described herein, a cell (or population of cells) described herein, or a carrier described herein, and a pharmaceutically acceptable excipient (see, e.g., Remington's Pharmaceutical Sciences (1990) Mack Publishing Co., Easton, PA, the entire contents of which is incorporated by reference herein for all purposes).

Also provided herein are pharmaceutical compositions comprising a CD161 binding protein described herein, a fusion protein described herein, a conjugate described herein, a nucleic acid molecule described herein, a vector described herein, a cell (or population of cells) described herein, or a carrier described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

In one aspect, also provided herein are methods of making pharmaceutical compositions described herein comprising providing a CD161 binding protein described herein, a fusion protein described herein, a conjugate, a nucleic acid molecule described herein, a vector described herein, a cell (or population of cells) described herein, or a carrier described herein, and formulating it into a pharmaceutically acceptable composition by the addition of one or more pharmaceutically acceptable excipient.

Acceptable excipients are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

A pharmaceutical composition may be formulated for any route of administration to a subject. The skilled person knows the various possibilities to administer a pharmaceutical composition described herein a in order to induce an immune response to the immunogen(s) in the pharmaceutical composition. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.12 Methods of Use

Provided herein are various methods of utilizing the CD161 binding proteins, fusion proteins described herein, conjugates described herein, nucleic acid molecules described herein, vectors described herein, cells (or population of cells) described herein, carriers described herein, and pharmaceutical compositions described herein.

As described below, in some aspects and embodiments, the methods comprise administration of a CD161 binding protein described herein (e.g., an anti-CD161 antibody described herein), a polynucleotide described herein (e.g., a polynucleotide encoding a CD161 binding protein described herein (e.g., an anti-CD161 antibody described herein)), a vector described herein (e.g., a vector comprising a polynucleotide described herein (e.g., a polynucleotide encoding a CD161 binding protein described herein (e.g., an anti-CD161 antibody described herein))), a cell (or population of cells) described herein (e.g., a cell (or population of cells) comprising a CD161 binding protein described herein (e.g., an anti-CD161 antibody described herein), a polynucleotide described herein (e.g., a polynucleotide encoding a CD161 binding protein described herein (e.g., an anti-CD161 antibody described herein)), a vector described herein (e.g., a vector comprising a polynucleotide described herein (e.g., a polynucleotide encoding a CD161 binding protein described herein (e.g., an anti-CD161 antibody described herein)))), or a carrier described herein (e.g., a carrier comprising a CD161 binding protein described herein (e.g., an anti-CD161 antibody described herein), a polynucleotide described herein (e.g., a polynucleotide encoding a CD161 binding protein described herein (e.g., an anti-CD161 antibody described herein)), a vector described herein (e.g., a vector comprising a polynucleotide described herein (e.g., a polynucleotide encoding a CD161 binding protein described herein (e.g., an anti-CD161 antibody described herein))), or a cell (or population of cells) described herein) to a subject.

Exemplary subjects include mammals, e.g., humans, non-human mammals, e.g., non-human primates. In some embodiments, the subject is a human.

The dosage of a CD161 binding protein described herein, a fusion protein described herein, a conjugate described herein, a nucleic acid molecule described herein, a vector described herein, a cell (or population of cells) described herein, a carrier described herein, or a pharmaceutical composition described herein to be administered to a subject in accordance with any of the methods described herein can be determined in accordance with standard techniques known to those of ordinary skill in the art, including the route of administration, the age and weight of the subject.

### 5.12.1 Samples

Some of the methods described herein utilize a sample from a subject. A suitable sample source, size, etc. can be determined by a person of ordinary skill in the art in accordance with use in the selected method. Exemplary subject samples include, but are not limited to, tissue, cell, blood, plasma, saliva sample, and nasal swab. Other samples include, but are not limited to, semen, sputum, mucous, sweat, urine, and feces. In some embodiments, the sample is a blood, cell, or tissue sample. In some embodiments, the sample is a cell sample. In some embodiments, the sample is a tissue sample. In some embodiments, the sample is a blood or plasma sample. In some embodiments, the sample is a biopsy. In some embodiments, the sample is a tumor cell sample. In some embodiments, the sample is a tumor tissue sample. In some embodiments, the sample is a tumor biopsy.

In some embodiments, the sample comprises a population of cells. In some embodiments, the sample comprises a population of immune cells. In some embodiments, the sample comprises a population of tumor cells. In some embodiments, the sample comprises a population of immune cells and a population of tumor cells. Exemplary immune cells include, but are not limited to, T cells, natural killer (NK) cells. In some embodiments, the sample comprises T cells. In some embodiments, the sample comprises NK cells. In some embodiments, the sample comprises tumor infiltrating T cells. In some embodiments, the sample comprises tumor infiltrating NK cells. In some embodiments, the sample comprises T cells (e.g., tumor infiltrating T cells) and NK cells (e.g., tumor infiltrating NK cells). In some embodiments, the sample comprises T cells (e.g., tumor infiltrating T cells), NK cells (e.g., tumor infiltrating NK cells), and tumor cells.

In some embodiments, the sample has been manipulated after obtaining. In some embodiments, the sample has been fixed, embedded in paraffin, sectioned, deparaffinized, and transferred to a slide. In some embodiments, the sample is a formalin-fixed paraffin-embedded (FFPE) tissue sample.

### 5.12.2 Cancers

Some of the methods described herein relate to a cancer. In some embodiments, the cancer is a solid tumor. In some embodiments, the cancer is a carcinoma, adenocarcinoma, or sarcoma. In some embodiments, the cancer is a carcinoma. In some embodiments, the cancer is an adenocarcinoma. In some embodiments, the cancer is a squamous cell carcinoma. In some embodiments, the cancer is a cutaneous squamous cell carcinoma.

In some embodiments, the cancer is head cancer, neck cancer, head or neck cancer, lung cancer, colon cancer, rectal cancer, colorectal cancer, renal cancer, ovarian cancer, breast cancer, endometrial cancer, uterine cancer, cervical cancer, anal cancer, prostate cancer, bladder cancer, liver cancer, pancreatic cancer, thyroid cancer, thymus cancer, bronchus cancer, skin cancer, brain cancer, spinal cord cancer, lip cancer, bowel cancer (e.g., small bowel cancer, large bowel cancer), or oral cavity cancer. In some embodiments, the cancer is head cancer, neck cancer, head and neck cancer, lung cancer, colon cancer, rectal cancer, colorectal cancer (CRC), renal cancer, or ovarian cancer. In some embodiments, the cancer is head and neck squamous cell carcinoma (HNSCC), non-small cell lung cancer (NSCLC), colorectal carcinoma, renal cell carcinoma, cutaneous squamous cell carcinoma. In some embodiments, the cancer is non-small cell lung cancer, head and neck squamous cell carcinoma, triple negative breast cancer, cutaneous squamous cell carcinoma, hormone receptor positive breast carcinoma, small bowel cancer, esophageal cancer, or colorectal cancer.

In some embodiments, the cancer is a hematological cancer. In some embodiments, the cancer is a lymphoma, leukemia, or myeloma. In some embodiments, the cancer is a lymphoma. In some embodiments, the cancer is diffuse large B cell lymphoma, Hodgkin's lymphoma, Burkitt lymphoma, or T-cell lymphoma. In some embodiments, the cancer is a leukemia (e.g., acute leukemia, acute lymphoblastic, leukemia (ALL) (e.g., B-cell ALL, T-cell ALL), acute myeloid leukemia (AML), chronic myelocytic leukemia (CML), chronic lymphocytic leukemia (CLL), hairy cell leukemia.

In specific embodiments, the cancer is head and neck squamous cell carcinoma (HNSCC), colorectal cancer (CRC), or non-small cell lung cancer (NSCLC). In specific embodiments, the cancer is squamous cell lung cancer. In specific embodiments, the cancer is head and neck squamous cell carcinoma (HNSCC). In specific embodiments, the cancer is HPV positive head and neck squamous cell carcinoma (HNSCC). In specific embodiments, the cancer is HPV negative head and neck squamous cell carcinoma (HNSCC). In specific embodiments, the cancer is colorectal cancer (CRC). In specific embodiments, the cancer is non-small cell lung cancer (NSCLC). In specific embodiments, the cancer is Hodgkin's lymphoma. In specific embodiments, the cancer is cutaneous squamous cell carcinoma. In specific embodiments, the cancer is breast cancer. In specific embodiments, the cancer is breast cancer characterized as expressing a genetic alteration in BRCA1 and/or BRCA2. In specific embodiments, the cancer is triple negative breast cancer. In specific embodiments, the cancer is diffuse large B cell lymphoma. In specific embodiments, the cancer is esophageal cancer.

### 5.12.3 Agents that Inhibits the Interaction of CD161 to CLEC2D

Some of the methods described herein utilize an agent (e.g., anti-CD161 antibody (e.g., an anti-CD161 antibody described herein)) that inhibits the interaction of CD161 to CLEC2D. In some embodiments, the agent is a therapeutic agent (e.g., a therapeutic anti-CD161 antibody (e.g., described herein)).

In some embodiments, the agent is a CD161 binding protein (e.g., antibody) described herein. In some embodiments, the agent is a CD161 binding protein (e.g., antibody) (or a functional fragment or variant thereof) set forth in Table 3 herein. In some embodiments, the agent is a CD161 binding protein described herein. In some embodiments, the agent is a CD161 binding protein (e.g., antibody) (or a functional fragment or variant thereof) set forth in Table 4 of WO2023028501, the entire contents of which is incorporated herein by reference for all purposes.

In some embodiments, the agent comprises a CD161 binding protein. In some embodiments, the CD161 binding protein is an antibody. In some embodiments, the CD161 binding protein comprises an antibody. In some embodiments, the CD161 binding protein consists of an antibody. In some embodiments, the agent is a CD161 binding protein described in § 5.2 herein, the entire contents of said section are incorporated herein.

In some embodiments, the CD161 binding protein (e.g., antibody) comprises a CD161 binding protein provided in Table 3. In some embodiments, the CD161 binding protein comprises CD161 binding protein Ab-2 (see Table 3). In some embodiments, the CD161 binding protein comprises CD161 binding protein Ab-3 (see Table 3). In some embodiments, the CD161 binding protein comprises CD161 binding protein Ab-4 (see Table 3). In some embodiments, the CD161 binding protein comprises CD161 binding protein Ab-5 (see Table 3). In some embodiments, the CD161 binding protein comprises CD161 binding protein Ab-6 (see Table 3). In some embodiments, the CD161 binding protein comprises CD161 binding protein Ab-7 (see Table 3). In some embodiments, the CD161 binding protein comprises CD161 binding protein Ab-8 (see Table 3). In some embodiments, the CD161 binding protein comprises CD161 binding protein Ab-9 (see Table 3). In some embodiments, the CD161 binding protein comprises CD161 binding protein Ab-10 (see Table 3). In some embodiments, the CD161 binding protein comprises CD161 binding protein Ab-11 (see Table 3). In some embodiments, the CD161 binding protein comprises CD161 binding protein Ab-12 (see Table 3). In some embodiments, the CD161 binding protein comprises CD161 binding protein Ab-13 (see Table 3). In some embodiments, the CD161 binding protein comprises CD161 binding protein Ab-14 (see Table 3). In some embodiments, the CD161 binding protein comprises CD161 binding protein Ab-15 (see Table 3). In some embodiments, the CD161 binding protein comprises CD161 binding protein Ab-16 (see Table 3). In some embodiments, the CD161 binding protein comprises CD161 binding protein Ab-17 (see Table 3). In some embodiments, the CD161 binding protein comprises CD161 binding protein Ab-18 (see Table 3). In some embodiments, the CD161 binding protein comprises CD161 binding protein Ab-19 (see Table 3). In some embodiments, the CD161 binding protein comprises CD161 binding protein Ab-20 (see Table 3). In some embodiments, the CD161 binding protein comprises CD161 binding protein Ab-21 (see Table 3).

In some embodiments, the CD161 binding protein (e.g., anti-CD161 antibody) comprises a VH that comprises: a CDR-H1, a CDR-H2, and a CDR-H3. In some embodiments, the CD161 binding protein comprises a VL that comprises: CDR-L1, CDR-L2, and CDR-L3. In some embodiments, the CD161 binding protein comprises a VH that comprises: a CDR-H1, a CDR-H2, and a CDR-H3; and a VL that comprises: CDR-L1, CDR-L2, and CDR-L3.

In some embodiments, the amino acid sequence of CDR-H1 comprises the amino acid sequence of CDR-H1 of a CD161 binding protein (e.g., antibody) named or incorporated by reference herein, or the amino acid sequence of the CDR-H1 of the CD161 binding protein (e.g., antibody) named or incorporated by reference herein comprising 1, 2, or 3 amino acid variations (e.g., substitution, deletion, addition, etc.); the amino acid sequence of CDR-H2 comprises the amino acid sequence of CDR-H2 of the CD161 binding protein (e.g., antibody) named or incorporated by reference herein, or the amino acid sequence of CDR-H2 of the CD161 binding protein (e.g., antibody) named or incorporated by reference herein comprising 1, 2, or 3 amino acid variations (e.g., substitution, deletion, addition, etc.); the amino acid sequence of CDR-H3 comprises the amino acid sequence of CDR-H3 of the CD161 binding protein (e.g., antibody) named or incorporated by reference herein, or the amino acid sequence of CDR-H3 of the CD161 binding protein (e.g., antibody) named or incorporated by reference herein comprising 1, 2, or 3 amino acid variations (e.g., substitution, deletion, addition, etc.); the amino acid sequence of CDR-L1 comprises the amino acid sequence of CDR-L1 of the CD161 binding protein (e.g., antibody) named or incorporated by reference herein, or the amino acid sequence of CDR-L1 of the CD161 binding protein (e.g., antibody) named or incorporated by reference herein comprising 1, 2, or 3 amino acid variations (e.g., substitution, deletion, addition, etc.); the amino acid sequence of CDR-L2 comprises the amino acid sequence of CDR-L2 of the CD161 binding protein (e.g., antibody) named or incorporated by reference herein, or the amino acid sequence of CDR-L2 of the CD161 binding protein (e.g., antibody) named or incorporated by reference herein comprising 1, 2, or 3 amino acid variations (e.g., substitution, deletion, addition, etc.); and the amino acid sequence of CDR-L3 comprises the amino acid sequence of the CDR-L3 of the CD161 binding protein (e.g., antibody) named or incorporated by reference herein, or the amino acid sequence of the CDR-L3 of the CD161 binding protein (e.g., antibody) named or incorporated by reference herein comprising 1, 2, or 3 amino acid variations (e.g., substitution, deletion, addition, etc.).

In some embodiments, the amino acid sequence of CDR-H1 comprises the amino acid sequence of CDR-H1 of a CD161 binding protein (e.g., antibody) named or incorporated by reference herein, or the amino acid sequence of CDR-H1 of the CD161 binding protein (e.g., antibody) named or incorporated by reference herein, comprising no more than 1, 2, or 3 amino acid variations (e.g., substitution, deletion, addition, etc.); the amino acid sequence of CDR-H2 comprises the amino acid sequence of CDR-H2 of the CD161 binding protein (e.g., antibody) named or incorporated by reference herein, or the amino acid sequence of CDR-H2 of the CD161 binding protein (e.g., antibody) named or incorporated by reference herein comprising no more than 1, 2, or 3 amino acid variations (e.g., substitution, deletion, addition, etc.); the amino acid sequence of CDR-H3 comprises of CDR-H3 of the CD161 binding protein (e.g., antibody) named or incorporated by reference herein, or the amino acid sequence of CDR-H3 of the CD161 binding protein (e.g., antibody) named or incorporated by reference herein comprising no more than 1, 2, or 3 amino acid variations (e.g., substitution, deletion, addition, etc.); the amino acid sequence of CDR-L1 comprises the amino acid sequence of CDR-L1 of the CD161 binding protein (e.g., antibody) named or incorporated by reference herein, or the amino acid sequence of CDR-L1 of the CD161 binding protein (e.g., antibody) named or incorporated by reference herein comprising no more than 1, 2, or 3 amino acid variations (e.g., substitution, deletion, addition, etc.); the amino acid sequence of CDR-L2 comprises the amino acid sequence of CDR-L2 of the CD161 binding protein (e.g., antibody) named or incorporated by reference herein, or the amino acid sequence of CDR-L2 of the CD161 binding protein (e.g., antibody) named or incorporated by reference herein comprising no more than 1, 2, or 3 amino acid variations (e.g., substitution, deletion, addition, etc.); and the amino acid sequence of CDR-L3 comprises the amino acid sequence of CDR-L3 of the CD161 binding protein (e.g., antibody) named or incorporated by reference herein, or the amino acid sequence of CDR-L3 of the CD161 binding protein (e.g., antibody) named or incorporated by reference herein comprising no more than 1, 2, or 3 amino acid variations (e.g., substitution, deletion, addition, etc.).

In some embodiments, the amino acid sequence of CDR-H1 comprises the amino acid sequence of CDR-H1 of a CD161 binding protein (e.g., antibody) named or incorporated by reference herein; the amino acid sequence of CDR-H2 comprises the amino acid sequence of CDR-H2 of the CD161 binding protein (e.g., antibody) named or incorporated by reference herein; the amino acid sequence of CDR-H3 comprises CDR-H3 of the CD161 binding protein (e.g., antibody) named or incorporated by reference herein; the amino acid sequence of CDR-L1 comprises the amino acid sequence of CDR-L1 of the CD161 binding protein (e.g., antibody) named or incorporated by reference herein; the amino acid sequence of CDR-L2 comprises the amino acid sequence of CDR-L2 of the CD161 binding protein (e.g., antibody) named or incorporated by reference herein; and the amino acid sequence of CDR-L3 comprises the amino acid sequence of CDR-L3 of the CD161 binding protein (e.g., antibody) named or incorporated by reference herein.

In some embodiments, the amino acid sequence of CDR-H1 comprises the amino acid sequence of CDR-H1 of a CD161 binding protein (e.g., antibody) named or incorporated by reference herein comprising no more than 1, 2, or 3 amino acid variations (e.g., substitution, deletion, addition, etc.); the amino acid sequence of CDR-H2 comprises the amino acid sequence CDR-H2 of the CD161 binding protein (e.g., antibody) named or incorporated by reference herein comprising no more than 1, 2, or 3 amino acid variations (e.g., substitution, deletion, addition, etc.); the amino acid sequence of CDR-H3 comprises the amino acid sequence CDR-H3 of the CD161 binding protein (e.g., antibody) named or incorporated by reference herein comprising no more than 1, 2, or 3 amino acid variations (e.g., substitution, deletion, addition, etc.); the amino acid sequence of CDR-L1 comprises the amino acid sequence CDR-L1 of the CD161 binding protein (e.g., antibody) named or incorporated by reference herein comprising no more than 1, 2, or 3 amino acid variations (e.g., substitution, deletion, addition, etc.); the amino acid sequence of a CDR-L2 comprises the amino acid sequence CDR-L2 of the CD161 binding protein (e.g., antibody) named or incorporated by reference herein comprising no more than 1, 2, or 3 amino acid variations (e.g., substitution, deletion, addition, etc.); and the amino acid sequence of a CDR-L3 comprises the amino acid sequence CDR-L3 of the CD161 binding protein (e.g., antibody) named or incorporated by reference herein comprising no more than 1, 2, or 3 amino acid variations (e.g., substitution, deletion, addition, etc.).

In some embodiments, the amino acid sequence of CDR-H1 consists of the amino acid sequence of CDR-H1 of a CD161 binding protein (e.g., antibody) named or incorporated by reference herein, or the amino acid sequence of CDR-H1 of the CD161 binding protein (e.g., antibody) named or incorporated by reference herein, comprising 1, 2, or 3 amino acid variations (e.g., substitution, deletion, addition, etc.); the amino acid sequence of CDR-H2 consists of the amino acid sequence of CDR-H2 of the CD161 binding protein (e.g., antibody) named or incorporated by reference herein, or the amino acid sequence of CDR-H2 of the CD161 binding protein (e.g., antibody) named or incorporated by reference herein comprising 1, 2, or 3 amino acid variations (e.g., substitution, deletion, addition, etc.); the amino acid sequence of CDR-H3 consists of the amino acid sequence of CDR-H3 of the CD161 binding protein (e.g., antibody) named or incorporated by reference herein, or the amino acid sequence of CDR-H3 of the CD161 binding protein (e.g., antibody) named or incorporated by reference herein comprising 1, 2, or 3 amino acid variations (e.g., substitution, deletion, addition, etc.); the amino acid sequence of CDR-L1 consists of the amino acid sequence of CDR-L1 of the CD161 binding protein (e.g., antibody) named or incorporated by reference herein, or the amino acid sequence of CDR-L1 of the CD161 binding protein (e.g., antibody) named or incorporated by reference herein comprising 1, 2, or 3 amino acid variations (e.g., substitution, deletion, addition, etc.); the amino acid sequence of CDR-L2 consists of the amino acid sequence of CDR-L2 of the CD161 binding protein (e.g., antibody) named or incorporated by reference herein, or the amino acid sequence of CDR-L2 of the CD161 binding protein (e.g., antibody) named or incorporated by reference herein comprising 1, 2, or 3 amino acid variations (e.g., substitution, deletion, addition, etc.); and the amino acid sequence of CDR-L3 consists of the amino acid sequence of CDR-L3 of the CD161 binding protein (e.g., antibody) named or incorporated by reference herein, or the amino acid sequence of CDR-L3 of the CD161 binding protein (e.g., antibody) named or incorporated by reference herein comprising 1, 2, or 3 amino acid variations (e.g., substitution, deletion, addition, etc.).

In some embodiments, the amino acid sequence of CDR-H1 consists of the amino acid sequence of CDR-H1 of a CD161 binding protein (e.g., antibody) named or incorporated by reference herein, or the amino acid sequence of CDR-H1 of the CD161 binding protein (e.g., antibody) named or incorporated by reference herein, comprising no more than 1, 2, or 3 amino acid variations (e.g., substitution, deletion, addition, etc.); the amino acid sequence of CDR-H2 consists of the amino acid sequence of CDR-H2 of the CD161 binding protein (e.g., antibody) named or incorporated by reference herein, or the amino acid sequence of CDR-H2 of the CD161 binding protein (e.g., antibody) named or incorporated by reference herein comprising no more than 1, 2, or 3 amino acid variations (e.g., substitution, deletion, addition, etc.); the amino acid sequence of CDR-H3 consists of the amino acid sequence of CDR-H3 of the CD161 binding protein (e.g., antibody) named or incorporated by reference herein, or the amino acid sequence of CDR-H3 of the CD161 binding protein (e.g., antibody) named or incorporated by reference herein comprising no more than 1, 2, or 3 amino acid variations (e.g., substitution, deletion, addition, etc.); the amino acid sequence of CDR-L1 consists of the amino acid sequence of CDR-L1 of the CD161 binding protein (e.g., antibody) named or incorporated by reference herein, or the amino acid sequence of CDR-L1 of the CD161 binding protein (e.g., antibody) named or incorporated by reference herein comprising no more than 1, 2, or 3 amino acid variations (e.g., substitution, deletion, addition, etc.); the amino acid sequence of CDR-L2 consists of the amino acid sequence of CDR-L2 of the CD161 binding protein (e.g., antibody) named or incorporated by reference herein, or the amino acid sequence of CDR-L2 of the CD161 binding protein (e.g., antibody) named or incorporated by reference herein comprising no more than 1, 2, or 3 amino acid variations (e.g., substitution, deletion, addition, etc.); and the amino acid sequence of CDR-L3 consists of the amino acid sequence of CDR-L3 of the CD161 binding protein (e.g., antibody) named or incorporated by reference herein, or the amino acid sequence of CDR-L3 of the CD161 binding protein (e.g., antibody) named or incorporated by reference herein comprising no more than 1, 2, or 3 amino acid variations (e.g., substitution, deletion, addition, etc.).

In some embodiments, the amino acid sequence of CDR-H1 consists of the amino acid sequence of CDR-H1 of a CD161 binding protein (e.g., antibody) named or incorporated by reference herein; the amino acid sequence of CDR-H2 consists of the amino acid sequence of CDR-H2 of the CD161 binding protein (e.g., antibody) named or incorporated by reference herein; the amino acid sequence of CDR-H3 consists of CDR-H3 of the CD161 binding protein (e.g., antibody) named or incorporated by reference herein; the amino acid sequence of CDR-L1 consists of the amino acid sequence of CDR-L1 of the CD161 binding protein (e.g., antibody) named or incorporated by reference herein; the amino acid sequence of CDR-L2 consists of the amino acid sequence of CDR-L2 of the CD161 binding protein (e.g., antibody) named or incorporated by reference herein; and the amino acid sequence of CDR-L3 consists of the amino acid sequence of CDR-L3 of the CD161 binding protein (e.g., antibody) named or incorporated by reference herein.

In some embodiments, the amino acid sequence of CDR-H1 consists of the amino acid sequence of CDR-H1 of a CD161 binding protein (e.g., antibody) named or incorporated by reference herein comprising no more than 1, 2, or 3 amino acid variations (e.g., substitution, deletion, addition, etc.); the amino acid sequence of CDR-H2 consists of the amino acid sequence of CDR-H2 of the CD161 binding protein (e.g., antibody) named or incorporated by reference herein comprising no more than 1, 2, or 3 amino acid variations (e.g., substitution, deletion, addition, etc.); the amino acid sequence of CDR-H3 consists of the amino acid sequence of CDR-H3 of the CD161 binding protein (e.g., antibody) named or incorporated by reference herein comprising no more than 1, 2, or 3 amino acid variations (e.g., substitution, deletion, addition, etc.); the amino acid sequence of CDR-L1 consists of the amino acid sequence of CDR-L1 of the CD161 binding protein (e.g., antibody) named or incorporated by reference herein comprising no more than 1, 2, or 3 amino acid variations (e.g., substitution, deletion, addition, etc.); the amino acid sequence of CDR-L2 consists of the amino acid sequence of CDR-L2 of the CD161 binding protein (e.g., antibody) named or incorporated by reference herein comprising no more than 1, 2, or 3 amino acid variations (e.g., substitution, deletion, addition, etc.); and the amino acid sequence of CDR-L3 consists of the amino acid sequence of CDR-L3 of the CD161 binding protein (e.g., antibody) named or incorporated by reference herein comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VH of a CD161 binding protein (e.g., antibody) named or incorporated by reference herein; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of the CD161 binding protein (e.g., antibody) named or incorporated by reference herein. In some embodiments, the amino acid sequence of the VH comprises an amino acid sequence at least 90%, 91%, 92%, 93%, 94%, 95%, 96%, 97%, 98%, 99%, or 100% identical to the amino acid sequence of a VH of a CD161 binding protein (e.g., antibody) named or incorporated by reference herein; and the amino acid sequence of the VL comprises an amino acid sequence at least 90%, 91%, 92%, 93%, 94%, 95%, 96%, 97%, 98%, 99%, or 100% identical to the amino acid sequence of the VL of the CD161 binding protein (e.g., antibody) named or incorporated by reference herein. In some embodiments, the amino acid sequence of the VH comprises an amino acid sequence at least 95%, 96%, 97%, 98%, 99%, or 100% identical to the amino acid sequence of a VH of a CD161 binding protein (e.g., antibody) named or incorporated by reference herein; and the amino acid sequence of the VL comprises an amino acid sequence at least 95%, 96%, 97%, 98%, 99%, or 100% identical to the amino acid sequence of the VL of the CD161 binding protein (e.g., antibody) named or incorporated by reference herein. In some embodiments, the amino acid sequence of the VH comprises the amino acid sequence of a VH of a CD161 binding protein (e.g., antibody) named or incorporated by reference herein; and the amino acid sequence of the VL comprises the amino acid sequence of the VL of the CD161 binding protein (e.g., antibody) named or incorporated by reference herein.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH of a CD161 binding protein (e.g., antibody) named or incorporated by reference herein; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of the CD161 binding protein (e.g., antibody) named or incorporated by reference herein. In some embodiments, the amino acid sequence of the VH consists of an amino acid sequence at least 90%, 91%, 92%, 93%, 94%, 95%, 96%, 97%, 98%, 99%, or 100% identical to the amino acid sequence of a VH of a CD161 binding protein (e.g., antibody) named or incorporated by reference herein; and the amino acid sequence of the VL consists of an amino acid sequence at least 90%, 91%, 92%, 93%, 94%, 95%, 96%, 97%, 98%, 99%, or 100% identical to the amino acid sequence of the VL of the CD161 binding protein (e.g., antibody) named or incorporated by reference herein. In some embodiments, the amino acid sequence of the VH consists of an amino acid sequence at least 95%, 96%, 97%, 98%, 99%, or 100% identical to the amino acid sequence of a VH of a CD161 binding protein (e.g., antibody) named or incorporated by reference herein; and the amino acid sequence of the VL consists of an amino acid sequence at least 95%, 96%, 97%, 98%, 99%, or 100% identical to the amino acid sequence of the VL of the CD161 binding protein (e.g., antibody) named or incorporated by reference herein. In some embodiments, the amino acid sequence of the VH consists of the amino acid sequence of a VH of a CD161 binding protein (e.g., antibody) named or incorporated by reference herein; and the amino acid sequence of the VL consists of the amino acid sequence of the VL of the CD161 binding protein (e.g., antibody) named or incorporated by reference herein.

In some embodiments, the amino acid sequence of CDR-H1 comprises the amino acid sequence of CDR-H1 of a CD161 binding protein (e.g., antibody) set forth in Table 3 herein, or the amino acid sequence of the CDR-H1 of the CD161 binding protein (e.g., antibody) set forth in Table 3 herein comprising 1, 2, or 3 amino acid variations (e.g., substitution, deletion, addition, etc.); the amino acid sequence of CDR-H2 comprises the amino acid sequence of CDR-H2 of the CD161 binding protein (e.g., antibody) set forth in Table 3 herein, or the amino acid sequence of CDR-H2 of the CD161 binding protein (e.g., antibody) set forth in Table 3 herein comprising 1, 2, or 3 amino acid variations (e.g., substitution, deletion, addition, etc.); the amino acid sequence of CDR-H3 comprises the amino acid sequence of CDR-H3 of the CD161 binding protein (e.g., antibody) set forth in Table 3 herein, or the amino acid sequence of CDR-H3 of the CD161 binding protein (e.g., antibody) set forth in Table 3 herein comprising 1, 2, or 3 amino acid variations (e.g., substitution, deletion, addition, etc.); the amino acid sequence of CDR-L1 comprises the amino acid sequence of CDR-L1 of the CD161 binding protein (e.g., antibody) set forth in Table 3 herein, or the amino acid sequence of CDR-L1 of the CD161 binding protein (e.g., antibody) set forth in Table 3 herein comprising 1, 2, or 3 amino acid variations (e.g., substitution, deletion, addition, etc.); the amino acid sequence of CDR-L2 comprises the amino acid sequence of CDR-L2 of the CD161 binding protein (e.g., antibody) set forth in Table 3 herein, or the amino acid sequence of CDR-L2 of the CD161 binding protein (e.g., antibody) set forth in Table 3 herein comprising 1, 2, or 3 amino acid variations (e.g., substitution, deletion, addition, etc.); and the amino acid sequence of CDR-L3 comprises the amino acid sequence of the CDR-L3 of the CD161 binding protein (e.g., antibody) set forth in Table 3 herein, or the amino acid sequence of the CDR-L3 of the CD161 binding protein (e.g., antibody) set forth in Table 3 herein comprising 1, 2, or 3 amino acid variations (e.g., substitution, deletion, addition, etc.).

In some embodiments, the amino acid sequence of CDR-H1 comprises the amino acid sequence of CDR-H1 of a CD161 binding protein (e.g., antibody) set forth in Table 3 herein, or the amino acid sequence of CDR-H1 of the CD161 binding protein (e.g., antibody) set forth in Table 3 herein, comprising no more than 1, 2, or 3 amino acid variations (e.g., substitution, deletion, addition, etc.); the amino acid sequence of CDR-H2 comprises the amino acid sequence of CDR-H2 of the CD161 binding protein (e.g., antibody) set forth in Table 3 herein, or the amino acid sequence of CDR-H2 of the CD161 binding protein (e.g., antibody) set forth in Table 3 herein comprising no more than 1, 2, or 3 amino acid variations (e.g., substitution, deletion, addition, etc.); the amino acid sequence of CDR-H3 comprises of CDR-H3 of the CD161 binding protein (e.g., antibody) set forth in Table 3 herein, or the amino acid sequence of CDR-H3 of the CD161 binding protein (e.g., antibody) set forth in Table 3 herein comprising no more than 1, 2, or 3 amino acid variations (e.g., substitution, deletion, addition, etc.); the amino acid sequence of CDR-L1 comprises the amino acid sequence of CDR-L1 of the CD161 binding protein (e.g., antibody) set forth in Table 3 herein, or the amino acid sequence of CDR-L1 of the CD161 binding protein (e.g., antibody) set forth in Table 3 herein comprising no more than 1, 2, or 3 amino acid variations (e.g., substitution, deletion, addition, etc.); the amino acid sequence of CDR-L2 comprises the amino acid sequence of CDR-L2 of the CD161 binding protein (e.g., antibody) set forth in Table 3 herein, or the amino acid sequence of CDR-L2 of the CD161 binding protein (e.g., antibody) set forth in Table 3 herein comprising no more than 1, 2, or 3 amino acid variations (e.g., substitution, deletion, addition, etc.); and the amino acid sequence of CDR-L3 comprises the amino acid sequence of CDR-L3 of the CD161 binding protein (e.g., antibody) set forth in Table 3 herein, or the amino acid sequence of CDR-L3 of the CD161 binding protein (e.g., antibody) set forth in Table 3 herein comprising no more than 1, 2, or 3 amino acid variations (e.g., substitution, deletion, addition, etc.).

In some embodiments, the amino acid sequence of CDR-H1 comprises the amino acid sequence of CDR-H1 of a CD161 binding protein (e.g., antibody) set forth in Table 3 herein; the amino acid sequence of CDR-H2 comprises the amino acid sequence of CDR-H2 of the CD161 binding protein (e.g., antibody) set forth in Table 3 herein; the amino acid sequence of CDR-H3 comprises CDR-H3 of the CD161 binding protein (e.g., antibody) set forth in Table 3 herein; the amino acid sequence of CDR-L1 comprises the amino acid sequence of CDR-L1 of the CD161 binding protein (e.g., antibody) set forth in Table 3 herein; the amino acid sequence of CDR-L2 comprises the amino acid sequence of CDR-L2 of the CD161 binding protein (e.g., antibody) set forth in Table 3 herein; and the amino acid sequence of CDR-L3 comprises the amino acid sequence of CDR-L3 of the CD161 binding protein (e.g., antibody) set forth in Table 3 herein.

In some embodiments, the amino acid sequence of CDR-H1 comprises the amino acid sequence of CDR-H1 of a CD161 binding protein (e.g., antibody) set forth in Table 3 herein comprising no more than 1, 2, or 3 amino acid variations (e.g., substitution, deletion, addition, etc.); the amino acid sequence of CDR-H2 comprises the amino acid sequence CDR-H2 of the CD161 binding protein (e.g., antibody) set forth in Table 3 herein comprising no more than 1, 2, or 3 amino acid variations (e.g., substitution, deletion, addition, etc.); the amino acid sequence of CDR-H3 comprises the amino acid sequence CDR-H3 of the CD161 binding protein (e.g., antibody) set forth in Table 3 herein comprising no more than 1, 2, or 3 amino acid variations (e.g., substitution, deletion, addition, etc.); the amino acid sequence of CDR-L1 comprises the amino acid sequence CDR-L1 of the CD161 binding protein (e.g., antibody) set forth in Table 3 herein comprising no more than 1, 2, or 3 amino acid variations (e.g., substitution, deletion, addition, etc.); the amino acid sequence of a CDR-L2 comprises the amino acid sequence CDR-L2 of the CD161 binding protein (e.g., antibody) set forth in Table 3 herein comprising no more than 1, 2, or 3 amino acid variations (e.g., substitution, deletion, addition, etc.); and the amino acid sequence of a CDR-L3 comprises the amino acid sequence CDR-L3 of the CD161 binding protein (e.g., antibody) set forth in Table 3 herein comprising no more than 1, 2, or 3 amino acid variations (e.g., substitution, deletion, addition, etc.).

In some embodiments, the amino acid sequence of CDR-H1 consists of the amino acid sequence of CDR-H1 of a CD161 binding protein (e.g., antibody) set forth in Table 3 herein, or the amino acid sequence of CDR-H1 of the CD161 binding protein (e.g., antibody) set forth in Table 3 herein, comprising 1, 2, or 3 amino acid variations (e.g., substitution, deletion, addition, etc.); the amino acid sequence of CDR-H2 consists of the amino acid sequence of CDR-H2 of the CD161 binding protein (e.g., antibody) set forth in Table 3 herein, or the amino acid sequence of CDR-H2 of the CD161 binding protein (e.g., antibody) set forth in Table 3 herein comprising 1, 2, or 3 amino acid variations (e.g., substitution, deletion, addition, etc.); the amino acid sequence of CDR-H3 consists of the amino acid sequence of CDR-H3 of the CD161 binding protein (e.g., antibody) set forth in Table 3 herein, or the amino acid sequence of CDR-H3 of the CD161 binding protein (e.g., antibody) set forth in Table 3 herein comprising 1, 2, or 3 amino acid variations (e.g., substitution, deletion, addition, etc.); the amino acid sequence of CDR-L1 consists of the amino acid sequence of CDR-L1 of the CD161 binding protein (e.g., antibody) set forth in Table 3 herein, or the amino acid sequence of CDR-L1 of the CD161 binding protein (e.g., antibody) set forth in Table 3 herein comprising 1, 2, or 3 amino acid variations (e.g., substitution, deletion, addition, etc.); the amino acid sequence of CDR-L2 consists of the amino acid sequence of CDR-L2 of the CD161 binding protein (e.g., antibody) set forth in Table 3 herein, or the amino acid sequence of CDR-L2 of the CD161 binding protein (e.g., antibody) set forth in Table 3 herein comprising 1, 2, or 3 amino acid variations (e.g., substitution, deletion, addition, etc.); and the amino acid sequence of CDR-L3 consists of the amino acid sequence of CDR-L3 of the CD161 binding protein (e.g., antibody) set forth in Table 3 herein, or the amino acid sequence of CDR-L3 of the CD161 binding protein (e.g., antibody) set forth in Table 3 herein comprising 1, 2, or 3 amino acid variations (e.g., substitution, deletion, addition, etc.).

In some embodiments, the amino acid sequence of CDR-H1 consists of the amino acid sequence of CDR-H1 of a CD161 binding protein (e.g., antibody) set forth in Table 3 herein, or the amino acid sequence of CDR-H1 of the CD161 binding protein (e.g., antibody) set forth in Table 3 herein, comprising no more than 1, 2, or 3 amino acid variations (e.g., substitution, deletion, addition, etc.); the amino acid sequence of CDR-H2 consists of the amino acid sequence of CDR-H2 of the CD161 binding protein (e.g., antibody) set forth in Table 3 herein, or the amino acid sequence of CDR-H2 of the CD161 binding protein (e.g., antibody) set forth in Table 3 herein comprising no more than 1, 2, or 3 amino acid variations (e.g., substitution, deletion, addition, etc.); the amino acid sequence of CDR-H3 consists of the amino acid sequence of CDR-H3 of the CD161 binding protein (e.g., antibody) set forth in Table 3 herein, or the amino acid sequence of CDR-H3 of the CD161 binding protein (e.g., antibody) set forth in Table 3 herein comprising no more than 1, 2, or 3 amino acid variations (e.g., substitution, deletion, addition, etc.); the amino acid sequence of CDR-L1 consists of the amino acid sequence of CDR-L1 of the CD161 binding protein (e.g., antibody) set forth in Table 3 herein, or the amino acid sequence of CDR-L1 of the CD161 binding protein (e.g., antibody) set forth in Table 3 herein comprising no more than 1, 2, or 3 amino acid variations (e.g., substitution, deletion, addition, etc.); the amino acid sequence of CDR-L2 consists of the amino acid sequence of CDR-L2 of the CD161 binding protein (e.g., antibody) set forth in Table 3 herein, or the amino acid sequence of CDR-L2 of the CD161 binding protein (e.g., antibody) set forth in Table 3 herein comprising no more than 1, 2, or 3 amino acid variations (e.g., substitution, deletion, addition, etc.); and the amino acid sequence of CDR-L3 consists of the amino acid sequence of CDR-L3 of the CD161 binding protein (e.g., antibody) set forth in Table 3 herein, or the amino acid sequence of CDR-L3 of the CD161 binding protein (e.g., antibody) set forth in Table 3 herein comprising no more than 1, 2, or 3 amino acid variations (e.g., substitution, deletion, addition, etc.).

In some embodiments, the amino acid sequence of CDR-H1 consists of the amino acid sequence of CDR-H1 of a CD161 binding protein (e.g., antibody) set forth in Table 3 herein; the amino acid sequence of CDR-H2 consists of the amino acid sequence of CDR-H2 of the CD161 binding protein (e.g., antibody) set forth in Table 3 herein; the amino acid sequence of CDR-H3 consists of CDR-H3 of the CD161 binding protein (e.g., antibody) set forth in Table 3 herein; the amino acid sequence of CDR-L1 consists of the amino acid sequence of CDR-L1 of the CD161 binding protein (e.g., antibody) set forth in Table 3 herein; the amino acid sequence of CDR-L2 consists of the amino acid sequence of CDR-L2 of the CD161 binding protein (e.g., antibody) set forth in Table 3 herein; and the amino acid sequence of CDR-L3 consists of the amino acid sequence of CDR-L3 of the CD161 binding protein (e.g., antibody) set forth in Table 3 herein.

In some embodiments, the amino acid sequence of CDR-H1 consists of the amino acid sequence of CDR-H1 of a CD161 binding protein (e.g., antibody) set forth in Table 3 herein comprising no more than 1, 2, or 3 amino acid variations (e.g., substitution, deletion, addition, etc.); the amino acid sequence of CDR-H2 consists of the amino acid sequence of CDR-H2 of the CD161 binding protein (e.g., antibody) set forth in Table 3 herein comprising no more than 1, 2, or 3 amino acid variations (e.g., substitution, deletion, addition, etc.); the amino acid sequence of CDR-H3 consists of the amino acid sequence of CDR-H3 of the CD161 binding protein (e.g., antibody) set forth in Table 3 herein comprising no more than 1, 2, or 3 amino acid variations (e.g., substitution, deletion, addition, etc.); the amino acid sequence of CDR-L1 consists of the amino acid sequence of CDR-L1 of the CD161 binding protein (e.g., antibody) set forth in Table 3 herein comprising no more than 1, 2, or 3 amino acid variations (e.g., substitution, deletion, addition, etc.); the amino acid sequence of CDR-L2 consists of the amino acid sequence of CDR-L2 of the CD161 binding protein (e.g., antibody) set forth in Table 3 herein comprising no more than 1, 2, or 3 amino acid variations (e.g., substitution, deletion, addition, etc.); and the amino acid sequence of CDR-L3 consists of the amino acid sequence of CDR-L3 of the CD161 binding protein (e.g., antibody) set forth in Table 3 herein comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VH of a CD161 binding protein (e.g., antibody) set forth in Table 3 herein; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of the CD161 binding protein (e.g., antibody) set forth in Table 3 herein. In some embodiments, the amino acid sequence of the VH comprises an amino acid sequence at least 90%, 91%, 92%, 93%, 94%, 95%, 96%, 97%, 98%, 99%, or 100% identical to the amino acid sequence of a VH of a CD161 binding protein (e.g., antibody) set forth in Table 3 herein; and the amino acid sequence of the VL comprises an amino acid sequence at least 90%, 91%, 92%, 93%, 94%, 95%, 96%, 97%, 98%, 99%, or 100% identical to the amino acid sequence of the VL of the CD161 binding protein (e.g., antibody) set forth in Table 3 herein. In some embodiments, the amino acid sequence of the VH comprises an amino acid sequence at least 95%, 96%, 97%, 98%, 99%, or 100% identical to the amino acid sequence of a VH of a CD161 binding protein (e.g., antibody) set forth in Table 3 herein; and the amino acid sequence of the VL comprises an amino acid sequence at least 95%, 96%, 97%, 98%, 99%, or 100% identical to the amino acid sequence of the VL of the CD161 binding protein (e.g., antibody) set forth in Table 3 herein. In some embodiments, the amino acid sequence of the VH comprises the amino acid sequence of a VH of a CD161 binding protein (e.g., antibody) set forth in Table 3 herein; and the amino acid sequence of the VL comprises the amino acid sequence of the VL of the CD161 binding protein (e.g., antibody) set forth in Table 3 herein.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH of a CD161 binding protein (e.g., antibody) set forth in Table 3 herein; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of the CD161 binding protein (e.g., antibody) set forth in Table 3 herein. In some embodiments, the amino acid sequence of the VH consists of an amino acid sequence at least 90%, 91%, 92%, 93%, 94%, 95%, 96%, 97%, 98%, 99%, or 100% identical to the amino acid sequence of a VH of a CD161 binding protein (e.g., antibody) set forth in Table 3 herein; and the amino acid sequence of the VL consists of an amino acid sequence at least 90%, 91%, 92%, 93%, 94%, 95%, 96%, 97%, 98%, 99%, or 100% identical to the amino acid sequence of the VL of the CD161 binding protein (e.g., antibody) set forth in Table 3 herein. In some embodiments, the amino acid sequence of the VH consists of an amino acid sequence at least 95%, 96%, 97%, 98%, 99%, or 100% identical to the amino acid sequence of a VH of a CD161 binding protein (e.g., antibody) set forth in Table 3 herein; and the amino acid sequence of the VL consists of an amino acid sequence at least 95%, 96%, 97%, 98%, 99%, or 100% identical to the amino acid sequence of the VL of the CD161 binding protein (e.g., antibody) set forth in Table 3 herein. In some embodiments, the amino acid sequence of the VH consists of the amino acid sequence of a VH of a CD161 binding protein (e.g., antibody) set forth in Table 3 herein; and the amino acid sequence of the VL consists of the amino acid sequence of the VL of the CD161 binding protein (e.g., antibody) set forth in Table 3 herein.

In some embodiments, the amino acid sequence of CDR-H1 comprises the amino acid sequence of CDR-H1 of Ab-2, or the amino acid sequence of the CDR-H1 of Ab-2 comprising 1, 2, or 3 amino acid variations (e.g., substitution, deletion, addition, etc.); the amino acid sequence of CDR-H2 comprises the amino acid sequence of CDR-H2 of Ab-2, or the amino acid sequence of CDR-H2 of Ab-2 comprising 1, 2, or 3 amino acid variations (e.g., substitution, deletion, addition, etc.); the amino acid sequence of CDR-H3 comprises the amino acid sequence of CDR-H3 of Ab-2, or the amino acid sequence of CDR-H3 of Ab-2 comprising 1, 2, or 3 amino acid variations (e.g., substitution, deletion, addition, etc.); the amino acid sequence of CDR-L1 comprises the amino acid sequence of CDR-L1 of Ab-2, or the amino acid sequence of CDR-L1 of Ab-2 comprising 1, 2, or 3 amino acid variations (e.g., substitution, deletion, addition, etc.); the amino acid sequence of CDR-L2 comprises the amino acid sequence of a CDR-L2 of Ab-2, or the amino acid sequence of a CDR-L2 of Ab-2 comprising 1, 2, or 3 amino acid variations (e.g., substitution, deletion, addition, etc.); and the amino acid sequence of CDR-L3 comprises the amino acid sequence of CDR-L3 of Ab-2, or the amino acid sequence of CDR-L3 of Ab-2 comprising 1, 2, or 3 amino acid variations (e.g., substitution, deletion, addition, etc.).

In some embodiments, the amino acid sequence of CDR-H1 comprises the amino acid sequence of CDR-H1 of Ab-2, or the amino acid sequence of CDR-H1 of Ab-2 comprising no more than 1, 2, or 3 amino acid variations (e.g., substitution, deletion, addition, etc.); the amino acid sequence of CDR-H2 comprises the amino acid sequence of CDR-H2 of Ab-2, or the amino acid sequence of CDR-H2 of Ab-2 comprising no more than 1, 2, or 3 amino acid variations (e.g., substitution, deletion, addition, etc.); the amino acid sequence of CDR-H3 comprises of CDR-H3 of Ab-2, or the amino acid sequence of CDR-H3 of Ab-2 comprising no more than 1, 2, or 3 amino acid variations (e.g., substitution, deletion, addition, etc.); the amino acid sequence of CDR-L1 comprises the amino acid sequence of CDR-L1 of Ab-2, or the amino acid sequence of CDR-L1 of Ab-2 comprising no more than 1, 2, or 3 amino acid variations (e.g., substitution, deletion, addition, etc.); the amino acid sequence of CDR-L2 comprises the amino acid sequence of CDR-L2 of Ab-2, or the amino acid sequence of CDR-L2 of Ab-2 comprising no more than 1, 2, or 3 amino acid variations (e.g., substitution, deletion, addition, etc.); and the amino acid sequence of CDR-L3 comprises the amino acid sequence of CDR-L3 of Ab-2, or the amino acid sequence of CDR-L3 of Ab-2 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-2; the amino acid sequence of CDR-H2 comprises the amino acid sequence of CDR-H2 of Ab-2; the amino acid sequence of CDR-H3 comprises CDR-H3 of Ab-2; the amino acid sequence of CDR-L1 comprises the amino acid sequence of CDR-L1 of Ab-2; the amino acid sequence of CDR-L2 comprises the amino acid sequence of CDR-L2 of Ab-2; and the amino acid sequence of CDR-L3 comprises the amino acid sequence of CDR-L3 of Ab-2.

In some embodiments, the amino acid sequence of CDR-H1 comprises the amino acid sequence of CDR-H1 of Ab-2 comprising no more than 1, 2, or 3 amino acid variations (e.g., substitution, deletion, addition, etc.); the amino acid sequence of CDR-H2 comprises the amino acid sequence CDR-H2 of Ab-2 comprising no more than 1, 2, or 3 amino acid variations (e.g., substitution, deletion, addition, etc.); the amino acid sequence of CDR-H3 comprises the amino acid sequence CDR-H3 of Ab-2 comprising no more than 1, 2, or 3 amino acid variations (e.g., substitution, deletion, addition, etc.); the amino acid sequence of CDR-L1 comprises the amino acid sequence CDR-L1 of Ab-2 comprising no more than 1, 2, or 3 amino acid variations (e.g., substitution, deletion, addition, etc.); the amino acid sequence of a CDR-L2 comprises the amino acid sequence CDR-L2 of Ab-2 comprising no more than 1, 2, or 3 amino acid variations (e.g., substitution, deletion, addition, etc.); and the amino acid sequence of a CDR-L3 comprises the amino acid sequence CDR-L3 of Ab-2 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-2; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-2. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-2; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-2. In some embodiments, the amino acid sequence of the VH comprises an amino acid sequence at least 95%, 96%, 97%, 98%, 99%, or 100% identical to the amino acid sequence of the VH of Ab-2; and the amino acid sequence of the VL comprises an amino acid sequence at least 95%, 96%, 97%, 98%, 99%, or 100% identical to the amino acid sequence of the VL of Ab-2. In some embodiments, the amino acid sequence of the VH comprises the amino acid sequence of the VH of Ab-2; and the amino acid sequence of the VL comprises the amino acid sequence of the VL of Ab-2.

In some embodiments, the amino acid sequence of CDR-H1 comprises the amino acid sequence of CDR-H1 of Ab-3, or the amino acid sequence of the CDR-H1 of Ab-3 comprising 1, 2, or 3 amino acid variations (e.g., substitution, deletion, addition, etc.); the amino acid sequence of CDR-H2 comprises the amino acid sequence of CDR-H2 of Ab-3, or the amino acid sequence of CDR-H2 of Ab-3 comprising 1, 2, or 3 amino acid variations (e.g., substitution, deletion, addition, etc.); the amino acid sequence of CDR-H3 comprises the amino acid sequence of CDR-H3 of Ab-3, or the amino acid sequence of CDR-H3 of Ab-3 comprising 1, 2, or 3 amino acid variations (e.g., substitution, deletion, addition, etc.); the amino acid sequence of CDR-L1 comprises the amino acid sequence of CDR-L1 of Ab-3, or the amino acid sequence of CDR-L1 of Ab-3 comprising 1, 2, or 3 amino acid variations (e.g., substitution, deletion, addition, etc.); the amino acid sequence of CDR-L2 comprises the amino acid sequence of a CDR-L2 of Ab-3, or the amino acid sequence of a CDR-L2 of Ab-3 comprising 1, 2, or 3 amino acid variations (e.g., substitution, deletion, addition, etc.); and the amino acid sequence of CDR-L3 comprises the amino acid sequence of CDR-L3 of Ab-3, or the amino acid sequence of CDR-L3 of Ab-3 comprising 1, 2, or 3 amino acid variations (e.g., substitution, deletion, addition, etc.).

In some embodiments, the amino acid sequence of CDR-H1 comprises the amino acid sequence of CDR-H1 of Ab-3, or the amino acid sequence of CDR-H1 of Ab-3 comprising no more than 1, 2, or 3 amino acid variations (e.g., substitution, deletion, addition, etc.); the amino acid sequence of CDR-H2 comprises the amino acid sequence of CDR-H2 of Ab-3, or the amino acid sequence of CDR-H2 of Ab-3 comprising no more than 1, 2, or 3 amino acid variations (e.g., substitution, deletion, addition, etc.); the amino acid sequence of CDR-H3 comprises of CDR-H3 of Ab-3, or the amino acid sequence of CDR-H3 of Ab-3 comprising no more than 1, 2, or 3 amino acid variations (e.g., substitution, deletion, addition, etc.); the amino acid sequence of CDR-L1 comprises the amino acid sequence of CDR-L1 of Ab-3, or the amino acid sequence of CDR-L1 of Ab-3 comprising no more than 1, 2, or 3 amino acid variations (e.g., substitution, deletion, addition, etc.); the amino acid sequence of CDR-L2 comprises the amino acid sequence of CDR-L2 of Ab-3, or the amino acid sequence of CDR-L2 of Ab-3 comprising no more than 1, 2, or 3 amino acid variations (e.g., substitution, deletion, addition, etc.); and the amino acid sequence of CDR-L3 comprises the amino acid sequence of CDR-L3 of Ab-3, or the amino acid sequence of CDR-L3 of Ab-3 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-3; the amino acid sequence of CDR-H2 comprises the amino acid sequence of CDR-H2 of Ab-3; the amino acid sequence of CDR-H3 comprises CDR-H3 of Ab-3; the amino acid sequence of CDR-L1 comprises the amino acid sequence of CDR-L1 of Ab-3; the amino acid sequence of CDR-L2 comprises the amino acid sequence of CDR-L2 of Ab-3; and the amino acid sequence of CDR-L3 comprises the amino acid sequence of CDR-L3 of Ab-3.

In some embodiments, the amino acid sequence of CDR-H1 comprises the amino acid sequence of CDR-H1 of Ab-3 comprising no more than 1, 2, or 3 amino acid variations (e.g., substitution, deletion, addition, etc.); the amino acid sequence of CDR-H2 comprises the amino acid sequence CDR-H2 of Ab-3 comprising no more than 1, 2, or 3 amino acid variations (e.g., substitution, deletion, addition, etc.); the amino acid sequence of CDR-H3 comprises the amino acid sequence CDR-H3 of Ab-3 comprising no more than 1, 2, or 3 amino acid variations (e.g., substitution, deletion, addition, etc.); the amino acid sequence of CDR-L1 comprises the amino acid sequence CDR-L1 of Ab-3 comprising no more than 1, 2, or 3 amino acid variations (e.g., substitution, deletion, addition, etc.); the amino acid sequence of a CDR-L2 comprises the amino acid sequence CDR-L2 of Ab-3 comprising no more than 1, 2, or 3 amino acid variations (e.g., substitution, deletion, addition, etc.); and the amino acid sequence of a CDR-L3 comprises the amino acid sequence CDR-L3 of Ab-3 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-3; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-3. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-3; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-3. In some embodiments, the amino acid sequence of the VH comprises an amino acid sequence at least 95%, 96%, 97%, 98%, 99%, or 100% identical to the amino acid sequence of the VH of Ab-3; and the amino acid sequence of the VL comprises an amino acid sequence at least 95%, 96%, 97%, 98%, 99%, or 100% identical to the amino acid sequence of the VL of Ab-3. In some embodiments, the amino acid sequence of the VH comprises the amino acid sequence of the VH of Ab-3; and the amino acid sequence of the VL comprises the amino acid sequence of the VL of Ab-3.

In some embodiments, the amino acid sequence of CDR-H1 comprises the amino acid sequence of CDR-H1 of Ab-4, or the amino acid sequence of the CDR-H1 of Ab-4 comprising 1, 2, or 3 amino acid variations (e.g., substitution, deletion, addition, etc.); the amino acid sequence of CDR-H2 comprises the amino acid sequence of CDR-H2 of Ab-4, or the amino acid sequence of CDR-H2 of Ab-4 comprising 1, 2, or 3 amino acid variations (e.g., substitution, deletion, addition, etc.); the amino acid sequence of CDR-H3 comprises the amino acid sequence of CDR-H3 of Ab-4, or the amino acid sequence of CDR-H3 of Ab-4 comprising 1, 2, or 3 amino acid variations (e.g., substitution, deletion, addition, etc.); the amino acid sequence of CDR-L1 comprises the amino acid sequence of CDR-L1 of Ab-4, or the amino acid sequence of CDR-L1 of Ab-4 comprising 1, 2, or 3 amino acid variations (e.g., substitution, deletion, addition, etc.); the amino acid sequence of CDR-L2 comprises the amino acid sequence of a CDR-L2 of Ab-4, or the amino acid sequence of a CDR-L2 of Ab-4 comprising 1, 2, or 3 amino acid variations (e.g., substitution, deletion, addition, etc.); and the amino acid sequence of CDR-L3 comprises the amino acid sequence of CDR-L3 of Ab-4, or the amino acid sequence of CDR-L3 of Ab-4 comprising 1, 2, or 3 amino acid variations (e.g., substitution, deletion, addition, etc.).

In some embodiments, the amino acid sequence of CDR-H1 comprises the amino acid sequence of CDR-H1 of Ab-4, or the amino acid sequence of CDR-H1 of Ab-4 comprising no more than 1, 2, or 3 amino acid variations (e.g., substitution, deletion, addition, etc.); the amino acid sequence of CDR-H2 comprises the amino acid sequence of CDR-H2 of Ab-4, or the amino acid sequence of CDR-H2 of Ab-4 comprising no more than 1, 2, or 3 amino acid variations (e.g., substitution, deletion, addition, etc.); the amino acid sequence of CDR-H3 comprises of CDR-H3 of Ab-4, or the amino acid sequence of CDR-H3 of Ab-4 comprising no more than 1, 2, or 3 amino acid variations (e.g., substitution, deletion, addition, etc.); the amino acid sequence of CDR-L1 comprises the amino acid sequence of CDR-L1 of Ab-4, or the amino acid sequence of CDR-L1 of Ab-4 comprising no more than 1, 2, or 3 amino acid variations (e.g., substitution, deletion, addition, etc.); the amino acid sequence of CDR-L2 comprises the amino acid sequence of CDR-L2 of Ab-4, or the amino acid sequence of CDR-L2 of Ab-4 comprising no more than 1, 2, or 3 amino acid variations (e.g., substitution, deletion, addition, etc.); and the amino acid sequence of CDR-L3 comprises the amino acid sequence of CDR-L3 of Ab-4, or the amino acid sequence of CDR-L3 of Ab-4 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-4; the amino acid sequence of CDR-H2 comprises the amino acid sequence of CDR-H2 of Ab-4; the amino acid sequence of CDR-H3 comprises CDR-H3 of Ab-4; the amino acid sequence of CDR-L1 comprises the amino acid sequence of CDR-L1 of Ab-4; the amino acid sequence of CDR-L2 comprises the amino acid sequence of CDR-L2 of Ab-4; and the amino acid sequence of CDR-L3 comprises the amino acid sequence of CDR-L3 of Ab-4.

In some embodiments, the amino acid sequence of CDR-H1 comprises the amino acid sequence of CDR-H1 of Ab-4 comprising no more than 1, 2, or 3 amino acid variations (e.g., substitution, deletion, addition, etc.); the amino acid sequence of CDR-H2 comprises the amino acid sequence CDR-H2 of Ab-4 comprising no more than 1, 2, or 3 amino acid variations (e.g., substitution, deletion, addition, etc.); the amino acid sequence of CDR-H3 comprises the amino acid sequence CDR-H3 of Ab-4 comprising no more than 1, 2, or 3 amino acid variations (e.g., substitution, deletion, addition, etc.); the amino acid sequence of CDR-L1 comprises the amino acid sequence CDR-L1 of Ab-4 comprising no more than 1, 2, or 3 amino acid variations (e.g., substitution, deletion, addition, etc.); the amino acid sequence of a CDR-L2 comprises the amino acid sequence CDR-L2 of Ab-4 comprising no more than 1, 2, or 3 amino acid variations (e.g., substitution, deletion, addition, etc.); and the amino acid sequence of a CDR-L3 comprises the amino acid sequence CDR-L3 of Ab-4 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-4; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-4. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-4; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-4. In some embodiments, the amino acid sequence of the VH comprises an amino acid sequence at least 95%, 96%, 97%, 98%, 99%, or 100% identical to the amino acid sequence of the VH of Ab-4; and the amino acid sequence of the VL comprises an amino acid sequence at least 95%, 96%, 97%, 98%, 99%, or 100% identical to the amino acid sequence of the VL of Ab-4. In some embodiments, the amino acid sequence of the VH comprises the amino acid sequence of the VH of Ab-4; and the amino acid sequence of the VL comprises the amino acid sequence of the VL of Ab-4.

In some embodiments, the amino acid sequence of CDR-H1 comprises the amino acid sequence of CDR-H1 of Ab-5, or the amino acid sequence of the CDR-H1 of Ab-5 comprising 1, 2, or 3 amino acid variations (e.g., substitution, deletion, addition, etc.); the amino acid sequence of CDR-H2 comprises the amino acid sequence of CDR-H2 of Ab-5, or the amino acid sequence of CDR-H2 of Ab-5 comprising 1, 2, or 3 amino acid variations (e.g., substitution, deletion, addition, etc.); the amino acid sequence of CDR-H3 comprises the amino acid sequence of CDR-H3 of Ab-5, or the amino acid sequence of CDR-H3 of Ab-5 comprising 1, 2, or 3 amino acid variations (e.g., substitution, deletion, addition, etc.); the amino acid sequence of CDR-L1 comprises the amino acid sequence of CDR-L1 of Ab-5, or the amino acid sequence of CDR-L1 of Ab-5 comprising 1, 2, or 3 amino acid variations (e.g., substitution, deletion, addition, etc.); the amino acid sequence of CDR-L2 comprises the amino acid sequence of a CDR-L2 of Ab-5, or the amino acid sequence of a CDR-L2 of Ab-5 comprising 1, 2, or 3 amino acid variations (e.g., substitution, deletion, addition, etc.); and the amino acid sequence of CDR-L3 comprises the amino acid sequence of CDR-L3 of Ab-5, or the amino acid sequence of CDR-L3 of Ab-5 comprising 1, 2, or 3 amino acid variations (e.g., substitution, deletion, addition, etc.).

In some embodiments, the amino acid sequence of CDR-H1 comprises the amino acid sequence of CDR-H1 of Ab-5, or the amino acid sequence of CDR-H1 of Ab-5 comprising no more than 1, 2, or 3 amino acid variations (e.g., substitution, deletion, addition, etc.); the amino acid sequence of CDR-H2 comprises the amino acid sequence of CDR-H2 of Ab-5, or the amino acid sequence of CDR-H2 of Ab-5 comprising no more than 1, 2, or 3 amino acid variations (e.g., substitution, deletion, addition, etc.); the amino acid sequence of CDR-H3 comprises of CDR-H3 of Ab-5, or the amino acid sequence of CDR-H3 of Ab-5 comprising no more than 1, 2, or 3 amino acid variations (e.g., substitution, deletion, addition, etc.); the amino acid sequence of CDR-L1 comprises the amino acid sequence of CDR-L1 of Ab-5, or the amino acid sequence of CDR-L1 of Ab-5 comprising no more than 1, 2, or 3 amino acid variations (e.g., substitution, deletion, addition, etc.); the amino acid sequence of CDR-L2 comprises the amino acid sequence of CDR-L2 of Ab-5, or the amino acid sequence of CDR-L2 of Ab-5 comprising no more than 1, 2, or 3 amino acid variations (e.g., substitution, deletion, addition, etc.); and the amino acid sequence of CDR-L3 comprises the amino acid sequence of CDR-L3 of Ab-5, or the amino acid sequence of CDR-L3 of Ab-5 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-5; the amino acid sequence of CDR-H2 comprises the amino acid sequence of CDR-H2 of Ab-5; the amino acid sequence of CDR-H3 comprises CDR-H3 of Ab-5; the amino acid sequence of CDR-L1 comprises the amino acid sequence of CDR-L1 of Ab-5; the amino acid sequence of CDR-L2 comprises the amino acid sequence of CDR-L2 of Ab-5; and the amino acid sequence of CDR-L3 comprises the amino acid sequence of CDR-L3 of Ab-5.

In some embodiments, the amino acid sequence of CDR-H1 comprises the amino acid sequence of CDR-H1 of Ab-5 comprising no more than 1, 2, or 3 amino acid variations (e.g., substitution, deletion, addition, etc.); the amino acid sequence of CDR-H2 comprises the amino acid sequence CDR-H2 of Ab-5 comprising no more than 1, 2, or 3 amino acid variations (e.g., substitution, deletion, addition, etc.); the amino acid sequence of CDR-H3 comprises the amino acid sequence CDR-H3 of Ab-5 comprising no more than 1, 2, or 3 amino acid variations (e.g., substitution, deletion, addition, etc.); the amino acid sequence of CDR-L1 comprises the amino acid sequence CDR-L1 of Ab-5 comprising no more than 1, 2, or 3 amino acid variations (e.g., substitution, deletion, addition, etc.); the amino acid sequence of a CDR-L2 comprises the amino acid sequence CDR-L2 of Ab-5 comprising no more than 1, 2, or 3 amino acid variations (e.g., substitution, deletion, addition, etc.); and the amino acid sequence of a CDR-L3 comprises the amino acid sequence CDR-L3 of Ab-5 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-5; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-5. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-5; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-5. In some embodiments, the amino acid sequence of the VH comprises an amino acid sequence at least 95%, 96%, 97%, 98%, 99%, or 100% identical to the amino acid sequence of the VH of Ab-5; and the amino acid sequence of the VL comprises an amino acid sequence at least 95%, 96%, 97%, 98%, 99%, or 100% identical to the amino acid sequence of the VL of Ab-5. In some embodiments, the amino acid sequence of the VH comprises the amino acid sequence of the VH of Ab-5; and the amino acid sequence of the VL comprises the amino acid sequence of the VL of Ab-5.

In some embodiments, the amino acid sequence of CDR-H1 comprises the amino acid sequence of CDR-H1 of Ab-6, or the amino acid sequence of the CDR-H1 of Ab-6 comprising 1, 2, or 3 amino acid variations (e.g., substitution, deletion, addition, etc.); the amino acid sequence of CDR-H2 comprises the amino acid sequence of CDR-H2 of Ab-6, or the amino acid sequence of CDR-H2 of Ab-6 comprising 1, 2, or 3 amino acid variations (e.g., substitution, deletion, addition, etc.); the amino acid sequence of CDR-H3 comprises the amino acid sequence of CDR-H3 of Ab-6, or the amino acid sequence of CDR-H3 of Ab-6 comprising 1, 2, or 3 amino acid variations (e.g., substitution, deletion, addition, etc.); the amino acid sequence of CDR-L1 comprises the amino acid sequence of CDR-L1 of Ab-6, or the amino acid sequence of CDR-L1 of Ab-6 comprising 1, 2, or 3 amino acid variations (e.g., substitution, deletion, addition, etc.); the amino acid sequence of CDR-L2 comprises the amino acid sequence of a CDR-L2 of Ab-6, or the amino acid sequence of a CDR-L2 of Ab-6 comprising 1, 2, or 3 amino acid variations (e.g., substitution, deletion, addition, etc.); and the amino acid sequence of CDR-L3 comprises the amino acid sequence of CDR-L3 of Ab-6, or the amino acid sequence of CDR-L3 of Ab-6 comprising 1, 2, or 3 amino acid variations (e.g., substitution, deletion, addition, etc.).

In some embodiments, the amino acid sequence of CDR-H1 comprises the amino acid sequence of CDR-H1 of Ab-6, or the amino acid sequence of CDR-H1 of Ab-6 comprising no more than 1, 2, or 3 amino acid variations (e.g., substitution, deletion, addition, etc.); the amino acid sequence of CDR-H2 comprises the amino acid sequence of CDR-H2 of Ab-6, or the amino acid sequence of CDR-H2 of Ab-6 comprising no more than 1, 2, or 3 amino acid variations (e.g., substitution, deletion, addition, etc.); the amino acid sequence of CDR-H3 comprises of CDR-H3 of Ab-6, or the amino acid sequence of CDR-H3 of Ab-6 comprising no more than 1, 2, or 3 amino acid variations (e.g., substitution, deletion, addition, etc.); the amino acid sequence of CDR-L1 comprises the amino acid sequence of CDR-L1 of Ab-6, or the amino acid sequence of CDR-L1 of Ab-6 comprising no more than 1, 2, or 3 amino acid variations (e.g., substitution, deletion, addition, etc.); the amino acid sequence of CDR-L2 comprises the amino acid sequence of CDR-L2 of Ab-6, or the amino acid sequence of CDR-L2 of Ab-6 comprising no more than 1, 2, or 3 amino acid variations (e.g., substitution, deletion, addition, etc.); and the amino acid sequence of CDR-L3 comprises the amino acid sequence of CDR-L3 of Ab-6, or the amino acid sequence of CDR-L3 of Ab-6 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-6; the amino acid sequence of CDR-H2 comprises the amino acid sequence of CDR-H2 of Ab-6; the amino acid sequence of CDR-H3 comprises CDR-H3 of Ab-6; the amino acid sequence of CDR-L1 comprises the amino acid sequence of CDR-L1 of Ab-6; the amino acid sequence of CDR-L2 comprises the amino acid sequence of CDR-L2 of Ab-6; and the amino acid sequence of CDR-L3 comprises the amino acid sequence of CDR-L3 of Ab-6.

In some embodiments, the amino acid sequence of CDR-H1 comprises the amino acid sequence of CDR-H1 of Ab-6 comprising no more than 1, 2, or 3 amino acid variations (e.g., substitution, deletion, addition, etc.); the amino acid sequence of CDR-H2 comprises the amino acid sequence CDR-H2 of Ab-6 comprising no more than 1, 2, or 3 amino acid variations (e.g., substitution, deletion, addition, etc.); the amino acid sequence of CDR-H3 comprises the amino acid sequence CDR-H3 of Ab-6 comprising no more than 1, 2, or 3 amino acid variations (e.g., substitution, deletion, addition, etc.); the amino acid sequence of CDR-L1 comprises the amino acid sequence CDR-L1 of Ab-6 comprising no more than 1, 2, or 3 amino acid variations (e.g., substitution, deletion, addition, etc.); the amino acid sequence of a CDR-L2 comprises the amino acid sequence CDR-L2 of Ab-6 comprising no more than 1, 2, or 3 amino acid variations (e.g., substitution, deletion, addition, etc.); and the amino acid sequence of a CDR-L3 comprises the amino acid sequence CDR-L3 of Ab-6 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-6; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-6. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-6; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-6. In some embodiments, the amino acid sequence of the VH comprises an amino acid sequence at least 95%, 96%, 97%, 98%, 99%, or 100% identical to the amino acid sequence of the VH of Ab-6; and the amino acid sequence of the VL comprises an amino acid sequence at least 95%, 96%, 97%, 98%, 99%, or 100% identical to the amino acid sequence of the VL of Ab-6. In some embodiments, the amino acid sequence of the VH comprises the amino acid sequence of the VH of Ab-6; and the amino acid sequence of the VL comprises the amino acid sequence of the VL of Ab-6.

In some embodiments, the amino acid sequence of CDR-H1 comprises the amino acid sequence of CDR-H1 of Ab-7, or the amino acid sequence of the CDR-H1 of Ab-7 comprising 1, 2, or 3 amino acid variations (e.g., substitution, deletion, addition, etc.); the amino acid sequence of CDR-H2 comprises the amino acid sequence of CDR-H2 of Ab-7, or the amino acid sequence of CDR-H2 of Ab-7 comprising 1, 2, or 3 amino acid variations (e.g., substitution, deletion, addition, etc.); the amino acid sequence of CDR-H3 comprises the amino acid sequence of CDR-H3 of Ab-7, or the amino acid sequence of CDR-H3 of Ab-7 comprising 1, 2, or 3 amino acid variations (e.g., substitution, deletion, addition, etc.); the amino acid sequence of CDR-L1 comprises the amino acid sequence of CDR-L1 of Ab-7, or the amino acid sequence of CDR-L1 of Ab-7 comprising 1, 2, or 3 amino acid variations (e.g., substitution, deletion, addition, etc.); the amino acid sequence of CDR-L2 comprises the amino acid sequence of a CDR-L2 of Ab-7, or the amino acid sequence of a CDR-L2 of Ab-7 comprising 1, 2, or 3 amino acid variations (e.g., substitution, deletion, addition, etc.); and the amino acid sequence of CDR-L3 comprises the amino acid sequence of CDR-L3 of Ab-7, or the amino acid sequence of CDR-L3 of Ab-7 comprising 1, 2, or 3 amino acid variations (e.g., substitution, deletion, addition, etc.).

In some embodiments, the amino acid sequence of CDR-H1 comprises the amino acid sequence of CDR-H1 of Ab-7, or the amino acid sequence of CDR-H1 of Ab-7 comprising no more than 1, 2, or 3 amino acid variations (e.g., substitution, deletion, addition, etc.); the amino acid sequence of CDR-H2 comprises the amino acid sequence of CDR-H2 of Ab-7, or the amino acid sequence of CDR-H2 of Ab-7 comprising no more than 1, 2, or 3 amino acid variations (e.g., substitution, deletion, addition, etc.); the amino acid sequence of CDR-H3 comprises of CDR-H3 of Ab-7, or the amino acid sequence of CDR-H3 of Ab-7 comprising no more than 1, 2, or 3 amino acid variations (e.g., substitution, deletion, addition, etc.); the amino acid sequence of CDR-L1 comprises the amino acid sequence of CDR-L1 of Ab-7, or the amino acid sequence of CDR-L1 of Ab-7 comprising no more than 1, 2, or 3 amino acid variations (e.g., substitution, deletion, addition, etc.); the amino acid sequence of CDR-L2 comprises the amino acid sequence of CDR-L2 of Ab-7, or the amino acid sequence of CDR-L2 of Ab-7 comprising no more than 1, 2, or 3 amino acid variations (e.g., substitution, deletion, addition, etc.); and the amino acid sequence of CDR-L3 comprises the amino acid sequence of CDR-L3 of Ab-7, or the amino acid sequence of CDR-L3 of Ab-7 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-7; the amino acid sequence of CDR-H2 comprises the amino acid sequence of CDR-H2 of Ab-7; the amino acid sequence of CDR-H3 comprises CDR-H3 of Ab-7; the amino acid sequence of CDR-L1 comprises the amino acid sequence of CDR-L1 of Ab-7; the amino acid sequence of CDR-L2 comprises the amino acid sequence of CDR-L2 of Ab-7; and the amino acid sequence of CDR-L3 comprises the amino acid sequence of CDR-L3 of Ab-7.

In some embodiments, the amino acid sequence of CDR-H1 comprises the amino acid sequence of CDR-H1 of Ab-7 comprising no more than 1, 2, or 3 amino acid variations (e.g., substitution, deletion, addition, etc.); the amino acid sequence of CDR-H2 comprises the amino acid sequence CDR-H2 of Ab-7 comprising no more than 1, 2, or 3 amino acid variations (e.g., substitution, deletion, addition, etc.); the amino acid sequence of CDR-H3 comprises the amino acid sequence CDR-H3 of Ab-7 comprising no more than 1, 2, or 3 amino acid variations (e.g., substitution, deletion, addition, etc.); the amino acid sequence of CDR-L1 comprises the amino acid sequence CDR-L1 of Ab-7 comprising no more than 1, 2, or 3 amino acid variations (e.g., substitution, deletion, addition, etc.); the amino acid sequence of a CDR-L2 comprises the amino acid sequence CDR-L2 of Ab-7 comprising no more than 1, 2, or 3 amino acid variations (e.g., substitution, deletion, addition, etc.); and the amino acid sequence of a CDR-L3 comprises the amino acid sequence CDR-L3 of Ab-7 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-7; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-7. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-7; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-7. In some embodiments, the amino acid sequence of the VH comprises an amino acid sequence at least 95%, 96%, 97%, 98%, 99%, or 100% identical to the amino acid sequence of the VH of Ab-7; and the amino acid sequence of the VL comprises an amino acid sequence at least 95%, 96%, 97%, 98%, 99%, or 100% identical to the amino acid sequence of the VL of Ab-7. In some embodiments, the amino acid sequence of the VH comprises the amino acid sequence of the VH of Ab-7; and the amino acid sequence of the VL comprises the amino acid sequence of the VL of Ab-7.

In some embodiments, the amino acid sequence of CDR-H1 comprises the amino acid sequence of CDR-H1 of Ab-8, or the amino acid sequence of the CDR-H1 of Ab-8 comprising 1, 2, or 3 amino acid variations (e.g., substitution, deletion, addition, etc.); the amino acid sequence of CDR-H2 comprises the amino acid sequence of CDR-H2 of Ab-8, or the amino acid sequence of CDR-H2 of Ab-8 comprising 1, 2, or 3 amino acid variations (e.g., substitution, deletion, addition, etc.); the amino acid sequence of CDR-H3 comprises the amino acid sequence of CDR-H3 of Ab-8, or the amino acid sequence of CDR-H3 of Ab-8 comprising 1, 2, or 3 amino acid variations (e.g., substitution, deletion, addition, etc.); the amino acid sequence of CDR-L1 comprises the amino acid sequence of CDR-L1 of Ab-8, or the amino acid sequence of CDR-L1 of Ab-8 comprising 1, 2, or 3 amino acid variations (e.g., substitution, deletion, addition, etc.); the amino acid sequence of CDR-L2 comprises the amino acid sequence of a CDR-L2 of Ab-8, or the amino acid sequence of a CDR-L2 of Ab-8 comprising 1, 2, or 3 amino acid variations (e.g., substitution, deletion, addition, etc.); and the amino acid sequence of CDR-L3 comprises the amino acid sequence of CDR-L3 of Ab-8, or the amino acid sequence of CDR-L3 of Ab-8 comprising 1, 2, or 3 amino acid variations (e.g., substitution, deletion, addition, etc.).

In some embodiments, the amino acid sequence of CDR-H1 comprises the amino acid sequence of CDR-H1 of Ab-8, or the amino acid sequence of CDR-H1 of Ab-8 comprising no more than 1, 2, or 3 amino acid variations (e.g., substitution, deletion, addition, etc.); the amino acid sequence of CDR-H2 comprises the amino acid sequence of CDR-H2 of Ab-8, or the amino acid sequence of CDR-H2 of Ab-8 comprising no more than 1, 2, or 3 amino acid variations (e.g., substitution, deletion, addition, etc.); the amino acid sequence of CDR-H3 comprises of CDR-H3 of Ab-8, or the amino acid sequence of CDR-H3 of Ab-8 comprising no more than 1, 2, or 3 amino acid variations (e.g., substitution, deletion, addition, etc.); the amino acid sequence of CDR-L1 comprises the amino acid sequence of CDR-L1 of Ab-8, or the amino acid sequence of CDR-L1 of Ab-8 comprising no more than 1, 2, or 3 amino acid variations (e.g., substitution, deletion, addition, etc.); the amino acid sequence of CDR-L2 comprises the amino acid sequence of CDR-L2 of Ab-8, or the amino acid sequence of CDR-L2 of Ab-8 comprising no more than 1, 2, or 3 amino acid variations (e.g., substitution, deletion, addition, etc.); and the amino acid sequence of CDR-L3 comprises the amino acid sequence of CDR-L3 of Ab-8, or the amino acid sequence of CDR-L3 of Ab-8 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-8; the amino acid sequence of CDR-H2 comprises the amino acid sequence of CDR-H2 of Ab-8; the amino acid sequence of CDR-H3 comprises CDR-H3 of Ab-8; the amino acid sequence of CDR-L1 comprises the amino acid sequence of CDR-L1 of Ab-8; the amino acid sequence of CDR-L2 comprises the amino acid sequence of CDR-L2 of Ab-8; and the amino acid sequence of CDR-L3 comprises the amino acid sequence of CDR-L3 of Ab-8.

In some embodiments, the amino acid sequence of CDR-H1 comprises the amino acid sequence of CDR-H1 of Ab-8 comprising no more than 1, 2, or 3 amino acid variations (e.g., substitution, deletion, addition, etc.); the amino acid sequence of CDR-H2 comprises the amino acid sequence CDR-H2 of Ab-8 comprising no more than 1, 2, or 3 amino acid variations (e.g., substitution, deletion, addition, etc.); the amino acid sequence of CDR-H3 comprises the amino acid sequence CDR-H3 of Ab-8 comprising no more than 1, 2, or 3 amino acid variations (e.g., substitution, deletion, addition, etc.); the amino acid sequence of CDR-L1 comprises the amino acid sequence CDR-L1 of Ab-8 comprising no more than 1, 2, or 3 amino acid variations (e.g., substitution, deletion, addition, etc.); the amino acid sequence of a CDR-L2 comprises the amino acid sequence CDR-L2 of Ab-8 comprising no more than 1, 2, or 3 amino acid variations (e.g., substitution, deletion, addition, etc.); and the amino acid sequence of a CDR-L3 comprises the amino acid sequence CDR-L3 of Ab-8 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-8; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-8. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-8; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-8. In some embodiments, the amino acid sequence of the VH comprises an amino acid sequence at least 95%, 96%, 97%, 98%, 99%, or 100% identical to the amino acid sequence of the VH of Ab-8; and the amino acid sequence of the VL comprises an amino acid sequence at least 95%, 96%, 97%, 98%, 99%, or 100% identical to the amino acid sequence of the VL of Ab-8. In some embodiments, the amino acid sequence of the VH comprises the amino acid sequence of the VH of Ab-8; and the amino acid sequence of the VL comprises the amino acid sequence of the VL of Ab-8.

In some embodiments, the amino acid sequence of CDR-H1 comprises the amino acid sequence of CDR-H1 of Ab-9, or the amino acid sequence of the CDR-H1 of Ab-9 comprising 1, 2, or 3 amino acid variations (e.g., substitution, deletion, addition, etc.; the amino acid sequence of CDR-H2 comprises the amino acid sequence of CDR-H2 of Ab-9, or the amino acid sequence of CDR-H2 of Ab-9 comprising 1, 2, or 3 amino acid variations (e.g., substitution, deletion, addition, etc.); the amino acid sequence of CDR-H3 comprises the amino acid sequence of CDR-H3 of Ab-9, or the amino acid sequence of CDR-H3 of Ab-9 comprising 1, 2, or 3 amino acid variations (e.g., substitution, deletion, addition, etc.); the amino acid sequence of CDR-L1 comprises the amino acid sequence of CDR-L1 of Ab-9, or the amino acid sequence of CDR-L1 of Ab-9 comprising 1, 2, or 3 amino acid variations (e.g., substitution, deletion, addition, etc.); the amino acid sequence of CDR-L2 comprises the amino acid sequence of a CDR-L2 of Ab-9, or the amino acid sequence of a CDR-L2 of Ab-9 comprising 1, 2, or 3 amino acid variations (e.g., substitution, deletion, addition, etc.); and the amino acid sequence of CDR-L3 comprises the amino acid sequence of CDR-L3 of Ab-9, or the amino acid sequence of CDR-L3 of Ab-9 comprising 1, 2, or 3 amino acid variations (e.g., substitution, deletion, addition, etc.).

In some embodiments, the amino acid sequence of CDR-H1 comprises the amino acid sequence of CDR-H1 of Ab-9, or the amino acid sequence of CDR-H1 of Ab-9 comprising no more than 1, 2, or 3 amino acid variations (e.g., substitution, deletion, addition, etc.); the amino acid sequence of CDR-H2 comprises the amino acid sequence of CDR-H2 of Ab-9, or the amino acid sequence of CDR-H2 of Ab-9 comprising no more than 1, 2, or 3 amino acid variations (e.g., substitution, deletion, addition, etc.); the amino acid sequence of CDR-H3 comprises of CDR-H3 of Ab-9, or the amino acid sequence of CDR-H3 of Ab-9 comprising no more than 1, 2, or 3 amino acid variations (e.g., substitution, deletion, addition, etc.); the amino acid sequence of CDR-L1 comprises the amino acid sequence of CDR-L1 of Ab-9, or the amino acid sequence of CDR-L1 of Ab-9 comprising no more than 1, 2, or 3 amino acid variations (e.g., substitution, deletion, addition, etc.); the amino acid sequence of CDR-L2 comprises the amino acid sequence of CDR-L2 of Ab-9, or the amino acid sequence of CDR-L2 of Ab-9 comprising no more than 1, 2, or 3 amino acid variations (e.g., substitution, deletion, addition, etc.); and the amino acid sequence of CDR-L3 comprises the amino acid sequence of CDR-L3 of Ab-9, or the amino acid sequence of CDR-L3 of Ab-9 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-9; the amino acid sequence of CDR-H2 comprises the amino acid sequence of CDR-H2 of Ab-9; the amino acid sequence of CDR-H3 comprises CDR-H3 of Ab-9; the amino acid sequence of CDR-L1 comprises the amino acid sequence of CDR-L1 of Ab-9; the amino acid sequence of CDR-L2 comprises the amino acid sequence of CDR-L2 of Ab-9; and the amino acid sequence of CDR-L3 comprises the amino acid sequence of CDR-L3 of Ab-9.

In some embodiments, the amino acid sequence of CDR-H1 comprises the amino acid sequence of CDR-H1 of Ab-9 comprising no more than 1, 2, or 3 amino acid variations (e.g., substitution, deletion, addition, etc.); the amino acid sequence of CDR-H2 comprises the amino acid sequence CDR-H2 of Ab-9 comprising no more than 1, 2, or 3 amino acid variations (e.g., substitution, deletion, addition, etc.); the amino acid sequence of CDR-H3 comprises the amino acid sequence CDR-H3 of Ab-9 comprising no more than 1, 2, or 3 amino acid variations (e.g., substitution, deletion, addition, etc.); the amino acid sequence of CDR-L1 comprises the amino acid sequence CDR-L1 of Ab-9 comprising no more than 1, 2, or 3 amino acid variations (e.g., substitution, deletion, addition, etc.); the amino acid sequence of a CDR-L2 comprises the amino acid sequence CDR-L2 of Ab-9 comprising no more than 1, 2, or 3 amino acid variations (e.g., substitution, deletion, addition, etc.); and the amino acid sequence of a CDR-L3 comprises the amino acid sequence CDR-L3 of Ab-9 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-9; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-9. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-9; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-9. In some embodiments, the amino acid sequence of the VH comprises an amino acid sequence at least 95%, 96%, 97%, 98%, 99%, or 100% identical to the amino acid sequence of the VH of Ab-9; and the amino acid sequence of the VL comprises an amino acid sequence at least 95%, 96%, 97%, 98%, 99%, or 100% identical to the amino acid sequence of the VL of Ab-9. In some embodiments, the amino acid sequence of the VH comprises the amino acid sequence of the VH of Ab-9; and the amino acid sequence of the VL comprises the amino acid sequence of the VL of Ab-9.

In some embodiments, the amino acid sequence of CDR-H1 comprises the amino acid sequence of CDR-H1 of Ab-10, or the amino acid sequence of the CDR-H1 of Ab-10 comprising 1, 2, or 3 amino acid variations (e.g., substitution, deletion, addition, etc.); the amino acid sequence of CDR-H2 comprises the amino acid sequence of CDR-H2 of Ab-10, or the amino acid sequence of CDR-H2 of Ab-10 comprising 1, 2, or 3 amino acid variations (e.g., substitution, deletion, addition, etc.); the amino acid sequence of CDR-H3 comprises the amino acid sequence of CDR-H3 of Ab-10, or the amino acid sequence of CDR-H3 of Ab-10 comprising 1, 2, or 3 amino acid variations (e.g., substitution, deletion, addition, etc.); the amino acid sequence of CDR-L1 comprises the amino acid sequence of CDR-L1 of Ab-10, or the amino acid sequence of CDR-L1 of Ab-10 comprising 1, 2, or 3 amino acid variations (e.g., substitution, deletion, addition, etc.); the amino acid sequence of CDR-L2 comprises the amino acid sequence of a CDR-L2 of Ab-10, or the amino acid sequence of a CDR-L2 of Ab-10 comprising 1, 2, or 3 amino acid variations (e.g., substitution, deletion, addition, etc.); and the amino acid sequence of CDR-L3 comprises the amino acid sequence of CDR-L3 of Ab-10, or the amino acid sequence of CDR-L3 of Ab-10 comprising 1, 2, or 3 amino acid variations (e.g., substitution, deletion, addition, etc.).

In some embodiments, the amino acid sequence of CDR-H1 comprises the amino acid sequence of CDR-H1 of Ab-10, or the amino acid sequence of CDR-H1 of Ab-10 comprising no more than 1, 2, or 3 amino acid variations (e.g., substitution, deletion, addition, etc.); the amino acid sequence of CDR-H2 comprises the amino acid sequence of CDR-H2 of Ab-10, or the amino acid sequence of CDR-H2 of Ab-10 comprising no more than 1, 2, or 3 amino acid variations (e.g., substitution, deletion, addition, etc.); the amino acid sequence of CDR-H3 comprises of CDR-H3 of Ab-10, or the amino acid sequence of CDR-H3 of Ab-10 comprising no more than 1, 2, or 3 amino acid variations (e.g., substitution, deletion, addition, etc.); the amino acid sequence of CDR-L1 comprises the amino acid sequence of CDR-L1 of Ab-10, or the amino acid sequence of CDR-L1 of Ab-10 comprising no more than 1, 2, or 3 amino acid variations (e.g., substitution, deletion, addition, etc.); the amino acid sequence of CDR-L2 comprises the amino acid sequence of CDR-L2 of Ab-10, or the amino acid sequence of CDR-L2 of Ab-10 comprising no more than 1, 2, or 3 amino acid variations (e.g., substitution, deletion, addition, etc.); and the amino acid sequence of CDR-L3 comprises the amino acid sequence of CDR-L3 of Ab-10, or the amino acid sequence of CDR-L3 of Ab-10 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-10; the amino acid sequence of CDR-H2 comprises the amino acid sequence of CDR-H2 of Ab-10; the amino acid sequence of CDR-H3 comprises CDR-H3 of Ab-10; the amino acid sequence of CDR-L1 comprises the amino acid sequence of CDR-L1 of Ab-10; the amino acid sequence of CDR-L2 comprises the amino acid sequence of CDR-L2 of Ab-10; and the amino acid sequence of CDR-L3 comprises the amino acid sequence of CDR-L3 of Ab-10.

In some embodiments, the amino acid sequence of CDR-H1 comprises the amino acid sequence of CDR-H1 of Ab-10 comprising no more than 1, 2, or 3 amino acid variations (e.g., substitution, deletion, addition, etc.); the amino acid sequence of CDR-H2 comprises the amino acid sequence CDR-H2 of Ab-10 comprising no more than 1, 2, or 3 amino acid variations (e.g., substitution, deletion, addition, etc.); the amino acid sequence of CDR-H3 comprises the amino acid sequence CDR-H3 of Ab-10 comprising no more than 1, 2, or 3 amino acid variations (e.g., substitution, deletion, addition, etc.); the amino acid sequence of CDR-L1 comprises the amino acid sequence CDR-L1 of Ab-10 comprising no more than 1, 2, or 3 amino acid variations (e.g., substitution, deletion, addition, etc.); the amino acid sequence of a CDR-L2 comprises the amino acid sequence CDR-L2 of Ab-10 comprising no more than 1, 2, or 3 amino acid variations (e.g., substitution, deletion, addition, etc.); and the amino acid sequence of a CDR-L3 comprises the amino acid sequence CDR-L3 of Ab-10 comprising no more than 1, 2, or 3 amino acid variations (e.g., substitution, deletion, addition, etc.).

In some embodiments, the amino acid sequence of CDR-H1 consists of the amino acid sequence of CDR-H1 of Ab-10, or the amino acid sequence of CDR-H1 of Ab-10 comprising 1, 2, or 3 amino acid variations (e.g., substitution, deletion, addition, etc.); the amino acid sequence of CDR-H2 consists of the amino acid sequence of CDR-H2 of Ab-10, or the amino acid sequence of CDR-H2 of Ab-10 comprising 1, 2, or 3 amino acid variations (e.g., substitution, deletion, addition, etc.); the amino acid sequence of CDR-H3 consists of the amino acid sequence of CDR-H3 of Ab-10, or the amino acid sequence of CDR-H3 of Ab-10 comprising 1, 2, or 3 amino acid variations (e.g., substitution, deletion, addition, etc.); the amino acid sequence of CDR-L1 consists of the amino acid sequence of CDR-L1 of Ab-10, or the amino acid sequence of CDR-L1 of a Ab-10 comprising 1, 2, or 3 amino acid variations (e.g., substitution, deletion, addition, etc.); the amino acid sequence of CDR-L2 consists of the amino acid sequence of CDR-L2 of Ab-10, or the amino acid sequence of CDR-L2 of Ab-10 comprising 1, 2, or 3 amino acid variations (e.g., substitution, deletion, addition, etc.); and the amino acid sequence of CDR-L3 consists of the amino acid sequence of CDR-L3 of Ab-10, or the amino acid sequence of CDR-L3 of Ab-10 comprising 1, 2, or 3 amino acid variations (e.g., substitution, deletion, addition, etc.).

In some embodiments, the amino acid sequence of CDR-H1 consists of the amino acid sequence of CDR-H1 of Ab-10, or the amino acid sequence of CDR-H1 of Ab-10 comprising no more than 1, 2, or 3 amino acid variations (e.g., substitution, deletion, addition, etc.); the amino acid sequence of CDR-H2 consists of the amino acid sequence of CDR-H2 of Ab-10, or the amino acid sequence of CDR-H2 of Ab-10 comprising no more than 1, 2, or 3 amino acid variations (e.g., substitution, deletion, addition, etc.); the amino acid sequence of CDR-H3 consists of the amino acid sequence of CDR-H3 of Ab-10, or the amino acid sequence of CDR-H3 of Ab-10 comprising no more than 1, 2, or 3 amino acid variations (e.g., substitution, deletion, addition, etc.); the amino acid sequence of CDR-L1 consists of the amino acid sequence of CDR-L1 of Ab-10, or the amino acid sequence of CDR-L1 of Ab-10 comprising no more than 1, 2, or 3 amino acid variations (e.g., substitution, deletion, addition, etc.); the amino acid sequence of CDR-L2 consists of the amino acid sequence of CDR-L2 of Ab-10, or the amino acid sequence of CDR-L2 of Ab-10 comprising no more than 1, 2, or 3 amino acid variations (e.g., substitution, deletion, addition, etc.); and the amino acid sequence of CDR-L3 consists of the amino acid sequence of CDR-L3 of Ab-10, or the amino acid sequence of CDR-L3 of Ab-10 comprising no more than 1, 2, or 3 amino acid variations (e.g., substitution, deletion, addition, etc.).

In some embodiments, the amino acid sequence of CDR-H1 consists of the amino acid sequence of CDR-H1 of Ab-10; the amino acid sequence of CDR-H2 consists of the amino acid sequence of CDR-H2 of Ab-10; the amino acid sequence of CDR-H3 consists of CDR-H3 of Ab-10; the amino acid sequence of CDR-L1 consists of the amino acid sequence of CDR-L1 of Ab-10; the amino acid sequence of CDR-L2 consists of the amino acid sequence of CDR-L2 of Ab-10; and the amino acid sequence of CDR-L3 consists of the amino acid sequence of CDR-L3 of Ab-10.

In some embodiments, the amino acid sequence of CDR-H1 consists of the amino acid sequence of CDR-H1 of Ab-10 comprising no more than 1, 2, or 3 amino acid variations (e.g., substitution, deletion, addition, etc.); the amino acid sequence of CDR-H2 consists of the amino acid sequence of CDR-H2 of Ab-10 comprising no more than 1, 2, or 3 amino acid variations (e.g., substitution, deletion, addition, etc.); the amino acid sequence of CDR-H3 consists of the amino acid sequence of CDR-H3 of Ab-10 comprising no more than 1, 2, or 3 amino acid variations (e.g., substitution, deletion, addition, etc.); the amino acid sequence of CDR-L1 consists of the amino acid sequence of CDR-L1 of Ab-10 comprising no more than 1, 2, or 3 amino acid variations (e.g., substitution, deletion, addition, etc.); the amino acid sequence of CDR-L2 consists of the amino acid sequence of CDR-L2 of Ab-10 comprising no more than 1, 2, or 3 amino acid variations (e.g., substitution, deletion, addition, etc.); and the amino acid sequence of CDR-L3 consists of the amino acid sequence of CDR-L3 of Ab-10 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-10; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-10. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-10; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-10. In some embodiments, the amino acid sequence of the VH comprises an amino acid sequence at least 95%, 96%, 97%, 98%, 99%, or 100% identical to the amino acid sequence of the VH of Ab-10; and the amino acid sequence of the VL comprises an amino acid sequence at least 95%, 96%, 97%, 98%, 99%, or 100% identical to the amino acid sequence of the VL of Ab-10. In some embodiments, the amino acid sequence of the VH comprises the amino acid sequence of the VH of Ab-10; and the amino acid sequence of the VL comprises the amino acid sequence of the VL of Ab-10.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-10; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-10. In some embodiments, the amino acid sequence of the VH consists of an amino acid sequence at least 90%, 91%, 92%, 93%, 94%, 95%, 96%, 97%, 98%, 99%, or 100% identical to the amino acid sequence of the VH of Ab-10; and the amino acid sequence of the VL consists of an amino acid sequence at least 90%, 91%, 92%, 93%, 94%, 95%, 96%, 97%, 98%, 99%, or 100% identical to the amino acid sequence of the VL of Ab-10. In some embodiments, the amino acid sequence of the VH consists of an amino acid sequence at least 95%, 96%, 97%, 98%, 99%, or 100% identical to the amino acid sequence of the VH of Ab-10; and the amino acid sequence of the VL consists of an amino acid sequence at least 95%, 96%, 97%, 98%, 99%, or 100% identical to the amino acid sequence of the VL of Ab-10. In some embodiments, the amino acid sequence of the VH consists of the amino acid sequence of the VH of Ab-10; and the amino acid sequence of the VL consists of the amino acid sequence of the VL of Ab-10.

In some embodiments, the amino acid sequence of CDR-H1 comprises the amino acid sequence of CDR-H1 of Ab-11, or the amino acid sequence of the CDR-H1 of Ab-11 comprising 1, 2, or 3 amino acid variations (e.g., substitution, deletion, addition, etc.); the amino acid sequence of CDR-H2 comprises the amino acid sequence of CDR-H2 of Ab-11, or the amino acid sequence of CDR-H2 of Ab-11 comprising 1, 2, or 3 amino acid variations (e.g., substitution, deletion, addition, etc.); the amino acid sequence of CDR-H3 comprises the amino acid sequence of CDR-H3 of Ab-11, or the amino acid sequence of CDR-H3 of Ab-11 comprising 1, 2, or 3 amino acid variations (e.g., substitution, deletion, addition, etc.); the amino acid sequence of CDR-L1 comprises the amino acid sequence of CDR-L1 of Ab-11, or the amino acid sequence of CDR-L1 of Ab-11 comprising 1, 2, or 3 amino acid variations (e.g., substitution, deletion, addition, etc.); the amino acid sequence of CDR-L2 comprises the amino acid sequence of a CDR-L2 of Ab-11, or the amino acid sequence of a CDR-L2 of Ab-11 comprising 1, 2, or 3 amino acid variations (e.g., substitution, deletion, addition, etc.); and the amino acid sequence of CDR-L3 comprises the amino acid sequence of CDR-L3 of Ab-11, or the amino acid sequence of CDR-L3 of Ab-11 comprising 1, 2, or 3 amino acid variations (e.g., substitution, deletion, addition, etc.).

In some embodiments, the amino acid sequence of CDR-H1 comprises the amino acid sequence of CDR-H1 of Ab-11, or the amino acid sequence of CDR-H1 of Ab-11 comprising no more than 1, 2, or 3 amino acid variations (e.g., substitution, deletion, addition, etc.); the amino acid sequence of CDR-H2 comprises the amino acid sequence of CDR-H2 of Ab-11, or the amino acid sequence of CDR-H2 of Ab-11 comprising no more than 1, 2, or 3 amino acid variations (e.g., substitution, deletion, addition, etc.); the amino acid sequence of CDR-H3 comprises of CDR-H3 of Ab-11, or the amino acid sequence of CDR-H3 of Ab-11 comprising no more than 1, 2, or 3 amino acid variations (e.g., substitution, deletion, addition, etc.); the amino acid sequence of CDR-L1 comprises the amino acid sequence of CDR-L1 of Ab-11, or the amino acid sequence of CDR-L1 of Ab-11 comprising no more than 1, 2, or 3 amino acid variations (e.g., substitution, deletion, addition, etc.); the amino acid sequence of CDR-L2 comprises the amino acid sequence of CDR-L2 of Ab-11, or the amino acid sequence of CDR-L2 of Ab-11 comprising no more than 1, 2, or 3 amino acid variations (e.g., substitution, deletion, addition, etc.); and the amino acid sequence of CDR-L3 comprises the amino acid sequence of CDR-L3 of Ab-11, or the amino acid sequence of CDR-L3 of Ab-11 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-11; the amino acid sequence of CDR-H2 comprises the amino acid sequence of CDR-H2 of Ab-11; the amino acid sequence of CDR-H3 comprises CDR-H3 of Ab-11; the amino acid sequence of CDR-L1 comprises the amino acid sequence of CDR-L1 of Ab-11; the amino acid sequence of CDR-L2 comprises the amino acid sequence of CDR-L2 of Ab-11; and the amino acid sequence of CDR-L3 comprises the amino acid sequence of CDR-L3 of Ab-11.

In some embodiments, the amino acid sequence of CDR-H1 comprises the amino acid sequence of CDR-H1 of Ab-11 comprising no more than 1, 2, or 3 amino acid variations (e.g., substitution, deletion, addition, etc.); the amino acid sequence of CDR-H2 comprises the amino acid sequence CDR-H2 of Ab-11 comprising no more than 1, 2, or 3 amino acid variations (e.g., substitution, deletion, addition, etc.); the amino acid sequence of CDR-H3 comprises the amino acid sequence CDR-H3 of Ab-11 comprising no more than 1, 2, or 3 amino acid variations (e.g., substitution, deletion, addition, etc.); the amino acid sequence of CDR-L1 comprises the amino acid sequence CDR-L1 of Ab-11 comprising no more than 1, 2, or 3 amino acid variations (e.g., substitution, deletion, addition, etc.); the amino acid sequence of a CDR-L2 comprises the amino acid sequence CDR-L2 of Ab-11 comprising no more than 1, 2, or 3 amino acid variations (e.g., substitution, deletion, addition, etc.); and the amino acid sequence of a CDR-L3 comprises the amino acid sequence CDR-L3 of Ab-11 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-11; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-11. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-11; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-11. In some embodiments, the amino acid sequence of the VH comprises an amino acid sequence at least 95%, 96%, 97%, 98%, 99%, or 100% identical to the amino acid sequence of the VH of Ab-11; and the amino acid sequence of the VL comprises an amino acid sequence at least 95%, 96%, 97%, 98%, 99%, or 100% identical to the amino acid sequence of the VL of Ab-11. In some embodiments, the amino acid sequence of the VH comprises the amino acid sequence of the VH of Ab-11; and the amino acid sequence of the VL comprises the amino acid sequence of the VL of Ab-11.

In some embodiments, the amino acid sequence of CDR-H1 comprises the amino acid sequence of CDR-H1 of Ab-12, or the amino acid sequence of the CDR-H1 of Ab-12 comprising 1, 2, or 3 amino acid variations (e.g., substitution, deletion, addition, etc.); the amino acid sequence of CDR-H2 comprises the amino acid sequence of CDR-H2 of Ab-12, or the amino acid sequence of CDR-H2 of Ab-12 comprising 1, 2, or 3 amino acid variations (e.g., substitution, deletion, addition, etc.); the amino acid sequence of CDR-H3 comprises the amino acid sequence of CDR-H3 of Ab-12, or the amino acid sequence of CDR-H3 of Ab-12 comprising 1, 2, or 3 amino acid variations (e.g., substitution, deletion, addition, etc.); the amino acid sequence of CDR-L1 comprises the amino acid sequence of CDR-L1 of Ab-12, or the amino acid sequence of CDR-L1 of Ab-12 comprising 1, 2, or 3 amino acid variations (e.g., substitution, deletion, addition, etc.); the amino acid sequence of CDR-L2 comprises the amino acid sequence of a CDR-L2 of Ab-12, or the amino acid sequence of a CDR-L2 of Ab-12 comprising 1, 2, or 3 amino acid variations (e.g., substitution, deletion, addition, etc.); and the amino acid sequence of CDR-L3 comprises the amino acid sequence of CDR-L3 of Ab-12, or the amino acid sequence of CDR-L3 of Ab-12 comprising 1, 2, or 3 amino acid variations (e.g., substitution, deletion, addition, etc.).

In some embodiments, the amino acid sequence of CDR-H1 comprises the amino acid sequence of CDR-H1 of Ab-12, or the amino acid sequence of CDR-H1 of Ab-12 comprising no more than 1, 2, or 3 amino acid variations (e.g., substitution, deletion, addition, etc.); the amino acid sequence of CDR-H2 comprises the amino acid sequence of CDR-H2 of Ab-12, or the amino acid sequence of CDR-H2 of Ab-12 comprising no more than 1, 2, or 3 amino acid variations (e.g., substitution, deletion, addition, etc.); the amino acid sequence of CDR-H3 comprises of CDR-H3 of Ab-12, or the amino acid sequence of CDR-H3 of Ab-12 comprising no more than 1, 2, or 3 amino acid variations (e.g., substitution, deletion, addition, etc.); the amino acid sequence of CDR-L1 comprises the amino acid sequence of CDR-L1 of Ab-12, or the amino acid sequence of CDR-L1 of Ab-12 comprising no more than 1, 2, or 3 amino acid variations (e.g., substitution, deletion, addition, etc.); the amino acid sequence of CDR-L2 comprises the amino acid sequence of CDR-L2 of Ab-12, or the amino acid sequence of CDR-L2 of Ab-12 comprising no more than 1, 2, or 3 amino acid variations (e.g., substitution, deletion, addition, etc.); and the amino acid sequence of CDR-L3 comprises the amino acid sequence of CDR-L3 of Ab-12, or the amino acid sequence of CDR-L3 of Ab-12 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-12; the amino acid sequence of CDR-H2 comprises the amino acid sequence of CDR-H2 of Ab-12; the amino acid sequence of CDR-H3 comprises CDR-H3 of Ab-12; the amino acid sequence of CDR-L1 comprises the amino acid sequence of CDR-L1 of Ab-12; the amino acid sequence of CDR-L2 comprises the amino acid sequence of CDR-L2 of Ab-12; and the amino acid sequence of CDR-L3 comprises the amino acid sequence of CDR-L3 of Ab-12.

In some embodiments, the amino acid sequence of CDR-H1 comprises the amino acid sequence of CDR-H1 of Ab-12 comprising no more than 1, 2, or 3 amino acid variations (e.g., substitution, deletion, addition, etc.); the amino acid sequence of CDR-H2 comprises the amino acid sequence CDR-H2 of Ab-12 comprising no more than 1, 2, or 3 amino acid variations (e.g., substitution, deletion, addition, etc.); the amino acid sequence of CDR-H3 comprises the amino acid sequence CDR-H3 of Ab-12 comprising no more than 1, 2, or 3 amino acid variations (e.g., substitution, deletion, addition, etc.); the amino acid sequence of CDR-L1 comprises the amino acid sequence CDR-L1 of Ab-12 comprising no more than 1, 2, or 3 amino acid variations (e.g., substitution, deletion, addition, etc.); the amino acid sequence of a CDR-L2 comprises the amino acid sequence CDR-L2 of Ab-12 comprising no more than 1, 2, or 3 amino acid variations (e.g., substitution, deletion, addition, etc.); and the amino acid sequence of a CDR-L3 comprises the amino acid sequence CDR-L3 of Ab-12 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-12; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-12. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-12; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-12. In some embodiments, the amino acid sequence of the VH comprises an amino acid sequence at least 95%, 96%, 97%, 98%, 99%, or 100% identical to the amino acid sequence of the VH of Ab-12; and the amino acid sequence of the VL comprises an amino acid sequence at least 95%, 96%, 97%, 98%, 99%, or 100% identical to the amino acid sequence of the VL of Ab-12. In some embodiments, the amino acid sequence of the VH comprises the amino acid sequence of the VH of Ab-12; and the amino acid sequence of the VL comprises the amino acid sequence of the VL of Ab-12.

In some embodiments, the amino acid sequence of CDR-H1 comprises the amino acid sequence of CDR-H1 of Ab-13, or the amino acid sequence of the CDR-H1 of Ab-13 comprising 1, 2, or 3 amino acid variations (e.g., substitution, deletion, addition, etc.); the amino acid sequence of CDR-H2 comprises the amino acid sequence of CDR-H2 of Ab-13, or the amino acid sequence of CDR-H2 of Ab-13 comprising 1, 2, or 3 amino acid variations (e.g., substitution, deletion, addition, etc.); the amino acid sequence of CDR-H3 comprises the amino acid sequence of CDR-H3 of Ab-13, or the amino acid sequence of CDR-H3 of Ab-13 comprising 1, 2, or 3 amino acid variations (e.g., substitution, deletion, addition, etc.); the amino acid sequence of CDR-L1 comprises the amino acid sequence of CDR-L1 of Ab-13, or the amino acid sequence of CDR-L1 of Ab-13 comprising 1, 2, or 3 amino acid variations (e.g., substitution, deletion, addition, etc.); the amino acid sequence of CDR-L2 comprises the amino acid sequence of a CDR-L2 of Ab-13, or the amino acid sequence of a CDR-L2 of Ab-13 comprising 1, 2, or 3 amino acid variations (e.g., substitution, deletion, addition, etc.); and the amino acid sequence of CDR-L3 comprises the amino acid sequence of CDR-L3 of Ab-13, or the amino acid sequence of CDR-L3 of Ab-13 comprising 1, 2, or 3 amino acid variations (e.g., substitution, deletion, addition, etc.).

In some embodiments, the amino acid sequence of CDR-H1 comprises the amino acid sequence of CDR-H1 of Ab-13, or the amino acid sequence of CDR-H1 of Ab-13 comprising no more than 1, 2, or 3 amino acid variations (e.g., substitution, deletion, addition, etc.); the amino acid sequence of CDR-H2 comprises the amino acid sequence of CDR-H2 of Ab-13, or the amino acid sequence of CDR-H2 of Ab-13 comprising no more than 1, 2, or 3 amino acid variations (e.g., substitution, deletion, addition, etc.); the amino acid sequence of CDR-H3 comprises of CDR-H3 of Ab-13, or the amino acid sequence of CDR-H3 of Ab-13 comprising no more than 1, 2, or 3 amino acid variations (e.g., substitution, deletion, addition, etc.); the amino acid sequence of CDR-L1 comprises the amino acid sequence of CDR-L1 of Ab-13, or the amino acid sequence of CDR-L1 of Ab-13 comprising no more than 1, 2, or 3 amino acid variations (e.g., substitution, deletion, addition, etc.); the amino acid sequence of CDR-L2 comprises the amino acid sequence of CDR-L2 of Ab-13, or the amino acid sequence of CDR-L2 of Ab-13 comprising no more than 1, 2, or 3 amino acid variations (e.g., substitution, deletion, addition, etc.); and the amino acid sequence of CDR-L3 comprises the amino acid sequence of CDR-L3 of Ab-13, or the amino acid sequence of CDR-L3 of Ab-13 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-13; the amino acid sequence of CDR-H2 comprises the amino acid sequence of CDR-H2 of Ab-13; the amino acid sequence of CDR-H3 comprises CDR-H3 of Ab-13; the amino acid sequence of CDR-L1 comprises the amino acid sequence of CDR-L1 of Ab-13; the amino acid sequence of CDR-L2 comprises the amino acid sequence of CDR-L2 of Ab-13; and the amino acid sequence of CDR-L3 comprises the amino acid sequence of CDR-L3 of Ab-13.

In some embodiments, the amino acid sequence of CDR-H1 comprises the amino acid sequence of CDR-H1 of Ab-13 comprising no more than 1, 2, or 3 amino acid variations (e.g., substitution, deletion, addition, etc.); the amino acid sequence of CDR-H2 comprises the amino acid sequence CDR-H2 of Ab-13 comprising no more than 1, 2, or 3 amino acid variations (e.g., substitution, deletion, addition, etc.); the amino acid sequence of CDR-H3 comprises the amino acid sequence CDR-H3 of Ab-13 comprising no more than 1, 2, or 3 amino acid variations (e.g., substitution, deletion, addition, etc.); the amino acid sequence of CDR-L1 comprises the amino acid sequence CDR-L1 of Ab-13 comprising no more than 1, 2, or 3 amino acid variations (e.g., substitution, deletion, addition, etc.); the amino acid sequence of a CDR-L2 comprises the amino acid sequence CDR-L2 of Ab-13 comprising no more than 1, 2, or 3 amino acid variations (e.g., substitution, deletion, addition, etc.); and the amino acid sequence of a CDR-L3 comprises the amino acid sequence CDR-L3 of Ab-13 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-13; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-13. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-13; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-13. In some embodiments, the amino acid sequence of the VH comprises an amino acid sequence at least 95%, 96%, 97%, 98%, 99%, or 100% identical to the amino acid sequence of the VH of Ab-13; and the amino acid sequence of the VL comprises an amino acid sequence at least 95%, 96%, 97%, 98%, 99%, or 100% identical to the amino acid sequence of the VL of Ab-13. In some embodiments, the amino acid sequence of the VH comprises the amino acid sequence of the VH of Ab-13; and the amino acid sequence of the VL comprises the amino acid sequence of the VL of Ab-13.

In some embodiments, the amino acid sequence of CDR-H1 comprises the amino acid sequence of CDR-H1 of Ab-14, or the amino acid sequence of the CDR-H1 of Ab-14 comprising 1, 2, or 3 amino acid variations (e.g., substitution, deletion, addition, etc.); the amino acid sequence of CDR-H2 comprises the amino acid sequence of CDR-H2 of Ab-14, or the amino acid sequence of CDR-H2 of Ab-14 comprising 1, 2, or 3 amino acid variations (e.g., substitution, deletion, addition, etc.); the amino acid sequence of CDR-H3 comprises the amino acid sequence of CDR-H3 of Ab-14, or the amino acid sequence of CDR-H3 of Ab-14 comprising 1, 2, or 3 amino acid variations (e.g., substitution, deletion, addition, etc.); the amino acid sequence of CDR-L1 comprises the amino acid sequence of CDR-L1 of Ab-14, or the amino acid sequence of CDR-L1 of Ab-14 comprising 1, 2, or 3 amino acid variations (e.g., substitution, deletion, addition, etc.); the amino acid sequence of CDR-L2 comprises the amino acid sequence of a CDR-L2 of Ab-14, or the amino acid sequence of a CDR-L2 of Ab-14 comprising 1, 2, or 3 amino acid variations (e.g., substitution, deletion, addition, etc.); and the amino acid sequence of CDR-L3 comprises the amino acid sequence of CDR-L3 of Ab-14, or the amino acid sequence of CDR-L3 of Ab-14 comprising 1, 2, or 3 amino acid variations (e.g., substitution, deletion, addition, etc.).

In some embodiments, the amino acid sequence of CDR-H1 comprises the amino acid sequence of CDR-H1 of Ab-14, or the amino acid sequence of CDR-H1 of Ab-14 comprising no more than 1, 2, or 3 amino acid variations (e.g., substitution, deletion, addition, etc.); the amino acid sequence of CDR-H2 comprises the amino acid sequence of CDR-H2 of Ab-14, or the amino acid sequence of CDR-H2 of Ab-14 comprising no more than 1, 2, or 3 amino acid variations (e.g., substitution, deletion, addition, etc.); the amino acid sequence of CDR-H3 comprises of CDR-H3 of Ab-14, or the amino acid sequence of CDR-H3 of Ab-14 comprising no more than 1, 2, or 3 amino acid variations (e.g., substitution, deletion, addition, etc.); the amino acid sequence of CDR-L1 comprises the amino acid sequence of CDR-L1 of Ab-14, or the amino acid sequence of CDR-L1 of Ab-14 comprising no more than 1, 2, or 3 amino acid variations (e.g., substitution, deletion, addition, etc.); the amino acid sequence of CDR-L2 comprises the amino acid sequence of CDR-L2 of Ab-14, or the amino acid sequence of CDR-L2 of Ab-14 comprising no more than 1, 2, or 3 amino acid variations (e.g., substitution, deletion, addition, etc.); and the amino acid sequence of CDR-L3 comprises the amino acid sequence of CDR-L3 of Ab-14, or the amino acid sequence of CDR-L3 of Ab-14 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-14; the amino acid sequence of CDR-H2 comprises the amino acid sequence of CDR-H2 of Ab-14; the amino acid sequence of CDR-H3 comprises CDR-H3 of Ab-14; the amino acid sequence of CDR-L1 comprises the amino acid sequence of CDR-L1 of Ab-14; the amino acid sequence of CDR-L2 comprises the amino acid sequence of CDR-L2 of Ab-14; and the amino acid sequence of CDR-L3 comprises the amino acid sequence of CDR-L3 of Ab-14.

In some embodiments, the amino acid sequence of CDR-H1 comprises the amino acid sequence of CDR-H1 of Ab-14 comprising no more than 1, 2, or 3 amino acid variations (e.g., substitution, deletion, addition, etc.); the amino acid sequence of CDR-H2 comprises the amino acid sequence CDR-H2 of Ab-14 comprising no more than 1, 2, or 3 amino acid variations (e.g., substitution, deletion, addition, etc.); the amino acid sequence of CDR-H3 comprises the amino acid sequence CDR-H3 of Ab-14 comprising no more than 1, 2, or 3 amino acid variations (e.g., substitution, deletion, addition, etc.); the amino acid sequence of CDR-L1 comprises the amino acid sequence CDR-L1 of Ab-14 comprising no more than 1, 2, or 3 amino acid variations (e.g., substitution, deletion, addition, etc.); the amino acid sequence of a CDR-L2 comprises the amino acid sequence CDR-L2 of Ab-14 comprising no more than 1, 2, or 3 amino acid variations (e.g., substitution, deletion, addition, etc.); and the amino acid sequence of a CDR-L3 comprises the amino acid sequence CDR-L3 of Ab-14 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-14; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-14. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-14; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-14. In some embodiments, the amino acid sequence of the VH comprises an amino acid sequence at least 95%, 96%, 97%, 98%, 99%, or 100% identical to the amino acid sequence of the VH of Ab-14; and the amino acid sequence of the VL comprises an amino acid sequence at least 95%, 96%, 97%, 98%, 99%, or 100% identical to the amino acid sequence of the VL of Ab-14. In some embodiments, the amino acid sequence of the VH comprises the amino acid sequence of the VH of Ab-14; and the amino acid sequence of the VL comprises the amino acid sequence of the VL of Ab-14.

In some embodiments, the amino acid sequence of CDR-H1 comprises the amino acid sequence of CDR-H1 of Ab-15, or the amino acid sequence of the CDR-H1 of Ab-15 comprising 1, 2, or 3 amino acid variations (e.g., substitution, deletion, addition, etc.); the amino acid sequence of CDR-H2 comprises the amino acid sequence of CDR-H2 of Ab-15, or the amino acid sequence of CDR-H2 of Ab-15 comprising 1, 2, or 3 amino acid variations (e.g., substitution, deletion, addition, etc.); the amino acid sequence of CDR-H3 comprises the amino acid sequence of CDR-H3 of Ab-15, or the amino acid sequence of CDR-H3 of Ab-15 comprising 1, 2, or 3 amino acid variations (e.g., substitution, deletion, addition, etc.); the amino acid sequence of CDR-L1 comprises the amino acid sequence of CDR-L1 of Ab-15, or the amino acid sequence of CDR-L1 of Ab-15 comprising 1, 2, or 3 amino acid variations (e.g., substitution, deletion, addition, etc.); the amino acid sequence of CDR-L2 comprises the amino acid sequence of a CDR-L2 of Ab-15, or the amino acid sequence of a CDR-L2 of Ab-15 comprising 1, 2, or 3 amino acid variations (e.g., substitution, deletion, addition, etc.); and the amino acid sequence of CDR-L3 comprises the amino acid sequence of CDR-L3 of Ab-15, or the amino acid sequence of CDR-L3 of Ab-15 comprising 1, 2, or 3 amino acid variations (e.g., substitution, deletion, addition, etc.).

In some embodiments, the amino acid sequence of CDR-H1 comprises the amino acid sequence of CDR-H1 of Ab-15, or the amino acid sequence of CDR-H1 of Ab-15 comprising no more than 1, 2, or 3 amino acid variations (e.g., substitution, deletion, addition, etc.); the amino acid sequence of CDR-H2 comprises the amino acid sequence of CDR-H2 of Ab-15, or the amino acid sequence of CDR-H2 of Ab-15 comprising no more than 1, 2, or 3 amino acid variations (e.g., substitution, deletion, addition, etc.); the amino acid sequence of CDR-H3 comprises of CDR-H3 of Ab-15, or the amino acid sequence of CDR-H3 of Ab-15 comprising no more than 1, 2, or 3 amino acid variations (e.g., substitution, deletion, addition, etc.); the amino acid sequence of CDR-L1 comprises the amino acid sequence of CDR-L1 of Ab-15, or the amino acid sequence of CDR-L1 of Ab-15 comprising no more than 1, 2, or 3 amino acid variations (e.g., substitution, deletion, addition, etc.); the amino acid sequence of CDR-L2 comprises the amino acid sequence of CDR-L2 of Ab-15, or the amino acid sequence of CDR-L2 of Ab-15 comprising no more than 1, 2, or 3 amino acid variations (e.g., substitution, deletion, addition, etc.); and the amino acid sequence of CDR-L3 comprises the amino acid sequence of CDR-L3 of Ab-15, or the amino acid sequence of CDR-L3 of Ab-15 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-15; the amino acid sequence of CDR-H2 comprises the amino acid sequence of CDR-H2 of Ab-15; the amino acid sequence of CDR-H3 comprises CDR-H3 of Ab-15; the amino acid sequence of CDR-L1 comprises the amino acid sequence of CDR-L1 of Ab-15; the amino acid sequence of CDR-L2 comprises the amino acid sequence of CDR-L2 of Ab-15; and the amino acid sequence of CDR-L3 comprises the amino acid sequence of CDR-L3 of Ab-15.

In some embodiments, the amino acid sequence of CDR-H1 comprises the amino acid sequence of CDR-H1 of Ab-15 comprising no more than 1, 2, or 3 amino acid variations (e.g., substitution, deletion, addition, etc.); the amino acid sequence of CDR-H2 comprises the amino acid sequence CDR-H2 of Ab-15 comprising no more than 1, 2, or 3 amino acid variations (e.g., substitution, deletion, addition, etc.); the amino acid sequence of CDR-H3 comprises the amino acid sequence CDR-H3 of Ab-15 comprising no more than 1, 2, or 3 amino acid variations (e.g., substitution, deletion, addition, etc.); the amino acid sequence of CDR-L1 comprises the amino acid sequence CDR-L1 of Ab-15 comprising no more than 1, 2, or 3 amino acid variations (e.g., substitution, deletion, addition, etc.); the amino acid sequence of a CDR-L2 comprises the amino acid sequence CDR-L2 of Ab-15 comprising no more than 1, 2, or 3 amino acid variations (e.g., substitution, deletion, addition, etc.); and the amino acid sequence of a CDR-L3 comprises the amino acid sequence CDR-L3 of Ab-15 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-15; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-15. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-15; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-15. In some embodiments, the amino acid sequence of the VH comprises an amino acid sequence at least 95%, 96%, 97%, 98%, 99%, or 100% identical to the amino acid sequence of the VH of Ab-15; and the amino acid sequence of the VL comprises an amino acid sequence at least 95%, 96%, 97%, 98%, 99%, or 100% identical to the amino acid sequence of the VL of Ab-15. In some embodiments, the amino acid sequence of the VH comprises the amino acid sequence of the VH of Ab-15; and the amino acid sequence of the VL comprises the amino acid sequence of the VL of Ab-15.

In some embodiments, the amino acid sequence of CDR-H1 comprises the amino acid sequence of CDR-H1 of Ab-16, or the amino acid sequence of the CDR-H1 of Ab-16 comprising 1, 2, or 3 amino acid variations (e.g., substitution, deletion, addition, etc.); the amino acid sequence of CDR-H2 comprises the amino acid sequence of CDR-H2 of Ab-16, or the amino acid sequence of CDR-H2 of Ab-16 comprising 1, 2, or 3 amino acid variations (e.g., substitution, deletion, addition, etc.); the amino acid sequence of CDR-H3 comprises the amino acid sequence of CDR-H3 of Ab-16, or the amino acid sequence of CDR-H3 of Ab-16 comprising 1, 2, or 3 amino acid variations (e.g., substitution, deletion, addition, etc.); the amino acid sequence of CDR-L1 comprises the amino acid sequence of CDR-L1 of Ab-16, or the amino acid sequence of CDR-L1 of Ab-16 comprising 1, 2, or 3 amino acid variations (e.g., substitution, deletion, addition, etc.); the amino acid sequence of CDR-L2 comprises the amino acid sequence of a CDR-L2 of Ab-16, or the amino acid sequence of a CDR-L2 of Ab-16 comprising 1, 2, or 3 amino acid variations (e.g., substitution, deletion, addition, etc.); and the amino acid sequence of CDR-L3 comprises the amino acid sequence of CDR-L3 of Ab-16, or the amino acid sequence of CDR-L3 of Ab-16 comprising 1, 2, or 3 amino acid variations (e.g., substitution, deletion, addition, etc.).

In some embodiments, the amino acid sequence of CDR-H1 comprises the amino acid sequence of CDR-H1 of Ab-16, or the amino acid sequence of CDR-H1 of Ab-16 comprising no more than 1, 2, or 3 amino acid variations (e.g., substitution, deletion, addition, etc.); the amino acid sequence of CDR-H2 comprises the amino acid sequence of CDR-H2 of Ab-16, or the amino acid sequence of CDR-H2 of Ab-16 comprising no more than 1, 2, or 3 amino acid variations (e.g., substitution, deletion, addition, etc.); the amino acid sequence of CDR-H3 comprises of CDR-H3 of Ab-16, or the amino acid sequence of CDR-H3 of Ab-16 comprising no more than 1, 2, or 3 amino acid variations (e.g., substitution, deletion, addition, etc.); the amino acid sequence of CDR-L1 comprises the amino acid sequence of CDR-L1 of Ab-16, or the amino acid sequence of CDR-L1 of Ab-16 comprising no more than 1, 2, or 3 amino acid variations (e.g., substitution, deletion, addition, etc.); the amino acid sequence of CDR-L2 comprises the amino acid sequence of CDR-L2 of Ab-16, or the amino acid sequence of CDR-L2 of Ab-16 comprising no more than 1, 2, or 3 amino acid variations (e.g., substitution, deletion, addition, etc.); and the amino acid sequence of CDR-L3 comprises the amino acid sequence of CDR-L3 of Ab-16, or the amino acid sequence of CDR-L3 of Ab-16 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-16; the amino acid sequence of CDR-H2 comprises the amino acid sequence of CDR-H2 of Ab-16; the amino acid sequence of CDR-H3 comprises CDR-H3 of Ab-16; the amino acid sequence of CDR-L1 comprises the amino acid sequence of CDR-L1 of Ab-16; the amino acid sequence of CDR-L2 comprises the amino acid sequence of CDR-L2 of Ab-16; and the amino acid sequence of CDR-L3 comprises the amino acid sequence of CDR-L3 of Ab-16.

In some embodiments, the amino acid sequence of CDR-H1 comprises the amino acid sequence of CDR-H1 of Ab-16 comprising no more than 1, 2, or 3 amino acid variations (e.g., substitution, deletion, addition, etc.); the amino acid sequence of CDR-H2 comprises the amino acid sequence CDR-H2 of Ab-16 comprising no more than 1, 2, or 3 amino acid variations (e.g., substitution, deletion, addition, etc.); the amino acid sequence of CDR-H3 comprises the amino acid sequence CDR-H3 of Ab-16 comprising no more than 1, 2, or 3 amino acid variations (e.g., substitution, deletion, addition, etc.); the amino acid sequence of CDR-L1 comprises the amino acid sequence CDR-L1 of Ab-16 comprising no more than 1, 2, or 3 amino acid variations (e.g., substitution, deletion, addition, etc.); the amino acid sequence of a CDR-L2 comprises the amino acid sequence CDR-L2 of Ab-16 comprising no more than 1, 2, or 3 amino acid variations (e.g., substitution, deletion, addition, etc.); and the amino acid sequence of a CDR-L3 comprises the amino acid sequence CDR-L3 of Ab-16 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-16; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-16. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-16; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-16. In some embodiments, the amino acid sequence of the VH comprises an amino acid sequence at least 95%, 96%, 97%, 98%, 99%, or 100% identical to the amino acid sequence of the VH of Ab-16; and the amino acid sequence of the VL comprises an amino acid sequence at least 95%, 96%, 97%, 98%, 99%, or 100% identical to the amino acid sequence of the VL of Ab-16. In some embodiments, the amino acid sequence of the VH comprises the amino acid sequence of the VH of Ab-16; and the amino acid sequence of the VL comprises the amino acid sequence of the VL of Ab-16.

In some embodiments, the amino acid sequence of CDR-H1 comprises the amino acid sequence of CDR-H1 of Ab-17, or the amino acid sequence of the CDR-H1 of Ab-17 comprising 1, 2, or 3 amino acid variations (e.g., substitution, deletion, addition, etc.); the amino acid sequence of CDR-H2 comprises the amino acid sequence of CDR-H2 of Ab-17, or the amino acid sequence of CDR-H2 of Ab-17 comprising 1, 2, or 3 amino acid variations (e.g., substitution, deletion, addition, etc.); the amino acid sequence of CDR-H3 comprises the amino acid sequence of CDR-H3 of Ab-17, or the amino acid sequence of CDR-H3 of Ab-17 comprising 1, 2, or 3 amino acid variations (e.g., substitution, deletion, addition, etc.); the amino acid sequence of CDR-L1 comprises the amino acid sequence of CDR-L1 of Ab-17, or the amino acid sequence of CDR-L1 of Ab-17 comprising 1, 2, or 3 amino acid variations (e.g., substitution, deletion, addition, etc.); the amino acid sequence of CDR-L2 comprises the amino acid sequence of a CDR-L2 of Ab-17, or the amino acid sequence of a CDR-L2 of Ab-17 comprising 1, 2, or 3 amino acid variations (e.g., substitution, deletion, addition, etc.); and the amino acid sequence of CDR-L3 comprises the amino acid sequence of CDR-L3 of Ab-17, or the amino acid sequence of CDR-L3 of Ab-17 comprising 1, 2, or 3 amino acid variations (e.g., substitution, deletion, addition, etc.).

In some embodiments, the amino acid sequence of CDR-H1 comprises the amino acid sequence of CDR-H1 of Ab-17, or the amino acid sequence of CDR-H1 of Ab-17 comprising no more than 1, 2, or 3 amino acid variations (e.g., substitution, deletion, addition, etc.); the amino acid sequence of CDR-H2 comprises the amino acid sequence of CDR-H2 of Ab-17, or the amino acid sequence of CDR-H2 of Ab-17 comprising no more than 1, 2, or 3 amino acid variations (e.g., substitution, deletion, addition, etc.); the amino acid sequence of CDR-H3 comprises of CDR-H3 of Ab-17, or the amino acid sequence of CDR-H3 of Ab-17 comprising no more than 1, 2, or 3 amino acid variations (e.g., substitution, deletion, addition, etc.); the amino acid sequence of CDR-L1 comprises the amino acid sequence of CDR-L1 of Ab-17, or the amino acid sequence of CDR-L1 of Ab-17 comprising no more than 1, 2, or 3 amino acid variations (e.g., substitution, deletion, addition, etc.); the amino acid sequence of CDR-L2 comprises the amino acid sequence of CDR-L2 of Ab-17, or the amino acid sequence of CDR-L2 of Ab-17 comprising no more than 1, 2, or 3 amino acid variations (e.g., substitution, deletion, addition, etc.); and the amino acid sequence of CDR-L3 comprises the amino acid sequence of CDR-L3 of Ab-17, or the amino acid sequence of CDR-L3 of Ab-17 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-17; the amino acid sequence of CDR-H2 comprises the amino acid sequence of CDR-H2 of Ab-17; the amino acid sequence of CDR-H3 comprises CDR-H3 of Ab-17; the amino acid sequence of CDR-L1 comprises the amino acid sequence of CDR-L1 of Ab-17; the amino acid sequence of CDR-L2 comprises the amino acid sequence of CDR-L2 of Ab-17; and the amino acid sequence of CDR-L3 comprises the amino acid sequence of CDR-L3 of Ab-17.

In some embodiments, the amino acid sequence of CDR-H1 comprises the amino acid sequence of CDR-H1 of Ab-17 comprising no more than 1, 2, or 3 amino acid variations (e.g., substitution, deletion, addition, etc.); the amino acid sequence of CDR-H2 comprises the amino acid sequence CDR-H2 of Ab-17 comprising no more than 1, 2, or 3 amino acid variations (e.g., substitution, deletion, addition, etc.); the amino acid sequence of CDR-H3 comprises the amino acid sequence CDR-H3 of Ab-17 comprising no more than 1, 2, or 3 amino acid variations (e.g., substitution, deletion, addition, etc.); the amino acid sequence of CDR-L1 comprises the amino acid sequence CDR-L1 of Ab-17 comprising no more than 1, 2, or 3 amino acid variations (e.g., substitution, deletion, addition, etc.); the amino acid sequence of a CDR-L2 comprises the amino acid sequence CDR-L2 of Ab-17 comprising no more than 1, 2, or 3 amino acid variations (e.g., substitution, deletion, addition, etc.); and the amino acid sequence of a CDR-L3 comprises the amino acid sequence CDR-L3 of Ab-17 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-17; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-17. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-17; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-17. In some embodiments, the amino acid sequence of the VH comprises an amino acid sequence at least 95%, 96%, 97%, 98%, 99%, or 100% identical to the amino acid sequence of the VH of Ab-17; and the amino acid sequence of the VL comprises an amino acid sequence at least 95%, 96%, 97%, 98%, 99%, or 100% identical to the amino acid sequence of the VL of Ab-17. In some embodiments, the amino acid sequence of the VH comprises the amino acid sequence of the VH of Ab-17; and the amino acid sequence of the VL comprises the amino acid sequence of the VL of Ab-17.

In some embodiments, the amino acid sequence of CDR-H1 comprises the amino acid sequence of CDR-H1 of Ab-18, or the amino acid sequence of the CDR-H1 of Ab-18 comprising 1, 2, or 3 amino acid variations (e.g., substitution, deletion, addition, etc.); the amino acid sequence of CDR-H2 comprises the amino acid sequence of CDR-H2 of Ab-18, or the amino acid sequence of CDR-H2 of Ab-18 comprising 1, 2, or 3 amino acid variations (e.g., substitution, deletion, addition, etc.); the amino acid sequence of CDR-H3 comprises the amino acid sequence of CDR-H3 of Ab-18, or the amino acid sequence of CDR-H3 of Ab-18 comprising 1, 2, or 3 amino acid variations (e.g., substitution, deletion, addition, etc.); the amino acid sequence of CDR-L1 comprises the amino acid sequence of CDR-L1 of Ab-18, or the amino acid sequence of CDR-L1 of Ab-18 comprising 1, 2, or 3 amino acid variations (e.g., substitution, deletion, addition, etc.); the amino acid sequence of CDR-L2 comprises the amino acid sequence of a CDR-L2 of Ab-18, or the amino acid sequence of a CDR-L2 of Ab-18 comprising 1, 2, or 3 amino acid variations (e.g., substitution, deletion, addition, etc.); and the amino acid sequence of CDR-L3 comprises the amino acid sequence of CDR-L3 of Ab-18, or the amino acid sequence of CDR-L3 of Ab-18 comprising 1, 2, or 3 amino acid variations (e.g., substitution, deletion, addition, etc.).

In some embodiments, the amino acid sequence of CDR-H1 comprises the amino acid sequence of CDR-H1 of Ab-18, or the amino acid sequence of CDR-H1 of Ab-18 comprising no more than 1, 2, or 3 amino acid variations (e.g., substitution, deletion, addition, etc.); the amino acid sequence of CDR-H2 comprises the amino acid sequence of CDR-H2 of Ab-18, or the amino acid sequence of CDR-H2 of Ab-18 comprising no more than 1, 2, or 3 amino acid variations (e.g., substitution, deletion, addition, etc.); the amino acid sequence of CDR-H3 comprises of CDR-H3 of Ab-18, or the amino acid sequence of CDR-H3 of Ab-18 comprising no more than 1, 2, or 3 amino acid variations (e.g., substitution, deletion, addition, etc.); the amino acid sequence of CDR-L1 comprises the amino acid sequence of CDR-L1 of Ab-18, or the amino acid sequence of CDR-L1 of Ab-18 comprising no more than 1, 2, or 3 amino acid variations (e.g., substitution, deletion, addition, etc.); the amino acid sequence of CDR-L2 comprises the amino acid sequence of CDR-L2 of Ab-18, or the amino acid sequence of CDR-L2 of Ab-18 comprising no more than 1, 2, or 3 amino acid variations (e.g., substitution, deletion, addition, etc.); and the amino acid sequence of CDR-L3 comprises the amino acid sequence of CDR-L3 of Ab-18, or the amino acid sequence of CDR-L3 of Ab-18 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-18; the amino acid sequence of CDR-H2 comprises the amino acid sequence of CDR-H2 of Ab-18; the amino acid sequence of CDR-H3 comprises CDR-H3 of Ab-18; the amino acid sequence of CDR-L1 comprises the amino acid sequence of CDR-L1 of Ab-18; the amino acid sequence of CDR-L2 comprises the amino acid sequence of CDR-L2 of Ab-18; and the amino acid sequence of CDR-L3 comprises the amino acid sequence of CDR-L3 of Ab-18.

In some embodiments, the amino acid sequence of CDR-H1 comprises the amino acid sequence of CDR-H1 of Ab-18 comprising no more than 1, 2, or 3 amino acid variations (e.g., substitution, deletion, addition, etc.); the amino acid sequence of CDR-H2 comprises the amino acid sequence CDR-H2 of Ab-18 comprising no more than 1, 2, or 3 amino acid variations (e.g., substitution, deletion, addition, etc.); the amino acid sequence of CDR-H3 comprises the amino acid sequence CDR-H3 of Ab-18 comprising no more than 1, 2, or 3 amino acid variations (e.g., substitution, deletion, addition, etc.); the amino acid sequence of CDR-L1 comprises the amino acid sequence CDR-L1 of Ab-18 comprising no more than 1, 2, or 3 amino acid variations (e.g., substitution, deletion, addition, etc.); the amino acid sequence of a CDR-L2 comprises the amino acid sequence CDR-L2 of Ab-18 comprising no more than 1, 2, or 3 amino acid variations (e.g., substitution, deletion, addition, etc.); and the amino acid sequence of a CDR-L3 comprises the amino acid sequence CDR-L3 of Ab-18 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-18; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-18. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-18; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-18. In some embodiments, the amino acid sequence of the VH comprises an amino acid sequence at least 95%, 96%, 97%, 98%, 99%, or 100% identical to the amino acid sequence of the VH of Ab-18; and the amino acid sequence of the VL comprises an amino acid sequence at least 95%, 96%, 97%, 98%, 99%, or 100% identical to the amino acid sequence of the VL of Ab-18. In some embodiments, the amino acid sequence of the VH comprises the amino acid sequence of the VH of Ab-18; and the amino acid sequence of the VL comprises the amino acid sequence of the VL of Ab-18.

In some embodiments, the amino acid sequence of CDR-H1 comprises the amino acid sequence of CDR-H1 of Ab-19, or the amino acid sequence of the CDR-H1 of Ab-19 comprising 1, 2, or 3 amino acid variations (e.g., substitution, deletion, addition, etc.); the amino acid sequence of CDR-H2 comprises the amino acid sequence of CDR-H2 of Ab-19, or the amino acid sequence of CDR-H2 of Ab-19 comprising 1, 2, or 3 amino acid variations (e.g., substitution, deletion, addition, etc.); the amino acid sequence of CDR-H3 comprises the amino acid sequence of CDR-H3 of Ab-19, or the amino acid sequence of CDR-H3 of Ab-19 comprising 1, 2, or 3 amino acid variations (e.g., substitution, deletion, addition, etc.); the amino acid sequence of CDR-L1 comprises the amino acid sequence of CDR-L1 of Ab-19, or the amino acid sequence of CDR-L1 of Ab-19 comprising 1, 2, or 3 amino acid variations (e.g., substitution, deletion, addition, etc.); the amino acid sequence of CDR-L2 comprises the amino acid sequence of a CDR-L2 of Ab-19, or the amino acid sequence of a CDR-L2 of Ab-19 comprising 1, 2, or 3 amino acid variations (e.g., substitution, deletion, addition, etc.); and the amino acid sequence of CDR-L3 comprises the amino acid sequence of CDR-L3 of Ab-19, or the amino acid sequence of CDR-L3 of Ab-19 comprising 1, 2, or 3 amino acid variations (e.g., substitution, deletion, addition, etc.).

In some embodiments, the amino acid sequence of CDR-H1 comprises the amino acid sequence of CDR-H1 of Ab-19, or the amino acid sequence of CDR-H1 of Ab-19 comprising no more than 1, 2, or 3 amino acid variations (e.g., substitution, deletion, addition, etc.); the amino acid sequence of CDR-H2 comprises the amino acid sequence of CDR-H2 of Ab-19, or the amino acid sequence of CDR-H2 of Ab-19 comprising no more than 1, 2, or 3 amino acid variations (e.g., substitution, deletion, addition, etc.); the amino acid sequence of CDR-H3 comprises of CDR-H3 of Ab-19, or the amino acid sequence of CDR-H3 of Ab-19 comprising no more than 1, 2, or 3 amino acid variations (e.g., substitution, deletion, addition, etc.); the amino acid sequence of CDR-L1 comprises the amino acid sequence of CDR-L1 of Ab-19, or the amino acid sequence of CDR-L1 of Ab-19 comprising no more than 1, 2, or 3 amino acid variations (e.g., substitution, deletion, addition, etc.); the amino acid sequence of CDR-L2 comprises the amino acid sequence of CDR-L2 of Ab-19, or the amino acid sequence of CDR-L2 of Ab-19 comprising no more than 1, 2, or 3 amino acid variations (e.g., substitution, deletion, addition, etc.); and the amino acid sequence of CDR-L3 comprises the amino acid sequence of CDR-L3 of Ab-19, or the amino acid sequence of CDR-L3 of Ab-19 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-19; the amino acid sequence of CDR-H2 comprises the amino acid sequence of CDR-H2 of Ab-19; the amino acid sequence of CDR-H3 comprises CDR-H3 of Ab-19; the amino acid sequence of CDR-L1 comprises the amino acid sequence of CDR-L1 of Ab-19; the amino acid sequence of CDR-L2 comprises the amino acid sequence of CDR-L2 of Ab-19; and the amino acid sequence of CDR-L3 comprises the amino acid sequence of CDR-L3 of Ab-19.

In some embodiments, the amino acid sequence of CDR-H1 comprises the amino acid sequence of CDR-H1 of Ab-19 comprising no more than 1, 2, or 3 amino acid variations (e.g., substitution, deletion, addition, etc.); the amino acid sequence of CDR-H2 comprises the amino acid sequence CDR-H2 of Ab-19 comprising no more than 1, 2, or 3 amino acid variations (e.g., substitution, deletion, addition, etc.); the amino acid sequence of CDR-H3 comprises the amino acid sequence CDR-H3 of Ab-19 comprising no more than 1, 2, or 3 amino acid variations (e.g., substitution, deletion, addition, etc.); the amino acid sequence of CDR-L1 comprises the amino acid sequence CDR-L1 of Ab-19 comprising no more than 1, 2, or 3 amino acid variations (e.g., substitution, deletion, addition, etc.); the amino acid sequence of a CDR-L2 comprises the amino acid sequence CDR-L2 of Ab-19 comprising no more than 1, 2, or 3 amino acid variations (e.g., substitution, deletion, addition, etc.); and the amino acid sequence of a CDR-L3 comprises the amino acid sequence CDR-L3 of Ab-19 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-19; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-19. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-19; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-19. In some embodiments, the amino acid sequence of the VH comprises an amino acid sequence at least 95%, 96%, 97%, 98%, 99%, or 100% identical to the amino acid sequence of the VH of Ab-19; and the amino acid sequence of the VL comprises an amino acid sequence at least 95%, 96%, 97%, 98%, 99%, or 100% identical to the amino acid sequence of the VL of Ab-19. In some embodiments, the amino acid sequence of the VH comprises the amino acid sequence of the VH of Ab-19; and the amino acid sequence of the VL comprises the amino acid sequence of the VL of Ab-19.

In some embodiments, the amino acid sequence of CDR-H1 comprises the amino acid sequence of CDR-H1 of Ab-20, or the amino acid sequence of the CDR-H1 of Ab-20 comprising 1, 2, or 3 amino acid variations (e.g., substitution, deletion, addition, etc.); the amino acid sequence of CDR-H2 comprises the amino acid sequence of CDR-H2 of Ab-20, or the amino acid sequence of CDR-H2 of Ab-20 comprising 1, 2, or 3 amino acid variations (e.g., substitution, deletion, addition, etc.); the amino acid sequence of CDR-H3 comprises the amino acid sequence of CDR-H3 of Ab-20, or the amino acid sequence of CDR-H3 of Ab-20 comprising 1, 2, or 3 amino acid variations (e.g., substitution, deletion, addition, etc.); the amino acid sequence of CDR-L1 comprises the amino acid sequence of CDR-L1 of Ab-20, or the amino acid sequence of CDR-L1 of Ab-20 comprising 1, 2, or 3 amino acid variations (e.g., substitution, deletion, addition, etc.); the amino acid sequence of CDR-L2 comprises the amino acid sequence of a CDR-L2 of Ab-20, or the amino acid sequence of a CDR-L2 of Ab-20 comprising 1, 2, or 3 amino acid variations (e.g., substitution, deletion, addition, etc.); and the amino acid sequence of CDR-L3 comprises the amino acid sequence of CDR-L3 of Ab-20, or the amino acid sequence of CDR-L3 of Ab-20 comprising 1, 2, or 3 amino acid variations (e.g., substitution, deletion, addition, etc.).

In some embodiments, the amino acid sequence of CDR-H1 comprises the amino acid sequence of CDR-H1 of Ab-20, or the amino acid sequence of CDR-H1 of Ab-20 comprising no more than 1, 2, or 3 amino acid variations (e.g., substitution, deletion, addition, etc.); the amino acid sequence of CDR-H2 comprises the amino acid sequence of CDR-H2 of Ab-20, or the amino acid sequence of CDR-H2 of Ab-20 comprising no more than 1, 2, or 3 amino acid variations (e.g., substitution, deletion, addition, etc.); the amino acid sequence of CDR-H3 comprises of CDR-H3 of Ab-20, or the amino acid sequence of CDR-H3 of Ab-20 comprising no more than 1, 2, or 3 amino acid variations (e.g., substitution, deletion, addition, etc.); the amino acid sequence of CDR-L1 comprises the amino acid sequence of CDR-L1 of Ab-20, or the amino acid sequence of CDR-L1 of Ab-20 comprising no more than 1, 2, or 3 amino acid variations (e.g., substitution, deletion, addition, etc.); the amino acid sequence of CDR-L2 comprises the amino acid sequence of CDR-L2 of Ab-20, or the amino acid sequence of CDR-L2 of Ab-20 comprising no more than 1, 2, or 3 amino acid variations (e.g., substitution, deletion, addition, etc.); and the amino acid sequence of CDR-L3 comprises the amino acid sequence of CDR-L3 of Ab-20, or the amino acid sequence of CDR-L3 of Ab-20 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-20; the amino acid sequence of CDR-H2 comprises the amino acid sequence of CDR-H2 of Ab-20; the amino acid sequence of CDR-H3 comprises CDR-H3 of Ab-20; the amino acid sequence of CDR-L1 comprises the amino acid sequence of CDR-L1 of Ab-20; the amino acid sequence of CDR-L2 comprises the amino acid sequence of CDR-L2 of Ab-20; and the amino acid sequence of CDR-L3 comprises the amino acid sequence of CDR-L3 of Ab-20.

In some embodiments, the amino acid sequence of CDR-H1 comprises the amino acid sequence of CDR-H1 of Ab-20 comprising no more than 1, 2, or 3 amino acid variations (e.g., substitution, deletion, addition, etc.); the amino acid sequence of CDR-H2 comprises the amino acid sequence CDR-H2 of Ab-20 comprising no more than 1, 2, or 3 amino acid variations (e.g., substitution, deletion, addition, etc.); the amino acid sequence of CDR-H3 comprises the amino acid sequence CDR-H3 of Ab-20 comprising no more than 1, 2, or 3 amino acid variations (e.g., substitution, deletion, addition, etc.); the amino acid sequence of CDR-L1 comprises the amino acid sequence CDR-L1 of Ab-20 comprising no more than 1, 2, or 3 amino acid variations (e.g., substitution, deletion, addition, etc.); the amino acid sequence of a CDR-L2 comprises the amino acid sequence CDR-L2 of Ab-20 comprising no more than 1, 2, or 3 amino acid variations (e.g., substitution, deletion, addition, etc.); and the amino acid sequence of a CDR-L3 comprises the amino acid sequence CDR-L3 of Ab-20 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-20; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-20. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-20; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-20. In some embodiments, the amino acid sequence of the VH comprises an amino acid sequence at least 95%, 96%, 97%, 98%, 99%, or 100% identical to the amino acid sequence of the VH of Ab-20; and the amino acid sequence of the VL comprises an amino acid sequence at least 95%, 96%, 97%, 98%, 99%, or 100% identical to the amino acid sequence of the VL of Ab-20. In some embodiments, the amino acid sequence of the VH comprises the amino acid sequence of the VH of Ab-20; and the amino acid sequence of the VL comprises the amino acid sequence of the VL of Ab-20.

In some embodiments, the amino acid sequence of CDR-H1 comprises the amino acid sequence of CDR-H1 of Ab-21, or the amino acid sequence of the CDR-H1 of Ab-21 comprising 1, 2, or 3 amino acid variations (e.g., substitution, deletion, addition, etc.); the amino acid sequence of CDR-H2 comprises the amino acid sequence of CDR-H2 of Ab-21, or the amino acid sequence of CDR-H2 of Ab-21 comprising 1, 2, or 3 amino acid variations (e.g., substitution, deletion, addition, etc.); the amino acid sequence of CDR-H3 comprises the amino acid sequence of CDR-H3 of Ab-21, or the amino acid sequence of CDR-H3 of Ab-21 comprising 1, 2, or 3 amino acid variations (e.g., substitution, deletion, addition, etc.); the amino acid sequence of CDR-L1 comprises the amino acid sequence of CDR-L1 of Ab-21, or the amino acid sequence of CDR-L1 of Ab-21 comprising 1, 2, or 3 amino acid variations (e.g., substitution, deletion, addition, etc.); the amino acid sequence of CDR-L2 comprises the amino acid sequence of a CDR-L2 of Ab-21, or the amino acid sequence of a CDR-L2 of Ab-21 comprising 1, 2, or 3 amino acid variations (e.g., substitution, deletion, addition, etc.); and the amino acid sequence of CDR-L3 comprises the amino acid sequence of CDR-L3 of Ab-21, or the amino acid sequence of CDR-L3 of Ab-21 comprising 1, 2, or 3 amino acid variations (e.g., substitution, deletion, addition, etc.).

In some embodiments, the amino acid sequence of CDR-H1 comprises the amino acid sequence of CDR-H1 of Ab-21, or the amino acid sequence of CDR-H1 of Ab-21 comprising no more than 1, 2, or 3 amino acid variations (e.g., substitution, deletion, addition, etc.); the amino acid sequence of CDR-H2 comprises the amino acid sequence of CDR-H2 of Ab-21, or the amino acid sequence of CDR-H2 of Ab-21 comprising no more than 1, 2, or 3 amino acid variations (e.g., substitution, deletion, addition, etc.); the amino acid sequence of CDR-H3 comprises of CDR-H3 of Ab-21, or the amino acid sequence of CDR-H3 of Ab-21 comprising no more than 1, 2, or 3 amino acid variations (e.g., substitution, deletion, addition, etc.); the amino acid sequence of CDR-L1 comprises the amino acid sequence of CDR-L1 of Ab-21, or the amino acid sequence of CDR-L1 of Ab-21 comprising no more than 1, 2, or 3 amino acid variations (e.g., substitution, deletion, addition, etc.); the amino acid sequence of CDR-L2 comprises the amino acid sequence of CDR-L2 of Ab-21, or the amino acid sequence of CDR-L2 of Ab-21 comprising no more than 1, 2, or 3 amino acid variations (e.g., substitution, deletion, addition, etc.); and the amino acid sequence of CDR-L3 comprises the amino acid sequence of CDR-L3 of Ab-21, or the amino acid sequence of CDR-L3 of Ab-21 comprising no more than 1, 2, or 3 amino acid variations (e.g., substitution, deletion, addition, etc.).

In some embodiments, the amino acid sequence of CDR-H1 comprises the amino acid sequence of CDR-H1 of Ab-21; the amino acid sequence of CDR-H2 comprises the amino acid sequence of CDR-H2 of Ab-21; the amino acid sequence of CDR-H3 comprises CDR-H3 of Ab-21; the amino acid sequence of CDR-L1 comprises the amino acid sequence of CDR-L1 of Ab-21; the amino acid sequence of CDR-L2 comprises the amino acid sequence of CDR-L2 of Ab-21; and the amino acid sequence of CDR-L3 comprises the amino acid sequence of CDR-L3 of Ab-21.

In some embodiments, the amino acid sequence of CDR-H1 comprises the amino acid sequence of CDR-H1 of Ab-21 comprising no more than 1, 2, or 3 amino acid variations (e.g., substitution, deletion, addition, etc.); the amino acid sequence of CDR-H2 comprises the amino acid sequence CDR-H2 of Ab-21 comprising no more than 1, 2, or 3 amino acid variations (e.g., substitution, deletion, addition, etc.); the amino acid sequence of CDR-H3 comprises the amino acid sequence CDR-H3 of Ab-21 comprising no more than 1, 2, or 3 amino acid variations (e.g., substitution, deletion, addition, etc.); the amino acid sequence of CDR-L1 comprises the amino acid sequence CDR-L1 of Ab-21 comprising no more than 1, 2, or 3 amino acid variations (e.g., substitution, deletion, addition, etc.); the amino acid sequence of a CDR-L2 comprises the amino acid sequence CDR-L2 of Ab-21 comprising no more than 1, 2, or 3 amino acid variations (e.g., substitution, deletion, addition, etc.); and the amino acid sequence of a CDR-L3 comprises the amino acid sequence CDR-L3 of Ab-21 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VH of Ab-21; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of the VL of Ab-21. In some embodiments, the amino acid sequence of the VH comprises an amino acid sequence at least 90%, 91%, 92%, 93%, 94%, 95%, 96%, 97%, 98%, 99%, or 100% identical to the amino acid sequence of the VH of Ab-21; and the amino acid sequence of the VL comprises an amino acid sequence at least 90%, 91%, 92%, 93%, 94%, 95%, 96%, 97%, 98%, 99%, or 100% identical to the amino acid sequence of the VL of Ab-21. In some embodiments, the amino acid sequence of the VH comprises an amino acid sequence at least 95%, 96%, 97%, 98%, 99%, or 100% identical to the amino acid sequence of the VH of Ab-21; and the amino acid sequence of the VL comprises an amino acid sequence at least 95%, 96%, 97%, 98%, 99%, or 100% identical to the amino acid sequence of the VL of Ab-21. In some embodiments, the amino acid sequence of the VH comprises the amino acid sequence of the VH of Ab-21; and the amino acid sequence of the VL comprises the amino acid sequence of the VL of Ab-21.

In some embodiments, the amino acid sequence of CDR-H1 comprises the amino acid sequence set forth in SEQ ID NO: 3, or the amino acid sequence set forth in SEQ ID NO: 3 comprising 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising 1, 2, or 3 amino acid variations (e.g., substitution, deletion, addition, etc.); the amino acid sequence of CDR-H3 comprises the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of CDR-L1 comprises the amino acid sequence set forth in SEQ ID NO: 6, or the amino acid sequence set forth in SEQ ID NO: 6 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of CDR-H1 comprises the amino acid sequence set forth in SEQ ID NO: 3, or the amino acid sequence set forth in SEQ ID NO: 3 comprising no more than 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of CDR-L1 comprises the amino acid sequence set forth in SEQ ID NO: 6, or the amino acid sequence set forth in SEQ ID NO: 6 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 3, or the amino acid sequence set forth in SEQ ID NO: 3 comprising no more than 1, 2, or 3 amino acid substitutions; the amino acid sequence of CDR-H2 comprises the amino acid sequence set forth in SEQ ID NO: 4, or the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 5, or the amino acid sequence set forth in SEQ ID NO: 5 comprising no more than 1, 2, or 3 amino acid substitutions; the amino acid sequence of CDR-L1 comprises the amino acid sequence set forth in SEQ ID NO: 6, or the amino acid sequence set forth in SEQ ID NO: 6 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 8, or the amino acid sequence set forth in SEQ ID NO: 8 comprising no more than 1, 2, or 3 amino acid substitutions.

In some embodiments, the amino acid sequence of CDR-H1 comprises the amino acid sequence set forth in SEQ ID NO: 3; the amino acid sequence of CDR-H2 comprises the amino acid sequence set forth in SEQ ID NO: 4; the amino acid sequence of CDR-H3 comprises the amino acid sequence set forth in SEQ ID NO: 5; the amino acid sequence of CDR-L1 comprises the amino acid sequence set forth in SEQ ID NO: 6; the amino acid sequence of CDR-L2 comprises the amino acid sequence set forth in SEQ ID NO: 7; and the amino acid sequence of CDR-L3 comprises the amino acid sequence set forth in SEQ ID NO: 8.

In some embodiments, the amino acid sequence of CDR-H1 comprises the amino acid sequence set forth in SEQ ID NO: 3 comprising no more than 1, 2, or 3 amino acid variations (e.g., substitution, deletion, addition, etc.); the amino acid sequence of CDR-H2 comprises the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 5 comprising no more than 1, 2, or 3 amino acid variations (e.g., substitution, deletion, addition, etc.); the amino acid sequence of CDR-L1 comprises the amino acid sequence set forth in SEQ ID NO: 6 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 8 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 3 comprising no more than 1, 2, or 3 amino acid substitutions; the amino acid sequence of CDR-H2 comprises the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 5 comprising no more than 1, 2, or 3 amino acid substitutions; the amino acid sequence of CDR-L1 comprises the amino acid sequence set forth in SEQ ID NO: 6 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 8 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 9; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH comprises an amino acid sequence at least 95%, 96%, 97%, 98%, 99%, or 100% identical to the amino acid sequence set forth in SEQ ID NO: 9; and the amino acid sequence of the VL comprises an amino acid sequence at least 95%, 96%, 97%, 98%, 99%, or 100% identical to the amino acid sequence set forth in SEQ ID NO: 10. In some embodiments, the amino acid sequence of the VH comprises the amino acid sequence set forth in SEQ ID NO: 9; and the amino acid sequence of the VL comprises the amino acid sequence set forth in SEQ ID NO: 10.

In some embodiments, the amino acid sequence of CDR-H1 comprises the amino acid sequence set forth in SEQ ID NO: 3, or the amino acid sequence set forth in SEQ ID NO: 3 comprising 1, 2, or 3 amino acid variations (e.g., substitution, deletion, addition, etc.); the amino acid sequence of CDR-H2 comprises the amino acid sequence set forth in SEQ ID NO: 11, or the amino acid sequence set forth in SEQ ID NO: 11 comprising 1, 2, or 3 amino acid variations (e.g., substitution, deletion, addition, etc.); the amino acid sequence of CDR-H3 comprises the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 13, or the amino acid sequence set forth in SEQ ID NO: 13 comprising 1, 2, or 3 amino acid variations (e.g., substitution, deletion, addition, etc.); and the amino acid sequence of CDR-L3 comprises the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of CDR-H1 comprises the amino acid sequence set forth in SEQ ID NO: 3, or the amino acid sequence set forth in SEQ ID NO: 3 comprising no more than 1, 2, or 3 amino acid variations (e.g., substitution, deletion, addition, etc.); the amino acid sequence of CDR-H2 comprises the amino acid sequence set forth in SEQ ID NO: 11, or the amino acid sequence set forth in SEQ ID NO: 11 comprising no more than 1, 2, or 3 amino acid variations (e.g., substitution, deletion, addition, etc.); the amino acid sequence of CDR-H3 comprises the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 13, or the amino acid sequence set forth in SEQ ID NO: 13 comprising no more than 1, 2, or 3 amino acid variations (e.g., substitution, deletion, addition, etc.); and the amino acid sequence of CDR-L3 comprises the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 3, or the amino acid sequence set forth in SEQ ID NO: 3 comprising no more than 1, 2, or 3 amino acid substitutions; the amino acid sequence of CDR-H2 comprises the amino acid sequence set forth in SEQ ID NO: 11, or the amino acid sequence set forth in SEQ ID NO: 11 comprising no more than 1, 2, or 3 amino acid substitutions; the amino acid sequence of CDR-H3 comprises the amino acid sequence set forth in SEQ ID NO: 5, or the amino acid sequence set forth in SEQ ID NO: 5 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 13, or the amino acid sequence set forth in SEQ ID NO: 13 comprising no more than 1, 2, or 3 amino acid substitutions; and the amino acid sequence of CDR-L3 comprises the amino acid sequence set forth in SEQ ID NO: 8, or the amino acid sequence set forth in SEQ ID NO: 8 comprising no more than 1, 2, or 3 amino acid substitutions.

In some embodiments, the amino acid sequence of CDR-H1 comprises the amino acid sequence set forth in SEQ ID NO: 3; the amino acid sequence of CDR-H2 comprises the amino acid sequence set forth in SEQ ID NO: 11; the amino acid sequence of CDR-H3 comprises the amino acid sequence set forth in SEQ ID NO: 5; the amino acid sequence of CDR-L1 comprises the amino acid sequence set forth in SEQ ID NO: 12; the amino acid sequence of CDR-L2 comprises the amino acid sequence set forth in SEQ ID NO: 13; and the amino acid sequence of CDR-L3 comprises the amino acid sequence set forth in SEQ ID NO: 8.

In some embodiments, the amino acid sequence of CDR-H1 comprises the amino acid sequence set forth in SEQ ID NO: 3 comprising no more than 1, 2, or 3 amino acid variations (e.g., substitution, deletion, addition, etc.); the amino acid sequence of CDR-H2 comprises the amino acid sequence set forth in SEQ ID NO: 11 comprising no more than 1, 2, or 3 amino acid variations (e.g., substitution, deletion, addition, etc.); the amino acid sequence of CDR-H3 comprises the amino acid sequence set forth in SEQ ID NO: 5 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 13 comprising no more than 1, 2, or 3 amino acid variations (e.g., substitution, deletion, addition, etc.); and the amino acid sequence of CDR-L3 comprises the amino acid sequence set forth in SEQ ID NO: 8 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 3 comprising no more than 1, 2, or 3 amino acid substitutions; the amino acid sequence of CDR-H2 comprises the amino acid sequence set forth in SEQ ID NO: 11 comprising no more than 1, 2, or 3 amino acid substitutions; the amino acid sequence of CDR-H3 comprises the amino acid sequence set forth in SEQ ID NO: 5 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 13 comprising no more than 1, 2, or 3 amino acid substitutions; and the amino acid sequence of CDR-L3 comprises the amino acid sequence set forth in SEQ ID NO: 8 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 14; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH comprises an amino acid sequence at least 95%, 96%, 97%, 98%, 99%, or 100% identical to the amino acid sequence set forth in SEQ ID NO: 14; and the amino acid sequence of the VL comprises an amino acid sequence at least 95%, 96%, 97%, 98%, 99%, or 100% identical to the amino acid sequence set forth in SEQ ID NO: 15. In some embodiments, the amino acid sequence of the VH comprises the amino acid sequence set forth in SEQ ID NO: 14; and the amino acid sequence of the VL comprises the amino acid sequence set forth in SEQ ID NO: 15.

In some embodiments, the amino acid sequence of CDR-H1 comprises the amino acid sequence set forth in SEQ ID NO: 16, or the amino acid sequence set forth in SEQ ID NO: 16 comprising 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising 1, 2, or 3 amino acid variations (e.g., substitution, deletion, addition, etc.); the amino acid sequence of CDR-L2 comprises the amino acid sequence set forth in SEQ ID NO: 19, or the amino acid sequence set forth in SEQ ID NO: 19 comprising 1, 2, or 3 amino acid variations (e.g., substitution, deletion, addition, etc.); and the amino acid sequence of CDR-L3 comprises the amino acid sequence set forth in SEQ ID NO: 20, or the amino acid sequence set forth in SEQ ID NO: 20 comprising 1, 2, or 3 amino acid variations (e.g., substitution, deletion, addition, etc.).

In some embodiments, the amino acid sequence of CDR-H1 comprises the amino acid sequence set forth in SEQ ID NO: 16, or the amino acid sequence set forth in SEQ ID NO: 16 comprising no more than 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 19, or the amino acid sequence set forth in SEQ ID NO: 19 comprising no more than 1, 2, or 3 amino acid variations (e.g., substitution, deletion, addition, etc.); and the amino acid sequence of CDR-L3 comprises the amino acid sequence set forth in SEQ ID NO: 20, or the amino acid sequence set forth in SEQ ID NO: 20 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 16, or the amino acid sequence set forth in SEQ ID NO: 16 comprising no more than 1, 2, or 3 amino acid substitutions; the amino acid sequence of CDR-H2 comprises the amino acid sequence set forth in SEQ ID NO: 4, or the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17, or the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 18, or the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 19, or the amino acid sequence set forth in SEQ ID NO: 19 comprising no more than 1, 2, or 3 amino acid substitutions; and the amino acid sequence of CDR-L3 comprises the amino acid sequence set forth in SEQ ID NO: 20, or the amino acid sequence set forth in SEQ ID NO: 20 comprising no more than 1, 2, or 3 amino acid substitutions.

In some embodiments, the amino acid sequence of CDR-H1 comprises the amino acid sequence set forth in SEQ ID NO: 16; the amino acid sequence of CDR-H2 comprises the amino acid sequence set forth in SEQ ID NO: 4; the amino acid sequence of CDR-H3 comprises the amino acid sequence set forth in SEQ ID NO: 17; the amino acid sequence of CDR-L1 comprises the amino acid sequence set forth in SEQ ID NO: 18; the amino acid sequence of CDR-L2 comprises the amino acid sequence set forth in SEQ ID NO: 19; and the amino acid sequence of CDR-L3 comprises the amino acid sequence set forth in SEQ ID NO: 20.

In some embodiments, the amino acid sequence of CDR-H1 comprises the amino acid sequence set forth in SEQ ID NO: 16 comprising no more than 1, 2, or 3 amino acid variations (e.g., substitution, deletion, addition, etc.); the amino acid sequence of CDR-H2 comprises the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 19 comprising no more than 1, 2, or 3 amino acid variations (e.g., substitution, deletion, addition, etc.); and the amino acid sequence of CDR-L3 comprises the amino acid sequence set forth in SEQ ID NO: 20 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 16 comprising no more than 1, 2, or 3 amino acid substitutions; the amino acid sequence of CDR-H2 comprises the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 19 comprising no more than 1, 2, or 3 amino acid substitutions; and the amino acid sequence of CDR-L3 comprises the amino acid sequence set forth in SEQ ID NO: 20 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 21; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 22. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 21; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 22. In some embodiments, the amino acid sequence of the VH comprises an amino acid sequence at least 95%, 96%, 97%, 98%, 99%, or 100% identical to the amino acid sequence set forth in SEQ ID NO: 21; and the amino acid sequence of the VL comprises an amino acid sequence at least 95%, 96%, 97%, 98%, 99%, or 100% identical to the amino acid sequence set forth in SEQ ID NO: 22. In some embodiments, the amino acid sequence of the VH comprises the amino acid sequence set forth in SEQ ID NO: 21; and the amino acid sequence of the VL comprises the amino acid sequence set forth in SEQ ID NO: 22.

In some embodiments, the amino acid sequence of CDR-H1 comprises the amino acid sequence set forth in SEQ ID NO: 16, or the amino acid sequence set forth in SEQ ID NO: 16 comprising 1, 2, or 3 amino acid variations (e.g., substitution, deletion, addition, etc.); the amino acid sequence of CDR-H2 comprises the amino acid sequence set forth in SEQ ID NO: 23, or the amino acid sequence set forth in SEQ ID NO: 23 comprising 1, 2, or 3 amino acid variations (e.g., substitution, deletion, addition, etc.); the amino acid sequence of CDR-H3 comprises the amino acid sequence set forth in SEQ ID NO: 24, or the amino acid sequence set forth in SEQ ID NO: 24 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 25, or the amino acid sequence set forth in SEQ ID NO: 25 comprising 1, 2, or 3 amino acid variations (e.g., substitution, deletion, addition, etc.); and the amino acid sequence of CDR-L3 comprises the amino acid sequence set forth in SEQ ID NO: 26, or the amino acid sequence set forth in SEQ ID NO: 26 comprising 1, 2, or 3 amino acid variations (e.g., substitution, deletion, addition, etc.).

In some embodiments, the amino acid sequence of CDR-H1 comprises the amino acid sequence set forth in SEQ ID NO: 16, or the amino acid sequence set forth in SEQ ID NO: 16 comprising no more than 1, 2, or 3 amino acid variations (e.g., substitution, deletion, addition, etc.); the amino acid sequence of CDR-H2 comprises the amino acid sequence set forth in SEQ ID NO: 23, or the amino acid sequence set forth in SEQ ID NO: 23 comprising no more than 1, 2, or 3 amino acid variations (e.g., substitution, deletion, addition, etc.); the amino acid sequence of CDR-H3 comprises the amino acid sequence set forth in SEQ ID NO: 24, or the amino acid sequence set forth in SEQ ID NO: 24 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 25, or the amino acid sequence set forth in SEQ ID NO: 25 comprising no more than 1, 2, or 3 amino acid variations (e.g., substitution, deletion, addition, etc.); and the amino acid sequence of CDR-L3 comprises the amino acid sequence set forth in SEQ ID NO: 26, or the amino acid sequence set forth in SEQ ID NO: 26 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 16, or the amino acid sequence set forth in SEQ ID NO: 16 comprising no more than 1, 2, or 3 amino acid substitutions; the amino acid sequence of CDR-H2 comprises the amino acid sequence set forth in SEQ ID NO: 23, or the amino acid sequence set forth in SEQ ID NO: 23 comprising no more than 1, 2, or 3 amino acid substitutions; the amino acid sequence of CDR-H3 comprises the amino acid sequence set forth in SEQ ID NO: 24, or the amino acid sequence set forth in SEQ ID NO: 24 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 25, or the amino acid sequence set forth in SEQ ID NO: 25 comprising no more than 1, 2, or 3 amino acid substitutions; and the amino acid sequence of CDR-L3 comprises the amino acid sequence set forth in SEQ ID NO: 26, or the amino acid sequence set forth in SEQ ID NO: 26 comprising no more than 1, 2, or 3 amino acid substitutions.

In some embodiments, the amino acid sequence of CDR-H1 comprises the amino acid sequence set forth in SEQ ID NO: 16; the amino acid sequence of CDR-H2 comprises the amino acid sequence set forth in SEQ ID NO: 23; the amino acid sequence of CDR-H3 comprises the amino acid sequence set forth in SEQ ID NO: 24; the amino acid sequence of CDR-L1 comprises the amino acid sequence set forth in SEQ ID NO: 12; the amino acid sequence of CDR-L2 comprises the amino acid sequence set forth in SEQ ID NO: 25; and the amino acid sequence of CDR-L3 comprises the amino acid sequence set forth in SEQ ID NO: 26.

In some embodiments, the amino acid sequence of CDR-H1 comprises the amino acid sequence set forth in SEQ ID NO: 16 comprising no more than 1, 2, or 3 amino acid variations (e.g., substitution, deletion, addition, etc.); the amino acid sequence of CDR-H2 comprises the amino acid sequence set forth in SEQ ID NO: 23 comprising no more than 1, 2, or 3 amino acid variations (e.g., substitution, deletion, addition, etc.); the amino acid sequence of CDR-H3 comprises the amino acid sequence set forth in SEQ ID NO: 24 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 25 comprising no more than 1, 2, or 3 amino acid variations (e.g., substitution, deletion, addition, etc.); and the amino acid sequence of CDR-L3 comprises the amino acid sequence set forth in SEQ ID NO: 26 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 16 comprising no more than 1, 2, or 3 amino acid substitutions; the amino acid sequence of CDR-H2 comprises the amino acid sequence set forth in SEQ ID NO: 23 comprising no more than 1, 2, or 3 amino acid substitutions; the amino acid sequence of CDR-H3 comprises the amino acid sequence set forth in SEQ ID NO: 24 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 25 comprising no more than 1, 2, or 3 amino acid substitutions; and the amino acid sequence of CDR-L3 comprises the amino acid sequence set forth in SEQ ID NO: 26 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 27; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 28. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 27; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 28. In some embodiments, the amino acid sequence of the VH comprises an amino acid sequence at least 95%, 96%, 97%, 98%, 99%, or 100% identical to the amino acid sequence set forth in SEQ ID NO: 27; and the amino acid sequence of the VL comprises an amino acid sequence at least 95%, 96%, 97%, 98%, 99%, or 100% identical to the amino acid sequence set forth in SEQ ID NO: 28. In some embodiments, the amino acid sequence of the VH comprises the amino acid sequence set forth in SEQ ID NO: 27; and the amino acid sequence of the VL comprises the amino acid sequence set forth in SEQ ID NO: 28.

In some embodiments, the amino acid sequence of CDR-H1 comprises the amino acid sequence set forth in SEQ ID NO: 29, or the amino acid sequence set forth in SEQ ID NO: 29 comprising 1, 2, or 3 amino acid variations (e.g., substitution, deletion, addition, etc.); the amino acid sequence of CDR-H2 comprises the amino acid sequence set forth in SEQ ID NO: 30, or the amino acid sequence set forth in SEQ ID NO: 30 comprising 1, 2, or 3 amino acid variations (e.g., substitution, deletion, addition, etc.); the amino acid sequence of CDR-H3 comprises the amino acid sequence set forth in SEQ ID NO: 31, or the amino acid sequence set forth in SEQ ID NO: 31 comprising 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising 1, 2, or 3 amino acid variations (e.g., substitution, deletion, addition, etc.); the amino acid sequence of CDR-L2 comprises the amino acid sequence set forth in SEQ ID NO: 32, or the amino acid sequence set forth in SEQ ID NO: 32 comprising 1, 2, or 3 amino acid variations (e.g., substitution, deletion, addition, etc.); and the amino acid sequence of CDR-L3 comprises the amino acid sequence set forth in SEQ ID NO: 33, or the amino acid sequence set forth in SEQ ID NO: 33 comprising 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 30, or the amino acid sequence set forth in SEQ ID NO: 30 comprising no more than 1, 2, or 3 amino acid variations (e.g., substitution, deletion, addition, etc.); the amino acid sequence of CDR-H3 comprises the amino acid sequence set forth in SEQ ID NO: 31, or the amino acid sequence set forth in SEQ ID NO: 31 comprising no more than 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 32, or the amino acid sequence set forth in SEQ ID NO: 32 comprising no more than 1, 2, or 3 amino acid variations (e.g., substitution, deletion, addition, etc.); and the amino acid sequence of CDR-L3 comprises the amino acid sequence set forth in SEQ ID NO: 33, or the amino acid sequence set forth in SEQ ID NO: 33 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 30, or the amino acid sequence set forth in SEQ ID NO: 30 comprising no more than 1, 2, or 3 amino acid substitutions; the amino acid sequence of CDR-H3 comprises the amino acid sequence set forth in SEQ ID NO: 31, or the amino acid sequence set forth in SEQ ID NO: 31 comprising no more than 1, 2, or 3 amino acid substitutions; the amino acid sequence of CDR-L1 comprises the amino acid sequence set forth in SEQ ID NO: 18, or the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 32, or the amino acid sequence set forth in SEQ ID NO: 32 comprising no more than 1, 2, or 3 amino acid substitutions; and the amino acid sequence of CDR-L3 comprises the amino acid sequence set forth in SEQ ID NO: 33, or the amino acid sequence set forth in SEQ ID NO: 33 comprising no more than 1, 2, or 3 amino acid substitutions.

In some embodiments, the amino acid sequence of CDR-H1 comprises the amino acid sequence set forth in SEQ ID NO: 29; the amino acid sequence of CDR-H2 comprises the amino acid sequence set forth in SEQ ID NO: 30; the amino acid sequence of CDR-H3 comprises the amino acid sequence set forth in SEQ ID NO: 31; the amino acid sequence of CDR-L1 comprises the amino acid sequence set forth in SEQ ID NO: 18; the amino acid sequence of CDR-L2 comprises the amino acid sequence set forth in SEQ ID NO: 32; and the amino acid sequence of CDR-L3 comprises the amino acid sequence set forth in SEQ ID NO: 33.

In some embodiments, the amino acid sequence of CDR-H1 comprises the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 30 comprising no more than 1, 2, or 3 amino acid variations (e.g., substitution, deletion, addition, etc.); the amino acid sequence of CDR-H3 comprises the amino acid sequence set forth in SEQ ID NO: 31 comprising no more than 1, 2, or 3 amino acid variations (e.g., substitution, deletion, addition, etc.); the amino acid sequence of CDR-L1 comprises the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 32 comprising no more than 1, 2, or 3 amino acid variations (e.g., substitution, deletion, addition, etc.); and the amino acid sequence of CDR-L3 comprises the amino acid sequence set forth in SEQ ID NO: 33 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 30 comprising no more than 1, 2, or 3 amino acid substitutions; the amino acid sequence of CDR-H3 comprises the amino acid sequence set forth in SEQ ID NO: 31 comprising no more than 1, 2, or 3 amino acid substitutions; the amino acid sequence of CDR-L1 comprises the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 32 comprising no more than 1, 2, or 3 amino acid substitutions; and the amino acid sequence of CDR-L3 comprises the amino acid sequence set forth in SEQ ID NO: 33 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 34; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 35. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 34; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 35. In some embodiments, the amino acid sequence of the VH comprises an amino acid sequence at least 95%, 96%, 97%, 98%, 99%, or 100% identical to the amino acid sequence set forth in SEQ ID NO: 34; and the amino acid sequence of the VL comprises an amino acid sequence at least 95%, 96%, 97%, 98%, 99%, or 100% identical to the amino acid sequence set forth in SEQ ID NO: 35. In some embodiments, the amino acid sequence of the VH comprises the amino acid sequence set forth in SEQ ID NO: 34; and the amino acid sequence of the VL comprises the amino acid sequence set forth in SEQ ID NO: 35.

In some embodiments, the amino acid sequence of CDR-H1 comprises the amino acid sequence set forth in SEQ ID NO: 29, or the amino acid sequence set forth in SEQ ID NO: 29 comprising 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 36, or the amino acid sequence set forth in SEQ ID NO: 36 comprising 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 36, or the amino acid sequence set forth in SEQ ID NO: 36 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 4, or the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17, or the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 36, or the amino acid sequence set forth in SEQ ID NO: 36 comprising no more than 1, 2, or 3 amino acid substitutions.

In some embodiments, the amino acid sequence of CDR-H1 comprises the amino acid sequence set forth in SEQ ID NO: 29; the amino acid sequence of CDR-H2 comprises the amino acid sequence set forth in SEQ ID NO: 4; the amino acid sequence of CDR-H3 comprises the amino acid sequence set forth in SEQ ID NO: 17; the amino acid sequence of CDR-L1 comprises the amino acid sequence set forth in SEQ ID NO: 12; the amino acid sequence of CDR-L2 comprises the amino acid sequence set forth in SEQ ID NO: 7; and the amino acid sequence of CDR-L3 comprises the amino acid sequence set forth in SEQ ID NO: 36.

In some embodiments, the amino acid sequence of CDR-H1 comprises the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 36 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 36 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 37; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 38. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 37; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 38. In some embodiments, the amino acid sequence of the VH comprises an amino acid sequence at least 95%, 96%, 97%, 98%, 99%, or 100% identical to the amino acid sequence set forth in SEQ ID NO: 37; and the amino acid sequence of the VL comprises an amino acid sequence at least 95%, 96%, 97%, 98%, 99%, or 100% identical to the amino acid sequence set forth in SEQ ID NO: 38. In some embodiments, the amino acid sequence of the VH comprises the amino acid sequence set forth in SEQ ID NO: 37; and the amino acid sequence of the VL comprises the amino acid sequence set forth in SEQ ID NO: 38.

In some embodiments, the amino acid sequence of CDR-H1 comprises the amino acid sequence set forth in SEQ ID NO: 29, or the amino acid sequence set forth in SEQ ID NO: 29 comprising 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 39, or the amino acid sequence set forth in SEQ ID NO: 39 comprising 1, 2, or 3 amino acid variations (e.g., substitution, deletion, addition, etc.); and the amino acid sequence of CDR-L3 comprises the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 39, or the amino acid sequence set forth in SEQ ID NO: 39 comprising no more than 1, 2, or 3 amino acid variations (e.g., substitution, deletion, addition, etc.); and the amino acid sequence of CDR-L3 comprises the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 4, or the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17, or the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 39, or the amino acid sequence set forth in SEQ ID NO: 39 comprising no more than 1, 2, or 3 amino acid substitutions; and the amino acid sequence of CDR-L3 comprises the amino acid sequence set forth in SEQ ID NO: 8, or the amino acid sequence set forth in SEQ ID NO: 8 comprising no more than 1, 2, or 3 amino acid substitutions.

In some embodiments, the amino acid sequence of CDR-H1 comprises the amino acid sequence set forth in SEQ ID NO: 29; the amino acid sequence of CDR-H2 comprises the amino acid sequence set forth in SEQ ID NO: 4; the amino acid sequence of CDR-H3 comprises the amino acid sequence set forth in SEQ ID NO: 17; the amino acid sequence of CDR-L1 comprises the amino acid sequence set forth in SEQ ID NO: 12; the amino acid sequence of CDR-L2 comprises the amino acid sequence set forth in SEQ ID NO: 39; and the amino acid sequence of CDR-L3 comprises the amino acid sequence set forth in SEQ ID NO: 8.

In some embodiments, the amino acid sequence of CDR-H1 comprises the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 39 comprising no more than 1, 2, or 3 amino acid variations (e.g., substitution, deletion, addition, etc.); and the amino acid sequence of CDR-L3 comprises the amino acid sequence set forth in SEQ ID NO: 8 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 39 comprising no more than 1, 2, or 3 amino acid substitutions; and the amino acid sequence of CDR-L3 comprises the amino acid sequence set forth in SEQ ID NO: 8 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 37; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 40. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 37; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 40. In some embodiments, the amino acid sequence of the VH comprises an amino acid sequence at least 95%, 96%, 97%, 98%, 99%, or 100% identical to the amino acid sequence set forth in SEQ ID NO: 37; and the amino acid sequence of the VL comprises an amino acid sequence at least 95%, 96%, 97%, 98%, 99%, or 100% identical to the amino acid sequence set forth in SEQ ID NO: 40. In some embodiments, the amino acid sequence of the VH comprises the amino acid sequence set forth in SEQ ID NO: 37; and the amino acid sequence of the VL comprises the amino acid sequence set forth in SEQ ID NO: 40.

In some embodiments, the amino acid sequence of CDR-H1 comprises the amino acid sequence set forth in SEQ ID NO: 41, or the amino acid sequence set forth in SEQ ID NO: 41 comprising 1, 2, or 3 amino acid variations (e.g., substitution, deletion, addition, etc.); the amino acid sequence of CDR-H2 comprises the amino acid sequence set forth in SEQ ID NO: 11, or the amino acid sequence set forth in SEQ ID NO: 11 comprising 1, 2, or 3 amino acid variations (e.g., substitution, deletion, addition, etc.); the amino acid sequence of CDR-H3 comprises the amino acid sequence set forth in SEQ ID NO: 42, or the amino acid sequence set forth in SEQ ID NO: 42 comprising 1, 2, or 3 amino acid variations (e.g., substitution, deletion, addition, etc.); the amino acid sequence of CDR-L1 comprises the amino acid sequence set forth in SEQ ID NO: 43, or the amino acid sequence set forth in SEQ ID NO: 43 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 44, or the amino acid sequence set forth in SEQ ID NO: 44 comprising 1, 2, or 3 amino acid variations (e.g., substitution, deletion, addition, etc.).

In some embodiments, the amino acid sequence of CDR-H1 comprises the amino acid sequence set forth in SEQ ID NO: 41, or the amino acid sequence set forth in SEQ ID NO: 41 comprising no more than 1, 2, or 3 amino acid variations (e.g., substitution, deletion, addition, etc.); the amino acid sequence of CDR-H2 comprises the amino acid sequence set forth in SEQ ID NO: 11, or the amino acid sequence set forth in SEQ ID NO: 11 comprising no more than 1, 2, or 3 amino acid variations (e.g., substitution, deletion, addition, etc.); the amino acid sequence of CDR-H3 comprises the amino acid sequence set forth in SEQ ID NO: 42, or the amino acid sequence set forth in SEQ ID NO: 42 comprising no more than 1, 2, or 3 amino acid variations (e.g., substitution, deletion, addition, etc.); the amino acid sequence of CDR-L1 comprises the amino acid sequence set forth in SEQ ID NO: 43, or the amino acid sequence set forth in SEQ ID NO: 43 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 44, or the amino acid sequence set forth in SEQ ID NO: 44 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 41, or the amino acid sequence set forth in SEQ ID NO: 41 comprising no more than 1, 2, or 3 amino acid substitutions; the amino acid sequence of CDR-H2 comprises the amino acid sequence set forth in SEQ ID NO: 11, or the amino acid sequence set forth in SEQ ID NO: 11 comprising no more than 1, 2, or 3 amino acid substitutions; the amino acid sequence of CDR-H3 comprises the amino acid sequence set forth in SEQ ID NO: 42, or the amino acid sequence set forth in SEQ ID NO: 42 comprising no more than 1, 2, or 3 amino acid substitutions; the amino acid sequence of CDR-L1 comprises the amino acid sequence set forth in SEQ ID NO: 43, or the amino acid sequence set forth in SEQ ID NO: 43 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 44, or the amino acid sequence set forth in SEQ ID NO: 44 comprising no more than 1, 2, or 3 amino acid substitutions.

In some embodiments, the amino acid sequence of CDR-H1 comprises the amino acid sequence set forth in SEQ ID NO: 41; the amino acid sequence of CDR-H2 comprises the amino acid sequence set forth in SEQ ID NO: 11; the amino acid sequence of CDR-H3 comprises the amino acid sequence set forth in SEQ ID NO: 42; the amino acid sequence of CDR-L1 comprises the amino acid sequence set forth in SEQ ID NO: 43; the amino acid sequence of CDR-L2 comprises the amino acid sequence set forth in SEQ ID NO: 7; and the amino acid sequence of CDR-L3 comprises the amino acid sequence set forth in SEQ ID NO: 44.

In some embodiments, the amino acid sequence of CDR-H1 comprises the amino acid sequence set forth in SEQ ID NO: 41 comprising no more than 1, 2, or 3 amino acid variations (e.g., substitution, deletion, addition, etc.); the amino acid sequence of CDR-H2 comprises the amino acid sequence set forth in SEQ ID NO: 11 comprising no more than 1, 2, or 3 amino acid variations (e.g., substitution, deletion, addition, etc.); the amino acid sequence of CDR-H3 comprises the amino acid sequence set forth in SEQ ID NO: 42 comprising no more than 1, 2, or 3 amino acid variations (e.g., substitution, deletion, addition, etc.); the amino acid sequence of CDR-L1 comprises the amino acid sequence set forth in SEQ ID NO: 43 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 44 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 41 comprising no more than 1, 2, or 3 amino acid substitutions; the amino acid sequence of CDR-H2 comprises the amino acid sequence set forth in SEQ ID NO: 11 comprising no more than 1, 2, or 3 amino acid substitutions; the amino acid sequence of CDR-H3 comprises the amino acid sequence set forth in SEQ ID NO: 42 comprising no more than 1, 2, or 3 amino acid substitutions; the amino acid sequence of CDR-L1 comprises the amino acid sequence set forth in SEQ ID NO: 43 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 44 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 45; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 46. In some embodiments, the amino acid sequence of the VH comprises an amino acid sequence at least 95%, 96%, 97%, 98%, 99%, or 100% identical to the amino acid sequence set forth in SEQ ID NO: 45; and the amino acid sequence of the VL comprises an amino acid sequence at least 95%, 96%, 97%, 98%, 99%, or 100% identical to the amino acid sequence set forth in SEQ ID NO: 46. In some embodiments, the amino acid sequence of the VH comprises the amino acid sequence set forth in SEQ ID NO: 45; and the amino acid sequence of the VL comprises the amino acid sequence set forth in SEQ ID NO: 46.

In some embodiments, the amino acid sequence of CDR-H1 comprises the amino acid sequence set forth in SEQ ID NO: 47, or the amino acid sequence set forth in SEQ ID NO: 47 comprising 1, 2, or 3 amino acid variations (e.g., substitution, deletion, addition, etc.); the amino acid sequence of CDR-H2 comprises the amino acid sequence set forth in SEQ ID NO: 48, or the amino acid sequence set forth in SEQ ID NO: 48 comprising 1, 2, or 3 amino acid variations (e.g., substitution, deletion, addition, etc.); the amino acid sequence of CDR-H3 comprises the amino acid sequence set forth in SEQ ID NO: 42, or the amino acid sequence set forth in SEQ ID NO: 42 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 49, or the amino acid sequence set forth in SEQ ID NO: 49 comprising 1, 2, or 3 amino acid variations (e.g., substitution, deletion, addition, etc.).

In some embodiments, the amino acid sequence of CDR-H1 comprises the amino acid sequence set forth in SEQ ID NO: 47, or the amino acid sequence set forth in SEQ ID NO: 47 comprising no more than 1, 2, or 3 amino acid variations (e.g., substitution, deletion, addition, etc.); the amino acid sequence of CDR-H2 comprises the amino acid sequence set forth in SEQ ID NO: 48, or the amino acid sequence set forth in SEQ ID NO: 48 comprising no more than 1, 2, or 3 amino acid variations (e.g., substitution, deletion, addition, etc.); the amino acid sequence of CDR-H3 comprises the amino acid sequence set forth in SEQ ID NO: 42, or the amino acid sequence set forth in SEQ ID NO: 42 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 49, or the amino acid sequence set forth in SEQ ID NO: 49 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 47, or the amino acid sequence set forth in SEQ ID NO: 47 comprising no more than 1, 2, or 3 amino acid substitutions; the amino acid sequence of CDR-H2 comprises the amino acid sequence set forth in SEQ ID NO: 48, or the amino acid sequence set forth in SEQ ID NO: 48 comprising no more than 1, 2, or 3 amino acid substitutions; the amino acid sequence of CDR-H3 comprises the amino acid sequence set forth in SEQ ID NO: 42, or the amino acid sequence set forth in SEQ ID NO: 42 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 49, or the amino acid sequence set forth in SEQ ID NO: 49 comprising no more than 1, 2, or 3 amino acid substitutions.

In some embodiments, the amino acid sequence of CDR-H1 comprises the amino acid sequence set forth in SEQ ID NO: 47; the amino acid sequence of CDR-H2 comprises the amino acid sequence set forth in SEQ ID NO: 48; the amino acid sequence of CDR-H3 comprises the amino acid sequence set forth in SEQ ID NO: 42; the amino acid sequence of CDR-L1 comprises the amino acid sequence set forth in SEQ ID NO: 12; the amino acid sequence of CDR-L2 comprises the amino acid sequence set forth in SEQ ID NO: 7; and the amino acid sequence of CDR-L3 comprises the amino acid sequence set forth in SEQ ID NO: 49.

In some embodiments, the amino acid sequence of CDR-H1 comprises the amino acid sequence set forth in SEQ ID NO: 47 comprising no more than 1, 2, or 3 amino acid variations (e.g., substitution, deletion, addition, etc.); the amino acid sequence of CDR-H2 comprises the amino acid sequence set forth in SEQ ID NO: 48 comprising no more than 1, 2, or 3 amino acid variations (e.g., substitution, deletion, addition, etc.); the amino acid sequence of CDR-H3 comprises the amino acid sequence set forth in SEQ ID NO: 42 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 49 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 47 comprising no more than 1, 2, or 3 amino acid substitutions; the amino acid sequence of CDR-H2 comprises the amino acid sequence set forth in SEQ ID NO: 48 comprising no more than 1, 2, or 3 amino acid substitutions; the amino acid sequence of CDR-H3 comprises the amino acid sequence set forth in SEQ ID NO: 42 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 49 comprising no more than 1, 2, or 3 amino acid substitutions.

In some embodiments, the amino acid sequence of CDR-H1 consists of the amino acid sequence set forth in SEQ ID NO: 47, or the amino acid sequence set forth in SEQ ID NO: 47 comprising 1, 2, or 3 amino acid variations (e.g., substitution, deletion, addition, etc.); the amino acid sequence of CDR-H2 consists of the amino acid sequence set forth in SEQ ID NO: 48, or the amino acid sequence set forth in SEQ ID NO: 48 comprising 1, 2, or 3 amino acid variations (e.g., substitution, deletion, addition, etc.); the amino acid sequence of CDR-H3 consists of the amino acid sequence set forth in SEQ ID NO: 42, or the amino acid sequence set forth in SEQ ID NO: 42 comprising 1, 2, or 3 amino acid variations (e.g., substitution, deletion, addition, etc.); the amino acid sequence of CDR-L1 consists of the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 consists of the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 consists of the amino acid sequence set forth in SEQ ID NO: 49, or the amino acid sequence set forth in SEQ ID NO: 49 comprising 1, 2, or 3 amino acid variations (e.g., substitution, deletion, addition, etc.).

In some embodiments, the amino acid sequence of CDR-H1 consists of the amino acid sequence set forth in SEQ ID NO: 47, or the amino acid sequence set forth in SEQ ID NO: 47 comprising no more than 1, 2, or 3 amino acid variations (e.g., substitution, deletion, addition, etc.); the amino acid sequence of CDR-H2 consists of the amino acid sequence set forth in SEQ ID NO: 48, or the amino acid sequence set forth in SEQ ID NO: 48 comprising no more than 1, 2, or 3 amino acid variations (e.g., substitution, deletion, addition, etc.); the amino acid sequence of CDR-H3 consists of the amino acid sequence set forth in SEQ ID NO: 42, or the amino acid sequence set forth in SEQ ID NO: 42 comprising no more than 1, 2, or 3 amino acid variations (e.g., substitution, deletion, addition, etc.); the amino acid sequence of CDR-L1 consists of the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 consists of the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 consists of the amino acid sequence set forth in SEQ ID NO: 49, or the amino acid sequence set forth in SEQ ID NO: 49 comprising no more than 1, 2, or 3 amino acid variations (e.g., substitution, deletion, addition, etc.).

In some embodiments, the amino acid sequence of CDR-H1 consists of the amino acid sequence set forth in SEQ ID NO: 47, or the amino acid sequence set forth in SEQ ID NO: 47 comprising no more than 1, 2, or 3 amino acid substitutions; the amino acid sequence of CDR-H2 consists of the amino acid sequence set forth in SEQ ID NO: 48, or the amino acid sequence set forth in SEQ ID NO: 48 comprising no more than 1, 2, or 3 amino acid substitutions; the amino acid sequence of CDR-H3 consists of the amino acid sequence set forth in SEQ ID NO: 42, or the amino acid sequence set forth in SEQ ID NO: 42 comprising no more than 1, 2, or 3 amino acid substitutions; the amino acid sequence of CDR-L1 consists of the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 consists of the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 consists of the amino acid sequence set forth in SEQ ID NO: 49, or the amino acid sequence set forth in SEQ ID NO: 49 comprising no more than 1, 2, or 3 amino acid substitutions.

In some embodiments, the amino acid sequence of CDR-H1 consists of the amino acid sequence set forth in SEQ ID NO: 47; the amino acid sequence of CDR-H2 consists of the amino acid sequence set forth in SEQ ID NO: 48; the amino acid sequence of CDR-H3 consists of the amino acid sequence set forth in SEQ ID NO: 42; the amino acid sequence of CDR-L1 consists of the amino acid sequence set forth in SEQ ID NO: 12; the amino acid sequence of CDR-L2 consists of the amino acid sequence set forth in SEQ ID NO: 7; and the amino acid sequence of CDR-L3 consists of the amino acid sequence set forth in SEQ ID NO: 49.

In some embodiments, the amino acid sequence of CDR-H1 consists of the amino acid sequence set forth in SEQ ID NO: 47 comprising no more than 1, 2, or 3 amino acid variations (e.g., substitution, deletion, addition, etc.); the amino acid sequence of CDR-H2 consists of the amino acid sequence set forth in SEQ ID NO: 48 comprising no more than 1, 2, or 3 amino acid variations (e.g., substitution, deletion, addition, etc.); the amino acid sequence of CDR-H3 consists of the amino acid sequence set forth in SEQ ID NO: 42 comprising no more than 1, 2, or 3 amino acid variations (e.g., substitution, deletion, addition, etc.); the amino acid sequence of CDR-L1 consists of the amino acid sequence set forth in SEQ ID NO: 12 comprising no more than 1, 2, or 3 amino acid variations (e.g., substitution, deletion, addition, etc.); the amino acid sequence of CDR-L2 consists of the amino acid sequence set forth in SEQ ID NO: 7 comprising no more than 1, 2, or 3 amino acid variations (e.g., substitution, deletion, addition, etc.); and the amino acid sequence of CDR-L3 consists of the amino acid sequence set forth in SEQ ID NO: 49 comprising no more than 1, 2, or 3 amino acid variations (e.g., substitution, deletion, addition, etc.).

In some embodiments, the amino acid sequence of CDR-H1 consists of the amino acid sequence set forth in SEQ ID NO: 47 comprising no more than 1, 2, or 3 amino acid substitutions; the amino acid sequence of CDR-H2 consists of the amino acid sequence set forth in SEQ ID NO: 48 comprising no more than 1, 2, or 3 amino acid substitutions; the amino acid sequence of CDR-H3 consists of the amino acid sequence set forth in SEQ ID NO: 42 comprising no more than 1, 2, or 3 amino acid substitutions; the amino acid sequence of CDR-L1 consists of the amino acid sequence set forth in SEQ ID NO: 12 comprising no more than 1, 2, or 3 amino acid substitutions; the amino acid sequence of CDR-L2 consists of the amino acid sequence set forth in SEQ ID NO: 7 comprising no more than 1, 2, or 3 amino acid substitutions; and the amino acid sequence of CDR-L3 consists of the amino acid sequence set forth in SEQ ID NO: 49 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 50; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 51. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 50; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 51. In some embodiments, the amino acid sequence of the VH comprises an amino acid sequence at least 95%, 96%, 97%, 98%, 99%, or 100% identical to the amino acid sequence set forth in SEQ ID NO: 50; and the amino acid sequence of the VL comprises an amino acid sequence at least 95%, 96%, 97%, 98%, 99%, or 100% identical to the amino acid sequence set forth in SEQ ID NO: 51. In some embodiments, the amino acid sequence of the VH comprises the amino acid sequence set forth in SEQ ID NO: 50; and the amino acid sequence of the VL comprises the amino acid sequence set forth in SEQ ID NO: 51.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 50; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 51. In some embodiments, the amino acid sequence of the VH consists of an amino acid sequence at least 90%, 91%, 92%, 93%, 94%, 95%, 96%, 97%, 98%, 99%, or 100% identical to the amino acid sequence set forth in SEQ ID NO: 50; and the amino acid sequence of the VL consists of an amino acid sequence at least 90%, 91%, 92%, 93%, 94%, 95%, 96%, 97%, 98%, 99%, or 100% identical to the amino acid sequence set forth in SEQ ID NO: 51. In some embodiments, the amino acid sequence of the VH consists of an amino acid sequence at least 95%, 96%, 97%, 98%, 99%, or 100% identical to the amino acid sequence set forth in SEQ ID NO: 50; and the amino acid sequence of the VL consists of an amino acid sequence at least 95%, 96%, 97%, 98%, 99%, or 100% identical to the amino acid sequence set forth in SEQ ID NO: 51. In some embodiments, the amino acid sequence of the VH consists of the amino acid sequence set forth in SEQ ID NO: 50; and the amino acid sequence of the VL consists of the amino acid sequence set forth in SEQ ID NO: 51.

In some embodiments, the amino acid sequence of CDR-H1 comprises the amino acid sequence set forth in SEQ ID NO: 29, or the amino acid sequence set forth in SEQ ID NO: 29 comprising 1, 2, or 3 amino acid variations (e.g., substitution, deletion, addition, etc.); the amino acid sequence of CDR-H2 comprises the amino acid sequence set forth in SEQ ID NO: 52, or the amino acid sequence set forth in SEQ ID NO: 52 comprising 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising 1, 2, or 3 amino acid variations (e.g., substitution, deletion, addition, etc.); the amino acid sequence of CDR-L2 comprises the amino acid sequence set forth in SEQ ID NO: 19, or the amino acid sequence set forth in SEQ ID NO: 19 comprising 1, 2, or 3 amino acid variations (e.g., substitution, deletion, addition, etc.); and the amino acid sequence of CDR-L3 comprises the amino acid sequence set forth in SEQ ID NO: 53, or the amino acid sequence set forth in SEQ ID NO: 53 comprising 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 52, or the amino acid sequence set forth in SEQ ID NO: 52 comprising no more than 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 19, or the amino acid sequence set forth in SEQ ID NO: 19 comprising no more than 1, 2, or 3 amino acid variations (e.g., substitution, deletion, addition, etc.); and the amino acid sequence of CDR-L3 comprises the amino acid sequence set forth in SEQ ID NO: 53, or the amino acid sequence set forth in SEQ ID NO: 53 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 52, or the amino acid sequence set forth in SEQ ID NO: 52 comprising no more than 1, 2, or 3 amino acid substitutions; the amino acid sequence of CDR-H3 comprises the amino acid sequence set forth in SEQ ID NO: 17, or the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 18, or the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 19, or the amino acid sequence set forth in SEQ ID NO: 19 comprising no more than 1, 2, or 3 amino acid substitutions; and the amino acid sequence of CDR-L3 comprises the amino acid sequence set forth in SEQ ID NO: 53, or the amino acid sequence set forth in SEQ ID NO: 53 comprising no more than 1, 2, or 3 amino acid substitutions.

In some embodiments, the amino acid sequence of CDR-H1 comprises the amino acid sequence set forth in SEQ ID NO: 29; the amino acid sequence of CDR-H2 comprises the amino acid sequence set forth in SEQ ID NO: 52; the amino acid sequence of CDR-H3 comprises the amino acid sequence set forth in SEQ ID NO: 17; the amino acid sequence of CDR-L1 comprises the amino acid sequence set forth in SEQ ID NO: 18; the amino acid sequence of CDR-L2 comprises the amino acid sequence set forth in SEQ ID NO: 19; and the amino acid sequence of CDR-L3 comprises the amino acid sequence set forth in SEQ ID NO: 53.

In some embodiments, the amino acid sequence of CDR-H1 comprises the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 52 comprising no more than 1, 2, or 3 amino acid variations (e.g., substitution, deletion, addition, etc.); the amino acid sequence of CDR-H3 comprises the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 19 comprising no more than 1, 2, or 3 amino acid variations (e.g., substitution, deletion, addition, etc.); and the amino acid sequence of CDR-L3 comprises the amino acid sequence set forth in SEQ ID NO: 53 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 52 comprising no more than 1, 2, or 3 amino acid substitutions; the amino acid sequence of CDR-H3 comprises the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 19 comprising no more than 1, 2, or 3 amino acid substitutions; and the amino acid sequence of CDR-L3 comprises the amino acid sequence set forth in SEQ ID NO: 53 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 54; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 55. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 54; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 55. In some embodiments, the amino acid sequence of the VH comprises an amino acid sequence at least 95%, 96%, 97%, 98%, 99%, or 100% identical to the amino acid sequence set forth in SEQ ID NO: 54; and the amino acid sequence of the VL comprises an amino acid sequence at least 95%, 96%, 97%, 98%, 99%, or 100% identical to the amino acid sequence set forth in SEQ ID NO: 55. In some embodiments, the amino acid sequence of the VH comprises the amino acid sequence set forth in SEQ ID NO: 54; and the amino acid sequence of the VL comprises the amino acid sequence set forth in SEQ ID NO: 55.

In some embodiments, the amino acid sequence of CDR-H1 comprises the amino acid sequence set forth in SEQ ID NO: 29, or the amino acid sequence set forth in SEQ ID NO: 29 comprising 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising 1, 2, or 3 amino acid variations (e.g., substitution, deletion, addition, etc.); the amino acid sequence of CDR-L1 comprises the amino acid sequence set forth in SEQ ID NO: 56, or the amino acid sequence set forth in SEQ ID NO: 56 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 57, or the amino acid sequence set forth in SEQ ID NO: 57 comprising 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 4, or the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17, or the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 56, or the amino acid sequence set forth in SEQ ID NO: 56 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 57, or the amino acid sequence set forth in SEQ ID NO: 57 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29, or the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 4, or the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17, or the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 56, or the amino acid sequence set forth in SEQ ID NO: 56 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 57, or the amino acid sequence set forth in SEQ ID NO: 57 comprising no more than 1, 2, or 3 amino acid substitutions.

In some embodiments, the amino acid sequence of CDR-H1 comprises the amino acid sequence set forth in SEQ ID NO: 29; the amino acid sequence of CDR-H2 comprises the amino acid sequence set forth in SEQ ID NO: 4; the amino acid sequence of CDR-H3 comprises the amino acid sequence set forth in SEQ ID NO: 17; the amino acid sequence of CDR-L1 comprises the amino acid sequence set forth in SEQ ID NO: 56; the amino acid sequence of CDR-L2 comprises the amino acid sequence set forth in SEQ ID NO: 7; and the amino acid sequence of CDR-L3 comprises the amino acid sequence set forth in SEQ ID NO: 57.

In some embodiments, the amino acid sequence of CDR-H1 comprises the amino acid sequence set forth in SEQ ID NO: 29 comprising no more than 1, 2, or 3 amino acid variations (e.g., substitution, deletion, addition, etc.); the amino acid sequence of CDR-H2 comprises the amino acid sequence set forth in SEQ ID NO: 4 comprising no more than 1, 2, or 3 amino acid variations (e.g., substitution, deletion, addition, etc.); the amino acid sequence of CDR-H3 comprises the amino acid sequence set forth in SEQ ID NO: 17 comprising no more than 1, 2, or 3 amino acid variations (e.g., substitution, deletion, addition, etc.); the amino acid sequence of CDR-L1 comprises the amino acid sequence set forth in SEQ ID NO: 56 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 57 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 29 comprising no more than 1, 2, or 3 amino acid substitutions; the amino acid sequence of CDR-H2 comprises the amino acid sequence set forth in SEQ ID NO: 4 comprising no more than 1, 2, or 3 amino acid substitutions; the amino acid sequence of CDR-H3 comprises the amino acid sequence set forth in SEQ ID NO: 17 comprising no more than 1, 2, or 3 amino acid substitutions; the amino acid sequence of CDR-L1 comprises the amino acid sequence set forth in SEQ ID NO: 56 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 57 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 58; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 59. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 58; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 59. In some embodiments, the amino acid sequence of the VH comprises an amino acid sequence at least 95%, 96%, 97%, 98%, 99%, or 100% identical to the amino acid sequence set forth in SEQ ID NO: 58; and the amino acid sequence of the VL comprises an amino acid sequence at least 95%, 96%, 97%, 98%, 99%, or 100% identical to the amino acid sequence set forth in SEQ ID NO: 59. In some embodiments, the amino acid sequence of the VH comprises the amino acid sequence set forth in SEQ ID NO: 58; and the amino acid sequence of the VL comprises the amino acid sequence set forth in SEQ ID NO: 59.

In some embodiments, the amino acid sequence of CDR-H1 comprises the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of CDR-H2 comprises the amino acid sequence set forth in SEQ ID NO: 61, or the amino acid sequence set forth in SEQ ID NO: 61 comprising 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 63, or the amino acid sequence set forth in SEQ ID NO: 63 comprising 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 61, or the amino acid sequence set forth in SEQ ID NO: 61 comprising no more than 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 18, or the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 63, or the amino acid sequence set forth in SEQ ID NO: 63 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 61, or the amino acid sequence set forth in SEQ ID NO: 61 comprising no more than 1, 2, or 3 amino acid substitutions; the amino acid sequence of CDR-H3 comprises the amino acid sequence set forth in SEQ ID NO: 62, or the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 18, or the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 63, or the amino acid sequence set forth in SEQ ID NO: 63 comprising no more than 1, 2, or 3 amino acid substitutions.

In some embodiments, the amino acid sequence of CDR-H1 comprises the amino acid sequence set forth in SEQ ID NO: 60; the amino acid sequence of CDR-H2 comprises the amino acid sequence set forth in SEQ ID NO: 61; the amino acid sequence of CDR-H3 comprises the amino acid sequence set forth in SEQ ID NO: 62; the amino acid sequence of CDR-L1 comprises the amino acid sequence set forth in SEQ ID NO: 18; the amino acid sequence of CDR-L2 comprises the amino acid sequence set forth in SEQ ID NO: 7; and the amino acid sequence of CDR-L3 comprises the amino acid sequence set forth in SEQ ID NO: 63.

In some embodiments, the amino acid sequence of CDR-H1 comprises the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 61 comprising no more than 1, 2, or 3 amino acid variations (e.g., substitution, deletion, addition, etc.); the amino acid sequence of CDR-H3 comprises the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 18 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 63 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 61 comprising no more than 1, 2, or 3 amino acid substitutions; the amino acid sequence of CDR-H3 comprises the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 18 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 63 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 64; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 65. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 64; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 65. In some embodiments, the amino acid sequence of the VH comprises an amino acid sequence at least 95%, 96%, 97%, 98%, 99%, or 100% identical to the amino acid sequence set forth in SEQ ID NO: 64; and the amino acid sequence of the VL comprises an amino acid sequence at least 95%, 96%, 97%, 98%, 99%, or 100% identical to the amino acid sequence set forth in SEQ ID NO: 65. In some embodiments, the amino acid sequence of the VH comprises the amino acid sequence set forth in SEQ ID NO: 64; and the amino acid sequence of the VL comprises the amino acid sequence set forth in SEQ ID NO: 65.

In some embodiments, the amino acid sequence of CDR-H1 comprises the amino acid sequence set forth in SEQ ID NO: 66, or the amino acid sequence set forth in SEQ ID NO: 66 comprising 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising 1, 2, or 3 amino acid variations (e.g., substitution, deletion, addition, etc.); the amino acid sequence of CDR-L1 comprises the amino acid sequence set forth in SEQ ID NO: 68, or the amino acid sequence set forth in SEQ ID NO: 68 comprising 1, 2, or 3 amino acid variations (e.g., substitution, deletion, addition, etc.); the amino acid sequence of CDR-L2 comprises the amino acid sequence set forth in SEQ ID NO: 69, or the amino acid sequence set forth in SEQ ID NO: 69 comprising 1, 2, or 3 amino acid variations (e.g., substitution, deletion, addition, etc.); and the amino acid sequence of CDR-L3 comprises the amino acid sequence set forth in SEQ ID NO: 70, or the amino acid sequence set forth in SEQ ID NO: 70 comprising 1, 2, or 3 amino acid variations (e.g., substitution, deletion, addition, etc.).

In some embodiments, the amino acid sequence of CDR-H1 comprises the amino acid sequence set forth in SEQ ID NO: 66, or the amino acid sequence set forth in SEQ ID NO: 66 comprising no more than 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 68, or the amino acid sequence set forth in SEQ ID NO: 68 comprising no more than 1, 2, or 3 amino acid variations (e.g., substitution, deletion, addition, etc.); the amino acid sequence of CDR-L2 comprises the amino acid sequence set forth in SEQ ID NO: 69, or the amino acid sequence set forth in SEQ ID NO: 69 comprising no more than 1, 2, or 3 amino acid variations (e.g., substitution, deletion, addition, etc.); and the amino acid sequence of CDR-L3 comprises the amino acid sequence set forth in SEQ ID NO: 70, or the amino acid sequence set forth in SEQ ID NO: 70 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 66, or the amino acid sequence set forth in SEQ ID NO: 66 comprising no more than 1, 2, or 3 amino acid substitutions; the amino acid sequence of CDR-H2 comprises the amino acid sequence set forth in SEQ ID NO: 67, or the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 62, or the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 68, or the amino acid sequence set forth in SEQ ID NO: 68 comprising no more than 1, 2, or 3 amino acid substitutions; the amino acid sequence of CDR-L2 comprises the amino acid sequence set forth in SEQ ID NO: 69, or the amino acid sequence set forth in SEQ ID NO: 69 comprising no more than 1, 2, or 3 amino acid substitutions; and the amino acid sequence of CDR-L3 comprises the amino acid sequence set forth in SEQ ID NO: 70, or the amino acid sequence set forth in SEQ ID NO: 70 comprising no more than 1, 2, or 3 amino acid substitutions.

In some embodiments, the amino acid sequence of CDR-H1 comprises the amino acid sequence set forth in SEQ ID NO: 66; the amino acid sequence of CDR-H2 comprises the amino acid sequence set forth in SEQ ID NO: 67; the amino acid sequence of CDR-H3 comprises the amino acid sequence set forth in SEQ ID NO: 62; the amino acid sequence of CDR-L1 comprises the amino acid sequence set forth in SEQ ID NO: 68; the amino acid sequence of CDR-L2 comprises the amino acid sequence set forth in SEQ ID NO: 69; and the amino acid sequence of CDR-L3 comprises the amino acid sequence set forth in SEQ ID NO: 70.

In some embodiments, the amino acid sequence of CDR-H1 comprises the amino acid sequence set forth in SEQ ID NO: 66 comprising no more than 1, 2, or 3 amino acid variations (e.g., substitution, deletion, addition, etc.); the amino acid sequence of CDR-H2 comprises the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 68 comprising no more than 1, 2, or 3 amino acid variations (e.g., substitution, deletion, addition, etc.); the amino acid sequence of CDR-L2 comprises the amino acid sequence set forth in SEQ ID NO: 69 comprising no more than 1, 2, or 3 amino acid variations (e.g., substitution, deletion, addition, etc.); and the amino acid sequence of CDR-L3 comprises the amino acid sequence set forth in SEQ ID NO: 70 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 66 comprising no more than 1, 2, or 3 amino acid substitutions; the amino acid sequence of CDR-H2 comprises the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 68 comprising no more than 1, 2, or 3 amino acid substitutions; the amino acid sequence of CDR-L2 comprises the amino acid sequence set forth in SEQ ID NO: 69 comprising no more than 1, 2, or 3 amino acid substitutions; and the amino acid sequence of CDR-L3 comprises the amino acid sequence set forth in SEQ ID NO: 70 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 71; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 72. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 71; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 72. In some embodiments, the amino acid sequence of the VH comprises an amino acid sequence at least 95%, 96%, 97%, 98%, 99%, or 100% identical to the amino acid sequence set forth in SEQ ID NO: 71; and the amino acid sequence of the VL comprises an amino acid sequence at least 95%, 96%, 97%, 98%, 99%, or 100% identical to the amino acid sequence set forth in SEQ ID NO: 72. In some embodiments, the amino acid sequence of the VH comprises the amino acid sequence set forth in SEQ ID NO: 71; and the amino acid sequence of the VL comprises the amino acid sequence set forth in SEQ ID NO: 72.

In some embodiments, the amino acid sequence of CDR-H1 comprises the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising 1, 2, or 3 amino acid variations (e.g., substitution, deletion, addition, etc.); the amino acid sequence of CDR-L1 comprises the amino acid sequence set forth in SEQ ID NO: 74, or the amino acid sequence set forth in SEQ ID NO: 74 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 63, or the amino acid sequence set forth in SEQ ID NO: 63 comprising 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 74, or the amino acid sequence set forth in SEQ ID NO: 74 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 63, or the amino acid sequence set forth in SEQ ID NO: 63 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 67, or the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 73, or the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 74, or the amino acid sequence set forth in SEQ ID NO: 74 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 63, or the amino acid sequence set forth in SEQ ID NO: 63 comprising no more than 1, 2, or 3 amino acid substitutions.

In some embodiments, the amino acid sequence of CDR-H1 comprises the amino acid sequence set forth in SEQ ID NO: 60; the amino acid sequence of CDR-H2 comprises the amino acid sequence set forth in SEQ ID NO: 67; the amino acid sequence of CDR-H3 comprises the amino acid sequence set forth in SEQ ID NO: 73; the amino acid sequence of CDR-L1 comprises the amino acid sequence set forth in SEQ ID NO: 74; the amino acid sequence of CDR-L2 comprises the amino acid sequence set forth in SEQ ID NO: 7; and the amino acid sequence of CDR-L3 comprises the amino acid sequence set forth in SEQ ID NO: 63.

In some embodiments, the amino acid sequence of CDR-H1 comprises the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 74 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 63 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 74 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 63 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 75; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 76. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 75; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 76. In some embodiments, the amino acid sequence of the VH comprises an amino acid sequence at least 95%, 96%, 97%, 98%, 99%, or 100% identical to the amino acid sequence set forth in SEQ ID NO: 75; and the amino acid sequence of the VL comprises an amino acid sequence at least 95%, 96%, 97%, 98%, 99%, or 100% identical to the amino acid sequence set forth in SEQ ID NO: 76. In some embodiments, the amino acid sequence of the VH comprises the amino acid sequence set forth in SEQ ID NO: 75; and the amino acid sequence of the VL comprises the amino acid sequence set forth in SEQ ID NO: 76.

In some embodiments, the amino acid sequence of CDR-H1 comprises the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of CDR-H2 comprises the amino acid sequence set forth in SEQ ID NO: 77, or the amino acid sequence set forth in SEQ ID NO: 77 comprising 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 78, or the amino acid sequence set forth in SEQ ID NO: 78 comprising 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 77, or the amino acid sequence set forth in SEQ ID NO: 77 comprising no more than 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 78, or the amino acid sequence set forth in SEQ ID NO: 78 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 77, or the amino acid sequence set forth in SEQ ID NO: 77 comprising no more than 1, 2, or 3 amino acid substitutions; the amino acid sequence of CDR-H3 comprises the amino acid sequence set forth in SEQ ID NO: 73, or the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 78, or the amino acid sequence set forth in SEQ ID NO: 78 comprising no more than 1, 2, or 3 amino acid substitutions.

In some embodiments, the amino acid sequence of CDR-H1 comprises the amino acid sequence set forth in SEQ ID NO: 60; the amino acid sequence of CDR-H2 comprises the amino acid sequence set forth in SEQ ID NO: 77; the amino acid sequence of CDR-H3 comprises the amino acid sequence set forth in SEQ ID NO: 73; the amino acid sequence of CDR-L1 comprises the amino acid sequence set forth in SEQ ID NO: 12; the amino acid sequence of CDR-L2 comprises the amino acid sequence set forth in SEQ ID NO: 7; and the amino acid sequence of CDR-L3 comprises the amino acid sequence set forth in SEQ ID NO: 78.

In some embodiments, the amino acid sequence of CDR-H1 comprises the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 77 comprising no more than 1, 2, or 3 amino acid variations (e.g., substitution, deletion, addition, etc.); the amino acid sequence of CDR-H3 comprises the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 78 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 77 comprising no more than 1, 2, or 3 amino acid substitutions; the amino acid sequence of CDR-H3 comprises the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 78 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 79; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 80. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 79; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 80. In some embodiments, the amino acid sequence of the VH comprises an amino acid sequence at least 95%, 96%, 97%, 98%, 99%, or 100% identical to the amino acid sequence set forth in SEQ ID NO: 79; and the amino acid sequence of the VL comprises an amino acid sequence at least 95%, 96%, 97%, 98%, 99%, or 100% identical to the amino acid sequence set forth in SEQ ID NO: 80. In some embodiments, the amino acid sequence of the VH comprises the amino acid sequence set forth in SEQ ID NO: 79; and the amino acid sequence of the VL comprises the amino acid sequence set forth in SEQ ID NO: 80.

In some embodiments, the amino acid sequence of CDR-H1 comprises the amino acid sequence set forth in SEQ ID NO: 81, or the amino acid sequence set forth in SEQ ID NO: 81 comprising 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 25, or the amino acid sequence set forth in SEQ ID NO: 25 comprising 1, 2, or 3 amino acid variations (e.g., substitution, deletion, addition, etc.); and the amino acid sequence of CDR-L3 comprises the amino acid sequence set forth in SEQ ID NO: 82, or the amino acid sequence set forth in SEQ ID NO: 82 comprising 1, 2, or 3 amino acid variations (e.g., substitution, deletion, addition, etc.).

In some embodiments, the amino acid sequence of CDR-H1 comprises the amino acid sequence set forth in SEQ ID NO: 81, or the amino acid sequence set forth in SEQ ID NO: 81 comprising no more than 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 25, or the amino acid sequence set forth in SEQ ID NO: 25 comprising no more than 1, 2, or 3 amino acid variations (e.g., substitution, deletion, addition, etc.); and the amino acid sequence of CDR-L3 comprises the amino acid sequence set forth in SEQ ID NO: 82, or the amino acid sequence set forth in SEQ ID NO: 82 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 81, or the amino acid sequence set forth in SEQ ID NO: 81 comprising no more than 1, 2, or 3 amino acid substitutions; the amino acid sequence of CDR-H2 comprises the amino acid sequence set forth in SEQ ID NO: 67, or the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 62, or the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 25, or the amino acid sequence set forth in SEQ ID NO: 25 comprising no more than 1, 2, or 3 amino acid substitutions; and the amino acid sequence of CDR-L3 comprises the amino acid sequence set forth in SEQ ID NO: 82, or the amino acid sequence set forth in SEQ ID NO: 82 comprising no more than 1, 2, or 3 amino acid substitutions.

In some embodiments, the amino acid sequence of CDR-H1 comprises the amino acid sequence set forth in SEQ ID NO: 81; the amino acid sequence of CDR-H2 comprises the amino acid sequence set forth in SEQ ID NO: 67; the amino acid sequence of CDR-H3 comprises the amino acid sequence set forth in SEQ ID NO: 62; the amino acid sequence of CDR-L1 comprises the amino acid sequence set forth in SEQ ID NO: 12; the amino acid sequence of CDR-L2 comprises the amino acid sequence set forth in SEQ ID NO: 25; and the amino acid sequence of CDR-L3 comprises the amino acid sequence set forth in SEQ ID NO: 82.

In some embodiments, the amino acid sequence of CDR-H1 comprises the amino acid sequence set forth in SEQ ID NO: 81 comprising no more than 1, 2, or 3 amino acid variations (e.g., substitution, deletion, addition, etc.); the amino acid sequence of CDR-H2 comprises the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 25 comprising no more than 1, 2, or 3 amino acid variations (e.g., substitution, deletion, addition, etc.); and the amino acid sequence of CDR-L3 comprises the amino acid sequence set forth in SEQ ID NO: 82 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 81 comprising no more than 1, 2, or 3 amino acid substitutions; the amino acid sequence of CDR-H2 comprises the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 25 comprising no more than 1, 2, or 3 amino acid substitutions; and the amino acid sequence of CDR-L3 comprises the amino acid sequence set forth in SEQ ID NO: 82 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 83; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 84. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 83; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 84. In some embodiments, the amino acid sequence of the VH comprises an amino acid sequence at least 95%, 96%, 97%, 98%, 99%, or 100% identical to the amino acid sequence set forth in SEQ ID NO: 83; and the amino acid sequence of the VL comprises an amino acid sequence at least 95%, 96%, 97%, 98%, 99%, or 100% identical to the amino acid sequence set forth in SEQ ID NO: 84. In some embodiments, the amino acid sequence of the VH comprises the amino acid sequence set forth in SEQ ID NO: 83; and the amino acid sequence of the VL comprises the amino acid sequence set forth in SEQ ID NO: 84.

In some embodiments, the amino acid sequence of CDR-H1 comprises the amino acid sequence set forth in SEQ ID NO: 81, or the amino acid sequence set forth in SEQ ID NO: 81 comprising 1, 2, or 3 amino acid variations (e.g., substitution, deletion, addition, etc.); the amino acid sequence of CDR-H2 comprises the amino acid sequence set forth in SEQ ID NO: 77, or the amino acid sequence set forth in SEQ ID NO: 77 comprising 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of CDR-L3 comprises the amino acid sequence set forth in SEQ ID NO: 85, or the amino acid sequence set forth in SEQ ID NO: 85 comprising 1, 2, or 3 amino acid variations (e.g., substitution, deletion, addition, etc.).

In some embodiments, the amino acid sequence of CDR-H1 comprises the amino acid sequence set forth in SEQ ID NO: 81, or the amino acid sequence set forth in SEQ ID NO: 81 comprising no more than 1, 2, or 3 amino acid variations (e.g., substitution, deletion, addition, etc.); the amino acid sequence of CDR-H2 comprises the amino acid sequence set forth in SEQ ID NO: 77, or the amino acid sequence set forth in SEQ ID NO: 77 comprising no more than 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 85, or the amino acid sequence set forth in SEQ ID NO: 85 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 81, or the amino acid sequence set forth in SEQ ID NO: 81 comprising no more than 1, 2, or 3 amino acid substitutions; the amino acid sequence of CDR-H2 comprises the amino acid sequence set forth in SEQ ID NO: 77, or the amino acid sequence set forth in SEQ ID NO: 77 comprising no more than 1, 2, or 3 amino acid substitutions; the amino acid sequence of CDR-H3 comprises the amino acid sequence set forth in SEQ ID NO: 73, or the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7, or the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 85, or the amino acid sequence set forth in SEQ ID NO: 85 comprising no more than 1, 2, or 3 amino acid substitutions.

In some embodiments, the amino acid sequence of CDR-H1 comprises the amino acid sequence set forth in SEQ ID NO: 81; the amino acid sequence of CDR-H2 comprises the amino acid sequence set forth in SEQ ID NO: 77; the amino acid sequence of CDR-H3 comprises the amino acid sequence set forth in SEQ ID NO: 73; the amino acid sequence of CDR-L1 comprises the amino acid sequence set forth in SEQ ID NO: 12; the amino acid sequence of CDR-L2 comprises the amino acid sequence set forth in SEQ ID NO: 7; and the amino acid sequence of CDR-L3 comprises the amino acid sequence set forth in SEQ ID NO: 85.

In some embodiments, the amino acid sequence of CDR-H1 comprises the amino acid sequence set forth in SEQ ID NO: 81 comprising no more than 1, 2, or 3 amino acid variations (e.g., substitution, deletion, addition, etc.); the amino acid sequence of CDR-H2 comprises the amino acid sequence set forth in SEQ ID NO: 77 comprising no more than 1, 2, or 3 amino acid variations (e.g., substitution, deletion, addition, etc.); the amino acid sequence of CDR-H3 comprises the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 7 comprising no more than 1, 2, or 3 amino acid variations (e.g., substitution, deletion, addition, etc.); and the amino acid sequence of CDR-L3 comprises the amino acid sequence set forth in SEQ ID NO: 85 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 81 comprising no more than 1, 2, or 3 amino acid substitutions; the amino acid sequence of CDR-H2 comprises the amino acid sequence set forth in SEQ ID NO: 77 comprising no more than 1, 2, or 3 amino acid substitutions; the amino acid sequence of CDR-H3 comprises the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 7 comprising no more than 1, 2, or 3 amino acid substitutions; and the amino acid sequence of CDR-L3 comprises the amino acid sequence set forth in SEQ ID NO: 85 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 86; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 87. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 86; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 87. In some embodiments, the amino acid sequence of the VH comprises an amino acid sequence at least 95%, 96%, 97%, 98%, 99%, or 100% identical to the amino acid sequence set forth in SEQ ID NO: 86; and the amino acid sequence of the VL comprises an amino acid sequence at least 95%, 96%, 97%, 98%, 99%, or 100% identical to the amino acid sequence set forth in SEQ ID NO: 87. In some embodiments, the amino acid sequence of the VH comprises the amino acid sequence set forth in SEQ ID NO: 86; and the amino acid sequence of the VL comprises the amino acid sequence set forth in SEQ ID NO: 87.

In some embodiments, the amino acid sequence of CDR-H1 comprises the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 88, or the amino acid sequence set forth in SEQ ID NO: 88 comprising 1, 2, or 3 amino acid variations (e.g., substitution, deletion, addition, etc.); and the amino acid sequence of CDR-L3 comprises the amino acid sequence set forth in SEQ ID NO: 89, or the amino acid sequence set forth in SEQ ID NO: 89 comprising 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 88, or the amino acid sequence set forth in SEQ ID NO: 88 comprising no more than 1, 2, or 3 amino acid variations (e.g., substitution, deletion, addition, etc.); and the amino acid sequence of CDR-L3 comprises the amino acid sequence set forth in SEQ ID NO: 89, or the amino acid sequence set forth in SEQ ID NO: 89 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 67, or the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 62, or the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 88, or the amino acid sequence set forth in SEQ ID NO: 88 comprising no more than 1, 2, or 3 amino acid substitutions; and the amino acid sequence of CDR-L3 comprises the amino acid sequence set forth in SEQ ID NO: 89, or the amino acid sequence set forth in SEQ ID NO: 89 comprising no more than 1, 2, or 3 amino acid substitutions.

In some embodiments, the amino acid sequence of CDR-H1 comprises the amino acid sequence set forth in SEQ ID NO: 60; the amino acid sequence of CDR-H2 comprises the amino acid sequence set forth in SEQ ID NO: 67; the amino acid sequence of CDR-H3 comprises the amino acid sequence set forth in SEQ ID NO: 62; the amino acid sequence of CDR-L1 comprises the amino acid sequence set forth in SEQ ID NO: 12; the amino acid sequence of CDR-L2 comprises the amino acid sequence set forth in SEQ ID NO: 88; and the amino acid sequence of CDR-L3 comprises the amino acid sequence set forth in SEQ ID NO: 89.

In some embodiments, the amino acid sequence of CDR-H1 comprises the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 88 comprising no more than 1, 2, or 3 amino acid variations (e.g., substitution, deletion, addition, etc.); and the amino acid sequence of CDR-L3 comprises the amino acid sequence set forth in SEQ ID NO: 89 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 88 comprising no more than 1, 2, or 3 amino acid substitutions; and the amino acid sequence of CDR-L3 comprises the amino acid sequence set forth in SEQ ID NO: 89 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 90; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 91. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 90; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 91. In some embodiments, the amino acid sequence of the VH comprises an amino acid sequence at least 95%, 96%, 97%, 98%, 99%, or 100% identical to the amino acid sequence set forth in SEQ ID NO: 90; and the amino acid sequence of the VL comprises an amino acid sequence at least 95%, 96%, 97%, 98%, 99%, or 100% identical to the amino acid sequence set forth in SEQ ID NO: 91. In some embodiments, the amino acid sequence of the VH comprises the amino acid sequence set forth in SEQ ID NO: 90; and the amino acid sequence of the VL comprises the amino acid sequence set forth in SEQ ID NO: 91.

In some embodiments, the amino acid sequence of CDR-H1 comprises the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of CDR-H2 comprises the amino acid sequence set forth in SEQ ID NO: 77, or the amino acid sequence set forth in SEQ ID NO: 77 comprising 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 19, or the amino acid sequence set forth in SEQ ID NO: 19 comprising 1, 2, or 3 amino acid variations (e.g., substitution, deletion, addition, etc.); and the amino acid sequence of CDR-L3 comprises the amino acid sequence set forth in SEQ ID NO: 93, or the amino acid sequence set forth in SEQ ID NO: 93 comprising 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 77, or the amino acid sequence set forth in SEQ ID NO: 77 comprising no more than 1, 2, or 3 amino acid variations (e.g., substitution, deletion, addition, etc.); the amino acid sequence of CDR-H3 comprises the amino acid sequence set forth in SEQ ID NO: 62, or the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 19, or the amino acid sequence set forth in SEQ ID NO: 19 comprising no more than 1, 2, or 3 amino acid variations (e.g., substitution, deletion, addition, etc.); and the amino acid sequence of CDR-L3 comprises the amino acid sequence set forth in SEQ ID NO: 93, or the amino acid sequence set forth in SEQ ID NO: 93 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 77, or the amino acid sequence set forth in SEQ ID NO: 77 comprising no more than 1, 2, or 3 amino acid substitutions; the amino acid sequence of CDR-H3 comprises the amino acid sequence set forth in SEQ ID NO: 62, or the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 19, or the amino acid sequence set forth in SEQ ID NO: 19 comprising no more than 1, 2, or 3 amino acid substitutions; and the amino acid sequence of CDR-L3 comprises the amino acid sequence set forth in SEQ ID NO: 93, or the amino acid sequence set forth in SEQ ID NO: 93 comprising no more than 1, 2, or 3 amino acid substitutions.

In some embodiments, the amino acid sequence of CDR-H1 comprises the amino acid sequence set forth in SEQ ID NO: 60; the amino acid sequence of CDR-H2 comprises the amino acid sequence set forth in SEQ ID NO: 77; the amino acid sequence of CDR-H3 comprises the amino acid sequence set forth in SEQ ID NO: 62; the amino acid sequence of CDR-L1 comprises the amino acid sequence set forth in SEQ ID NO: 12; the amino acid sequence of CDR-L2 comprises the amino acid sequence set forth in SEQ ID NO: 19; and the amino acid sequence of CDR-L3 comprises the amino acid sequence set forth in SEQ ID NO: 93.

In some embodiments, the amino acid sequence of CDR-H1 comprises the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 77 comprising no more than 1, 2, or 3 amino acid variations (e.g., substitution, deletion, addition, etc.); the amino acid sequence of CDR-H3 comprises the amino acid sequence set forth in SEQ ID NO: 62 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 19 comprising no more than 1, 2, or 3 amino acid variations (e.g., substitution, deletion, addition, etc.); and the amino acid sequence of CDR-L3 comprises the amino acid sequence set forth in SEQ ID NO: 93 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 77 comprising no more than 1, 2, or 3 amino acid substitutions; the amino acid sequence of CDR-H3 comprises the amino acid sequence set forth in SEQ ID NO: 62 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 19 comprising no more than 1, 2, or 3 amino acid substitutions; and the amino acid sequence of CDR-L3 comprises the amino acid sequence set forth in SEQ ID NO: 93 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 94; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 176. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 94; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 176. In some embodiments, the amino acid sequence of the VH comprises an amino acid sequence at least 95%, 96%, 97%, 98%, 99%, or 100% identical to the amino acid sequence set forth in SEQ ID NO: 94; and the amino acid sequence of the VL comprises an amino acid sequence at least 95%, 96%, 97%, 98%, 99%, or 100% identical to the amino acid sequence set forth in SEQ ID NO: 176. In some embodiments, the amino acid sequence of the VH comprises the amino acid sequence set forth in SEQ ID NO: 94; and the amino acid sequence of the VL comprises the amino acid sequence set forth in SEQ ID NO: 176.

In some embodiments, the amino acid sequence of CDR-H1 comprises the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising 1, 2, or 3 amino acid variations (e.g., substitution, deletion, addition, etc.); the amino acid sequence of CDR-L2 comprises the amino acid sequence set forth in SEQ ID NO: 95, or the amino acid sequence set forth in SEQ ID NO: 95 comprising 1, 2, or 3 amino acid variations (e.g., substitution, deletion, addition, etc.); and the amino acid sequence of CDR-L3 comprises the amino acid sequence set forth in SEQ ID NO: 96, or the amino acid sequence set forth in SEQ ID NO: 96 comprising 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 67, or the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 73, or the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 12, or the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 95, or the amino acid sequence set forth in SEQ ID NO: 95 comprising no more than 1, 2, or 3 amino acid variations (e.g., substitution, deletion, addition, etc.); and the amino acid sequence of CDR-L3 comprises the amino acid sequence set forth in SEQ ID NO: 96, or the amino acid sequence set forth in SEQ ID NO: 96 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60, or the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 67, or the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 73, or the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 12, or the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 95, or the amino acid sequence set forth in SEQ ID NO: 95 comprising no more than 1, 2, or 3 amino acid substitutions; and the amino acid sequence of CDR-L3 comprises the amino acid sequence set forth in SEQ ID NO: 96, or the amino acid sequence set forth in SEQ ID NO: 96 comprising no more than 1, 2, or 3 amino acid substitutions.

In some embodiments, the amino acid sequence of CDR-H1 comprises the amino acid sequence set forth in SEQ ID NO: 60; the amino acid sequence of CDR-H2 comprises the amino acid sequence set forth in SEQ ID NO: 67; the amino acid sequence of CDR-H3 comprises the amino acid sequence set forth in SEQ ID NO: 73; the amino acid sequence of CDR-L1 comprises the amino acid sequence set forth in SEQ ID NO: 12; the amino acid sequence of CDR-L2 comprises the amino acid sequence set forth in SEQ ID NO: 95; and the amino acid sequence of CDR-L3 comprises the amino acid sequence set forth in SEQ ID NO: 96.

In some embodiments, the amino acid sequence of CDR-H1 comprises the amino acid sequence set forth in SEQ ID NO: 60 comprising no more than 1, 2, or 3 amino acid variations (e.g., substitution, deletion, addition, etc.); the amino acid sequence of CDR-H2 comprises the amino acid sequence set forth in SEQ ID NO: 67 comprising no more than 1, 2, or 3 amino acid variations (e.g., substitution, deletion, addition, etc.); the amino acid sequence of CDR-H3 comprises the amino acid sequence set forth in SEQ ID NO: 73 comprising no more than 1, 2, or 3 amino acid variations (e.g., substitution, deletion, addition, etc.); the amino acid sequence of CDR-L1 comprises the amino acid sequence set forth in SEQ ID NO: 12 comprising no more than 1, 2, or 3 amino acid variations (e.g., substitution, deletion, addition, etc.); the amino acid sequence of CDR-L2 comprises the amino acid sequence set forth in SEQ ID NO: 95 comprising no more than 1, 2, or 3 amino acid variations (e.g., substitution, deletion, addition, etc.); and the amino acid sequence of CDR-L3 comprises the amino acid sequence set forth in SEQ ID NO: 96 comprising no more than 1, 2, or 3 amino acid variations (e.g., substitution, deletion, addition, etc.).

In some embodiments, the amino acid sequence of CDR-H1 comprises the amino acid sequence set forth in SEQ ID NO: 60 comprising no more than 1, 2, or 3 amino acid substitutions; the amino acid sequence of CDR-H2 comprises the amino acid sequence set forth in SEQ ID NO: 67 comprising no more than 1, 2, or 3 amino acid substitutions; the amino acid sequence of CDR-H3 comprises the amino acid sequence set forth in SEQ ID NO: 73 comprising no more than 1, 2, or 3 amino acid substitutions; the amino acid sequence of CDR-L1 comprises the amino acid sequence set forth in SEQ ID NO: 12 comprising no more than 1, 2, or 3 amino acid substitutions; the amino acid sequence of CDR-L2 comprises the amino acid sequence set forth in SEQ ID NO: 95 comprising no more than 1, 2, or 3 amino acid substitutions; and the amino acid sequence of CDR-L3 comprises the amino acid sequence set forth in SEQ ID NO: 96 comprising no more than 1, 2, or 3 amino acid substitutions.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 97; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 98. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 97; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 98. In some embodiments, the amino acid sequence of the VH comprises an amino acid sequence at least 95%, 96%, 97%, 98%, 99%, or 100% identical to the amino acid sequence set forth in SEQ ID NO: 97; and the amino acid sequence of the VL comprises an amino acid sequence at least 95%, 96%, 97%, 98%, 99%, or 100% identical to the amino acid sequence set forth in SEQ ID NO: 98. In some embodiments, the amino acid sequence of the VH comprises the amino acid sequence set forth in SEQ ID NO: 97; and the amino acid sequence of the VL comprises the amino acid sequence set forth in SEQ ID NO: 98.

### 5.12.4 CD161+ Immune Cell Abundance Score

In some of the methods described herein a CD161+ immune cell abundance score (CD161 ICA score) is calculated (or had calculated) and/or utilized in making one or more determinations.

In specific embodiments, the CD161+ immune cell abundance score is determined utilizing an IHC assay that utilizes an anti-CD161 antibody provided herein (e.g., Table 2 herein) to detect expression of CD161 in a sample(s).

In specific embodiments, the range of the score is 0-2. In some embodiments, the scoring is as follows: 0=No positive immune cells observed/20× Field (<1 immune cell); 1=Low density of positive immune cells/20× Field (1-4 immune cells); 2=Moderate density of positive immune cells/20× Field (>5 immune cells).

In specific embodiments, the range of the score is 0-2. In some embodiments, the scoring is as follows: 0=No CD161+ positive immune cells within TIS/TCF observed/20× Field (<1 immune cell); 1=Low density of CD161+ positive immune cells within TIS/TCF/20× Field (1-4 immune cells); 2=Moderate density of CD161+ positive immune cells within TIS/TCF/20× Field (≥5 immune cells). In some embodiments, the tumor-induced stroma (TIS) is characterized as within the tumor and included immune cells that were in between tumor cells (IBTC) and within the tumor mass or tumor nests. In some embodiments, the tumor cell field (TCF) is characterized by as at the tumor/stroma interface and included immune cells within the tumor-induced stroma, which represented the parenchymal response to a tumor or the tumor microenvironment.

In specific embodiments, the CD161+ immune cell abundance score is determined as described in Example 5.

In some embodiments, the CD161+ immune cell abundance score is a single value. In some embodiments, the CD161+ immune cell abundance score is an average.

### 5.12.5 Methods of Delivery

Provided herein are methods of delivering (i) a CD161 binding protein described herein; (ii) a fusion protein described herein; (iii) a conjugate described herein; (iv); a nucleic acid molecule described herein; (v) a vector described herein; (vi) a carrier described herein; or (vii) a pharmaceutical composition described herein to a cell, the method comprising introducing into the cell the CD161 binding protein, the fusion protein, the conjugate, the nucleic acid molecule, the vector, the cell (or population of cells), the carrier, or the pharmaceutical composition to the subject, to thereby deliver the CD161 binding protein, the fusion protein, the conjugate, the nucleic acid molecule, the vector, the carrier, or the pharmaceutical composition, to the cell.

In some embodiments, the CD161 binding protein, the fusion protein, the conjugate, the nucleic acid molecule, the vector, the carrier, or the pharmaceutical composition is introduced in an amount and for a time sufficient to deliver the CD161 binding protein, the fusion protein, the conjugate, the nucleic acid molecule, the vector, the carrier, or the pharmaceutical composition to the cell. In some embodiments, the cell is in vitro, ex vivo, or in vivo. In some embodiments, the cell is in a subject (e.g., a human subject).

Provided herein are methods of delivering (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (viii) a pharmaceutical composition described herein to a subject, the method comprising administering to the subject the CD161 binding protein, the fusion protein, the conjugate, the nucleic acid molecule, the vector, the cell, the carrier, or the pharmaceutical composition to the subject, to thereby deliver the CD161 binding protein, the fusion protein, the conjugate, the nucleic acid molecule, the vector, the cell (or population of cells), the carrier, or the pharmaceutical composition, to the subject.

In some embodiments, the CD161 binding protein, the fusion protein, the conjugate, the nucleic acid molecule, the vector, the cell (or population of cells), the carrier, or the pharmaceutical composition is administered in an amount and for a time sufficient to deliver the CD161 binding protein, the fusion protein, the conjugate, the nucleic acid molecule, the vector, the cell (or population of cells), the carrier, or the pharmaceutical composition to the subject.

### 5.12.6 Methods of Inhibiting Binding of CD161 to CLEC2D

Provided herein are methods of inhibiting binding of CD161 expressed on the surface of a cell (e.g., an immune cell) to CLEC2D (e.g., expressed on the surface of a cell (e.g., a cancer cell)), the method comprising contacting the CD161 expressing cell with (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (viii) a pharmaceutical composition described herein, to thereby inhibit binding of CD161 to CLEC2D. In some embodiments, the CD161 binding protein, the fusion protein, the conjugate, the nucleic acid molecule, the vector, the cell (or population of cells), the carrier, or the pharmaceutical composition is contacted to the cell in an amount and for a time sufficient to inhibit binding of CD161 to CLEC2D. In some embodiments, the cell is in vitro, ex vivo, or in vivo. In some embodiments, the cell is in a subject (e.g., a human subject).

Provided herein are methods of inhibiting binding of CD161 expressed on the surface of a cell (e.g., an immune cell) to CLEC2D expressed on the surface of a cell (e.g., a cancer cell) in a subject, the method comprising administering to the subject (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (viii) a pharmaceutical composition described herein to the subject, to thereby inhibit binding of CD161 to CLEC2D in the subject. In some embodiments, the CD161 binding protein, the fusion protein, the conjugate, the nucleic acid molecule, the vector, the cell (or population of cells), the carrier, or the pharmaceutical composition is administered to the subject in an amount and for a time sufficient to inhibit binding of CD161 to CLEC2D.

Provided herein are methods of inhibiting binding of CD161 expressed on the surface of an immune cell (e.g., T cell, NK cell (e.g., tumor infiltrating T cell, tumor infiltrating NK cell)) to CLEC2D expressed on the surface of a cancer cell in a subject, the method comprising administering to the subject (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (viii) a pharmaceutical composition described herein to the subject, to thereby inhibit binding of CD161 to CLEC2D in the subject. In some embodiments, the CD161 binding protein, the fusion protein, the conjugate, the nucleic acid molecule, the vector, the cell (or population of cells), the carrier, or the pharmaceutical composition is administered to the subject in an amount and for a time sufficient to inhibit binding of CD161 to CLEC2D.

Standard binding assays to measure binding of CLEC2D and CD161 are known in the art and described herein. See, e.g., Wade M, Méndez J, Coussens N P, et al. Inhibition of Protein-Protein Interactions: Cell-Based Assays. 2017 Nov. 20. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK464632/; Arkin M R, Glicksman M A, Fu H, et al. Inhibition of Protein-Protein Interactions: Non-Cellular Assay Formats. 2012 Mar. 18 [Updated 2012 Oct. 1]. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK92000/; the entire contents of each of which are incorporated herein by reference for all purposes. For example, non-cell-based assays include, but are not limited to, ELISA. For further example, cell-based assays include, but are not limited to, energy transfer (FOrster resonance energy transfer and bioluminescence resonance energy transfer) and protein complementation (fluorescence or enzymatic, e.g., luciferase).

### 5.12.7 Methods of Treating a Disease

Provided herein are methods of treating, ameliorating, or preventing a disease in a subject in need thereof, the method comprising administering to the subject (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (viii) a pharmaceutical composition described herein, to thereby treat, ameliorate, or prevent the disease in the subject. In some embodiments, the CD161 binding protein, the fusion protein, the conjugate, the nucleic acid molecule, the vector, the cell (or population of cells), the carrier, or the pharmaceutical composition is administered to the subject in an amount and for a time sufficient to treat, ameliorate, or prevent the CD161 associated disease in the subject.

In some embodiments, the disease is cancer. In some embodiments, the disease is a proinflammatory disease (e.g., an autoimmune disease). In some embodiments, the disease is an autoimmune disease.

Provided herein are CD161 binding proteins, fusion proteins, conjugates, nucleic acid molecules, vectors, cell (or population of cells), or pharmaceutical compositions for use in the treatment of a disease (e.g., cancer) in a subject in need thereof.

Provided herein are CD161 binding proteins, fusion proteins, conjugates, nucleic acid molecules, vectors, cells, or pharmaceutical compositions for use as a medicament.

Provided herein are use of a CD161 binding protein described herein, fusion protein described herein, conjugate described herein, nucleic acid molecule described herein, vector described herein, cell (or population of cells) described herein, or pharmaceutical composition described herein for the manufacture of a medicament for the treatment of a disease (e.g., cancer) in a subject in need thereof.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); and (b) administering an anti-CD161 antibody.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); and (c) administering an anti-CD161 antibody.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); and (b) administering an anti-CD161 antibody if expression of CD161 is detected.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); and (c) administering an anti-CD161 antibody if a pre-determined an CD161 ICA score threshold is reached (e.g., a CD161 ICA score of greater than or equal to 1; a CD161 ICA score of greater than or equal to 1.5; a CD161 ICA score of 2).

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof) and (b) determining a CD161 ICA score (e.g., as described herein, e.g., as described in § 5.12.4 herein); and (c) administering an anti-CD161 antibody if a pre-determined an CD161 ICA score threshold is reached (e.g., a CD161 ICA score of greater than or equal to 1; a CD161 ICA score of greater than or equal to 1.5; a CD161 ICA score of 2).

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); and (b)(i) administering an anti-CD161 antibody to the subject if expression of CD161 is detected; or (b)(ii) withholding administration of an anti-CD161 antibody to the subject if expression of CD161 is not detected and optionally administering a different therapeutic agent to treat the disease that is not an anti-CD161 antibody.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); and (c)(i) administering an anti-CD161 antibody to the subject if a pre-determined an CD161 ICA score threshold is reached (e.g., a CD161 ICA score of greater than or equal to 1; a CD161 ICA score of greater than or equal to 1.5; a CD161 ICA score of 2); or (c)(ii) withholding administration of an anti-CD161 antibody to the subject if a pre-determined an CD161 ICA score threshold is not reached (e.g., a CD161 ICA score of greater than or equal to 1; a CD161 ICA score of greater than or equal to 1.5; a CD161 ICA score of 2); and optionally administering a different therapeutic agent to treat the disease that is not an anti-CD161 antibody.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); and (c)(i) administering an anti-CD161 antibody to the subject if a pre-determined an CD161 ICA score threshold is reached (e.g., a CD161 ICA score of greater than or equal to 1; a CD161 ICA score of greater than or equal to 1.5; a CD161 ICA score of 2); or (c)(ii) withholding administration of an anti-CD161 antibody to the subject if a pre-determined an CD161 ICA score threshold is not reached (e.g., a CD161 ICA score of greater than or equal to 1; a CD161 ICA score of greater than or equal to 1.5; a CD161 ICA score of 2) and optionally administering a different therapeutic agent to treat the disease that is not an anti-CD161 antibody.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); and (b) administering an anti-CD161 antibody to the subject based at least in part on the detection of CD161 expression in the sample from the subject.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); and (c) administering an anti-CD161 antibody to the subject based at least in part on the CD161 ICA score of the sample from the subject.

Provided herein are methods of treating a disease in a subject in need thereof, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); and (c) administering an anti-CD161 antibody to the subject based at least in part on the CD161 ICA score.

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

In some embodiments, the presence of CD161 expressing cells in the population of cells (e.g., immune cells (e.g., T cells, NK cells)) indicates that the subject is likely to respond to treatment with an anti-CD161 antibody.

In some embodiments, the presence of a percentage of CD161 expressing cells in the population of cells (e.g., immune cells (e.g., T cells, NK cells)) indicates that the subject is likely to respond to treatment with an anti-CD161 antibody.

In some embodiments, the method comprises withholding administration of an anti-CD161 antibody to the subject if the presence of CD161 expressing cells in the population of immune cells (e.g., T cells, NK cells) in the sample is not detected. In some embodiments, the method comprises administering an alternative therapeutic agent for treatment of the disease that is not an anti-CD161 antibody to the subject if the presence of CD161 expressing cells in the population of cells (e.g., immune cells (e.g., T cells, NK cells)) in the sample is not detected. In some embodiments, the method comprises the different therapeutic agent is a standard of care agent for the disease. In some embodiments, the method comprises the subject is undergoing or has undergone treatment with a different therapeutic agent for the disease that is not an anti-CD161 antibody). In some embodiments, the method comprises the treatment with the different therapeutic agent is discontinued if the presence of CD161 expressing cells in the population of cells (e.g., immune cells (e.g., T cells, NK cells)) in the sample is detected and/or the anti-CD161 antibody).

In some embodiments, the disease is cancer. In some embodiments, the disease is a proinflammatory disease (e.g., an autoimmune disease). In some embodiments, the disease is an autoimmune disease.

### 5.12.8 Methods of Treating Cancer

Provided herein are methods treating, ameliorating, or preventing cancer in a subject in need thereof, the method comprising administering to the subject (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (viii) a pharmaceutical composition described herein, to thereby treat, ameliorate, or prevent the cancer in the subject. In some embodiments, the CD161 binding protein, the fusion protein, the conjugate, the nucleic acid molecule, the vector, the cell (or population of cells), the carrier, or the pharmaceutical composition is administered to the subject in an amount and for a time sufficient to treat, ameliorate, or prevent the cancer in the subject.

Provided herein are methods of treating cancer in a subject in need thereof, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); and (b) administering an agent (e.g., anti-CD161 antibody (e.g., an anti-CD161 antibody described herein (e.g., set forth in Table 3))).

Provided herein are methods of treating cancer in a subject in need thereof, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); and (b) administering an agent (e.g., anti-CD161 antibody (e.g., an anti-CD161 antibody described herein (e.g., set forth in Table 3))) that inhibits the interaction of CD161 to CLEC2D to the subject if expression of CD161 is detected.

Provided herein are methods of treating cancer in a subject in need thereof, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); and (b)(i) administering an agent (e.g., anti-CD161 antibody (e.g., an anti-CD161 antibody described herein (e.g., set forth in Table 3))) that inhibits the interaction of CD161 to CLEC2D to the subject if expression of CD161 is detected; or (b)(ii) withholding administration of an agent (e.g., anti-CD161 antibody) that inhibits the interaction of CD161 to CLEC2D to the subject if expression of CD161 is not detected and optionally administering a different therapeutic agent to treat the cancer that is not an agent that that inhibits the interaction of CD161 to CLEC2D.

Provided herein are methods of treating cancer in a subject in need thereof, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); and (b) administering an agent (e.g., anti-CD161 antibody (e.g., an anti-CD161 antibody described herein (e.g., set forth in Table 3))) that inhibits the interaction of CD161 to CLEC2D to the subject based at least in part on the detection of CD161 expression in the sample from the subject.

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

In some embodiments, the presence of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody (e.g., an anti-CD161 antibody described herein (e.g., set forth in Table 3))) that inhibits the interaction of CD161 to CLEC2D (e.g., an anti-CD161 antibody).

In some embodiments, the presence of a percentage of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody (e.g., set forth in Table 3)) that inhibits the interaction of CD161 to CLEC2D (e.g., anti-CD161 antibody (e.g., an anti-CD161 antibody described herein)).

In some embodiments, the method comprises withholding administration of an agent (e.g., anti-CD161 antibody (e.g., set forth in Table 3)) that inhibits the interaction of CD161 to CLEC2D (e.g., anti-CD161 antibody (e.g., an anti-CD161 antibody described herein)) to the subject if the presence of CD161 expressing cells in the population of immune cells (e.g., T cells, NK cells (e.g., tumor infiltrating T cells, NK cells)) in the sample is not detected. In some embodiments, the method comprises administering an alternative therapeutic agent for treatment of the cancer that is not an agent (e.g., anti-CD161 antibody (e.g., an anti-CD161 antibody described herein (e.g., set forth in Table 3))) that inhibits the interaction of CD161 to CLEC2D (e.g., anti-CD161 antibody (e.g., an anti-CD161 antibody described herein)) to the subject if the presence of CD161 expressing cells in the population of cells (e.g., immune cells (e.g., T cells, NK cells (e.g., tumor infiltrating T cells, tumor infiltrating NK cells))) in the sample is not detected. In some embodiments, the method comprises the different therapeutic agent is a standard of care agent for the cancer. In some embodiments, the method comprises the subject is undergoing or has undergone treatment with a different therapeutic agent for the cancer that is not an agent (e.g., anti-CD161 antibody) that inhibits the interaction of CD161 to CLEC2D (e.g., an anti-CD161 antibody). In some embodiments, the method comprises the treatment with the different therapeutic agent is discontinued if the presence of CD161 expressing cells in the population of cells (e.g., immune cells (e.g., T cells, NK cells (e.g., tumor infiltrating T cells, tumor infiltrating NK cells))) in the sample is detected and/or the agent (e.g., anti-CD161 antibody) that inhibits the interaction of CD161 to CLEC2D (e.g., an anti-CD161 antibody).

In some embodiments, the cancer is a solid tumor. In some embodiments, the cancer is a carcinoma, adenocarcinoma, or sarcoma. In some embodiments, the cancer is a carcinoma.

In some embodiments, the cancer is head cancer, neck cancer, head and neck cancer, lung cancer, colon cancer, rectal cancer, colorectal cancer, renal cancer, ovarian cancer, breast cancer, endometrial cancer, uterine cancer, cervical cancer, anal cancer, prostate cancer, bladder cancer, liver cancer, pancreatic cancer, thyroid cancer, thymus cancer, bronchus cancer, skin cancer, brain cancer, spinal cord cancer, lip cancer, bowel cancer (e.g., small bowel cancer, large bowel cancer), or oral cavity cancer.

In some embodiments, the cancer is head cancer, neck cancer, head and neck cancer, lung cancer, colon cancer, rectal cancer, colorectal cancer, renal cancer, or ovarian cancer.

In some embodiments, the cancer is head and neck squamous cell carcinoma (HNSCC), non-small cell lung cancer, colorectal carcinoma, renal cell carcinoma, cutaneous squamous cell carcinoma.

In some embodiments, the cancer is non-small cell lung cancer, head and neck squamous cell carcinoma, triple negative breast cancer, cutaneous squamous cell carcinoma, hormone receptor positive breast carcinoma, small bowel cancer, esophageal cancer, or colorectal cancer.

In some embodiments, the cancer is a hematological cancer. In some embodiments, the cancer is a lymphoma, leukemia, or myeloma. In some embodiments, the cancer is a lymphoma. In some embodiments, the cancer is diffuse large B cell lymphoma, Hodgkin lymphoma, Burkitt lymphoma, or T-cell lymphoma.

In specific embodiments, the cancer is head and neck squamous cell carcinoma (HNSCC), colorectal cancer (CRC), or non-small cell lung cancer (NSCLC). In specific embodiments, the cancer is squamous cell lung cancer. In specific embodiments, the cancer is head and neck squamous cell carcinoma (HNSCC). In specific embodiments, the cancer is HPV positive head and neck squamous cell carcinoma (HNSCC). In specific embodiments, the cancer is HPV negative head and neck squamous cell carcinoma (HNSCC). In specific embodiments, the cancer is colorectal cancer (CRC). In specific embodiments, the cancer is non-small cell lung cancer (NSCLC). In specific embodiments, the cancer is Hodgkin's lymphoma. In specific embodiments, the cancer is cutaneous squamous cell carcinoma. In specific embodiments, the cancer is breast cancer. In specific embodiments, the cancer is breast cancer characterized as expressing a genetic alteration in BRCA1 and/or BRCA2. In specific embodiments, the cancer is triple negative breast cancer. In specific embodiments, the cancer is diffuse large B cell lymphoma. In specific embodiments, the cancer is esophageal cancer.

### 5.12.9 Methods of Identifying a Subject for Treatment

Provided herein are methods of identifying a subject having cancer for treatment with an anti-CD161 antibody, the method comprising (a) detecting (or having detected) the expression of CD161 by a population of cells (e.g., immune cells)) in a sample (e.g., a tissue sample) from the subject, wherein the detecting comprises the use of a CD161 binding protein described herein (e.g., set forth in Table 2) (or a fusion protein or conjugate thereof); (b) identifying the subject as a subject for treatment with an anti-CD161 antibody if expression of CD161 is detected; and (c) optionally administering an anti-CD161 antibody to the subject if the subject is identified as a subject for treatment with an anti-CD161 antibody that inhibits the interaction of CD161 to CLEC2D.

Provided herein are methods of identifying a subject having cancer for treatment with an anti-CD161 antibody, the method comprising (a) detecting (or having detected) the expression of CD161 by a population of cells (e.g., immune cells)) in a sample (e.g., a tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) identifying the subject as a subject for treatment with an anti-CD161 antibody if a pre-determined an CD161 ICA score threshold is reached (e.g., a CD161 ICA score of greater than or equal to 1; a CD161 ICA score of greater than or equal to 1.5; a CD161 ICA score of 2); and (d) optionally administering an anti-CD161 antibody to the subject if the subject is identified as a subject for treatment with an anti-CD161 antibody that inhibits the interaction of CD161 to CLEC2D.

Provided herein are methods of identifying a subject having a disease for treatment with an anti-CD161 antibody, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); (b) identifying the subject as a subject for treatment with an anti-CD161 antibody if expression of CD161 is detected; and (c) optionally administering an anti-CD161 antibody to the subject if the subject is identified as a subject for treatment with an anti-CD161 antibody.

Provided herein are methods of identifying a subject having a disease for treatment with an anti-CD161 antibody, the method comprising (a) detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) identifying the subject as a subject for treatment with an anti-CD161 antibody if a pre-determined an CD161 ICA score threshold is reached (e.g., a CD161 ICA score of greater than or equal to 1; a CD161 ICA score of greater than or equal to 1.5; a CD161 ICA score of 2); and (d) optionally administering an anti-CD161 antibody to the subject if the subject is identified as a subject for treatment with an anti-CD161 antibody.

In some embodiments, the presence of CD161 expressing cells in the population of cells (e.g., immune cells (e.g., T cells, NK cells)) indicates that the subject is likely to respond to treatment with an anti-CD161 antibody.

In some embodiments, the presence of a percentage of CD161 expressing cells in the population of cells (e.g., immune cells (e.g., T cells, NK cells)) indicates that the subject is likely to respond to treatment with an anti-CD161 antibody.

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

In some embodiments, the method comprises withholding administration of an anti-CD161 antibody to the subject if the presence of CD161 expressing cells in the population of immune cells (e.g., T cells, NK cells) in the sample is not detected. In some embodiments, the method comprises administering an alternative therapeutic agent for treatment of the disease that is not an anti-CD161 antibody to the subject if the presence of CD161 expressing cells in the population of cells (e.g., immune cells (e.g., T cells, NK cells)) in the sample is not detected. In some embodiments, the method comprises the different therapeutic agent is a standard of care agent for the disease. In some embodiments, the method comprises the subject is undergoing or has undergone treatment with a different therapeutic agent for the disease that is not an anti-CD161 antibody. In some embodiments, the method comprises the treatment with the different therapeutic agent is discontinued if the presence of CD161 expressing cells in the population of cells (e.g., immune cells (e.g., T cells, NK cells)) in the sample is detected and/or the anti-CD161 antibody.

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the disease is cancer. In some embodiments, the disease is a proinflammatory disease (e.g., an autoimmune disease). In some embodiments, the disease is an autoimmune disease.

Provided herein are methods of identifying a subject having cancer for treatment with an agent (e.g., anti-CD161 antibody (e.g., an anti-CD161 antibody described herein (e.g., set forth in Table 3))) that inhibits the interaction of CD161 to CLEC2D, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) identifying the subject as a subject for treatment with an agent (e.g., anti-CD161 antibody (e.g., an anti-CD161 antibody described herein)) that inhibits the interaction of CD161 to CLEC2D if expression of CD161 is detected; and (c) optionally administering an agent (e.g., anti-CD161 antibody (e.g., an anti-CD161 antibody described herein)) that inhibits the interaction of CD161 to CLEC2D to the subject if the subject is identified as a subject for treatment with an agent (e.g., anti-CD161 antibody (e.g., set forth in Table 3)) that inhibits the interaction of CD161 to CLEC2D.

Provided herein are methods of identifying a subject having cancer for treatment with an agent (e.g., anti-CD161 antibody (e.g., an anti-CD161 antibody described herein (e.g., set forth in Table 3))) that inhibits the interaction of CD161 to CLEC2D, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) identifying the subject as a subject for treatment with an agent (e.g., anti-CD161 antibody (e.g., an anti-CD161 antibody described herein)) that inhibits the interaction of CD161 to CLEC2D if a pre-determined an CD161 ICA score threshold is reached (e.g., a CD161 ICA score of greater than or equal to 1; a CD161 ICA score of greater than or equal to 1.5; a CD161 ICA score of 2); and (d) optionally administering an agent (e.g., anti-CD161 antibody (e.g., an anti-CD161 antibody described herein)) that inhibits the interaction of CD161 to CLEC2D to the subject if the subject is identified as a subject for treatment with an agent (e.g., anti-CD161 antibody (e.g., set forth in Table 3)) that inhibits the interaction of CD161 to CLEC2D.

Provided herein are methods of identifying a subject having cancer for treatment with an anti-CD161 antibody (e.g., an anti-CD161 antibody described herein (e.g., set forth in Table 3)), the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) identifying the subject as a subject for treatment with an anti-CD161 antibody (e.g., an anti-CD161 antibody described herein (e.g., set forth in Table 3)) if expression of CD161 is detected; and (c) optionally administering an agent (e.g., anti-CD161 antibody (e.g., an anti-CD161 antibody described herein)) that inhibits the interaction of CD161 to CLEC2D to the subject if the subject is identified as a subject for treatment with an agent (e.g., anti-CD161 antibody (e.g., set forth in Table 3)) that inhibits the interaction of CD161 to CLEC2D.

Provided herein are methods of identifying a subject having cancer for treatment with an anti-CD161 antibody (e.g., an anti-CD161 antibody described herein (e.g., set forth in Table 3)), the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) identifying the subject as a subject for treatment with an anti-CD161 antibody (e.g., an anti-CD161 antibody described herein (e.g., set forth in Table 3)) if a pre-determined an CD161 ICA score threshold is reached (e.g., a CD161 ICA score of greater than or equal to 1; a CD161 ICA score of greater than or equal to 1.5; a CD161 ICA score of 2); and (d) optionally administering an agent (e.g., anti-CD161 antibody (e.g., an anti-CD161 antibody described herein)) thatinhibits the interaction of CD161 to CLEC2D to the subject if the subject is identified as a subject for treatment with an agent (e.g., anti-CD161 antibody (e.g., set forth in Table 3)) that inhibits the interaction of CD161 to CLEC2D.

In some embodiments, the presence of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody (e.g., an anti-CD161 antibody described herein (e.g., set forth in Table 3))) that inhibits the interaction of CD161 to CLEC2D (e.g., an anti-CD161 antibody).

In some embodiments, the presence of a percentage of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody) that inhibits the interaction of CD161 to CLEC2D (e.g., anti-CD161 antibody (e.g., an anti-CD161 antibody described herein (e.g., set forth in Table 3))).

In some embodiments, the method comprises withholding administration of an agent (e.g., anti-CD161 antibody (e.g., set forth in Table 3)) that inhibits the interaction of CD161 to CLEC2D (e.g., anti-CD161 antibody (e.g., an anti-CD161 antibody described herein (e.g., set forth in Table 3))) to the subject if the presence of CD161 expressing cells in the population of immune cells (e.g., T cells, NK cells (e.g., tumor infiltrating T cells, NK cells)) in the sample is not detected. In some embodiments, the method comprises administering an alternative therapeutic agent for treatment of the cancer that is not an agent (e.g., anti-CD161 antibody (e.g., an anti-CD161 antibody described herein (e.g., set forth in Table 3))) that inhibits the interaction of CD161 to CLEC2D (e.g., anti-CD161 antibody (e.g., an anti-CD161 antibody described herein)) to the subject if the presence of CD161 expressing cells in the population of cells (e.g., immune cells (e.g., T cells, NK cells (e.g., tumor infiltrating T cells, tumor infiltrating NK cells))) in the sample is not detected. In some embodiments, the method comprises the different therapeutic agent is a standard of care agent for the cancer. In some embodiments, the method comprises the subject is undergoing or has undergone treatment with a different therapeutic agent for the cancer that is not an agent (e.g., anti-CD161 antibody (e.g., set forth in Table 3)) that inhibits the interaction of CD161 to CLEC2D (e.g., an anti-CD161 antibody (e.g., set forth in Table 3)). In some embodiments, the method comprises the treatment with the different therapeutic agent is discontinued if the presence of CD161 expressing cells in the population of cells (e.g., immune cells (e.g., T cells, NK cells (e.g., tumor infiltrating T cells, tumor infiltrating NK cells))) in the sample is detected and/or the agent (e.g., anti-CD161 antibody (e.g., set forth in Table 3)) that inhibits the interaction of CD161 to CLEC2D (e.g., an anti-CD161 antibody (e.g., set forth in Table 3)).

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

### 5.12.10 Methods of Identifying a Subject as a Responder

Provided herein are methods of identifying a subject having a disease who is likely to respond to a treatment with an anti-CD161 antibody (e.g., an anti-CD161 antibody described herein (e.g., set forth in Table 3))), the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample) from the subject, wherein the detecting comprises the use of a CD161 binding protein described herein (e.g., set forth in Table 2) (or a fusion protein or conjugate thereof); (b) identifying the subject as a subject likely to respond to treatment with an anti-CD161 antibody (e.g., an anti-CD161 antibody described herein); and (c) optionally administering an anti-CD161 antibody to the subject if the subject is identified as a subject for treatment with the anti-CD161 antibody.

Provided herein are methods of identifying a subject having a disease who is likely to respond to a treatment with an anti-CD161 antibody (e.g., an anti-CD161 antibody described herein (e.g., set forth in Table 3))), the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) identifying the subject as a subject likely to respond to treatment with an anti-CD161 antibody (e.g., an anti-CD161 antibody described herein); and (d) optionally administering an anti-CD161 antibody to the subject if the subject is identified as a subject for treatment with the anti-CD161 antibody.

In some embodiments, the presence of CD161 expressing cells in the population of cells (e.g., immune cells (e.g., T cells, NK cells)) indicates that the subject is likely to respond to treatment with an anti-CD161 antibody.

In some embodiments, the presence of a percentage of CD161 expressing cells in the population of cells (e.g., immune cells (e.g., T cells, NK cells)) indicates that the subject is likely to respond to treatment with an anti-CD161 antibody.

In some embodiments, the method comprises withholding administration of an anti-CD161 antibody to the subject if the presence of CD161 expressing cells in the population of immune cells (e.g., T cells, NK cells) in the sample is not detected. In some embodiments, the method comprises administering an alternative therapeutic agent for treatment of the disease that is not an anti-CD161 antibody to the subject if the presence of CD161 expressing cells in the population of cells (e.g., immune cells (e.g., T cells, NK cells)) in the sample is not detected. In some embodiments, the method comprises the different therapeutic agent is a standard of care agent for the disease. In some embodiments, the method comprises the subject is undergoing or has undergone treatment with a different therapeutic agent for the disease that is not an anti-CD161 antibody. In some embodiments, the method comprises the treatment with the different therapeutic agent is discontinued if the presence of CD161 expressing cells in the population of cells (e.g., immune cells (e.g., T cells, NK cells)) in the sample is detected and/or the anti-CD161 antibody.

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

In some embodiments, the disease is cancer. In some embodiments, the disease is a proinflammatory disease (e.g., an autoimmune disease). In some embodiments, the disease is an autoimmune disease.

Provided herein are methods of identifying a subject having cancer who is likely to respond to a treatment with an agent (e.g., anti-CD161 antibody (e.g., an anti-CD161 antibody described herein (e.g., set forth in Table 3))) that inhibits the interaction of CD161 to CLEC2D, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) identifying the subject as a subject likely to respond to treatment with an agent (e.g., anti-CD161 antibody (e.g., an anti-CD161 antibody described herein)) that inhibits the interaction of CD161 to CLEC2D if expression of CD161 is detected; and (c) optionally administering an agent (e.g., anti-CD161 antibody (e.g., an anti-CD161 antibody described herein (e.g., set forth in Table 3))) that inhibits the interaction of CD161 to CLEC2D to the subject if the subject is identified as a subject for treatment with an agent (e.g., anti-CD161 antibody (e.g., set forth in Table 3)) that inhibits the interaction of CD161 to CLEC2D.

Provided herein are methods of identifying a subject having cancer who is likely to respond to a treatment with an agent (e.g., anti-CD161 antibody (e.g., an anti-CD161 antibody described herein (e.g., set forth in Table 3))) that inhibits the interaction of CD161 to CLEC2D, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) identifying the subject as a subject likely to respond to treatment with an agent (e.g., anti-CD161 antibody (e.g., an anti-CD161 antibody described herein)) that inhibits the interaction of CD161 to CLEC2D if a pre-determined an CD161 ICA score threshold is reached (e.g., a CD161 ICA score of greater than or equal to 1; a CD161 ICA score of greater than or equal to 1.5; a CD161 ICA score of 2); and (d) optionally administering an agent (e.g., anti-CD161 antibody (e.g., an anti-CD161 antibody described herein (e.g., set forth in Table 3))) that inhibits the interaction of CD161 to CLEC2D to the subject if the subject is identified as a subject for treatment with an agent (e.g., anti-CD161 antibody (e.g., set forth in Table 3)) that inhibits the interaction of CD161 to CLEC2D.

In some embodiments, the presence of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody (e.g., an anti-CD161 antibody described herein (e.g., set forth in Table 3))) that inhibits the interaction of CD161 to CLEC2D (e.g., an anti-CD161 antibody (e.g., set forth in Table 3)).

In some embodiments, the presence of a percentage of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody) that inhibits the interaction of CD161 to CLEC2D (e.g., anti-CD161 antibody (e.g., an anti-CD161 antibody described herein (e.g., set forth in Table 3))).

In some embodiments, the method comprises withholding administration of an agent (e.g., anti-CD161 antibody (e.g., set forth in Table 3)) that inhibits the interaction of CD161 to CLEC2D (e.g., anti-CD161 antibody (e.g., an anti-CD161 antibody described herein (e.g., set forth in Table 3))) to the subject if the presence of CD161 expressing cells in the population of immune cells (e.g., T cells, NK cells (e.g., tumor infiltrating T cells, NK cells)) in the sample is not detected. In some embodiments, the method comprises administering an alternative therapeutic agent for treatment of the cancer that is not an agent (e.g., anti-CD161 antibody (e.g., an anti-CD161 antibody described herein (e.g., set forth in Table 3))) that inhibits the interaction of CD161 to CLEC2D (e.g., anti-CD161 antibody (e.g., an anti-CD161 antibody described herein (e.g., setforth in Table 3))) to the subject if the presence of CD161 expressing cells in the population of cells (e.g., immune cells (e.g., T cells, NK cells (e.g., tumor infiltrating T cells, tumor infiltrating NK cells))) in the sample is not detected. In some embodiments, the method comprises the different therapeutic agent is a standard of care agent for the cancer. In some embodiments, the method comprises the subject is undergoing or has undergone treatment with a different therapeutic agent for the cancer that is not an agent (e.g., anti-CD161 antibody (e.g., set forth in Table 3)) that inhibits the interaction of CD161 to CLEC2D (e.g., an anti-CD161 antibody (e.g., set forth in Table 3)). In some embodiments, the method comprises the treatment with the different therapeutic agent is discontinued if the presence of CD161 expressing cells in the population of cells (e.g., immune cells (e.g., T cells, NK cells (e.g., tumor infiltrating T cells, tumor infiltrating NK cells))) in the sample is detected and/or the agent (e.g., anti-CD161 antibody (e.g., set forth in Table 3)) that inhibits the interaction of CD161 to CLEC2D (e.g., an anti-CD161 antibody (e.g., set forth in Table 3)).

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

### 5.12.11 Methods of Selecting a Therapy

Provided herein are methods of selecting a therapy for a subject disease, the method comprising (a) detecting (or having detected) the expression of CD161 by a population of cells (e.g., immune cells (e.g., T cells, NK cells)) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) selecting a therapy comprising an anti-CD161 antibody if expression of CD161 is detected; and (c) optionally administering the therapy comprising an anti-CD161 antibody to the subject if the therapy is selected.

Provided herein are methods of selecting a therapy for a subject disease, the method comprising (a) detecting (or having detected) the expression of CD161 by a population of cells (e.g., immune cells (e.g., T cells, NK cells)) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) selecting a therapy comprising an anti-CD161 antibody if a pre-determined an CD161 ICA score threshold is reached (e.g., a CD161 ICA score of greater than or equal to 1; a CD161 ICA score of greater than or equal to 1.5; a CD161 ICA score of 2); and (d) optionally administering the therapy comprising an anti-CD161 antibody to the subject if the therapy is selected.

In some embodiments, the presence of CD161 expressing cells in the population of cells (e.g., immune cells (e.g., T cells, NK cells)) indicates that the subject is likely to respond to treatment with an anti-CD161 antibody.

In some embodiments, the presence of a percentage of CD161 expressing cells in the population of cells (e.g., immune cells (e.g., T cells, NK cells)) indicates that the subject is likely to respond to treatment with an anti-CD161 antibody.

In some embodiments, the method comprises withholding administration of an anti-CD161 antibody to the subject if the presence of CD161 expressing cells in the population of immune cells (e.g., T cells, NK cells) in the sample is not detected. In some embodiments, the method comprises administering an alternative therapeutic agent for treatment of the disease that is not an anti-CD161 antibody to the subject if the presence of CD161 expressing cells in the population of cells (e.g., immune cells (e.g., T cells, NK cells)) in the sample is not detected. In some embodiments, the method comprises the different therapeutic agent is a standard of care agent for the disease. In some embodiments, the method comprises the subject is undergoing or has undergone treatment with a different therapeutic agent for the disease that is not an anti-CD161 antibody. In some embodiments, the method comprises the treatment with the different therapeutic agent is discontinued if the presence of CD161 expressing cells in the population of cells (e.g., immune cells (e.g., T cells, NK cells)) in the sample is detected and/or the anti-CD161 antibody.

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

In some embodiments, the disease is cancer. In some embodiments, the disease is a proinflammatory disease (e.g., an autoimmune disease). In some embodiments, the disease is an autoimmune disease.

Provided herein are methods of selecting a therapy for a subject cancer, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) selecting a therapy comprising an agent (e.g., anti-CD161 antibody (e.g., an anti-CD161 antibody described herein (e.g., set forth in Table 3))) that inhibits the interaction of CD161 to CLEC2D if expression of CD161 is detected; and (c) optionally administering the therapy comprising an agent (e.g., anti-CD161 antibody) that inhibits the interaction of CD161 to CLEC2D to the subject if the therapy is selected.

Provided herein are methods of selecting a therapy for a subject cancer, the method comprising (a) detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof); (b) determining a CD161 ICA score (e.g., as described herein, e.g., as described in § 5.12.4 herein); (c) selecting a therapy comprising an agent (e.g., anti-CD161 antibody (e.g., an anti-CD161 antibody described herein (e.g., set forth in Table 3))) that inhibits the interaction of CD161 to CLEC2D if a pre-determined an CD161 ICA score threshold is reached (e.g., a CD161 ICA score of greater than or equal to 1; a CD161 ICA score of greater than or equal to 1.5; a CD161 ICA score of 2); and (d) optionally administering the therapy comprising an agent (e.g., anti-CD161 antibody) that inhibits the interaction of CD161 to CLEC2D to the subject if the therapy is selected.

In some embodiments, the presence of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody (e.g., an anti-CD161 antibody described herein (e.g., set forth in Table 3))) that inhibits the interaction of CD161 to CLEC2D (e.g., an anti-CD161 antibody (e.g., set forth in Table 3)).

In some embodiments, the presence of a percentage of CD161 expressing cells in the population of cells (e.g., immune cells (e.g., T cells, NK cells (e.g., tumor infiltrating T cells, tumor infiltrating NK cells))) indicates that the subject is likely to respond to treatment with an agent (e.g., anti-CD161 antibody (e.g., set forth in Table 3)) that inhibits the interaction of CD161 to CLEC2D (e.g., anti-CD161 antibody (e.g., an anti-CD161 antibody described herein (e.g., set forth in Table 3))).

In some embodiments, the method comprises withholding administration of an agent (e.g., anti-CD161 antibody (e.g., set forth in Table 3)) that inhibits the interaction of CD161 to CLEC2D (e.g., anti-CD161 antibody (e.g., an anti-CD161 antibody described herein (e.g., set forth in Table 3))) to the subject if the presence of CD161 expressing cells in the population of immune cells (e.g., T cells, NK cells (e.g., tumor infiltrating T cells, NK cells)) in the sample is not detected. In some embodiments, the method comprises administering an alternative therapeutic agent for treatment of the cancer that is not an agent (e.g., anti-CD161 antibody (e.g., an anti-CD161 antibody described herein (e.g., set forth in Table 3))) that inhibits the interaction of CD161 to CLEC2D (e.g., anti-CD161 antibody (e.g., an anti-CD161 antibody described herein (e.g., setforth in Table 3))) to the subject if the presence of CD161 expressing cells in the population of cells (e.g., immune cells (e.g., T cells, NK cells (e.g., tumor infiltrating T cells, tumor infiltrating NK cells))) in the sample is not detected. In some embodiments, the method comprises the different therapeutic agent is a standard of care agent for the cancer. In some embodiments, the method comprises the subject is undergoing or has undergone treatment with a different therapeutic agent for the cancer that is not an agent (e.g., anti-CD161 antibody (e.g., set forth in Table 3)) that inhibits the interaction of CD161 to CLEC2D (e.g., an anti-CD161 antibody). In some embodiments, the method comprises the treatment with the different therapeutic agent is discontinued if the presence of CD161 expressing cells in the population of cells (e.g., immune cells (e.g., T cells, NK cells (e.g., tumor infiltrating T cells, tumor infiltrating NK cells))) in the sample is detected and/or the agent (e.g., anti-CD161 antibody (e.g., set forth in Table 3)) that inhibits the interaction of CD161 to CLEC2D (e.g., an anti-CD161 antibody (e.g., set forth in Table 3)).

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

### 5.12.12 Methods of Stratifying Patient Populations

The CD161 binding proteins described herein (and fusion proteins and conjugates thereof (e.g., described herein)) are useful in stratifying patients who are more likely to respond to a therapy comprising an anti-CD161 antibody. For example, patients (e.g., including clinical trial subjects) can be stratified through detecting (or having detected) the expression of CD161 by a population of cells (e.g., immune cells (e.g., T cells, NK cells)) in a sample (e.g., a tissue sample) from the subject, wherein the detecting comprises the use of a CD161 binding protein described herein (e.g., set forth in Table 2) (or a fusion protein or conjugate thereof). The patients can be grouped according to the detected CD161 expression, e.g., into positive, negative, or equivocal groups. Optionally other diagnostic measures can also be included in the assessment, e.g., scoring methods, histology, and/or cytology. The methods can further comprise, e.g., assessing (or having assessed) clinical pattern outcomes relative to the detected expression of CD161 to the sample (e.g., both pre-treatment, archival) and stratifying (or having stratified) patients among or between disease type (e.g., cancer type) or severity.

In some embodiments, the disease is cancer. In some embodiments, the disease is a proinflammatory disease (e.g., an autoimmune disease). In some embodiments, the disease is an autoimmune disease.

The CD161 binding proteins described herein (and fusion proteins and conjugates thereof (e.g., described herein)) are useful in stratifying patients who are more likely to respond to a therapy comprising an agent (e.g., anti-CD161 antibody (e.g., set forth in Table 3)) that inhibits the interaction of CD161 to CLEC2D (e.g., anti-CD161 antibody (e.g., an anti-CD161 antibody described herein (e.g., set forth in Table 3))). For example, patients (e.g., including clinical trial subjects) can be stratified through detecting (or having detected) the expression of CD161 by a population of cells (e.g., immune cells (e.g., T cells, NK cells (e.g., tumor infiltrating T cells, tumor infiltrating NK cells))) in a sample (e.g., a tissue sample, e.g., a tumor tissue sample) from the subject, wherein the detecting comprises the use of a CD161 binding protein described herein (e.g., set forth in Table 2) (or a fusion protein or conjugate thereof). The patients can be grouped according to the detected CD161 expression, e.g., into positive, negative, or equivocal groups. Optionally other diagnostic measures can also be included in the assessment, e.g., scoring methods, histology, and/or cytology. The methods can further comprise, e.g., assessing (or having assessed) clinical pattern outcomes relative to the detected expression of CD161 to the sample (e.g., both pre-treatment, archival) and stratifying (or having stratified) patients among or between disease type (e.g., cancer type) or severity.

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

### 5.12.13 Methods of Characterizing a Population of Cells in a Sample

Provided herein are methods of characterizing a population of (e.g., immune cells (e.g., T cells, NK cells)) in a sample (e.g., a tissue sample) from a subject having a disease, the method comprising (a) isolating (and optionally purifying) a population of cells (or having a population of cells isolated and optionally purified)) (e.g., immune cells (e.g., T cells, NK cells)) from a sample (e.g., a tissue sample) from the subject; (b) analyzing the population of cells (e.g., immune cells (e.g., T cells, NK cells)); and (c) determining the expression of CD161, wherein the determining comprises the use of a CD161 binding protein described herein (e.g., set forth in Table 2) (or a fusion protein or conjugate thereof).

Provided herein are methods of characterizing a population of (e.g., immune cells (e.g., T cells, NK cells)) in a sample (e.g., a tissue sample) from a subject having a disease, the method comprising (a) isolating (and optionally purifying) a population of cells (or having a population of cells isolated and optionally purified)) (e.g., immune cells (e.g., T cells, NK cells)) from a sample (e.g., a tissue sample) from the subject; (b) analyzing the population of cells (e.g., immune cells (e.g., T cells, NK cells)); (c) determining the expression of CD161, wherein the determining comprises the use of a CD161 binding protein described herein (e.g., set forth in Table 2) (or a fusion protein or conjugate thereof); and (d) determining a CD161 ICA score (e.g., as described herein, e.g., as described in § 5.12.4 herein).

Provided herein are methods of characterizing a population of (e.g., immune cells (e.g., T cells, NK cells)) in a sample (e.g., a tissue sample, e.g., a tumor tissue sample) from a subject having a disease, the method comprising (a) isolating (and optionally purifying) a population of cells (or having a population of cells isolated and optionally purified)) (e.g., immune cells (e.g., T cells, NK cells)) from a sample (e.g., a tissue sample, e.g., a tumor tissue sample) from the subject; (b) detecting the expression of CD161, wherein the detecting comprises the use of a CD161 binding protein described herein (e.g., set forth in Table 2) (or a fusion protein or conjugate thereof); and (c) characterizing the disease as CD161 associated based on detection of CD161 expression or CD161 non-associated based on no detection of CD161 expression.

Provided herein are methods of characterizing a population of (e.g., immune cells (e.g., T cells, NK cells)) in a sample (e.g., a tissue sample, e.g., a tumor tissue sample) from a subject having a disease, the method comprising (a) isolating (and optionally purifying) a population of cells (or having a population of cells isolated and optionally purified)) (e.g., immune cells (e.g., T cells, NK cells)) from a sample (e.g., a tissue sample, e.g., a tumor tissue sample) from the subject; (b) detecting the expression of CD161, wherein the detecting comprises the use of a CD161 binding protein described herein (e.g., set forth in Table 2) (or a fusion protein or conjugate thereof); (c) determining a CD161 ICA score (e.g., as described herein, e.g., as described in § 5.12.4 herein); and (d) characterizing the disease as CD161 associated based on CD161 ICA score.

Provided herein are in vitro methods for detecting a population of CD161-expressing cells (e.g., a population of cells (e.g., immune cells (e.g., T cells, NK cells)) expressing CD161 on the cell surface) in a subject, the method comprising (a) contacting a sample (e.g., a tissue sample, e.g., a tumor tissue sample) comprising a population of cells (e.g., immune cells (e.g., T cells, NK cells)) from the subject with a CD161 binding protein described herein (e.g., set forth in Table 2) (or a fusion protein or conjugate thereof); and (b) detecting the binding of the CD161 binding protein, the conjugate, or the fusion protein to a population of cells in the sample; wherein the binding of the CD161 binding protein, the conjugate, or the fusion protein to a population of cells within the sample indicates the presence of a population of CD161-expressing cells (e.g., a population of cells expressing CD161 on the cell surface).

Provided herein are in vitro methods for detecting a population of CD161-expressing cells (e.g., a population of cells (e.g., immune cells (e.g., T cells, NK cells)) expressing CD161 on the cell surface) in a subject, the method comprising (a) contacting a sample (e.g., a tissue sample, e.g., a tumor tissue sample) comprising a population of cells (e.g., immune cells (e.g., T cells, NK cells)) from the subject with a CD161 binding protein described herein (e.g., set forth in Table 2) (or a fusion protein or conjugate thereof); (b) detecting the binding of the CD161 binding protein, the conjugate, or the fusion protein to a population of cells in the sample; wherein the binding of the CD161 binding protein, the conjugate, or the fusion protein to a population of cells within the sample indicates the presence of a population of CD161-expressing cells (e.g., a population of cells expressing CD161 on the cell surface); and (c) determining a CD161 ICA score (e.g., as described herein, e.g., as described in § 5.12.4 herein).

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the detecting (or having detected) comprises the use of a CD161 binding protein described herein (e.g., set forth in Table 2) (or a fusion protein or conjugate thereof) in an IHC assay. In some embodiments, detecting comprises determining a CD161 ICA score (as described herein). In some embodiments, the CD161 ICA score is determined as described in § 5.12.4 herein.

In some embodiments, the CD161 binding protein, the conjugate, or the fusion protein comprises a detectable label (e.g., a fluorescent tag).

In some embodiments, the disease is cancer. In some embodiments, the disease is a proinflammatory disease (e.g., an autoimmune disease). In some embodiments, the disease is an autoimmune disease.

Provided herein are methods of characterizing a population of (e.g., immune cells (e.g., T cells, NK cells (e.g., tumor infiltrating T cells, tumor infiltrating NK cells))) in a sample (e.g., a tissue sample, e.g., a tumor tissue sample) from a subject having cancer, the method comprising (a) isolating (and optionally purifying) a population of cells (or having a population of cells isolated and optionally purified)) (e.g., immune cells (e.g., T cells, NK cells (e.g., tumor infiltrating T cells, tumor infiltrating NK cells))) from a sample (e.g., a tissue sample, e.g., a tumor tissue sample) from the subject; (b) analyzing the population of cells (e.g., immune cells (e.g., T cells, NK cells (e.g., tumor infiltrating T cells, tumor infiltrating NK cells))); and (c) determining the expression of CD161, wherein the determining comprises the use of a CD161 binding protein described herein (e.g., set forth in Table 2) (or a fusion protein or conjugate thereof).

Provided herein are methods of characterizing a population of (e.g., immune cells (e.g., T cells, NK cells (e.g., tumor infiltrating T cells, tumor infiltrating NK cells))) in a sample (e.g., a tissue sample, e.g., a tumor tissue sample) from a subject having cancer, the method comprising (a) isolating (and optionally purifying) a population of cells (or having a population of cells isolated and optionally purified)) (e.g., immune cells (e.g., T cells, NK cells (e.g., tumor infiltrating T cells, tumor infiltrating NK cells))) from a sample (e.g., a tissue sample, e.g., a tumor tissue sample) from the subject; (b) analyzing the population of cells (e.g., immune cells (e.g., T cells, NK cells (e.g., tumor infiltrating T cells, tumor infiltrating NK cells))); (c) determining the expression of CD161, wherein the determining comprises the use of a CD161 binding protein described herein (e.g., set forth in Table 2) (or a fusion protein or conjugate thereof); and (d) determining a CD161 ICA score (e.g., as described herein, e.g., as described in § 5.12.4 herein).

Provided herein are methods of characterizing a population of (e.g., immune cells (e.g., T cells, NK cells (e.g., tumor infiltrating T cells, tumor infiltrating NK cells))) in a sample (e.g., a tissue sample, e.g., a tumor tissue sample) from a subject having cancer, the method comprising (a) isolating (and optionally purifying) a population of cells (or having a population of cells isolated and optionally purified)) (e.g., immune cells (e.g., T cells, NK cells (e.g., tumor infiltrating T cells, tumor infiltrating NK cells))) from a sample (e.g., a tissue sample, e.g., a tumor tissue sample) from the subject; (b) detecting the expression of CD161, wherein the detecting comprises the use of a CD161 binding protein described herein (e.g., set forth in Table 2) (or a fusion protein or conjugate thereof); and (c) characterizing the cancer as CD161 associated based on detection of CD161 expression or CD161 non-associated based on no detection of CD161 expression.

Provided herein are methods of characterizing a population of (e.g., immune cells (e.g., T cells, NK cells (e.g., tumor infiltrating T cells, tumor infiltrating NK cells))) in a sample (e.g., a tissue sample, e.g., a tumor tissue sample) from a subject having cancer, the method comprising (a) isolating (and optionally purifying) a population of cells (or having a population of cells isolated and optionally purified)) (e.g., immune cells (e.g., T cells, NK cells (e.g., tumor infiltrating T cells, tumor infiltrating NK cells))) from a sample (e.g., a tissue sample, e.g., a tumor tissue sample) from the subject; (b) detecting the expression of CD161, wherein the detecting comprises the use of a CD161 binding protein described herein (e.g., set forth in Table 2) (or a fusion protein or conjugate thereof); (c) determining a CD161 ICA score (e.g., as described herein, e.g., as described in § 5.12.4 herein); (d) characterizing the cancer as CD161 associated based on the CD161 ICA score.

Provided herein are in vitro methods for detecting a population of CD161-expressing cells (e.g., a population of cells (e.g., immune cells (e.g., T cells, NK cells (e.g., tumor infiltrating T cells, tumor infiltrating NK cells))) expressing CD161 on the cell surface) in a subject, the method comprising (a) contacting a sample (e.g., a tissue sample, e.g., a tumor tissue sample) comprising a population of cells (e.g., immune cells (e.g., T cells, NK cells (e.g., tumor infiltrating T cells, tumor infiltrating NK cells))) from the subject with a CD161 binding protein described herein (e.g., set forth in Table 2) (or a fusion protein or conjugate thereof); and (b) detecting the binding of the CD161 binding protein, the conjugate, or the fusion protein to a population of cells in the sample; wherein the binding of the CD161 binding protein, the conjugate, or the fusion protein to a population of cells within the sample indicates the presence of a population of CD161-expressing cells (e.g., a population of cells expressing CD161 on the cell surface).

Provided herein are in vitro methods for detecting a population of CD161-expressing cells (e.g., a population of cells (e.g., immune cells (e.g., T cells, NK cells (e.g., tumor infiltrating T cells, tumor infiltrating NK cells))) expressing CD161 on the cell surface) in a subject, the method comprising (a) contacting a sample (e.g., a tissue sample, e.g., a tumor tissue sample) comprising a population of cells (e.g., immune cells (e.g., T cells, NK cells (e.g., tumor infiltrating T cells, tumor infiltrating NK cells))) from the subject with a CD161 binding protein described herein (e.g., set forth in Table 2) (or a fusion protein or conjugate thereof); and (b) detecting the binding of the CD161 binding protein, the conjugate, or the fusion protein to a population of cells in the sample; wherein the binding of the CD161 binding protein, the conjugate, or the fusion protein to a population of cells within the sample indicates the presence of a population of CD161-expressing cells (e.g., a population of cells expressing CD161 on the cell surface); and (c) determining a CD161 ICA score (e.g., as described herein, e.g., as described in § 5.12.4 herein).

In some embodiments, the method comprises obtaining (or having obtained) the sample from the subject. In some embodiments, the method comprises processing (or having processed) the sample. In some embodiments, the processing comprises any one or more of: fixing the sample (e.g., formalin fixation); and/or embedding the sample (e.g., paraffin embedding).

In some embodiments, the CD161 binding protein, the conjugate, or the fusion protein comprises a detectable label (e.g., a fluorescent tag).

### 5.13 Assays

The CD161 binding proteins described herein (e.g., set forth in Table 2) (or a fusion protein or conjugate thereof) can be utilized in various assays (e.g., diagnostic assays, screening assays, etc.). Exemplary assays include immunoassays, e.g., immunohistochemistry (IHC) assays, enzyme-linked immunosorbent assays (ELSAs), and western blots.

For example, the CD161 binding proteins described herein (e.g., set forth in Table 2) (or a fusion protein or conjugate thereof) can be utilized in immunohistochemistry (IHC) assays. IHC assays can be performed in a variety of formats, known in the art. For example, a typical IHC assay uses formalin-fixed, paraffin-embedded (FFPE) tissue sample of about 3-4 millimeters, mounted and dried on a microscope slide and comprises, e.g., (1) subjecting the tissue sample to deparaffinization and hydration, contacting the rehydrated tissue section with a CD161 binding protein (e.g., antibody) described herein (e.g., set forth in Table 2); and (2) detecting the CD161 binding protein (e.g., antibody) on the surface of one or more cells in the tissue sample. If the CD161 binding protein (e.g., antibody) itself is detectably labeled (e.g., as described herein), it can be detected directly. Alternatively, the CD161 binding protein (e.g., antibody) may be bound by a detectably labeled secondary antibody which is detected.

The CD161 binding proteins described herein (e.g., set forth in Table 2) (or a fusion protein or conjugate thereof) can be utilized in various imaging techniques. For example, SPECT imaging (single photon emission computed tomography) or PET imaging (positron emission tomography). Labels include e.g., iodine-123 (¹²³I) and technetium-99m (⁹⁹mTc), e.g., in conjunction with SPECT imaging or C, ¹³N, ¹⁵O or ¹⁸F, e.g., in conjunction with PET imaging or Indium-111 (See, e.g., Gordon et al., (2005) International Rev. Neurobiol. 67:385-440, the entire contents of which are incorporated herein by reference for all purposes).

### 5.14 Kits

In a one aspect, provided herein are kits (e.g., diagnostic kits, therapeutic kits) comprising any one or more of a CD161 binding protein described herein; a fusion protein described herein; a conjugate described herein; a nucleic acid molecule described herein; a vector described herein; a cell described herein; a carrier described herein; and/or a pharmaceutical composition described herein. In addition, the kit may comprise a liquid vehicle for solubilizing or diluting, and/or technical instructions. The technical instructions of the kit may contain information about administration and dosage and subject groups.

In some embodiments, the CD161 binding protein described herein, the fusion protein described herein, the conjugate described herein, the nucleic acid molecule described herein, the vector described herein, the cell described herein, the carrier described herein, or the pharmaceutical composition described herein is provided in a separate part of the kit, wherein the CD161 binding protein described herein, the fusion protein described herein, the conjugate described herein, the nucleic acid molecule described herein, the vector described herein, the cell described herein, the carrier described herein, or the pharmaceutical composition described hereinis optionally lyophilized, spray-dried, or spray-freeze dried. The kit may further contain as a part a vehicle (e.g., buffer solution) for solubilizing the dried or lyophilized a CD161 binding protein described herein; a fusion protein described herein; a conjugate described herein; a nucleic acid molecule described herein; a vector described herein; a cell described herein; a carrier described herein; and/or a pharmaceutical composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container. The kit may further contain technical instructions for mixing the adjuvant prior to administration or for co-administration.

In some embodiments, the kit comprises a CD161 binding protein described herein; a fusion protein described herein; or a conjugate described herein. In some embodiments, CD161 binding protein described herein, the fusion protein described herein, or the conjugate described herein comprises a detectable label (e.g., a tag (e.g., a fluorescent tag)) to aid in detection. In some embodiments, the kit comprises one or more reagent (e.g., a buffer) for a sample described herein. In some embodiments, the kit comprises a secondary antibody comprising a detectable label that specifically binds the CD161 binding protein described herein, the fusion protein described herein, or the conjugate described herein. In some embodiments, the kit is for use in a method of determining the presence of a population of CD161-expressing cells in a sample. In some embodiments, the kit comprises a packaged combination of reagents in predetermined amounts with instructions for performing the diagnostic or detection assay. In some embodiments, the kit comprises a reagent or set of reagents for detecting a complex of CD161 bound to a CD161 binding protein described herein; a fusion protein described herein; or a conjugate described herein.

Any of the kits described herein may be used in any of the methods described herein (see, e.g., § 5.12).

### 6. EXAMPLES

### Table of Contents

6.1 Example 1. Generation of Human CD161 Binding Proteins.
6.2 Example 2. Expression of hCD161 & hCLEC2D by Normal Human Tissues.
6.3 Example 3. Expression of hCD161 by Human Lymphoma Tissue Microarrays.
6.4 Example 4. Expression of hCD161 by Human Solid Tumor Tissue Microarrays.
6.5 Example 5. Correlation Between the Level of CD161 Expressing Cells in the TME and Responsiveness of the Tumor to Therapeutic Anti-CD161 Antibody Therapy.
6.6 Example 6. Expression of hCD161 by Human Tumor Tissues.
6.1 Example 1. Generation of Human CD161 Binding Proteins.

Murine antibodies that specifically bind human CD161 (hCD161) were generated utilizing hybridoma technology. Briefly, mice were immunized with three synthetic peptides comprising a portion of the extracellular domain of hCD161. Splenocytes were purified from the mice and fused with myeloma cells to obtain hybridoma monoclones. The amino acid sequence of the generated antibody (i.e., CD161 binding protein A (SEQ ID NOS: 177-178)) is set forth in Table 2 herein.

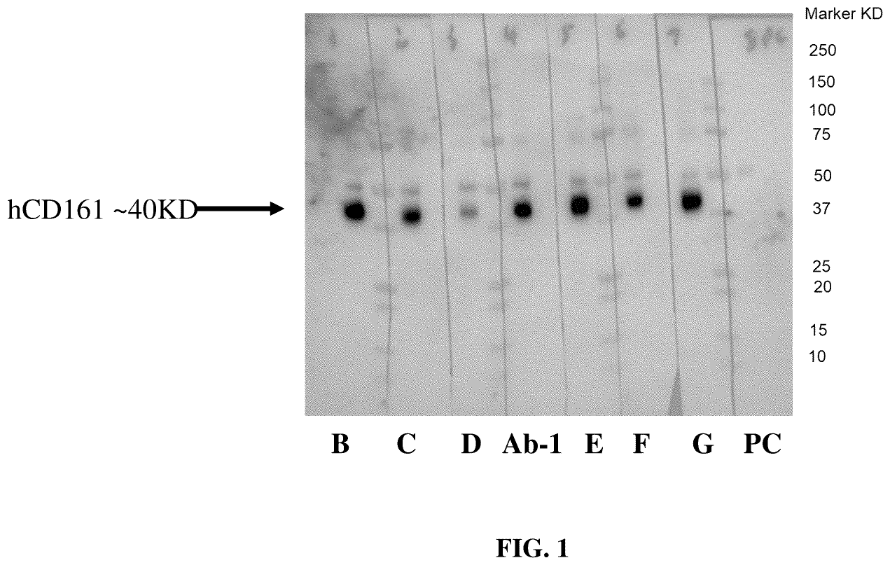

A western blot assay was employed to determine the ability of antibodies produced by each of the hybridoma clones to bind to hCD161. Briefly, cell lysate of cells expressing human CD161 was diluted in SDS-PAGE sample buffer with 50 mM DTT, and then boiled at 95° C. for 5 minutes. The cell lysate was loaded at 1μg/lane on 4-12% Bis-Tris western blot gel. MES SDS running buffer was used and running conditions according to manufacturer's instructions. A molecular weight marker was utilized (Precision Plus All blue Standards (Bio-Rad)). After SDS-PAGE separation, the gel was transferred to PVDF membrane using Bio-Rad Mini Trans-Blot Cell. The transferred PVDF membrane was blocked with 3% non-fat milk in PBS for 1 hr at room temperature, and then blotted with culture supernatants and an anti-hCD161 antibody (Ab-1 (see Table 2 herein), B, C, D, E, or F) diluted at 10μg/ml overnight at 4° C. An HRP-goat anti-mouse IgG antibody at 1:4000 dilution was used as a secondary antibody. The PVDF membrane was washed 3 times with 1×PBST, and then developed using ECL substrate. The image was captured using UVP and processed using LabWorks System. As shown in FIG. 1, antibodies produced by six of the hybridoma clones showed binding to hCD161.

### 6.2 Example 2. Expression of hCD161 & hCLEC2D by Normal Human Tissues

The expression of hCD161 and hCLEC2D on normal human tissues was assessed utilizing an immunohistochemistry (IHC) assay.

Briefly, CD161 binding protein Ab-1 (SEQ ID NOS: 177-178) (see Table 2 herein) and an anti-CLEC2D antibody were used to stain normal tissue microarrays (TMAs) from 35 normal human tissues. The experimental tissues examined included: adrenal gland, aorta, artery, brain/cerebrum, breast, bladder, cervix, cornea, esophagus, fallopian tube, intestine (large), intestine (small), heart, kidney, liver, lymph node, ling, ovary, pancreas, pituitary gland, placenta, prostate, salivary gland, saphenous vein, skeletal muscle, skin, spinal cord, spleen, stomach, testis, thyroid, tonsil, ureter, uterus/endometrium, and uterus/endomyometrium. Normal human tonsil tissue was used as positive control. Images of the TMAs stained for hematoxylin and eosin (H&E), CLEC2D, and CD161 were evaluated by pathologist for determination of cell type and level of staining for hCLEC2D and hCD161.

As stated above, IHC staining was carried out utilizing the CD161 binding protein Ab-1 (SEQ ID NOS: 177-178) (see Table 2 herein) and an anti-CLEC2D antibody. Briefly, formalin-fixed, paraffin-embedded (FFPE) patient samples were deparaffinized, and heat induced epitope retrieval was carried out using a citric buffer for 30 minutes. The samples were treated with a peroxide blocking solution (Leica Microsystems LOT 60485) for 5 minutes at room temperature and subsequently washed 3 times in a Bond wash solution (Leica Microsystems). The samples were treated with CD161 binding protein Ab-1 (SEQ ID NOS: 177-178) (see Table 2 herein) at a 1:400 dilution of 1 mg/ml CD161 binding protein Ab-1 stock for 30 minutes at room temperature and subsequently washed 3 times in a Bond Wash Solution (Leica Microsystems). The samples were treated with polymer (Leica Microsystems LOT 60485) for 8 minutes at room temperature and subsequently washed 5 times in a Bond wash solution (Leica Microsystems). The samples were rinsed with deionized water. The samples were mixed with DAB Refine (Leica Microsystems LOT 60485) and incubated for 10 minutes at room temperature. The samples were subsequently rinsed with deionized water 3 times. The samples were treated with DAB enhancer (Leica Bond DAB Enhancer) for 5 minutes and subsequently rinsed with deionized water 3 times. The samples were treated with Hematoxylin (Leica Microsystems LOT 60485) for 5 minutes and subsequently rinsed with deionized water. The samples were washed with Bond Wash Solution (Leica Microsystems) and subsequently rinsed with deionized water. IHC imaging was performed on a Leica BOND RX. Images were captured using Vectra® Polaris™.

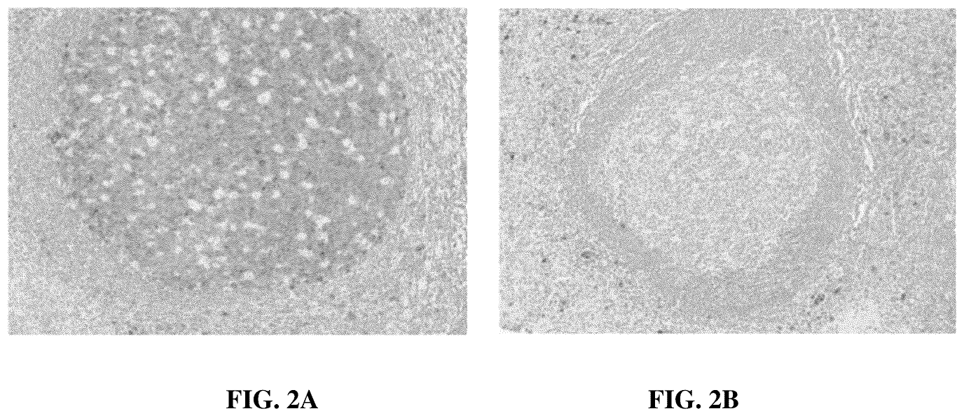

As shown in FIGS. 2A-2B, the tonsil tissue (positive control) shows positive staining of lymphocytes by the anti-CD161 antibody (Ab-1) (FIG. 2A) and anti-CLEC2D (FIG. 2B) antibody. The anti-CLEC2D antibody shows staining in the germinal center within the secondary lymphoid follicle (FIG. 2A) and the anti-hCD161 antibody (Ab-1) showed staining of T cells and NK cells in the T cell zone around the lymphoid follicle in the tonsil tissue (FIG. 2B). No definitive staining of the various normal tissues was observed for either hCLEC2D or hCD161 (data not shown).

### 6.3 Example 3. Expression of hCD161 by Human Lymphoma Tissue Microarrays

The expression of hCD161 in different hematological malignancies was assessed utilizing an IHC assay.

Briefly, CD161 binding protein Ab-1 (SEQ ID NOS: 177-178) (sese Table 2 herein) and an anti-CLEC2D antibody were used to stain two lymphoma tissue microarrays (TMAs). One multi-tumor TMA contained tumor cores from follicular lymphoma, Hodgkin's lymphoma, mantle cell lymphoma, marginal zone lymphoma, and chronic lymphocytic leukemia. The second TMA contained tumor cores from pre-treatment diffuse large B cell lymphoma (DLBCL) and post R-CHOP (rituximab, cyclophosphamide, doxorubicin, vincristine, andprednisolone) treated relapsed DLBCL. The pre- and post-treatment samples were not patient matched. Control tonsil tissue was used as positive control.

As stated above, IHC staining was carried out utilizing CD161 binding protein Ab-1 (SEQ ID NOS: 177-178) (see Table 2 herein) and an anti-CLEC2D antibody. IHC imaging was performed on a Leica BOND RX. Images were captured using Vectra® Polaris™. Staining was assessed using inForm® software, and CD161 staining as assessed in all cells by measuring mean fluorescence, 4-bin scoring of fluorescence levels and an H-score. IHC images are not shown.

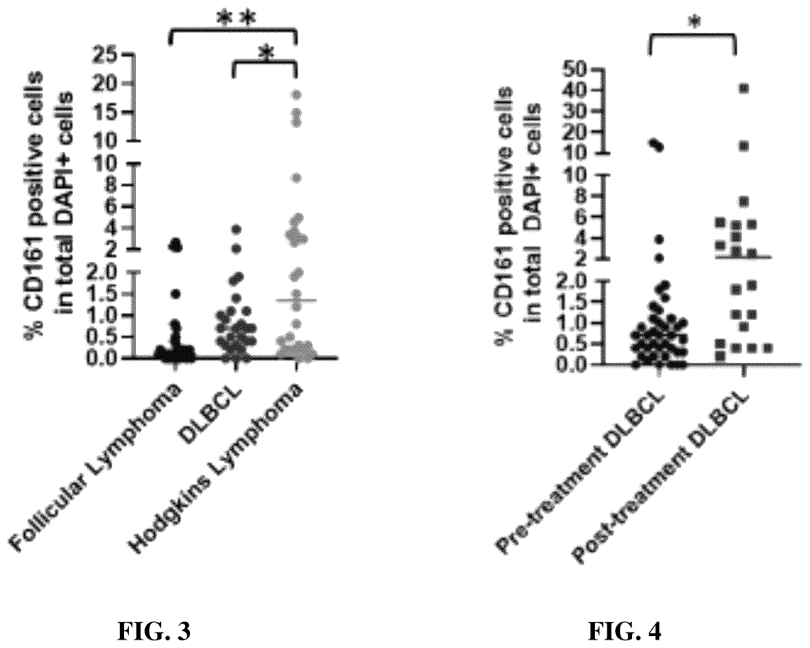

FIG. 3 shows the percent of CD161 expressing cells in total DAPI+ cells (which includes the malignant cell population) for follicular lymphoma, DLBCL, and Hodgkin's lymphoma (as determined by IHC). FIG. 4 shows the percent of CD161 expressing cells in total DAPI+ cells in pre- and post R-CHOP treatment relapsed DLBCL (as determined by IHC).

The data showed, inter alia, that lymphomas (e.g., follicular lymphoma, DLBCL, and Hodgkin's lymphoma) and R-CHOP treated relapsed DLBCL patient tumors show a significant percentage of CD161 expressing cells in the tumor microenvironment that were able to be detected using CD161 binding protein Ab-1 (SEQ ID NOS: 177-178) (see Table 2 herein).

### 6.4 Example 4. Expression of hCD161 by Human Solid Tumor Tissue Microarrays

The expression of hCD161 in different solid tumors was assessed utilizing an IHC assay.

Briefly, CD161 binding protein Ab-1 (SEQ ID NOS: 177-178) (see Table 2 herein) and an anti-CLEC2D antibody were used to stain five solid tumor tissue microarrays (TMAs). The tumor cores for each of the 5 solid tumor TMAs are set forth in Table 10.

TABLE 10
Composition of Solid Tumor TMAs.

| TMA | Tumor Type |
|---|---|
| TMA1 | Breast Cancer ER+ |
| | Breast Cancer Her2+ |
| | Triple Negative Breast Cancer (TNBC) |
| | Non-Small Cell Lung Cancer Squamous Cell Carcinoma |
| | (NSCLC-SCC) |
| | Non-Small Cell Lung Cancer Squamous Cell Adenocarcinoma |
| | (NSCLC-ADC) |
| | Small Cell Lung Cancer (SCLC) |
| TMA2 | Prostate Adenocarcinoma (ADC) |
| | Ovarian Cancer |
| | Endometrial Cancer |
| | Hormone refractory prostate cancer (HRPC) |
| | Renal cell carcinoma (RCC) |
| | Cervical Cancer |
| TMA3 | Sarcoma |
| | Gastrointestinal stromal tumor (GIST) |
| | Gall Bladder Cancer |
| | Head And Neck Squamous Cell Carcinoma (HNSCC) |
| | Melanoma |
| | Glioblastoma (GBM) |
| | Thyroid Cancer |
| TMA4 | Colorectal Cancer (CRC) |
| | Pancreatic Adenocarcinoma (ADC) |
| | Gastric Adenocarcinoma (ADC) |
| | Cholangiocarcinoma |
| | Esophageal/Gastro oesophageal junction (GOJ) Cancer |
| TMA5 | Hepatocellular carcinoma (HCC) |
| | Small Bowel Cancer |
| | Transitional Cell Carcinoma (TCC)/Upper Urinary Tract (UUT) |
| | Cutaneous squamous cell carcinoma (SCC) |
| | Mesothelioma |
| | Bladder Cancer |

One slide from each of the five solid tumor TMAs was stained for a panel of targets set forth in Table 11, with a DAPI nuclear counterstain. A second slide from each of the five solid tumor TMAs was stained as an autofluorescence control. Normal human tonsil tissue and non-small cell lung cancer tissue were used as positive controls.

TABLE 11
Fluorescence Panel for Solid Tumor Sample Analysis.

| Target Role | Target Marker | Cell Type |
|---|---|---|
| Phenotyping | CD3 | Pan T Cell |
| | CD8 | Cytotoxic T Cell |
| | CD56 | NK Cell |
| | CD68 | Monocyte/Macrophage |
| | PDL1 | Checkpoint |
| Expression | CLEC2D | |
| | CD161 | |
| Segmentation | CK | Tumor Cell |

For CD161 staining, the CD161 binding protein Ab-1 (SEQ ID NOS: 177-178) was diluted at ratio of 1:500 (1 mg/ml stock solution) in DAKO antibody diluent and incubated for 30 minutes. A secondary fluorescently labeled anti-mouse IgG antibody was utilized for detection. All staining was performed on the Leica BOND RX and images were captured using the Vectra® Polaris™ using the im3 workflow. IHC images are not shown. Results of the IHC analysis are summarized in Table 12.

TABLE 12
Summary of Solid Tumor IHC Analysis.

| | Multiplexed Immunofluorescence Analysis | | |
|---|---|---|---|
| | | % tumor | % tumor |
| | | cores with | cores with |
| | % tumor cores with | CD3+ | CD161+ |
| | CLEC2D+ cells/mm² | cells/mm² | cells/mm² |
| Tumor Type | above 100 | above 1000 | above 50 |
| Non-small cell | ~70% | ~60% | ~70% |
| lung cancer | (CD3+T cells, CD68+ | | |
| (NSCLC)- | macrophages, CK+ tumor) | | |
| squamous cell | | | |
| carcinoma (SCC) | | | |
| (NSCLC) | ~30% | ~60% | ~70% |
| adenocarcinoma | (primarily CD3+) | | |
| (ADC) | | | |
| Head & neck | ~50% | ~60% | ~70% |
| squamous cell | (primarily CD3+, CD68+ | | |
| carcinoma | macrophages) | | |
| (HNSCC) | | | |
| (HPV negative) | | | |
| Triple negative | ~80% | ~60% | ~70% |
| breast cancer | (CD3+ T cells, CD68+ | | |
| (TNBC) | macrophages; CK+ tumor) | | |
| Cutaneous | ~80% | ~60% | ~70% |
| Squamous cell | (CD3+T cells, CD68+ | | |
| Carcinoma | macrophages; CK+ tumor) | | |
| (CSCC) | | | |

As shown above in Table 12, analysis of the microscopy images showed, inter alia, NSCLC-SCC, NSCLC-ADC, HNSCC (HPV negative), TNBC, and CSCC all showed at least 30% tumor cores with CLEC2D+ cells/mm² above 100, about 60% tumor cores with CD3+ cells/mm² above 1000, and about 70% tumor cores with CD161+ cells/mm² above 50. The CD161 positive cells were able to be detected using CD161 binding protein Ab-1 (SEQ ID NOS: 177-178) (see Table 2 herein).

### 6.5 Example 5. Correlation Between the Level of CD161 Expressing Cells in the TME and Responsiveness of the Tumor to Therapeutic Anti-CD161 Antibody Therapy

The correlation between the level of CD161 expressing immune cells (including NK cells, CD4+ T cells, and CD8+ T cells) in the tumor microenvironment (TME) (determined by IHC) of a subject (prior to treatment of the subject with an anti-CD161 antibody therapy) and responsiveness of the cancer in the subject to the anti-CD161 antibody therapy was assessed.

Briefly, tumor biopsies were obtained from human subjects with a primary diagnosis of colon cancer and CD161 expression by immune cells (including NK cells, CD4+ T cells, and CD8+ T cells) was determined pre-treatment with anti-CD161 antibody (Ab-10, see Table 3 herein) by IHC using Ab-1 (see Table 2 herein).

Briefly, IHC staining for CD161 was performed on Leica BOND III automated IHC staining platform. Testing for CD161 used the primary antibody Ab-1 (see Table 2 herein) for detection in formalin-fixed, paraffin-embedded (FFPE) human tissues. Briefly, FFPE tissue blocks were cut at 4-5 μm thickness and sections were mounted onto Fisher Superfrost positively-charged glass slides. Slides were air-dried overnight and baked on the Leica BOND III IHC staining platform for 30 min at 60° C. prior to testing. Tissue sections were dewaxed using the Leica dewax protocol on the BOND III platform with Leica's BOND Dewax solution. Heat induced epitope retrieval (HIER) was performed on the BOND III platform after tissue sections were dewaxed wherein high pH (Tris-EDTA, pH 9) Leica BOND Epitope Retrieval 2 (ER2) solution was heat tested without subsequent digestion by Proteinase K (PTK) enzyme. The samples were blocked using Peroxidase Block (Leica Refine) for 5 minutes. The samples were treated with primary antibody Ab-1 (see Table 2 herein) at 2.0 g/mL for 15 minutes. The samples were subsequently treated with Post Primary anti-Mouse IgG Linker for 8 minutes, followed by Horseradish Peroxidase (HRP) Polymer for 20 minutes, DAB (3,3′-diaminobenzidine) Chromogen for 10 minutes, and Hematoxylin for 5 minutes. All steps in the sequence were performed on the Leica BOND III platform and includes intervening washes with BOND wash buffer. Slides were rinsed in distilled water and dehydrated in an alcohol series (95%, 100% ethanol) and in organic solvent (100% xylene, four changes). After dehydration, slides were permanently coverslipped using a non-aqueous mounting media and examined under a microscope to assess staining.

MultiOmyx technology was utilized to evaluate the expression of a 20-biomarker panel including CD3, CD4, CD8, CD56, and CD161. The staining was performed using a single 4 uM FFPE slide of patient tumor samples. Within each staining round, two cyanine dye-labeled (Cy3, Cy5) antibodies were paired together. The staining signal was then imaged and followed by novel dye inactivation, enabling repeated rounds of staining. Quantitative analyses of co-expressing cells was performed and reported in the graphs.

A CD161 score (as detected by IHC described above) was determined for each formalin-fixed, paraffin-embedded (FFPE) tissue sample. Briefly, CD161 was evaluated semi-quantitatively for reactivity in immune cells for each tissue tested. Only definite cytoplasmic staining in immune cells was scored. If observed, CD161 nuclear staining was not scored. For each tumor sample, numeric scoring excluded any signal in adjacent normal tissue and areas of ischemic changes, thermal artifact, and necrosis were not scored. When solid tumor samples were assessed, the total number of reactive immune cells at any intensity above background in the tumor-induced stroma (TIS) and the tumor cell field (TCF) were combined for each 20× field. TCF was characterized as within the tumor and included immune cells that were in between tumor cells (IBTC) and within the tumor mass or tumor nests. TIS was characterized as at the tumor/stroma interface and included immune cells within the tumor-induced stroma, which represented the parenchymal response to a tumor or the tumor microenvironment. A relative immune cell abundance score for CD161-reactive immune cells within TIS/TCF was estimated within the entire sample tissue. The abundance score was determined by evaluating regions of the tissue at 20× magnification and scoring on a relative abundance scale from 0-2 as described below. When possible, at least three fields were assessed per sample, therefore the abundance score was an average. Abundance of immune cells in TIS/TCF with CD161 staining was evaluated on a relative 0-2 scale as follows: 0=No positive immune cells observed/20× Field (<1 immune cell); 1=Low density of positive immune cells/20× Field (1-4 immune cells); 2=Moderate density of positive immune cells/20× Field (≥5 immune cells).

Post treatment with anti-CD161 antibody (Ab-10, see Table 3 herein), each subject was evaluated and determined to be a responder, partial responder, or non-responder to the anti-CD161 antibody therapy (Ab-10, see Table 3 herein). A non-responder was defined as a patient with a 20% increase in target lesions or new lesions had formed; a responder was defined as a patient with ≥30% decrease in target lesions; and a partial responder was defined as patients falling between the criteria of a non-responder and responder (above).

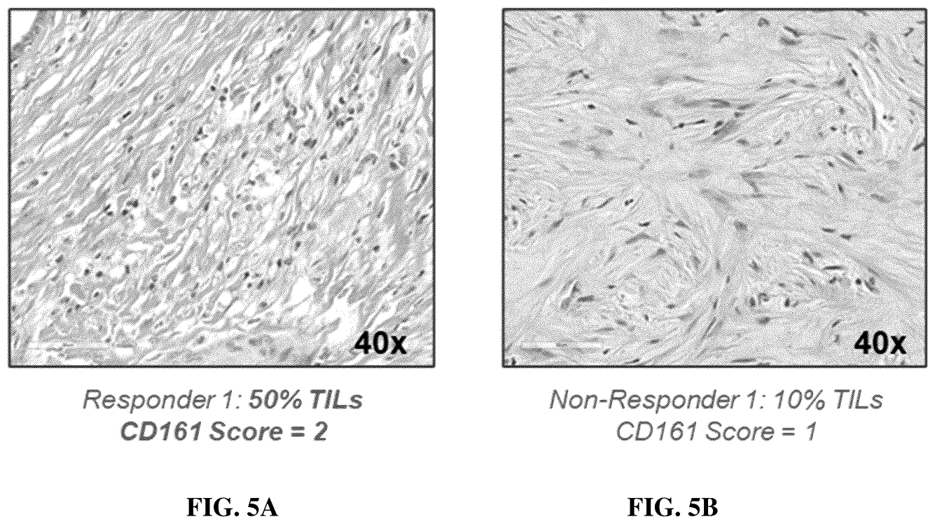

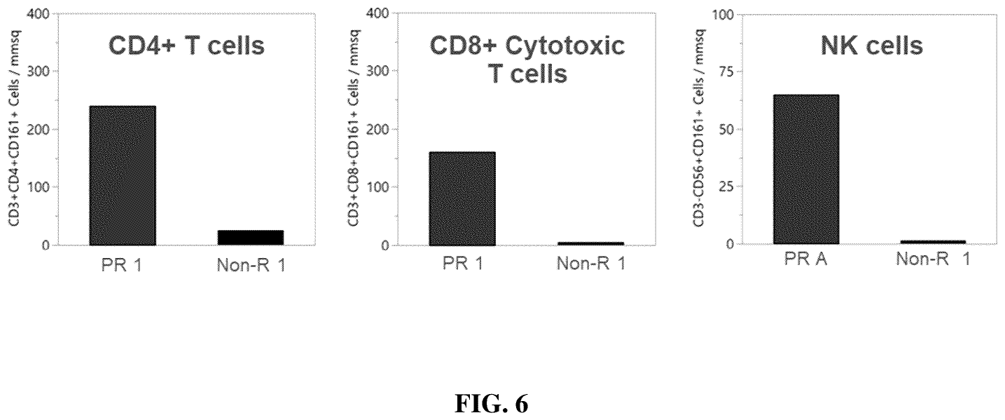

As shown in FIG. 5A-5B and FIG. 6, a confirmed responder to anti-CD161 antibody therapy (Ab-10, see Table 3 herein) had a higher pre-treatment CD161 score (determined using Ab-1) compared to the confirmed non-responder. More specifically, as shown in FIG. 6, the confirmed responder had more intratumoral CD161 expressing CD4+ T cells, CD161 expressing CD8+ T cells, and CD161 expressing NK cells (pre-treatment with the therapeutic anti-CD161 antibody).

### 6.6 Example 6. Expression of hCD161 by Human Tumor Tissues

The level of CD161 expressing mononuclear immune cells across multiple human tumor types was assessed by IHC and CD161 expression scoring.

Briefly, sensitivity testing for CD161 included a total of 356 formalin-fixed, paraffin-embedded (FFPE) evaluable human cancer tissues sourced from BioChain Institute Inc., Discovery's IHC tissue bank, and Discovery's biorepository. Sensitivity testing was carried out for the following human cancers (the number of evaluable cases in parentheses): head and neck (H&N) human pappiloma virus (HPV) positive (H&N HPV+) (27), H&N HPV− (29), non-small cell lung cancer (NSCLC) adenocarcinoma (adeno) (33), NSCLC squamous cell carcinoma (SCC) (33), colorectal cancer (CRC) high microsatellite instability (MSI-H) (29), CRC microsatellite instability (MSI-S) (29), esophageal cancer (33), cutaneous squamous cell carcinoma (SCC) (25), Her2-BRCA (28), triple negative breast cancer (TNBC) (30), diffuse large B-cell lymphoma (DLBCL) (31), and Hodgkin's lymphoma (29). IHC staining for CD161 was performed as described in Example 5. CD161 scoring was performed as described in Example 5.

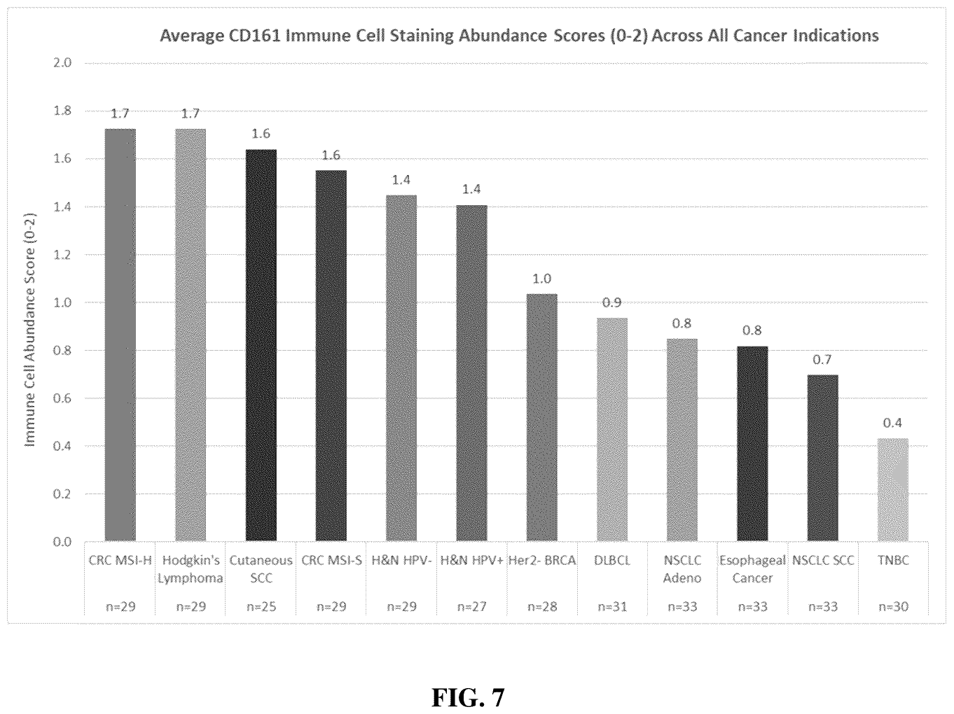

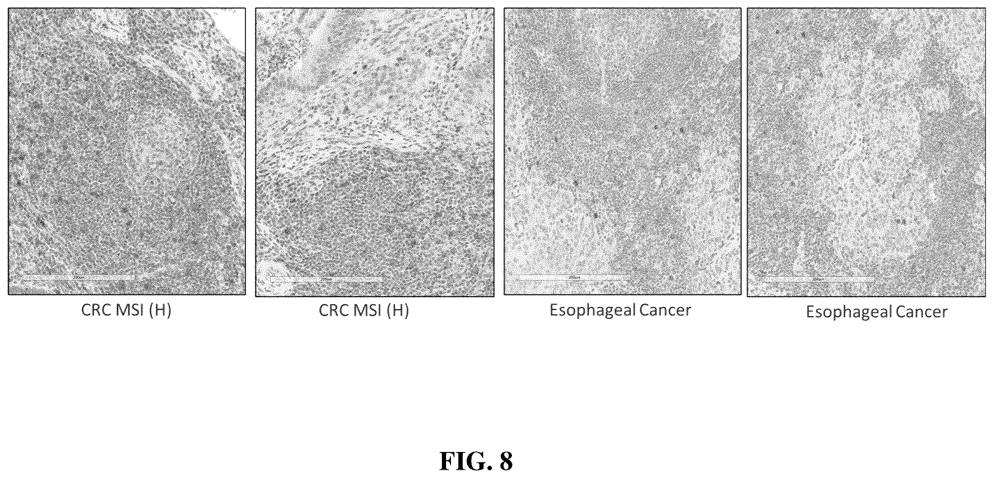

As shown in FIG. 7, each of the tumor types examined (both solid tumors and hematological malignancies) exhibited an average CD161 immune cell abundance score ranging from 0.4-1.7. Exemplary corresponding IHC images are presented in FIG. 8 showing CD161 staining in CRC MSI (H) and esophageal cancer samples.

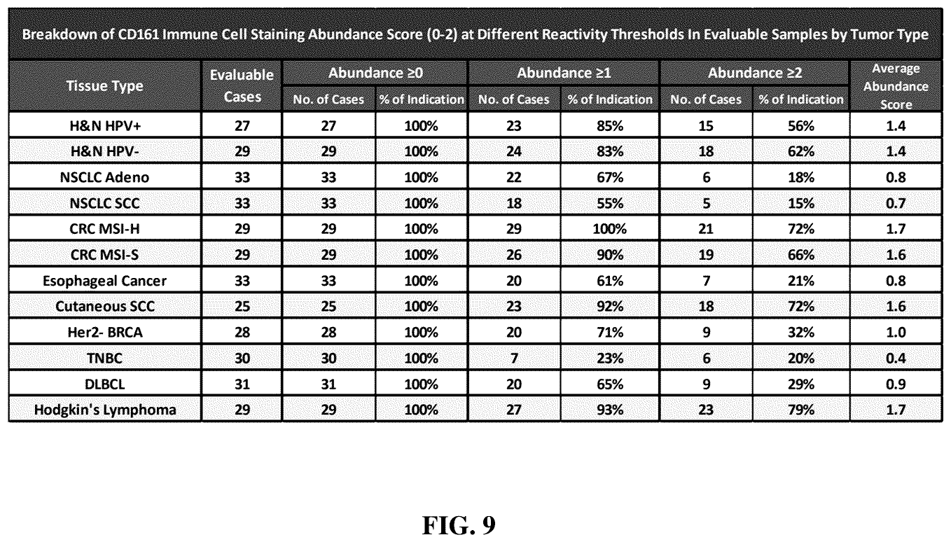

FIG. 9 further shows a summary table based on the CD161 immune cell staining abundance scores determined above, that divides the CD161 immune cell staining abundance scores according to different theoretical positivity thresholds. These breakdown tables can be utilized in selecting positivity cut-offs for clinical testing. FIG. 9 lists the number of cases and the percentage of the cancer type that would be positive at each theoretical cut-off for CD161 immune cell staining abundance scores at: ≥0, ≥1, and ≥2 in a range of tumor proportions.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. A CD161 binding protein comprising a binding domain that specifically binds CD161, wherein the binding domain comprises:
(a) a VH region comprising (i) a CDR-H1 comprising the amino acid sequence set forth in SEQ ID NO: 169, or the amino acid sequence set forth in SEQ ID NO: 169 comprising 1, 2, or 3 amino acid variations; (ii) a CDR-H2 comprising the amino acid sequence set forth in SEQ ID NO: 170, or the amino acid sequence set forth in SEQ ID NO: 170 comprising 1, 2, or 3 amino acid variations; and (iii) a CDR-H3 comprising the amino acid sequence set forth in SEQ ID NO: 171, or the amino acid sequence set forth in SEQ ID NO: 171 comprising 1, 2, or 3 amino acid variations; and
(b) a VL region comprising (i) a CDR-L1 comprising the amino acid sequence set forth in SEQ ID NO: 172, or the amino acid sequence set forth in SEQ ID NO: 172 comprising 1, 2, or 3 amino acid variations; (ii) a CDR-L2 comprising the amino acid sequence set forth in SEQ ID NO: 173, or the amino acid sequence set forth in SEQ ID NO: 173 comprising 1, 2, or 3 amino acid variations; and (iii) a CDR-L3 comprising the amino acid sequence set forth in SEQ ID NO: 174, or the amino acid sequence set forth in SEQ ID NO: 174 comprising 1, 2, or 3 amino acid variations.

2. The CD161 binding protein of claim 1, wherein the binding domain comprises:
(a) a VH region comprising (i) a CDR-H1 comprising the amino acid sequence set forth in SEQ ID NO: 169; (ii) a CDR-H2 comprising the amino acid sequence set forth in SEQ ID NO: 170; and (iii) a CDR-H3 comprising the amino acid sequence set forth in SEQ ID NO: 171; and
(b) a VL region comprising (i) a CDR-L1 comprising the amino acid sequence set forth in SEQ ID NO: 172; (ii) a CDR-L2 comprising the amino acid sequence set forth in SEQ ID NO: 173; and (iii) a CDR-L3 comprising the amino acid sequence set forth in SEQ ID NO: 174.

3.-41. (canceled)

42. A conjugate comprising the CD161 binding protein of claim 1 and a heterologous moiety.

43.-47. (canceled)

48. The conjugate of claim 42, wherein the heterologous moiety is a peptide, polypeptide, carbohydrate, lipid, polymer, or small molecule.

49. The conjugate of claim 42, wherein the heterologous moiety is a detectable tag.

50. A fusion protein comprising the CD161 binding protein of claim 1 and a heterologous protein.

51. The fusion protein of claim 50, wherein the heterologous protein comprises a cytokine (or functional fragment or functional variant thereof), a chemokine (or a functional fragment or functional variant thereof), or an antibody (or a functional fragment or functional variant thereof).

52.-56. (canceled)

57. A nucleic acid molecule encoding the CD161 binding protein of claim 1.

58.-60. (canceled)

61. A vector comprising the nucleic acid molecule of claim 57.

62.-63. (canceled)

64. A carrier comprising the CD161 binding protein of claim 1.

65. (canceled)

66. A cell (or population of cells) comprising the CD161 binding protein of claim 1.

67. A pharmaceutical composition comprising the CD161 binding protein of claim 1, and a pharmaceutically acceptable excipient.

68. A kit comprising the CD161 binding protein of any one of claim 1; and optionally instructions for using any one or more of the foregoing.

69. A kit comprising the CD161 binding protein of claim 1; and a labeled secondary antibody that specifically binds the CD161 binding protein.

70. A method of manufacturing a CD161 binding protein, the method comprising:
introducing into a cell the nucleic acid molecule of claim 57;
culturing the cell under conditions that allow for expression of the CD161 binding protein; and
optionally recovering the expressed the CD161 binding protein from the culture; and
optionally purifying the expressed the CD161 binding protein from the culture.

71. (canceled)

72. A method of delivering a CD161 binding protein to a subject, the method comprising administering to the subject the CD161 binding protein of claim 1, to thereby deliver the CD161 binding protein to the subject.

73. (canceled)

74. A method of inhibiting binding of CLEC2D to CD161 in a subject, the method comprising administering to the subject the CD161 binding protein of claim 1, to thereby inhibit binding of CLEC2D to CD161.

75. A method of treating, ameliorating, or preventing a disease in a subject in need thereof, the method comprising administering to the subject the CD161 binding protein of claim 1, to thereby treat, ameliorate, or prevent the disease in the subject.

76. (canceled)

77. A method of treating cancer in a subject in need thereof, the method comprising
(a) detecting (or having detected) the expression of CD161 by a population of cells in a sample from the subject, wherein the detecting comprises the use of the CD161 binding protein of claim 1; and
(b) administering an agent that inhibits the interaction of CD161 to CLEC2D to the subject if expression of CD161 is detected.

78. A method of treating cancer in a subject in need thereof, the method comprising
(a) detecting (or having detected) the expression of CD161 by a population of cells in a sample from the subject, wherein the detecting comprises the use of the CD161 binding protein of claim 1; and
(b)(i) administering an agent that inhibits the interaction of CD161 to CLEC2D to the subject if expression of CD161 is detected; or (b)(ii) withholding administration of an agent that inhibits the interaction of CD161 to CLEC2D to the subject if expression of CD161 is not detected and optionally administering a different therapeutic agent to treat the cancer that is not an agent that that inhibits the interaction of CD161 to CLEC2D.

79. A method of treating cancer in a subject in need thereof, the method comprising
(a) detecting (or having detected) the expression of CD161 by a population of cells in a sample from the subject, wherein the detecting comprises the use of the CD161 binding protein of claim 1; and
(b) administering an agent that inhibits the interaction of CD161 to CLEC2D to the subject based at least in part on the detection of CD161 expression in the sample from the subject.

80. A method of identifying a subject having cancer for treatment with an agent that inhibits the interaction of CD161 to CLEC2D, the method comprising
(a) detecting (or having detected) the expression of CD161 by a population of cells (e.g., immune cells in a sample from the subject, wherein the detecting comprises the use of the CD161 binding protein of claim 1;
(b) identifying the subject as a subject for treatment with an agent that inhibits the interaction of CD161 to CLEC2D if expression of CD161 is detected; and
(c) optionally administering an agent that inhibits the interaction of CD161 to CLEC2D to the subject if the subject is identified as a subject for treatment with an agent that inhibits the interaction of CD161 to CLEC2D.

81. A method of identifying a subject having cancer who is likely to respond to a treatment with an agent that inhibits the interaction of CD161 to CLEC2D, the method comprising
(a) detecting (or having detected) the expression of CD161 by a population of cells in a sample from the subject, wherein the detecting comprises the use of the CD161 binding protein of claim 1;
(b) identifying the subject as a subject likely to respond to treatment with an agent that inhibits the interaction of CD161 to CLEC2D if expression of CD161 is detected; and
(c) optionally administering an agent (e.g., anti-CD161 antibody (e.g., an anti-CD161 antibody described herein)) that inhibits the interaction of CD161 to CLEC2D to the subject if the subject is identified as a subject for treatment with an agent (e.g., anti-CD161 antibody) that inhibits the interaction of CD161 to CLEC2D.

82. A method of selecting a therapy for a subject cancer, the method comprising
(a) detecting (or having detected) the expression of CD161 by a population of cells in a sample from the subject, wherein the detecting comprises the use of the CD161 binding protein of claim 1;
(b) selecting a therapy comprising an agent that inhibits the interaction of CD161 to CLEC2D if expression of CD161 is detected; and
(c) optionally administering the therapy comprising an agent (e.g., anti-CD161 antibody) that inhibits the interaction of CD161 to CLEC2D to the subject if the therapy is selected.

83. A method of characterizing a population of cells in a sample from a subject having cancer, the method comprising
(a) isolating (and optionally purifying) a population of cells (or having a population of cells isolated and optionally purified)) from a sample (e.g., a tissue sample, e.g., a tumor tissue sample) from the subject;
(b) analyzing the population of cells; and
(c) determining the expression of CD161, wherein the determining comprises the use of the CD161 binding protein of claim 1.

84. A method of characterizing a population of cells in a sample from a subject having cancer, the method comprising
(a) isolating (and optionally purifying) a population of cells (or having a population of cells isolated and optionally purified) from a sample from the subject;
(b) detecting the expression of CD161, wherein the detecting comprises the use of the CD161 binding protein of claim 1; and
(c) characterizing the cancer as CD161 associated based on detection of CD161 expression or CD161 non-associated based on no detection of CD161 expression.

85. An in vitro method for detecting a population of CD161-expressing cells in a subject, the method comprising
(a) contacting a sample comprising a population of cells from the subject with the CD161 binding protein of claim 1; and
(b) detecting the binding of the CD161 binding protein, the conjugate, or the fusion protein to a population of cells in the sample;
wherein the binding of the CD161 binding protein, the conjugate, or the fusion protein to a population of cells within the sample indicates the presence of a population of CD161-expressing cells.

86.-117. (canceled)


SEQUENCE LISTING (178 sequences)

SEQ ID NO: 1 - protein, 800 aa, Homo sapiens
MERLWGLFQRAQQLSPRSSQTVYQRVEGPRKGHLEEEEEDGEEGAETLAHFCPMELRGPEPLGSRPRQPNLIPWAAAGRRAAPYLVLTALLIFTGAFLLGYVAFRGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 2 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 3 - protein, 5 aa, synthetic construct
TYAMS

SEQ ID NO: 4 - protein, 17 aa, synthetic construct
AISAAGGTTYYADSVKG

SEQ ID NO: 5 - protein, 12 aa, synthetic construct
PLDSSLWADFDL

SEQ ID NO: 6 - protein, 11 aa, synthetic construct
RASQGIDSWLA

SEQ ID NO: 7 - protein, 7 aa, synthetic construct
AASSLQS

SEQ ID NO: 8 - protein, 9 aa, synthetic construct
QQASVLPIT

SEQ ID NO: 9 - protein, 121 aa, synthetic construct
EVQLLESGGGLVQPGGSLRLSCAASGFAFSTYAMSWVRQAPGKGLEWVSAISAAGGTTYYADSVKGRFTISRDNSKNTLYLQMNSLRAEDTAVYYCAKPLDSSLWADFDLWGRGTLVTVSS

SEQ ID NO: 10 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQASVLPITFGGGTKVEIK

SEQ ID NO: 11 - protein, 17 aa, synthetic construct
AISGVGGTTYYADSVKG

SEQ ID NO: 12 - protein, 11 aa, synthetic construct
RASQGISSWLA

SEQ ID NO: 13 - protein, 7 aa, synthetic construct
YASSLQD

SEQ ID NO: 14 - protein, 121 aa, synthetic construct
EVQLLESGGGLVQPGGSLRLSCAASGFAFSTYAMSWVRQAPGKGLEWVSAISGVGGTTYYADSVKGRFTISRDNSKNTLYLQMNSLRAEDTAVYYCAKPLDSSLWADFDLWGRGTLVTVSS

SEQ ID NO: 15 - protein, 107 aa, synthetic construct
DIQLTQSPSSVSASVGDRVTITCRASQGISSWLAWYQQKPGKAPKLLIYYASSLQDGVPSRFSGSGSGTDFTLTISSLQPEDFATYYCQQASVLPITFGGGTKVEIK

SEQ ID NO: 16 - protein, 5 aa, synthetic construct
RYAMS

SEQ ID NO: 17 - protein, 12 aa, synthetic construct
PLDSSQWADFDL

SEQ ID NO: 18 - protein, 11 aa, synthetic construct
RASQDISSWLA

SEQ ID NO: 19 - protein, 7 aa, synthetic construct
AASALQS

SEQ ID NO: 20 - protein, 9 aa, synthetic construct
QQALVLPIT

SEQ ID NO: 21 - protein, 121 aa, synthetic construct
EVQLLESGGGLVQPGGSLRLSCAASGFTFERYAMSWVRQAPGKGLEWVSAISAAGGTTYYADSVKGRFTISRDNSKNTLYLQMNSLRAEDTAVYYCAKPLDSSQWADFDLWGRGTLVTVSS

SEQ ID NO: 22 - protein, 107 aa, synthetic construct
DIQLTQSPSSVSASVGDRVTITCRASQDISSWLAWYQQKPGKAPKFLIYAASALQSGVPSRFSGSGSGTDFTLTISSLQPEDFATYYCQQALVLPITFGGGTKVEIK

SEQ ID NO: 23 - protein, 17 aa, synthetic construct
AISAVGGTTKYADSVKG

SEQ ID NO: 24 - protein, 12 aa, synthetic construct
PLDSSLWADFDA

SEQ ID NO: 25 - protein, 7 aa, synthetic construct
AASGLQS

SEQ ID NO: 26 - protein, 9 aa, synthetic construct
QQASYLPIT

SEQ ID NO: 27 - protein, 121 aa, synthetic construct
EVQLLESGGGLVQPGGSLRLSCAASGFTFERYAMSWVRQAPGKGLEWVSAISAVGGTTKYADSVKGRFTISRDNSKNTLYLQMNSLRAEDTAVYYCAKPLDSSLWADFDAWGRGTLVTVSS

SEQ ID NO: 28 - protein, 107 aa, synthetic construct
DIQLTQSPSSVSASVGDRVTITCRASQGISSWLAWYQQKPGKAPKLLIYAASGLQSGVPSRFSGSGSGTDFTLTISSLQPEDFATYYCQQASYLPITFGGGTKVEIK

SEQ ID NO: 29 - protein, 5 aa, synthetic construct
QYAMS

SEQ ID NO: 30 - protein, 17 aa, synthetic construct
AISAVGGTTAYADSVKG

SEQ ID NO: 31 - protein, 12 aa, synthetic construct
PLDSSLWADFQL

SEQ ID NO: 32 - protein, 7 aa, synthetic construct
FASSLQS

SEQ ID NO: 33 - protein, 9 aa, synthetic construct
QQASKLPIT

SEQ ID NO: 34 - protein, 121 aa, synthetic construct
EVQLLESGGGLVQPGGSLRLSCAASGFTFGQYAMSWVRQAPGKGLEWVSAISAVGGTTAYADSVKGRFTISRDNSKNTLYLQMNSLRAEDTAVYYCAKPLDSSLWADFQLWGRGTLVTVSS

SEQ ID NO: 35 - protein, 107 aa, synthetic construct
DIQLTQSPSSVSASVGDRVTITCRASQDISSWLAWYQQKPGKAPKLLIYFASSLQSGVPSRFSGSGSGTDFTLTISSLQPEDFATYYCQQASKLPITFGGGTKVEIK

SEQ ID NO: 36 - protein, 9 aa, synthetic construct
QQAWVLPIT

SEQ ID NO: 37 - protein, 121 aa, synthetic construct
EVQLLESGGGLVQPGGSLRLSCAASGFTFGQYAMSWVRQAPGKGLEWVSAISAAGGTTYYADSVKGRFTISRDNSKNTLYLQMNSLRAEDTAVYYCAKPLDSSQWADFDLWGRGTLVTVSS

SEQ ID NO: 38 - protein, 107 aa, synthetic construct
DIQLTQSPSSVSASVGDRVTITCRASQGISSWLAWYQQKPGKAPKLLIYAASSLQSGVPSRFSGSGSGTDFTLTINSLQPEDFATYYCQQAWVLPITFGGGTKVEIK

SEQ ID NO: 39 - protein, 7 aa, synthetic construct
AASFLQS

SEQ ID NO: 40 - protein, 107 aa, synthetic construct
DIQLTQSPSSVSASVGDRVTITCRASQGISSWLAWYQQKPGKAPKLLIYAASFLQSGVPSRFSGSGSGTDFTLTISSLQPEDFATYYCQQASVLPITFGGGTKVEIK

SEQ ID NO: 41 - protein, 5 aa, synthetic construct
TFAMS

SEQ ID NO: 42 - protein, 12 aa, synthetic construct
PLDSSFWADFDL

SEQ ID NO: 43 - protein, 11 aa, synthetic construct
RASQTISSWLA

SEQ ID NO: 44 - protein, 9 aa, synthetic construct
QQQSVLPIT

SEQ ID NO: 45 - protein, 121 aa, synthetic construct
EVQLLESGGGLVQPGGSLRLSCAASGFTFGTFAMSWVRQAPGKGLEWVSAISGVGGTTYYADSVKGRFTISRDNSKNTLYLQMNSLRAEDTAVYYCAKPLDSSFWADFDLWGRGTLVTVSS

SEQ ID NO: 46 - protein, 107 aa, synthetic construct
DIQLTQSPSSVSASVGDRVTITCRASQTISSWLAWYQQKPGKAPKLLIYAASSLQSGVPSRFSGSGSGTDFTLTISSLQPEDFATYYCQQQSVLPITFGGGTKVEIK

SEQ ID NO: 47 - protein, 5 aa, synthetic construct
PYAMS

SEQ ID NO: 48 - protein, 17 aa, synthetic construct
AISASGGTTYYADSVKG

SEQ ID NO: 49 - protein, 9 aa, synthetic construct
QQHSVLPIT

SEQ ID NO: 50 - protein, 121 aa, synthetic construct
EVQLLESGGGLVQPGGSLRLSCAASGFTFSPYAMSWVRQAPGKGLEWVSAISASGGTTYYADSVKGRFTISRDNSKNTLYLQMNSLRAEDTAVYYCAKPLDSSFWADFDLWGRGTLVTVSS

SEQ ID NO: 51 - protein, 107 aa, synthetic construct
DIQLTQSPSSVSASVGDRVTITCRASQGISSWLAWYQQKPGKAPKLLIYAASSLQSGVPSRFSGSGSGTDFTLTISSLQPEDFATYYCQQHSVLPITFGGGTKVEIK

SEQ ID NO: 52 - protein, 17 aa, synthetic construct
AISAVGGSTYYADSVKG

SEQ ID NO: 53 - protein, 9 aa, synthetic construct
QQADVLPIT

SEQ ID NO: 54 - protein, 121 aa, synthetic construct
EVQLLESGGGLVQPGGSLRLSCAASGFTFSQYAMSWVRQAPGKGLEWVSAISAVGGSTYYADSVKGRFTISRDNSKNTLYLQMNSLRAEDTAVYYCAKPLDSSQWADFDLWGRGTLVTVSS

SEQ ID NO: 55 - protein, 107 aa, synthetic construct
DIQLTQSPSSVSASVGDRVTITCRASQDISSWLAWYQQKPGKAPKLLIYAASALQSGVPSRFSGSGSGTDFTLTISSLQPEDFATYYCQQADVLPITFGGGTKVEIK

SEQ ID NO: 56 - protein, 11 aa, synthetic construct
RASQGIYSWLA

SEQ ID NO: 57 - protein, 9 aa, synthetic construct
QQASDLPIT

SEQ ID NO: 58 - protein, 121 aa, synthetic construct
EVQLLESGGGLVQPGGSLRLSCAASGFTFSQYAMSWVRQAPGKGLEWVSAISAAGGTTYYADSVKGRFTISRDNSKNTLYLQMNSLRAEDTAVYYCAKPLDSSQWADFDLWGRGTLVTVSS

SEQ ID NO: 59 - protein, 107 aa, synthetic construct
DIQLTQSPSSVSASVGDRVTITCRASQGIYSWLAWYQQKPGKAPKLLIYAASSLQSGVPSRFSGSGSGTDFTLTISSLQPEDFATYYCQQASDLPITFGGGTKVEIK

SEQ ID NO: 60 - protein, 5 aa, synthetic construct
QYYMS

SEQ ID NO: 61 - protein, 17 aa, synthetic construct
YISPSGSTIAYADSVKG

SEQ ID NO: 62 - protein, 13 aa, synthetic construct
SLMATGTHLYFDL

SEQ ID NO: 63 - protein, 10 aa, synthetic construct
QQVTSFPPYT

SEQ ID NO: 64 - protein, 122 aa, synthetic construct
QVQLVESGGGLVKPGGSLRLSCAASGFTFAQYYMSWIRQAPGKGLEWVSYISPSGSTIAYADSVKGRFTISRDNAKNSLYLQMNSLRAEDTAVYYCARSLMATGTHLYFDLWGRGTLVTVSS

SEQ ID NO: 65 - protein, 108 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQVTSFPPYTFGGGTKVEIK

SEQ ID NO: 66 - protein, 5 aa, synthetic construct
NYYMS

SEQ ID NO: 67 - protein, 17 aa, synthetic construct
YISPSGATIAYADSVKG

SEQ ID NO: 68 - protein, 11 aa, synthetic construct
RASSGISSWLA

SEQ ID NO: 69 - protein, 7 aa, synthetic construct
AASELQS

SEQ ID NO: 70 - protein, 10 aa, synthetic construct
QQATSFPPYT

SEQ ID NO: 71 - protein, 122 aa, synthetic construct
QVQLVESGGGLVKPGGSLRLSCAASGFTFANYYMSWIRQAPGKGLEWVSYISPSGATIAYADSVKGRFTISRDNAKNSLYLQMNSLRAEDTAVYYCARSLMATGTHLYFDLWGRGTLVTVSS

SEQ ID NO: 72 - protein, 108 aa, synthetic construct
DIQLTQSPSSVSASVGDRVTITCRASSGISSWLAWYQQKPGKAPKLLIYAASELQSGVPSRFSGSGSGTDFTLTISSLQPEDFATYYCQQATSFPPYTFGGGTKVEIK

SEQ ID NO: 73 - protein, 13 aa, synthetic construct
SLMSTGTHLYFDL

SEQ ID NO: 74 - protein, 11 aa, synthetic construct
RASQGISDWLA

SEQ ID NO: 75 - protein, 122 aa, synthetic construct
QVQLVESGGGLVKPGGSLRLSCAASGFTFGQYYMSWIRQAPGKGLEWVSYISPSGATIAYADSVKGRFTISRDNAKNSLYLQMNSLRAEDTAVYYCARSLMSTGTHLYFDLWGRGTLVTVSS

SEQ ID NO: 76 - protein, 108 aa, synthetic construct
DIQLTQSPSSVSASVGDRVTITCRASQGISDWLAWYQQKPGKAPKLLIYAASSLQSGVPSRFSGSGSGTDFTLTISSLQPEDFATYYCQQVTSFPPYTFGGGTKVEIK

SEQ ID NO: 77 - protein, 17 aa, synthetic construct
YISPSGATIYYADSVKG

SEQ ID NO: 78 - protein, 10 aa, synthetic construct
QQVTSTPPYT

SEQ ID NO: 79 - protein, 122 aa, synthetic construct
QVQLVESGGGLVKPGGSLRLSCAASGFTFPQYYMSWIRQAPGKGLEWVSYISPSGATIYYADSVKGRFTISRDNAKNSLYLQMNSLRAEDTAVYYCARSLMSTGTHLYFDLWGRGTLVTVSS

SEQ ID NO: 80 - protein, 108 aa, synthetic construct
DIQLTQSPSSVSASVGDRVTITCRASQGISSWLAWYQQKPGKAPKLLIYAASSLQSGVPSRFSGSGSGTDFTLTISSLQPEDFATYYCQQVTSTPPYTFGGGTKVEIK

SEQ ID NO: 81 - protein, 5 aa, synthetic construct
DYYMS

SEQ ID NO: 82 - protein, 10 aa, synthetic construct
QQSTSFPPYT

SEQ ID NO: 83 - protein, 122 aa, synthetic construct
QVQLVESGGGLVKPGGSLRLSCAASGFTFSDYYMSWIRQAPGKGLEWVSYISPSGATIAYADSVKGRFTISRDNSKNTLYLQMNSLRAEDTAVYYCARSLMATGTHLYFDLWGRGTLVTVSS

SEQ ID NO: 84 - protein, 108 aa, synthetic construct
DIQLTQSPSSVSASVGDRVTITCRASQGISSWLAWYQQKPGKAPKLLIYAASGLQSGVPSRFSGSGSGTDFTLTISSLQPEDFATYYCQQSTSFPPYTFGGGTKVEIK

SEQ ID NO: 85 - protein, 10 aa, synthetic construct
QQVTSVPPYT

SEQ ID NO: 86 - protein, 122 aa, synthetic construct
QVQLVESGGGLVKPGGSLRLSCAASGFTFSDYYMSWIRQAPGKGLEWVSYISPSGATIYYADSVKGRFTISRDNAKNSLYLQMNSLRAEDTAVYYCARSLMSTGTHLYFDLWGRGTLVTVSS

SEQ ID NO: 87 - protein, 108 aa, synthetic construct
DIQLTQSPSSVSASVGDRVTITCRASQGISSWLAWYQQKPGKAPKLLIYAASSLQSGVPSRFSGSGSGTDFTLTISSLQPEDFATYYCQQVTSVPPYTFGGGTKVEIK

SEQ ID NO: 88 - protein, 7 aa, synthetic construct
AAESLQS

SEQ ID NO: 89 - protein, 10 aa, synthetic construct
QQVTSQPPYT

SEQ ID NO: 90 - protein, 122 aa, synthetic construct
QVQLVESGGGLVKPGGSLRLSCAASGFTFSQYYMSWIRQAPGKGLEWVSYISPSGATIAYADSVKGRFTISRDNAKNSLYLQMNSLRAEDTAVYYCARSLMATGTHLYFDLWGRGTLVTVSS

SEQ ID NO: 91 - protein, 108 aa, synthetic construct
DIQLTQSPSSVSASVGDRVTITCRASQGISSWLAWYQQKPGKAPKLLIYAAESLQSGVPSRFSGSGSGTDFTLTISSLQPEDFATYYCQQVTSQPPYTFGGGTKVEIK

SEQ ID NO: 92 - protein, 10 aa, synthetic construct
QQVTSAPPYT

SEQ ID NO: 93 - protein, 122 aa, synthetic construct
QVQLVESGGGLVKPGGSLRLSCAASGFTFSQYYMSWIRQAPGKGLEWVSYISPSGATIYYADSVKGRFTISRDNAKNSLYLQMNSLRAEDTAVYYCARSLMATGTHLYFDLWGRGTLVTVSS

SEQ ID NO: 94 - protein, 108 aa, synthetic construct
DIQLTQSPSSVSASVGDRVTITCRASQGISSWLAWYQQKPGKAPKLLIYAASALQSGVPSRFSGSGSGTDFTLTISSLQPEDFATYYCQQVTSAPPYTFGGGTKVEIK

SEQ ID NO: 95 - protein, 7 aa, synthetic construct
AASVLQS

SEQ ID NO: 96 - protein, 10 aa, synthetic construct
QQVTSFLPYT

SEQ ID NO: 97 - protein, 122 aa, synthetic construct
QVQLVESGGGLVQPGGSLRLSCAASGFTFSQYYMSWIRQAPGKGLEWVSYISPSGATIAYADSVKGRFTISRDNAKNSLYLQMNSLRAEDTAVYYCARSLMSTGTHLYFDLWGRGTLVTVSS

SEQ ID NO: 98 - protein, 108 aa, synthetic construct
DIQLTQSPSSVSASVGDRVTITCRASQGISSWLAWYQQKPGKAPKLLIYAASVLQSGVPSRFSGSGSGTDFTLTISSLQPEDFATYYCQQVTSFLPYTFGGGTKVEIK

SEQ ID NO: 99 - protein, 4 aa, synthetic construct
GGGS

SEQ ID NO: 100 - protein, 8 aa, synthetic construct
GGGSGGGS

SEQ ID NO: 101 - protein, 12 aa, synthetic construct
GGGSGGGSGGGS

SEQ ID NO: 102 - protein, 16 aa, synthetic construct
GGGSGGGSGGGSGGGS

SEQ ID NO: 103 - protein, 5 aa, synthetic construct
GGGGS

SEQ ID NO: 104 - protein, 10 aa, synthetic construct
GGGGSGGGGS

SEQ ID NO: 105 - protein, 15 aa, synthetic construct
GGGGSGGGGSGGGGS

SEQ ID NO: 106 - protein, 20 aa, synthetic construct
GGGGSGGGGSGGGGSGGGGS

SEQ ID NO: 107 - protein, 8 aa, synthetic construct
GGGGGGGS

SEQ ID NO: 108 - protein, 16 aa, synthetic construct
GGGGGGGSGGGGGGGS

SEQ ID NO: 109 - protein, 24 aa, synthetic construct
GGGGGGGSGGGGGGGSGGGGGGGS

SEQ ID NO: 110 - protein, 32 aa, synthetic construct
GGGGGGGSGGGGGGGSGGGGGGGSGGGGGGGS

SEQ ID NO: 111 - protein, 5 aa, synthetic construct
SGGGG

SEQ ID NO: 112 - protein, 10 aa, synthetic construct
SGGGGSGGGG

SEQ ID NO: 113 - protein, 15 aa, synthetic construct
SGGGGSGGGGSGGGG

SEQ ID NO: 114 - protein, 20 aa, synthetic construct
SGGGGSGGGGSGGGGSGGGG

SEQ ID NO: 115 - protein, 7 aa, synthetic construct
GGGGGGS

SEQ ID NO: 116 - protein, 14 aa, synthetic construct
GGGGGGSGGGGGGS

SEQ ID NO: 117 - protein, 21 aa, synthetic construct
GGGGGGSGGGGGGSGGGGGGS

SEQ ID NO: 118 - protein, 28 aa, synthetic construct
GGGGGGSGGGGGGSGGGGGGSGGGGGGS

SEQ ID NO: 119 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 120 - protein, 12 aa, synthetic construct
EPKSCDKTHTCP

SEQ ID NO: 121 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 122 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 106 aa, synthetic construct [sequence omitted]

SEQ ID NO: 124 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 330 aa, synthetic construct [sequence omitted]

SEQ ID NO: 131 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 133 - protein, 12 aa, synthetic construct
ESKYGPPCPSCP

SEQ ID NO: 134 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 135 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 106 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 137 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 216 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 139 - protein, 223 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 222 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 141 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 143 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 145 - protein, 107 aa, synthetic construct [sequence omitted]

SEQ ID NO: 146 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 147 - protein, 13 aa, synthetic construct
VPRDCGCKPCICT

SEQ ID NO: 148 - protein, 107 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTK

SEQ ID NO: 149 - protein, 107 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 150 - protein, 106 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 151 - protein, 214 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 152 - protein, 213 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 153 - protein, 227 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 154 - protein, 227 aa, synthetic construct; identical to SEQ ID NO: 153

SEQ ID NO: 155 - protein, 16 aa, synthetic construct
EPRGPTIKPCPPCKCP

SEQ ID NO: 156 - protein, 134 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVT

SEQ ID NO: 157 - protein, 83 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 158 - protein, 82 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 159 - protein, 217 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 160 - protein, 216 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 161 - protein, 233 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 162 - protein, 232 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 163 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 164 - protein, 20 aa, Homo sapiens
MYRMQLLSCIALSLALVTNS

SEQ ID NO: 165 - protein, 25 aa, Homo sapiens
MGVLLTQRTLLSLVLALLFPSMASM

SEQ ID NO: 166 - protein, 18 aa, Homo sapiens
MASLWLLSCFSLVGAAFG

SEQ ID NO: 167 - protein, 15 aa, Homo sapiens
MNLLLILTFVAAAVA

SEQ ID NO: 168 - protein, 24 aa, Homo sapiens
MALWMRLLPLLALLALWGPDPAAA

SEQ ID NO: 169 - protein, 5 aa, synthetic construct
SYALS

SEQ ID NO: 170 - protein, 17 aa, synthetic construct
YISSGGDYIYYADTVKG

SEQ ID NO: 171 - protein, 8 aa, synthetic construct
DEYYAVDY

SEQ ID NO: 172 - protein, 17 aa, synthetic construct
KSSQSLLYSSNQKNYLA

SEQ ID NO: 173 - protein, 7 aa, synthetic construct
WTSTRES

SEQ ID NO: 174 - protein, 9 aa, synthetic construct
QQYYSYPIT

SEQ ID NO: 175 - protein, 117 aa, synthetic construct
DVKLVESGEGLVKPGGSLKLSCAASGFTFGSYALSWVRQTPEKRLEWVAYISSGGDYIYYADTVKGRFTISRDNARNTLYLQMSSLKSEDTAMYYCTRDEYYAVDYWGQGTSVTVSS

SEQ ID NO: 176 - protein, 113 aa, synthetic construct
DIVMSQSPSSLAVSVGEKITMSCKSSQSLLYSSNQKNYLAWYQQKPGQSPKLLIYWTSTRESGVPDRFTGSGSGTDFTLTISSVKAEDLAVYYCQQYYSYPITFGSGTKLEIK

SEQ ID NO: 177 - protein, 441 aa, synthetic construct
DVKLVESGEGLVKPGGSLKLSCAASGFTFGSYALSWVRQTPEKRLEWVAYISSGGDYIYYADTVKGRFTISRDNARNTLYLQMSSLKSEDTAMYYCTRDEYYAVDYWGQGTSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 178 - protein, 220 aa, synthetic construct
DIVMSQSPSSLAVSVGEKITMSCKSSQSLLYSSNQKNYLAWYQQKPGQSPKLLIYWTSTRESGVPDRFTGSGSGTDFTLTISSVKAEDLAVYYCQQYYSYPITFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC